(19) United States
(12) Patent Application Publication
(10) Pub. No.: US 2023/0355622 A1
(43) Pub. Date: Nov. 9, 2023

(54) METHODS OF CHEMOVACCINATION AGAINST PLASMODIUM INFECTIONS

(71) Applicants: Justin A. BODDEY, Parkville, Victoria (AU); David B. OLSEN, West Point, PA (US); Ryan STEEL, Parkville, Victoria (AU); Merck Sharp & Dohme LLC, Rahway, NJ (US); The Walter and Eliza Hall Institute of Medical Research, Parkville, Bictoria (AU)

(72) Inventors: Justin A. Boddey, Victoria (AU); David B. Olsen, Lansdale, PA (US); Ryan Steel, Victoria (AU)

(73) Assignees: Merck Sharp & Dohme LLC, Rahway, NJ (US); The Walter and Eliza Hall Institute of Medical Research, Parkville, Victoria, AU (AU)

(21) Appl. No.: 18/245,210

(22) PCT Filed: Sep. 20, 2021
(86) PCT No.: PCT/US2021/051012

§ 371 Date: Mar. 14, 2023

Related U.S. Application Data
(60) Provisional application No. 63/082,049, filed on Sep. 23, 2020.

(51) Int. Cl. A61K 31/513; A61K 45/06; A61P 33/06

(52) U.S. Cl. CPC A61K 31/513; A61K 45/06; A61P 33/06

(57) ABSTRACT

A method of chemovaccination against Plasmodium infections comprising administering to a patient, an effective amount of a dual inhibitor of plasmepsin IX and X, or a pharmaceutically acceptable salt thereof.

### FIELD OF THE INVENTION

The present invention relates to methods of chemovaccination against Plasmodium infections. More specifically, the present invention relates to methods of chemovaccination against Plasmodium infections comprising administering to a patient, an effective amount of a dual inhibitor of plasmepsin IX and X, or a pharmaceutically acceptable salt thereof.

### BACKGROUND OF THE INVENTION

Malaria is a life-threatening disease that afflicts more than 200 million, and kills more than 400,000, people annually. Malaria-causing Plasmodium sporozoites are inoculated into the host by the bite of an infected mosquito. These sporozoites rapidly home to the liver and infect a hepatocyte, initiating an obligate but clinically silent phase of infection. In the hepatocyte the parasite rapidly transforms and grows into thousands of merozoites that later egress from the liver to infect red blood cells, causing malaria. Targeting liver parasites for elimination is an attractive antimalarial strategy since liver infection precedes malaria and therefore offers humans the opportunity to develop immunity to parasites before they take hold in the blood. The strategy is ideal, since a single parasite escaping elimination at the liver stage is sufficient to cause subsequent malaria disease.

Current malaria treatment relies primarily on drugs that target the disease-causing asexual blood stages (ABS) of Plasmodium parasites, the organisms responsible for human malaria. These drugs include the 4-aminoquinolines piperaquine and amodiaquine, the antifolates pymimethamine and sulfadoxine, and the endoperoxides artemisinin and its derivatives artesunate, artemether, and dihydroartemisinin. Artemisinin, usually in combination with partner drugs, have become a mainstay in the treatment and control of malaria. However, due to the increasing threat of artemisinin-based combination therapy (ACT) drug resistance, the development of new antimalarials with novel targets that inhibit multiple steps in the parasite life cycle is an urgent priority for the malaria control field.

Therefore, a much sought-after treatment for malaria is an antimalarial medicine which has a profile that includes chemovaccination. Chemovaccines typically work against the exoerythrocytic parasite forms that invade and develop in the liver and are responsible for the earliest asymptomatic stage of the infection. Such medicines could be formulated to provide long-acting prophylaxis, safeguarding individuals that are living near or traveling to areas that have parasites. Long-acting chemovaccination in endemic regions could also greatly reduce circulating parasite numbers and potentially replace a vaccine in an elimination campaign. Antonova-Koch, Y., et al., Open-source discovery of chemical leads for next-generation chemoprotective antimalarials.

Science, 2018, 362(6419): p. 9446.

### SUMMARY OF THE INVENTION

As shown in the Examples described below, dual inhibitors of the Plasmodium proteases plasmepsins IX and X (“PMIX/X” or, alternatively “PMIX/PMX”), cure Plasmodium liver infection with a unique mechanism of action. These compounds can serve as a causal prophylactic treatment by preventing the transition of parasite from liver to blood and therefore have the potential to prevent malaria disease, whereas many antimalarials just treat the disease-causing asexual blood stage parasites.

The present invention is directed to methods of chemovaccination against Plasmodium infections comprising administering to a patient, an effective amount of a dual inhibitor of plasmepsin IX and X, or a pharmaceutically acceptable salt thereof.

The present invention is also directed to the use of dual inhibitors of the Plasmodium proteases plasmepsins IX and X, or pharmaceutically acceptable salts thereof, to cure Plasmodium liver infection.

The present invention is also directed to methods of chemovaccination against Plasmodium infections comprising administering to a patient, an effective amount of a dual inhibitor of plasmepsin IX and X, or a pharmaceutically acceptable salt thereof, wherein the patient has a Plasmodium parasite infection.

The present invention is also directed to the use of a dual inhibitor of plasmepsin IX and X, or a pharmaceutically acceptable salt thereof, for chemovaccination against Plasmodium infections or malaria in a patient which has a Plasmodium parasite infection.

The present invention is also directed to methods of chemovaccination against Plasmodium infections comprising administering to a patient, an effective amount of a dual inhibitor of plasmepsin IX and X, or a pharmaceutically acceptable salt thereof, and an effective amount of one or more additional anti-malarial agents.

The present invention is also directed to the use of a dual inhibitor of plasmepsin IX and X, or a pharmaceutically acceptable salt thereof, for chemovaccination against Plasmodium infections or malaria and an effective amount of one or more additional anti-malarial agents, in a patient.

The present invention is also directed to methods of chemovaccination against Plasmodium infections comprising administering an effective amount of a dual inhibitor of plasmepsin IX and X, or a pharmaceutically acceptable salt thereof, and a wild-type Plasmodium parasite to a patient, wherein the patient does not have a Plasmodium parasite infection.

The present invention is also directed to methods of chemovaccination against Plasmodium infections comprising administering a long-acting injectable formulation comprising an effective amount of a dual inhibitor of plasmepsin IX and X, or a pharmaceutically acceptable salt thereof to a patient, wherein the patient does not have a Plasmodium parasite infection, and wherein the patient is later exposed to a wild-type Plasmodium parasite.

The present invention is also directed to methods of chemovaccination against Plasmodium infections comprising administering an effective amount of a dual inhibitor of plasmepsin IX and X, or a pharmaceutically acceptable salt thereof, and a genetically modified Plasmodium parasite to a patient, wherein the patient does not have a Plasmodium parasite infection.

The present invention is also directed to the use of a dual inhibitor of plasmepsin IX and X or a pharmaceutically acceptable salt thereof, and a wild-type Plasmodium parasite, for chemovaccination against Plasmodium infections in a patient, wherein the patient does not have a Plasmodium parasite infection.

The present invention is also directed to the use of a dual inhibitor of plasmepsin IX and X or a pharmaceutically acceptable salt thereof, for chemovaccination against Plasmodium infections or malaria in a patient, wherein the patient does not have a Plasmodium parasite infection, wherein the patient is later exposed to a Plasmodium parasite.

The present invention is also directed to the use of a dual inhibitor of plasmepsin IX and X or a pharmaceutically acceptable salt thereof, for chemovaccination against Plasmodium infections or malaria, and a genetically modified Plasmodium parasite, in a patient, wherein the patient does not have a Plasmodium parasite infection.

The present invention is also directed to the methods and uses described herein, wherein the dual inhibitor of plasmepsin IX and X is a compound of Formula (I′):

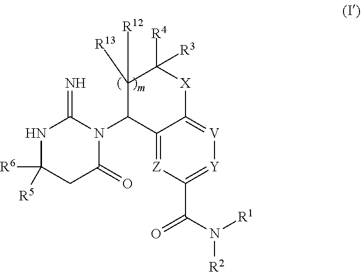

wherein X, V, Y, Z, R¹, R², R³, R⁴, R⁵, R⁶, R¹², R¹³ and m are described below.

Also described herein are compounds capable of inducing an immune response in a patient, wherein the compound is a compound of Formula (I′).

Also described herein are compounds capable of inducing an immune response in a patient, wherein the compounds are compounds of Formula (I′) and wherein compounds are capable of inducing an immune response to a Plasmodium parasite infection.

Also described herein are methods of inducing an immune response to a Plasmodium parasite infection, comprising administering to a patient, an effective amount of a compound, or a pharmaceutically acceptable salt thereof, wherein the compound is capable of inducing an immune response and the compound functions via a dual inhibitor mechanism of action of plasmepsin IX and X.

Also described herein are methods of inducing an immune response to a Plasmodium parasite infection, comprising administering to a patient an effective amount of a dual inhibitor of plasmepsin IX and X.

Also described herein are methods of inducing an immune response to a Plasmodium parasite infection, comprising administering to a patient an effective amount of a compound of Formula (I′), or a pharmaceutically acceptable salt thereof.

Also described herein are methods of inducing an immune response to a Plasmodium parasite infection, comprising administering to a patient an effective amount of a compound of Formula (I′), or a pharmaceutically acceptable salt thereof, wherein the patient has a Plasmodium parasite infection.

Also described herein are compositions capable of inducing an immune response comprising a dual inhibitor of plasmepsin IX and X, or a pharmaceutically acceptable salt thereof; and a pharmaceutically acceptable carrier or excipient.

Also described herein are compositions capable of inducing an immune response comprising a compound of Formula (I′), or a pharmaceutically acceptable salt thereof; and a pharmaceutically acceptable carrier or excipient.

Also described herein are compositions capable of inducing an immune response comprising a compound of Formula (I′), or a pharmaceutically acceptable salt thereof; and an adjuvant.

### BRIEF DESCRIPTION OF THE DRAWINGS

FIG. 1 shows female BALB/c mice infected with 40,000 P. berghei sporozoites expressing mCherry and Luciferase (PbmCherryLuci) and successfully treated with Example 1A.

FIG. 2A shows the immunity of the chemovaccinated mice of FIG. 1 rechallenged with 15 infected mosquito bites 6 months after being successfully treated with Example 1A.

FIG. 2B shows the immunity of the chemovaccinated mice of FIG. 1 rechallenged with 15 infected mosquito bites 12 months after being successfully treated with Example 1A.

FIG. 3 shows the mean from serum samples from immune and naïve mice, in which the five independent immune serum samples show a clear reduction of HepG2 cell infection by the PbmCherryLuci sporozoites compared to naïve serum in vitro.

FIG. 4 shows the results from the test conducted to determine the contribution of CD8 T-cells to the mechanism of immune protection.

FIG. 5A shows female BALB/c mice infected intravenously (i.v.) with increasing numbers of PbmCherryLuci sporozoites and cured with 2×100 mg/kg doses of Example 1A (bar graph).

FIG. 5B shows female BALB/c mice infected intravenously (i.v.) with increasing numbers of PbmCherryLuci sporozoites and cured with 2×100 mg/kg doses of Example 1A (line graph).

FIG. 6A shows the chemovaccinated mice of FIGS. 5A and 5B rechallenged after 3 months with 10 PbmCherryLuci infected mosquito bites (bar graph).

FIG. 6B shows the chemovaccinated mice of FIGS. 5A and 5B rechallenged after 3 months with 10 PbmCherryLuci infected mosquito bites (bar graph).

FIG. 7 shows the chemovaccinated mice of FIGS. 5A and 5B rechallenged after 12 months with 10 PbmCherryLuci infected mosquito bites.

FIG. 8 shows Example 1A and Example 75A inhibit transition from liver to blood infection in C57BL/6 mice.

FIG. 9 shows results of immunization of female C57BL/6 mice using a prime/boost regimen with Example 75A, challenged with the bites of 10 PbmCherryLuci infected mosquitoes.

FIG. 10 shows the results from human liver chimeric FRG mice (Yecuris, OR, USA) that were infected with P. falciparum salivary gland sporozoites. Three mice received no treatment, while one mouse received four oral treatments with 100 mg/kg of Example 1A during P. falciparum liver stage development.

### DETAILED DESCRIPTION OF THE INVENTION

The present invention is directed to methods of chemovaccination against Plasmodium infections comprising administering to a patient, an effective amount of a dual inhibitor of plasmepsin IX and X, or a pharmaceutically acceptable salt thereof. The present invention is also directed to methods of inducing an immune response to Plasmodium infections, comprising administering to a patient an effective amount of a dual inhibitor of plasmepsin IX and X, or a pharmaceutically acceptable salt thereof.

As shown in the experiments described herein, using dual inhibitors of the Plasmodium proteases plasmepsins IX and X (PMIX/X), Plasmodium liver infection has been cured with a unique mechanism of action. As shown below, mice were infected with Plasmodium berghei sporozoites and treated orally with PMIX/PMX dual inhibitors mid-way through liver infection. The mice were monitored in real time to see if the inhibitors cleared parasites from the liver or prevented their egress from the liver. The mice were also monitored to see if they developed a malaria-causing blood infection for 30 days post infection. If no infection was seen after 30 days the mice were considered to have been fully protected from malaria.

From these experiments, PMIX/X dual inhibitor compounds and doses capable of protecting mice from the malaria-causing blood infection when treated during liver infection were identified. It has been hypothesized that the mechanism by which these compounds cure the malaria infected mice is not through killing liver stage parasites or completely blocking their egress from the liver; rather, the mechanism of action is very late acting in that liver-derived (exoerythrocytic) merozoites that left the liver were non-infectious to erythrocytes. In this way the compounds serve as a causal prophylactic treatment by preventing liver to blood transition and malaria disease, as opposed to many antimalarials that treat the disease-causing asexual blood stage parasites. There are no known antimalarials either approved or in development that act with this mechanism at this late stage on liver parasites to completely prevent malaria disease.

Therefore, based on the data above and further described in the sections below, the present invention is directed to methods of chemovaccination against Plasmodium infections comprising administering to a patient, an effective amount of a dual inhibitor of plasmepsin IX and X, or a pharmaceutically acceptable salt thereof. Chemovaccination can take place in two forms, in one embodiment the dual inhibitors of plasmepsin IX and plasmepsin X, or a pharmaceutically acceptable salt thereof, is administered to a patient with an existing Plasmodium infection. In this embodiment, the infection is cured, and the patient is vaccinated against future infections. In a second embodiment, a patient who does not have an existing Plasmodium infection is administered an effective amount of a dual inhibitor of plasmepsin IX and X, or a pharmaceutically acceptable salt thereof and is then simultaneously or sequentially administered or exposed to a Plasmodium parasite.

Additionally, based on the data above and further described in the sections below, the present invention is directed to methods of inducing an immune response to Plasmodium infections, comprising administering to a patient an effective amount of a compound capable of inducing an immune response to Plasmodium infections. Additionally, based on the data above and further described in the sections below, the present invention is directed to methods of inducing an immune response to Plasmodium infections, comprising administering to a patient an effective amount of a dual inhibitor of plasmepsin IX and X, or a pharmaceutically acceptable salt thereof. Induction of an immune response can take place in two forms, in one embodiment the dual inhibitors of plasmepsin IX and plasmepsin X, or a pharmaceutically acceptable salt thereof, is administered to a patient with an existing Plasmodium infection. In this embodiment, the infection is cured, and the patient is vaccinated against future infections. In a second embodiment, a patient who does not have an existing Plasmodium infection is administered an effective amount of a dual inhibitor of plasmepsin IX and X, or a pharmaceutically acceptable salt thereof and is then simultaneously or sequentially administered or exposed to a Plasmodium parasite.

In the methods described herein, the Plasmodium infection can be caused by Plasmodium falciparum, Plasmodium vivax, Plasmodium ovale, Plasmodium malariae or Plasmodium knowlesi.

The present invention is also directed to the use of dual inhibitors of the Plasmodium proteases plasmepsins IX and X, or a pharmaceutically acceptable salt thereof, to cure Plasmodium liver infection.

The present invention is also directed to methods of chemovaccination against Plasmodium infections comprising administering to a patient, having a Plasmodium infection, an effective amount of a dual inhibitor of plasmepsin IX and X, or a pharmaceutically acceptable salt thereof.

The present invention is also directed to the use of a dual inhibitor of plasmepsin IX and X or a pharmaceutically acceptable salt thereof, for chemovaccination against Plasmodium infections or malaria, in a patient, wherein the patient has a Plasmodium infection.

The present invention is also directed to methods of chemovaccination against Plasmodium infections comprising administering an effective amount of a dual inhibitor of plasmepsin IX and X, or a pharmaceutically acceptable salt thereof, and a wild-type Plasmodium parasite, to a patient, wherein the patient does not have a Plasmodium parasite infection.

The present invention is also directed to methods of chemovaccination against Plasmodium infections comprising administering an effective amount of a dual inhibitor of plasmepsin IX and X, or a pharmaceutically acceptable salt thereof, and a genetically modified Plasmodium parasite, to a patient, wherein the patient does not have a Plasmodium parasite infection.

The present invention is also directed to the use of a dual inhibitor of plasmepsin IX and X, or a pharmaceutically acceptable salt thereof for chemovaccination against Plasmodium infections or malaria, and a wild-type Plasmodium parasite, in a patient, wherein the patient does not have a Plasmodium infection.

The present invention is also directed to the use of a dual inhibitor of plasmepsin IX and X or a pharmaceutically acceptable salt thereof for concurrent or sequential administration with a wild-type Plasmodium parasite, for chemovaccination against Plasmodium infections or malaria in a patient, wherein the patient does not have a Plasmodium infection.

The present invention is also directed to the use of a dual inhibitor of plasmepsin IX and X or a pharmaceutically acceptable salt thereof, for chemovaccination against Plasmodium infections or malaria, and a genetically modified parasite, in a patient, wherein the patient does not have a Plasmodium infection.

The present invention is also directed to methods of chemovaccination against Plasmodium infections comprising administering to a patient, an effective amount of a dual inhibitor of plasmepsin IX and X, or a pharmaceutically acceptable salt thereof, wherein the patient does not have a Plasmodium parasite infection and wherein the patient is later exposed to a Plasmodium parasite.

The present invention is also directed to the use of a dual inhibitor of plasmepsin IX and X for chemovaccination against Plasmodium infections or malaria, or a pharmaceutically acceptable salt thereof, in a patient, wherein the patient does not have a Plasmodium infection and wherein the patient is exposed to a wild-type Plasmodium parasite.

In certain embodiments, the exposure to a wild-type Plasmodium parasite is through a mosquito bite.

### Compounds for Chemovaccination

In certain embodiments described herein, the present invention is directed to methods of chemovaccination against Plasmodium infections comprising administering to a patient, an effective amount of a dual inhibitor of plasmepsin IX and X of Formula (I′):

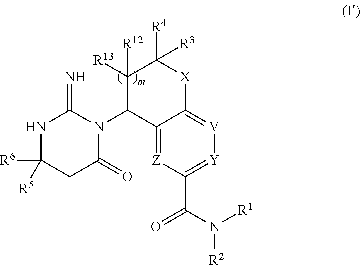

or a pharmaceutically acceptable salt thereof, wherein:
X is a bond, C(R′)₂, O, S, SO, SO₂ or NH;
Y is CR⁹ or N, wherein when Y is N, Z is CR¹¹ and V is CR¹⁰;
V is CR¹⁰ or N, wherein when V is N, Z is CR¹¹ and Y is CR⁹;
Z is CR¹¹ or N, wherein when Z is N, V is CR¹⁰ and Y is CR⁹;
R¹ is a heterocycloalkyl, C₃-C₁₂cycloalkyl, aryl, C₁-C₆alkylaryl or when taken with R², and the nitrogen which they are bonded, forms nitrogen-containing ring, wherein the heterocycloalkyl, C₃-C₂cycloalkyl, aryl, C₁-C₆alkylaryl or nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, aryl, C₁-C₆alkyl, C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R² is hydrogen, C₁-C₆alkylCOOH, COOH, C₃-C₆cycloalkyl, C₁-C₆alkyl, haloC₁-C₆alkyl or C₁-C₆alkylOH or when taken with R¹, and the nitrogen which they are bonded, forms a nitrogen-containing ring, wherein the nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl;
R⁴ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl;
R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁶ forms a C₃-C₆cycloalkyl or C₃-C₆heterocycloalkyl;
R⁷ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁵ forms a C₃-C₆cycloalkyl or C₃-C₆heterocycloalkyl;
R⁷ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁸ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹⁰ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹¹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹² is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸); R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
each occurrence of R⁴ is independently selected from the group consisting of hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
n is 1, 2, 3 or 4; and
m is 0, 1 or 2.

In other embodiments described herein, the present invention is directed to methods of chemovaccination against Plasmodium infections comprising administering to a patient, an effective amount of a dual inhibitor of plasmepsin IX and X of Formula (I):

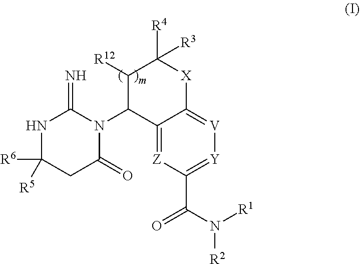

or a pharmaceutically acceptable salt thereof, wherein:
X is CH₂, O, S, SO, SO₂ or NH;
Y is CR⁹ or N, wherein when Y is N, Z is CR¹¹ and V is CR¹⁰;
V is CR¹⁰ or N, wherein when V is N, Z is CR¹¹ and Y is CR⁹;
Z is CR¹¹ or N, wherein when Z is N, V is CR¹⁰ and Y is CR⁹;
R¹ is a heterocycloalkyl, C₃-C₁₂cycloalkyl, aryl, C₁-C₆alkylaryl or when taken with R², and the nitrogen which they are bonded, forms nitrogen-containing ring, wherein the heterocycloalkyl, C₃-C₁₂cycloalkyl, aryl, C₁-C₆alkylaryl or nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R² is hydrogen, C₁-C₆alkylCOOH, COOH, C₃-C₆cycloalkyl, C₁-C₆alkyl, haloC₁-C₆alkyl or C₁-C₆alkylOH or when taken with R¹, and the nitrogen which they are bonded, forms a nitrogen-containing ring, wherein the nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁴ forms a C₃-C₆cycloalkyl or C₃-C₆heterocycloalkyl;
R⁴ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R³ forms a C₃-C₆cycloalkyl or C₃-C₆heterocycloalkyl;
R⁵ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁶ forms a C₃-C₆cycloalkyl or C₃-C₆heterocycloalkyl;
R⁶ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁵ forms a C₃-C₆cycloalkyl or C₃-C₆heterocycloalkyl;
R⁷ is hydrogen, C₁-C₆alkylCOOH, COOH, C₃-C₆cycloalkyl, C₁-C₆alkyl, haloC₁-C₆alkyl or C₁-C₆alkylOH;
R⁸ is hydrogen, C₁-C₆alkylCOOH, COOH, C₃-C₆cycloalkyl, C₁-C₆alkyl, haloC₁-C₆alkyl or C₁-C₆alkylOH;
R⁹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(RX)(R′) or C₁-C₆alkylN(R⁷)(R⁸);
R¹⁰ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹¹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹² is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸); and
m is 0, 1 or 2.

With regard to the compounds described herein, X is a bond, C(R¹⁴)₂, O, S, SO, SO₂ or NH. In certain embodiments described herein X is a bond. In certain embodiments, X is C(R¹⁴)₂, wherein R⁴ is discussed in further detail below. In certain embodiments, X is a bond, CH₂, CH(CH₃), C(CH₃)₂, O, CH(OCH₃), SO₂ or CF₂. In other embodiments X is CH₂, O, S, SO, SO₂ or NH. In certain embodiments, X is CH₂. In the embodiments described herein, X is O. In the embodiments described herein, X is S. In the embodiments described herein, X is SO. In the embodiments described herein, X is SO₂. In the embodiments described herein, X is NH. In the embodiments described herein, X is O or SO₂.

With regard to the compounds described herein, Y is CR⁹ or N. In certain embodiments, Y is CR⁹, wherein R⁹ is discussed in detail below. In certain embodiments, Y is N. In certain embodiments, Y is CH. In certain embodiments, wherein when Y is N, Z is CR¹¹ and V is CR¹⁰.

With regard to the compounds described herein, V is CR¹⁰ or N. In certain embodiments, V is CR¹⁰, R¹⁰ are discussed in detail below. In certain embodiments, V is N. In certain embodiments, V is CH. In certain embodiments, wherein when V is N, Z is CR¹¹ and Y is CR⁹.

With regard to the compounds described herein, Z is CR¹¹ or N. In certain embodiments, Z is CR¹¹, R¹¹ are discussed in detail below. In certain embodiments, Z is CH. In certain embodiments, Z is N. In certain embodiments, wherein when Z is N, V is CR¹⁰ and Y is CR⁹.

In certain embodiments, X is O, Y and V are each CH and Z is N. In certain embodiments, X is O, Y and Z are each CH and V is N. In certain embodiments, X is O and V, Y and Z are all simultaneously CH.

These embodiments are represented as Formulas IA-IC:

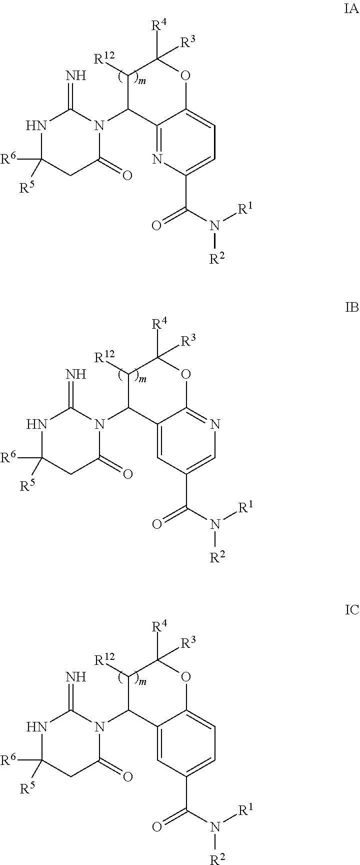

With regard to the compounds described herein, R¹ is a heterocycloalkyl, C₃-C₁₂cycloalkyl, aryl, C₁-C₆alkylaryl or when taken with R², and the nitrogen which they are bonded, forms nitrogen-containing ring, wherein the heterocycloalkyl, C₃-C₁₂cycloalkyl, aryl, C₁-C₆alkylaryl or nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, aryl, C₁-C₆alkyl, C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸).

With regard to the compounds described herein, R¹ is a heterocycloalkyl, C₃-C₁₂cycloalkyl, aryl, C₁-C₆alkylaryl or when taken with R², and the nitrogen which they are bonded, forms nitrogen-containing ring, wherein the heterocycloalkyl, C₃-C₁₂cycloalkyl, aryl, C₁-C₆alkylaryl or nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸).

In certain embodiments, R¹ is a bicyclic ring. In certain embodiments, R¹ is a bicyclic heterocycloalkyl, bicyclic C₃-C₁₂cycloalkyl, bicyclic aryl or when taken with R² and the nitrogen to which they are bonded, forms a bicyclic nitrogen-containing ring. In certain embodiments, R¹ is a bicyclic heterocycloalkyl, bicyclic C₃-C₁₂cycloalkyl, bicyclic aryl or when taken with R² and the nitrogen to which they are bonded, forms a bicyclic nitrogen-containing ring, wherein one of the rings is a benzene ring. In certain embodiments, R¹ is a bicyclic heterocycloalkyl, bicyclic C₃-C₁₂cycloalkyl, C₁-C₆alkylphenyl or when taken with R² and the nitrogen to which they are bonded, forms a bicyclic nitrogen-containing ring, wherein one of the rings of the bicyclic heterocycloalkyl, bicyclic C₃-C₁₂cycloalkyl or when taken with R² and the nitrogen to which they are bonded, is a benzene ring.

In certain embodiments, R¹ is a heterocycloalkyl. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. Non-limiting examples of bicyclic heterocycloalkyl groups include, but are not limited to,

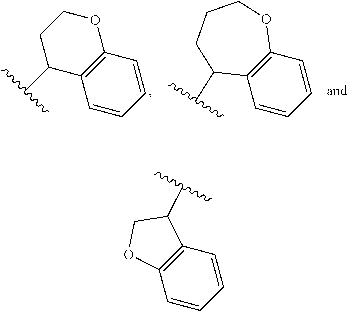 [SMILES: *C1CCOc2ccccc21 ; *C1CCCOc2ccccc21 ; *C1COc2ccccc21]

Non-limiting examples of bicyclic heterocycloalkyl groups include, but are not limited to,

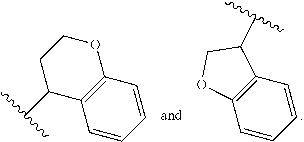 [SMILES: *C1CCOc2ccccc21 ; *C1COc2ccccc21]

In certain embodiments, R¹ is

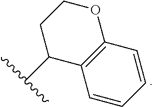 [SMILES: *C1CCOc2ccccc21]

In certain embodiments, R¹ is a C₃-C₁₂cycloalkyl. In certain embodiments, the cycloalkyl is a monocyclic cycloalkyl. Suitable examples of cycloalkyls include, but are not limited to, cyclopropyl, cyclobutyl, cyclopentyl and cyclohexyl. In certain embodiments, the cycloalkyl is a bicyclic cycloalkyl. Suitable examples of cycloalkyls include, but are not limited to:

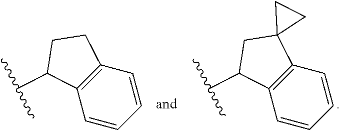 [SMILES: *C1CCc2ccccc21 ; *C1CC2(CC2)c2ccccc21]

Suitable examples of cycloalkyls include, but are not limited to:

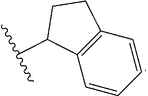 [SMILES: *C1CCc2ccccc21]

In certain embodiments, R¹ is an aryl ring. Suitable examples of aryls include, but are not limited to, monocyclic aryl groups such as, phenyl and bicyclic aryl groups such as naphthyl.

In certain embodiments, R¹ is a C₁-C₆alkylaryl ring. Suitable examples of C₁-C₆alkylaryls include, but are not limited to:

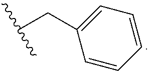 [SMILES: *Cc1ccccc1]

In certain embodiments, R¹ is taken with R² and forms a nitrogen-containing ring. Suitable examples of nitrogen-containing rings include, but are not limited to, aziridinyl, azirinyl, azetidinyl, azete, indoline, pyrrolidinyl, pyrrolyl, piperidinyl and pyridinyl. In certain embodiments, R¹ is taken with R² and forms a nitrogen-containing ring, wherein the nitrogen-containing ring is an indoline. In certain embodiments, R¹ is taken with R² and forms a nitrogen-containing ring, wherein the nitrogen-containing ring is:

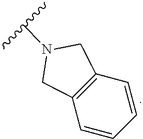 [SMILES: *N1Cc2ccccc2C1]

In certain embodiments, R¹ is unsubstituted. In other embodiments, R¹ is substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, aryl, C₁-C₆alkyl, C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸). In other embodiments, R¹ is substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸).

In certain embodiments, R¹ is substituted with 1 substituent. In certain embodiments, R¹ is substituted with 2 substituents. In certain embodiments, R¹ is substituted with 3 substituents. In certain embodiments, R¹ is substituted with 4 substituents. In certain embodiments, R¹ is substituted with 5 substituents.

In certain embodiments, R¹ is substituted with halogen. Examples of suitable halogens include chlorine, bromine, fluorine, and iodine. In certain embodiments, R¹ is substituted with CN. In certain embodiments, R¹ is substituted with OH. In certain embodiments, R¹ is substituted with an oxo group. In certain embodiments, R¹ is substituted with C₁-C₆alkoxy. Suitable alkoxys include, but are not limited to, methoxy, ethoxy, n-propoxy, isopropoxy and n-butoxy. In certain embodiments, R¹ is substituted with C₁-C₆alkylOC₁-C₆alkyl. In certain embodiments, R¹ is substituted with C₁-C₆alkylCOOH. In certain embodiments, R¹ is substituted with COOH. In certain embodiments, R¹ is substituted with C₁-C₆alkylCOOC₁-C₆alkyl.

In certain embodiments, R¹ is substituted with C₃-C₆cycloalkyl. Suitable examples of cycloalkyls include, but are not limited to, cyclopropyl, cyclobutyl, cyclopentyl and cyclohexyl. In certain embodiments, R¹ is substituted with C₁-C₆alkylC₃-C₆cycloalkyl. Suitable examples of cycloalkyls include, but are not limited to

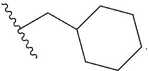 [SMILES: *CC1CCCCC1]

In certain embodiments, R¹ is substituted with aryl. Suitable examples of cycloalkyls include, but are no limited to, phenyl.

In certain embodiments, R¹ is substituted with C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R¹ is substituted with C₁-C₆alkylOhaloC₁-C₆alkyl. Suitable examples of haloalkyls include, but are not limited to,

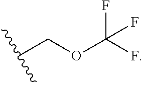 [SMILES: *COC(F)(F)F]

In certain embodiments, R¹ is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹ is substituted with C₁-C₆alkylOH. Examples of suitable alcohols include, but are not limited to, methanol, ethanol, propanol, butanol, and iso-butanol. In certain embodiments, R¹ is substituted with CON(R⁷)(R⁸). In certain embodiments, R¹ is substituted with N(R⁷)(R⁸). In certain embodiments, R¹ is substituted with C₁-C₆alkylN(R⁷)(R⁸), wherein R⁷ and R⁸ will be described in detail below.

In certain embodiments, R¹ is substituted with 1 to 4 substituents selected independently from the group consisting of bromine, fluorine, chlorine, methyl, OH, halogen, CN oxo, methoxymethyl, COOCH₂CH₃ and trifluoromethyl.

With regard to the compounds described herein, R² is hydrogen, C₁-C₆alkylCOOH, COOH, C₃-C₆cycloalkyl, C₁-C₆alkyl, haloC₁-C₆alkyl or C₁-C₆alkylOH or when taken with R¹, and the nitrogen which they are bonded, forms a nitrogen-containing ring, wherein the nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸).

In certain embodiments, R² is hydrogen. In certain embodiments, R² is C₁-C₆alkylCOOH. In certain embodiments, R² is COOH. In certain embodiments, R² is C₃-C₆cycloalkyl. Suitable examples of cycloalkyls include, but are not limited to, cyclopropyl, cyclobutyl, cyclopentyl and cyclohexyl. In certain embodiments, R² is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R² is C₁-C₆alkylOH. Examples of suitable alcohols include, but are not limited to, methanol, ethanol, propanol, butanol, and iso-butanol.

In certain embodiments, R² is taken with R¹ and forms a nitrogen-containing ring. Suitable examples of nitrogen-containing rings include, but are not limited to, aziridinyl, azirinyl, azetidinyl, azete, indoline, pyrrolidinyl, pyrrolyl, piperidinyl and pyridinyl. In certain embodiments, R² is taken with R¹ and forms an indoline.

In certain embodiments, the nitrogen-containing ring is unsubstituted. In other embodiments, the nitrogen-containing ring is substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸). In certain embodiments, the nitrogen-containing ring is substituted with 1 substituent. In certain embodiments, the nitrogen-containing ring is substituted with 2 substituents. In certain embodiments, the nitrogen-containing ring is substituted with 3 substituents. In certain embodiments, the nitrogen-containing ring is substituted with 4 substituents. In certain embodiments, the nitrogen-containing ring is substituted with 5 substituents.

In certain embodiments, the nitrogen-containing ring is substituted with halogen. Examples of suitable halogens include chlorine, bromine, fluorine, and iodine. In certain embodiments, the nitrogen-containing ring is substituted with CN. In certain embodiments, the nitrogen-containing ring is substituted with OH. In certain embodiments, the nitrogen-containing ring is substituted with alkoxy. Suitable alkoxys include, but are not limited to, methoxy, ethoxy, n-propoxy, isopropoxy and n-butoxy. In certain embodiments, the nitrogen-containing ring is substituted with C₁-C₆alkylalkoxy. In certain embodiments, the nitrogen-containing ring is substituted with C₁-C₆alkylCOOH. In certain embodiments, the nitrogen-containing ring is substituted with COOH.

In certain embodiments, the nitrogen-containing ring is substituted with C₃-C₆cycloalkyl. Suitable examples of cycloalkyls include, but are not limited to, cyclopropyl, cyclobutyl, cyclopentyl and cyclohexyl. In certain embodiments, the nitrogen-containing ring is substituted with C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹ is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹ is substituted with C₁-C₆alkylOH. Examples of suitable alcohols include, but are not limited to, methanol, ethanol, propanol, butanol, and iso-butanol. In certain embodiments, the nitrogen-containing ring is substituted with CON(R⁷)(R⁸). In certain embodiments, the nitrogen-containing ring is substituted with N(R⁷)(R⁸). In certain embodiments, the nitrogen-containing ring is substituted with C₁-C₆alkylN(R⁷)(R⁸), wherein R⁷ and R⁸ will be described in detail below.

In certain embodiments, R¹ is selected from the group consisting of:

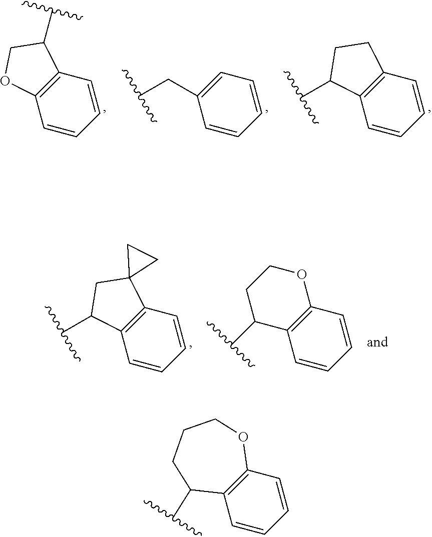

wherein R¹ is unsubstituted or substituted with 1 to 5 substituents selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₃-C₆cycloalkyl, C₁-C₆alkylC₃-C₆cycloalkyl, aryl, C₁-C₆alkyl, haloC₁-C₆alkyl, C₁-C₆alkylOhaloC₁-C₆alkyl, C₁-C₆alkylOH, CON(R⁷)(R⁸), N(R⁷)(R⁸) and C₁-C₆alkylN(R⁷)(R⁸);
R⁷ is hydrogen, C₁-C₆alkylCOOH, COOH, C₃-C₆cycloalkyl, C₁-C₆alkyl, haloC₁-C₆alkyl or C₁-C₆alkylOH; and
R⁸ is hydrogen, C₁-C₆alkylCOOH, COOH, C₃-C₆cycloalkyl, C₁-C₆alkyl, haloC₁-C₆alkyl or C₁-C₆alkylOH.

In certain embodiments, R² is hydrogen and R¹ is selected from the group consisting of:

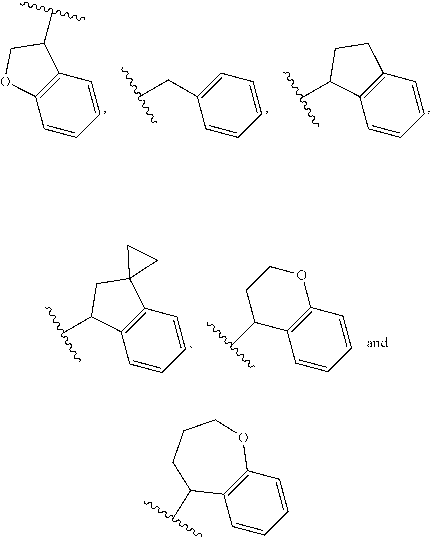

wherein R¹ is unsubstituted or substituted with 1 to 5 substituents selected from the group consisting of methyl, OH, NH₂, CH₂OH, methoxy, fluorine, phenyl, cyclohexyl, chlorine, trifluoromethyl, CH₂cyclohexyl, CH₂OCHF₂ and COOCH₂CH₃.

With regard to the compounds described herein, R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl. In certain embodiments of the compounds described herein, R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁴ forms a C₃-C₆cycloalkyl or C₃-C₆heterocycloalkyl. In certain embodiments, R³ is hydrogen. In certain embodiments, R³ is halogen. Suitable halogens include fluorine, chlorine, bromine, and iodine. In certain embodiments, R³ is CN. In certain embodiments, R³ is OH.

In certain embodiments, R³ is C₁-C₆alkoxy. Suitable alkoxys include, but are not limited to, methoxy, ethoxy, n-propoxy, isopropoxy and n-butoxy. In certain embodiments, R³ is C₁-C₆alkylOC₁-C₆alkyl. In certain embodiments, R³ is COOH. In certain embodiments, R³ is C₁-C₆alkylCOOH. In certain embodiments, R³ is C₃-C₆cycloalkyl. Suitable examples of cycloalkyls include, but are not limited to, cyclopropyl, cyclobutyl, cyclopentyl and cyclohexyl. In certain embodiments, R³ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R³ is C₁-C₆alkylOH. Examples of suitable alcohols include, but are not limited to, methanol, ethanol, propanol, butanol, and iso-butanol. In certain embodiments, R³ is CON(R⁷)(R⁸). Suitable examples of N(R⁷)(R⁸) include, but are not limited to, CONH₂ and CON(CH₃)₂. In certain embodiments, R³ is N(R⁷)(R⁸). Suitable examples of N(R⁷)(R⁸) include, but are not limited to, NH₂ and N(CH₃)₂. In certain embodiments, R³ is C₁-C₆alkylN(R⁷)(R⁸). Suitable examples of C₁-C₆alkylN(R⁷)(R⁸) include, but are not limited to,

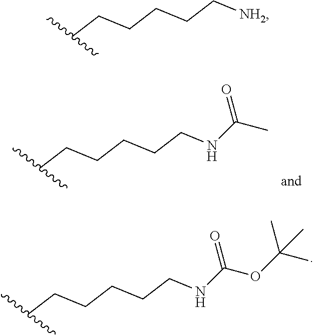

R⁷ and R⁸ are discussed in further detail below.

In certain embodiments, R³ is C₁-C₆alkylOhaloC₁-C₆alkyl. Suitable examples of haloalkyls include, but are not limited to,

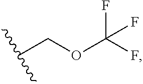 [SMILES: *COC(F)(F)F]

In certain embodiments, R³ is C₁-C₆alkyl(OCH₂CH₂)ₙN(R⁷)(R⁸). R⁷, R⁸ and n are discussed in detail below. Suitable examples of C₁-C₆alkyl(OCH₂CH₂)ₙN(R⁷)(R⁸) include, but are not limited to,

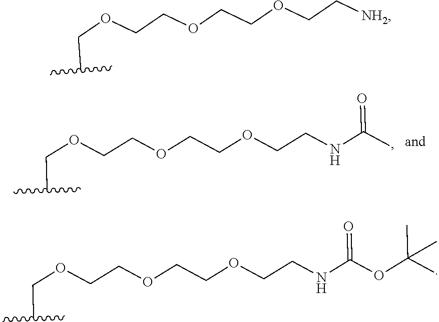

With regard to the compounds described herein, n is 1, 2, 3 or 4. In certain embodiments, n is 1. In certain embodiments, n is 2. In certain embodiments, n is 3. In certain embodiments, n is 4.

In certain embodiments, R³ is taken with R⁴ and forms a C₃-C₆cycloalkyl or C₃-C₆heterocycloalkyl. In certain embodiments, R³ is taken with R⁴ and forms a C₃-C₆cycloalkyl. Suitable examples of cycloalkyls include, but are not limited to, cyclopropyl, cyclobutyl, cyclopentyl and cyclohexyl. In certain embodiments, R³ is taken with R⁴ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R³ is hydrogen, fluorine, methyl, ethyl, OH, methoxy,

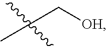 [SMILES: *CO]

CON(CH₃)₂,

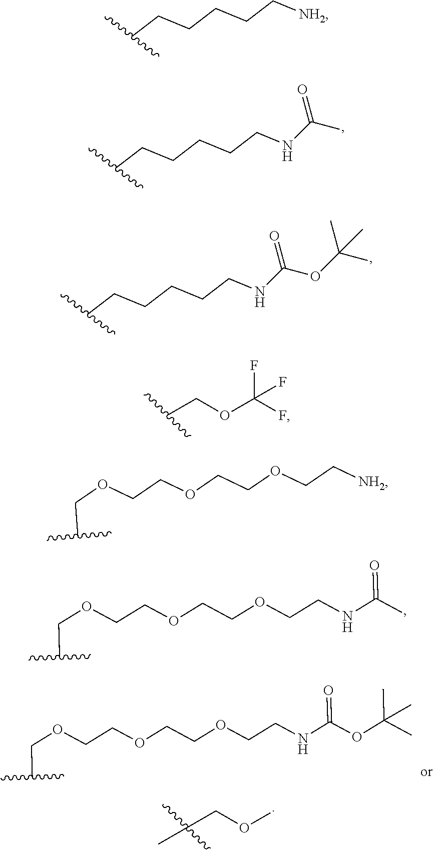

In certain embodiments, R³ is hydrogen, methyl, ethyl or

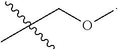

In certain embodiments, R³ is taken with R⁴ to form oxetanyl.

With regard to the compounds described herein, R⁴ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl. In certain embodiments of the compounds described herein, R⁴ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R³ forms a C₃-C₆cycloalkyl or C₃-C₆heterocycloalkyl. In certain embodiments, R⁴ is hydrogen. In certain embodiments, R⁴ is halogen. Suitable halogens include fluorine, chlorine, bromine, or iodine. In certain embodiments, R⁴ is CN. In certain embodiments, R⁴ is OH.

In certain embodiments, R⁴ is C₁-C₆alkoxy. Suitable alkoxys include, but are not limited to, methoxy, ethoxy, n-propoxy, isopropoxy and n-butoxy. In certain embodiments, R⁴ is C₁-C₆alkylOC₁-C₆alkyl. In certain embodiments, R⁴ is COOH. In certain embodiments, R⁴ is C₁-C₆alkylCOOH. In certain embodiments, R⁴ is C₃-C₆cycloalkyl. Suitable examples of cycloalkyls include, but are not limited to, cyclopropyl, cyclobutyl, cyclopentyl and cyclohexyl. In certain embodiments, R⁴ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁴ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁴ is C₁-C₆alkylOH. Examples of suitable alcohols include, but are not limited to, methanol, ethanol, propanol, butanol, and iso-butanol. In certain embodiments, R⁴ is CON(R⁷)(R⁸). Suitable examples of N(R⁷)(R⁸) include, but are not limited to, CONH₂ and CON(CH₃)₂. In certain embodiments, R⁴ is N(R⁷)(R⁸). Suitable examples of N(R⁷)(R⁸) include, but are not limited to, NH₂ and N(CH₃)₂. In certain embodiments, R⁴ is C₁-C₆alkylN(R⁷)(R⁸). Suitable examples of C₁-C₆alkylN(R⁷)(R⁸) include, but are not limited to,

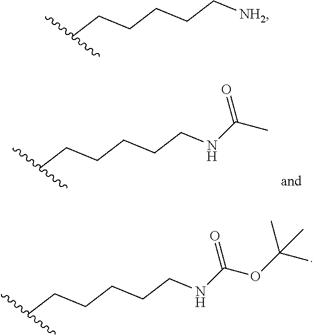

R⁷ and R⁸ are discussed in further detail below.

In certain embodiments, R⁴ is C₁-C₆alkylOhaloC₁-C₆alkyl. Suitable examples of haloalkyls include, but are not limited to,

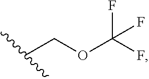 [SMILES: *COC(F)(F)F]

In certain embodiments, R⁴ is C₁-C₆alkyl(OCH₂CH₂)ₙN(R⁷)(R⁸). R⁷, R⁸ are discussed in detail below and n is discussed above. Suitable examples of C₁-C₆alkyl(OCH₂CH₂)ₙN(R⁷)(R⁸) include, but are not limited to,

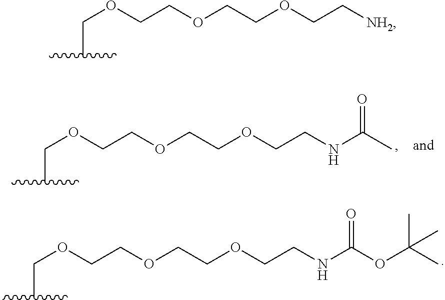 [SMILES: *COCCOCCOCCN ; *COCCOCCOCCNC(C)=O ; *COCCOCCOCCNC(=O)OC(C)(C)C]

In certain embodiments, R⁴ is taken with R³ and forms a C₃-C₆cycloalkyl or C₃-C₆heterocycloalkyl. In certain embodiments, R⁴ is taken with R³ and forms a C₃-C₆cycloalkyl. Suitable examples of cycloalkyls include, but are not limited to, cyclopropyl, cyclobutyl, cyclopentyl and cyclohexyl. In certain embodiments, R⁴ is taken with R³ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R⁴ is hydrogen or methyl. In certain embodiments, R⁴ is hydrogen, methyl, ethyl

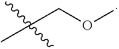

In certain embodiments, R⁴ is taken with R³ to form oxetanyl. In certain embodiments, R³ and R⁴ are both hydrogen, methyl or ethyl.

In certain embodiments, R³ is hydrogen and R⁴ is hydrogen, methyl, ethyl or

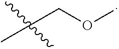

With regard to the compounds described herein, R⁵ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁶ forms a C₃-C₆cycloalkyl or C₃-C₆heterocycloalkyl. In certain embodiments, R⁵ is hydrogen. In certain embodiments, R³ is halogen. Suitable halogens include fluorine, chlorine, bromine, or iodine. In certain embodiments, R³ is CN. In certain embodiments, R⁵ is OH.

In certain embodiments, R⁵ is C₁-C₆alkoxy. Suitable alkoxys include, but are not limited to, methoxy, ethoxy, n-propoxy, isopropoxy and n-butoxy. In certain embodiments, R⁵ is C₁-C₆alkylOC₁-C₆alkyl. In certain embodiments, R⁵ is COOH. In certain embodiments, R⁵ is C₁-C₆alkylCOOH. In certain embodiments, R⁵ is C₃-C₆cycloalkyl. Suitable examples of cycloalkyls include, but are not limited to, cyclopropyl, cyclobutyl, cyclopentyl and cyclohexyl. In certain embodiments, R⁵ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁵ is C₁-C₆alkylOH. Examples of suitable alcohols include, but are not limited to, methanol, ethanol, propanol, butanol, and iso-butanol. In certain embodiments, R⁵ is CON(R⁷)(R⁸). In certain embodiments, R⁵ is N(R⁷)(R⁸). In certain embodiments, R⁵ is C₁-C₆alkylN(R⁷)(R⁸). R⁷ and R⁸ are discussed in detail below.

In certain embodiments, R⁵ is taken with R⁶ and forms a C₃-C₆cycloalkyl or C₃-C₆heterocycloalkyl. In certain embodiments, R⁵ is taken with R⁶ and forms a C₃-C₆cycloalkyl. Suitable examples of cycloalkyls include, but are not limited to, cyclopropyl, cyclobutyl, cyclopentyl and cyclohexyl. In certain embodiments, R⁵ is taken with R⁶ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R⁵ is methyl, ethyl or t-butyl.

With regard to the compounds described herein, R⁶ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁵ forms a C₃-C₆cycloalkyl or C₃-C₆heterocycloalkyl. In certain embodiments, R⁶ is hydrogen. In certain embodiments, R⁶ is halogen. Suitable halogens include fluorine, chlorine, bromine, or iodine. In certain embodiments, R⁶ is CN. In certain embodiments, R⁶ is OH.

In certain embodiments, R⁶ is C₁-C₆alkoxy. Suitable alkoxys include, but are not limited to, methoxy, ethoxy, n-propoxy, isopropoxy and n-butoxy. In certain embodiments, R⁶ is C₁-C₆alkylOC₁-C₆alkyl. In certain embodiments, R⁶ is COOH. In certain embodiments, R⁶ is C₁-C₆alkylCOOH. In certain embodiments, R⁶ is C₃-C₆cycloalkyl. Suitable examples of cycloalkyls include, but are not limited to, cyclopropyl, cyclobutyl, cyclopentyl and cyclohexyl. In certain embodiments, R⁶ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁶ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁶ is C₁-C₆alkylOH. Examples of suitable alcohols include, but are not limited to, methanol, ethanol, propanol, butanol, and iso-butanol. In certain embodiments, R⁶ is CON(R⁷)(R⁸). In certain embodiments, R⁶ is N(R⁷)(R⁸). In certain embodiments, R⁶ is C₁-C₆alkylN(R⁷)(R⁸). R⁷ and R⁸ are discussed in detail below.

In certain embodiments, R⁶ is taken with R⁵ and forms a C₃-C₆cycloalkyl or C₃-C₆heterocycloalkyl. In certain embodiments, R⁶ is taken with R⁵ and forms a C₃-C₆cycloalkyl. Suitable examples of cycloalkyls include, but are not limited to, cyclopropyl, cyclobutyl, cyclopentyl and cyclohexyl. In certain embodiments, R⁶ is taken with R⁵ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R⁶ is methyl, ethyl or t-butyl.

With regard to the compounds described herein, R⁷ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl. In certain embodiments, R⁷ is hydrogen, C₁-C₆alkylCOOH, COOH, C₃-C₆cycloalkyl, C₁-C₆alkyl, haloC₁-C₆alkyl or C₁-C₆alkylOH.

In certain embodiments, R⁷ is hydrogen. In certain embodiments, R⁷ is C₁-C₆alkylCOOH. In certain embodiments, R⁷ is COOH. In certain embodiments, R¹ is C₃-C₆cycloalkyl. Suitable examples of cycloalkyls include, but are not limited to, cyclopropyl, cyclobutyl, cyclopentyl and cyclohexyl. In certain embodiments, R⁷ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁷ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁷ is C₁-C₆alkylOH. Examples of suitable alcohols include, but are not limited to, methanol, ethanol, propanol, butanol, and iso-butanol.

In certain embodiments, R⁷ is COC₁-C₆alkyl. Suitable examples include, but are not limited to, COCH₃. In certain embodiments, R⁷ is COOC₁-C₆alkyl. Suitable examples include, but are not limited to, COOCH₃.

With regard to the compounds described herein, R⁸ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl. In certain embodiments, R⁸ is hydrogen, C₁-C₆alkylCOOH, COOH, C₃-C₆cycloalkyl, C₁-C₆alkyl, haloC₁-C₆alkyl or C₁-C₆alkylOH.

In certain embodiments, R⁸ is hydrogen. In certain embodiments, R⁸ is C₁-C₆alkylCOOH. In certain embodiments, R⁸ is COOH. In certain embodiments, R⁸ is C₃-C₆cycloalkyl. Suitable examples of cycloalkyls include, but are not limited to, cyclopropyl, cyclobutyl, cyclopentyl and cyclohexyl. In certain embodiments, R⁸ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁸ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹ is C₁-C₆alkylOH. Examples of suitable alcohols include, but are not limited to, methanol, ethanol, propanol, butanol, and iso-butanol.

In certain embodiments, R⁸ is COC₁-C₆alkyl. Suitable examples include, but are not limited to, COCH₃. In certain embodiments, R⁸ is COOC₁-C₆alkyl. Suitable examples include, but are not limited to, COOCH₃.

With regard to the compounds described herein, R⁷ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸) and N(R⁷)(R⁸). In certain embodiments, R¹ is hydrogen. In certain embodiments, R⁹ is halogen. Suitable halogens include fluorine, chlorine, bromine, or iodine. In certain embodiments, R⁹ is CN. In certain embodiments, R⁹ is OH.

In certain embodiments, R⁹ is C₁-C₆alkoxy. Suitable alkoxys include, but are not limited to, methoxy, ethoxy, n-propoxy, isopropoxy and n-butoxy. In certain embodiments, R⁹ is C₁-C₆alkylOC₁-C₆alkyl. In certain embodiments, R⁹ is COOH. In certain embodiments, R⁹ is C₁-C₆alkylCOOH. In certain embodiments, R⁹ is C₃-C₆cycloalkyl. Suitable examples of cycloalkyls include, but are not limited to, cyclopropyl, cyclobutyl, cyclopentyl and cyclohexyl. In certain embodiments, R⁹ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁹ is C₁-C₆alkylOH. Examples of suitable alcohols include, but are not limited to, methanol, ethanol, propanol, butanol, and iso-butanol. In certain embodiments, R⁹ is CON(R⁷)(R⁸). In certain embodiments, R⁹ is N(R⁷)(R⁸). In certain embodiments, R⁹ is C₁-C₆alkylN(R⁷)(R⁸).

With regard to the compounds described herein, R¹⁰ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸) and N(R⁷)(R⁸). In certain embodiments, R¹⁰ is hydrogen. In certain embodiments, R¹⁰ is halogen. Suitable halogens include fluorine, chlorine, bromine, or iodine. In certain embodiments, R¹⁰ is CN. In certain embodiments, R¹⁰ is OH.

In certain embodiments, R¹⁰ is C₁-C₆alkoxy. Suitable alkoxys include, but are not limited to, methoxy, ethoxy, n-propoxy, isopropoxy and n-butoxy. In certain embodiments, R¹⁰ is C₁-C₆alkylOC₁-C₆alkyl. In certain embodiments, R¹⁰ is COOH. In certain embodiments, R¹⁰ is C₁-C₆alkylCOOH. In certain embodiments, R¹⁰ is C₃-C₆cycloalkyl. Suitable examples of cycloalkyls include, but are not limited to, cyclopropyl, cyclobutyl, cyclopentyl and cyclohexyl. In certain embodiments, R¹⁰ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁰ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁰ is C₁-C₆alkylOH. Examples of suitable alcohols include, but are not limited to, methanol, ethanol, propanol, butanol, and iso-butanol. In certain embodiments, R¹⁰ is CON(R⁷)(R⁸). In certain embodiments, R¹⁰ is N(R⁷)(R⁸). In certain embodiments, R¹⁰ is C₁-C₆alkylN(R⁷)(R⁸).

With regard to the compounds described herein, R¹¹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸) and N(R⁷)(R⁸). In certain embodiments, R¹¹ is hydrogen. In certain embodiments, R¹¹ is halogen. Suitable halogens include fluorine, chlorine, bromine, or iodine. In certain embodiments, R¹¹ is CN. In certain embodiments, R¹¹ is OH.

In certain embodiments, R¹¹ is C₁-C₆alkoxy. Suitable alkoxys include, but are not limited to, methoxy, ethoxy, n-propoxy, isopropoxy and n-butoxy. In certain embodiments, R¹¹ is C₁-C₆alkylOC₁-C₆alkyl. In certain embodiments, R¹¹ is COOH. In certain embodiments, R¹¹ is C₁-C₆alkylCOOH. In certain embodiments, R¹¹ is C₃-C₆cycloalkyl. Suitable examples of cycloalkyls include, but are not limited to, cyclopropyl, cyclobutyl, cyclopentyl and cyclohexyl. In certain embodiments, R¹¹ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹¹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹¹ is C₁-C₆alkylOH. Examples of suitable alcohols include, but are not limited to, methanol, ethanol, propanol, butanol, and iso-butanol. In certain embodiments, R¹¹ is CON(R⁷)(R⁸). In certain embodiments, R¹¹ is N(R⁷)(R⁸). In certain embodiments, R¹¹ is C₁-C₆alkylN(R⁷)(R⁸).

With regard to the compounds described herein, R¹² is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸). In certain embodiments, R¹² is hydrogen. In certain embodiments, R¹² is halogen. Suitable halogens include fluorine, chlorine, bromine, or iodine. In certain embodiments, R¹² is CN. In certain embodiments, R¹² is OH.

In certain embodiments, R¹² is C₁-C₆alkoxy. Suitable alkoxy groups include, but are not limited to, methoxy, ethoxy, n-propoxy, isopropoxy and n-butoxy. In certain embodiments, R¹² is C₁-C₆alkylOC₁-C₆alkyl. In certain embodiments, R¹² is COOH. In certain embodiments, R¹² is C₁-C₆alkylCOOH. In certain embodiments, R¹² is C₃-C₆cycloalkyl. Suitable examples of cycloalkyls include, but are not limited to, cyclopropyl, cyclobutyl, cyclopentyl and cyclohexyl. In certain embodiments, R¹² is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹² is C₁-C₆alkylOH. Examples of suitable alcohols include, but are not limited to, methanol, ethanol, propanol, butanol, and iso-butanol. In certain embodiments, R² is CON(R⁷)(R⁸). In certain embodiments, R¹² is N(R⁷)(R⁸). In certain embodiments, R¹² is C₁-C₆alkylN(R⁷)(R⁸).

In certain embodiments, R¹² is hydrogen, methyl, ethyl, methoxy, OH or

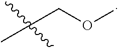

With regard to the compounds described herein, R¹³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸). In certain embodiments, R³ is hydrogen. In certain embodiments, R¹³ is halogen. Suitable halogens include fluorine, chlorine, bromine, or iodine. In certain embodiments, R¹³ is CN. In certain embodiments, R¹³ is OH.

In certain embodiments, R¹³ is C₁-C₆alkoxy. Suitable alkoxy groups include, but are not limited to, methoxy, ethoxy, n-propoxy, isopropoxy and n-butoxy. In certain embodiments, R¹³ is C₁-C₆alkylOC₁-C₆alkyl. In certain embodiments, R¹³ is COOH. In certain embodiments, R¹³ is C₁-C₆alkylCOOH. In certain embodiments, R¹³ is C₃-C₆cycloalkyl. Suitable examples of cycloalkyls include, but are not limited to, cyclopropyl, cyclobutyl, cyclopentyl and cyclohexyl. In certain embodiments, R¹³ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹³ is C₁-C₆alkylOH. Examples of suitable alcohols include, but are not limited to, methanol, ethanol, propanol, butanol, and iso-butanol. In certain embodiments, R¹³ is CON(R⁷)R⁸). In certain embodiments, R¹³ is N(R⁷)(R⁸). In certain embodiments, R¹³ is C₁-C₆alkylN(R⁷)(R⁸).

In certain embodiments, R¹³ is hydrogen, methyl, ethyl, methoxy, OH or

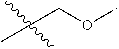

In certain embodiments, wherein m is 1 or 2, R¹² and R¹³ are independently selected from the group consisting of hydrogen, halogen, OH, C₁-C₆alkylOH, C₁-C₆alkylalkoxy and C₁-C₆alkylOC₁-C₆alkyl, C₁-C₆alkyl.

With regard to the compounds described herein, each occurrence of R¹⁴ is selected from the group consisting of hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸). In certain embodiments, R⁴ is hydrogen. In certain embodiments, R¹⁴ is halogen. Suitable halogens include fluorine, chlorine, bromine, or iodine. In certain embodiments, R⁴ is CN. In certain embodiments, R¹⁴ is OH.

In certain embodiments, R¹⁴ is C₁-C₆alkoxy. Suitable alkoxy groups include, but are not limited to, methoxy, ethoxy, n-propoxy, isopropoxy and n-butoxy. In certain embodiments, R¹⁴ is C₁-C₆alkylOC₁-C₆alkyl. In certain embodiments, R¹⁴ is COOH. In certain embodiments, R¹⁴ is C₁-C₆alkylCOOH. In certain embodiments, R¹⁴ is C₃-C₆cycloalkyl. Suitable examples of cycloalkyls include, but are not limited to, cyclopropyl, cyclobutyl, cyclopentyl and cyclohexyl. In certain embodiments, R¹⁴ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁴ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁴ is C₁-C₆alkylOH. Examples of suitable alcohols include, but are not limited to, methanol, ethanol, propanol, butanol, and iso-butanol. In certain embodiments, R⁴ is CON(R⁷)(R⁸). In certain embodiments, R¹⁴ is N(R⁷)(R⁸). In certain embodiments, R¹⁴ is C₁-C₆alkylN(R⁷)(R⁸).

In certain embodiments, wherein X is C(R¹⁴)₂, R¹⁴ is independently selected from the group consisting of hydrogen, halogen, OH, C₁-C₆alkylOH, C₁-C₆alkylalkoxy, C₁-C₆alkylOC₁-C₆alkyl and C₁-C₆alkyl.

In certain embodiments, R¹⁴ is hydrogen, methyl, ethyl, methoxy, OH or

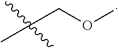

With regard to the compounds described herein, m is 0, 1 or 2. In certain embodiments, m is 0. In certain embodiments, m is 1. In certain embodiments, m is 2.
In certain embodiments, m is 1 and X is O.

In certain embodiments, m is 1 and X is CH₂.

In certain embodiments, m is 0 and X is O.

In certain embodiments, m is 1 and X is SO₂.

In certain embodiments, m is 0 and X is C(R¹⁴)₂, wherein each occurrence of R¹⁴ is independently selected from the group consisting of hydrogen, halogen, OH, C₁-C₆alkoxy and C₁-C₆alkyl.

In certain embodiments, m is 1 and X is C(R¹⁴)₂, wherein each occurrence of R¹⁴ is independently selected from the group consisting of hydrogen, halogen, OH, C₁-C₆alkoxy and C₁-C₆alkyl.

For example, in certain embodiments of Formula (I′), (I), (IA), (IB), and (IC), m is 1. In the following formula (ID), m is 0:

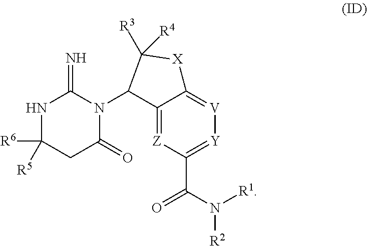

For example, in certain embodiments X is a bond and m is 0, as shown in Formula (IE)

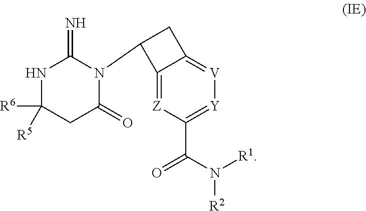

In each of the various embodiments of the invention, in the compounds used in the methods herein, each variable (including those in each of Formula (I′), (I), (IA), (IB), (IC), (ID) and (IE), and the various embodiments thereof) it shall be understood that each variable is to be selected independently of the others unless otherwise indicated.

In each of the various embodiments of the invention, the compounds described herein, including those in each of Formula (I′), (I), (IA), (IB), (IC), (ID) and (IE) and the various embodiments thereof, may exist in different forms of the compounds such as, for example, any solvates, hydrates, stereoisomers, and tautomers of said compounds and of any pharmaceutically acceptable salts thereof.

In certain embodiments described herein, the present invention is directed to methods of chemovaccination against Plasmodium infections comprising administering to a patient, an effective amount of a dual inhibitor of plasmepsin IX and X selected from the group consisting of:

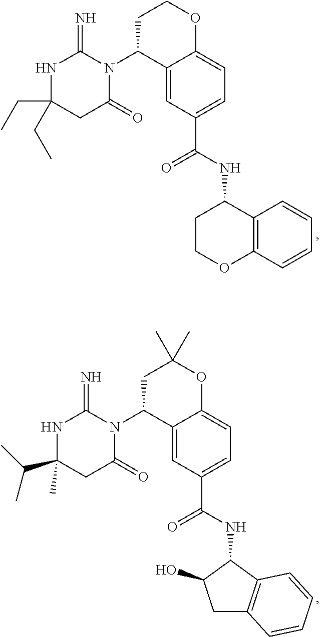 [SMILES: CCC1(CC)CC(=O)N([C@@H]2CCOc3ccc(C(=O)N[C@H]4CCOc5ccccc54)cc32)C(=N)N1 ; CC(C)[C@]1(C)CC(=O)N([C@@H]2CC(C)(C)Oc3ccc(C(=O)N[C@@H]4c5ccccc5C[C@H]4O)cc32)C(=N)N1]

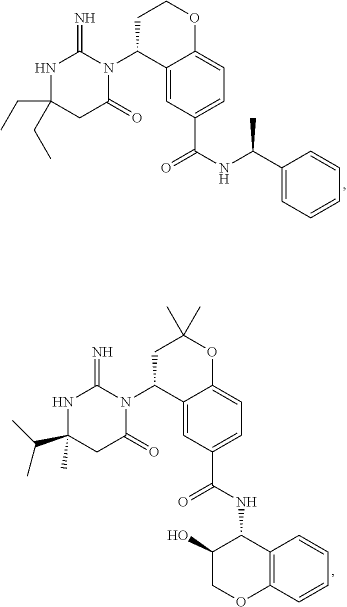 [SMILES: CCC1(CC)CC(=O)N([C@@H]2CCOc3ccc(C(=O)N[C@@H](C)c4ccccc4)cc32)C(=N)N1 ; CC(C)[C@]1(C)CC(=O)N([C@@H]2CC(C)(C)Oc3ccc(C(=O)N[C@@H]4c5ccccc5OC[C@H]4O)cc32)C(=N)N1]

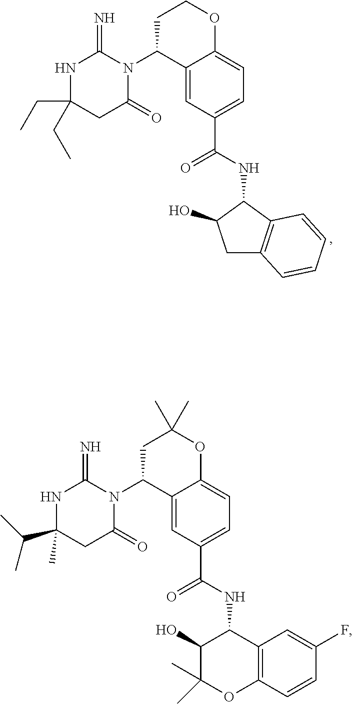 [SMILES: CCC1(CC)CC(=O)N([C@@H]2CCOc3ccc(C(=O)N[C@@H]4c5ccccc5C[C@H]4O)cc32)C(=N)N1 ; CC(C)[C@]1(C)CC(=O)N([C@@H]2CC(C)(C)Oc3ccc(C(=O)N[C@@H]4c5cc(F)ccc5OC(C)(C)[C@H]4O)cc32)C(=N)N1]

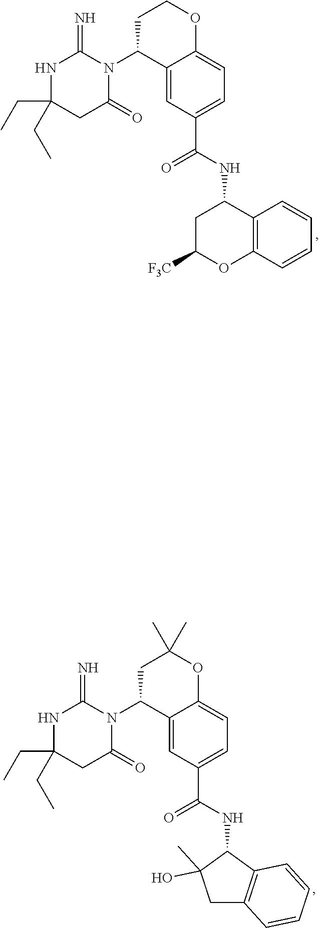 [SMILES: CCC1(CC)CC(=O)N([C@@H]2CCOc3ccc(C(=O)N[C@H]4C[C@H](C(F)(F)F)Oc5ccccc54)cc32)C(=N)N1 ; CCC1(CC)CC(=O)N([C@@H]2CC(C)(C)Oc3ccc(C(=O)N[C@@H]4c5ccccc5CC4(C)O)cc32)C(=N)N1]

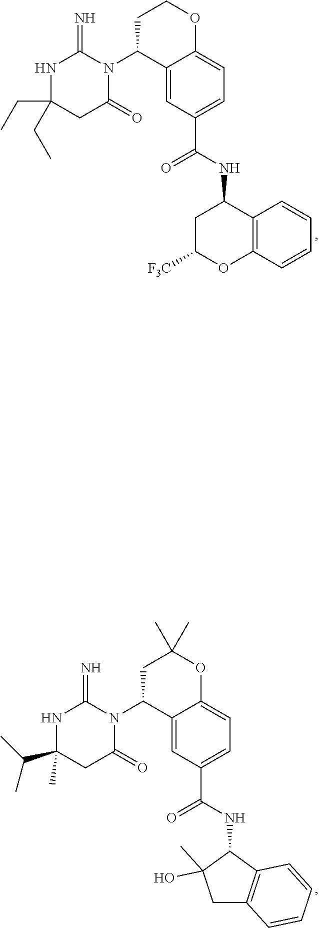 [SMILES: CCC1(CC)CC(=O)N([C@@H]2CCOc3ccc(C(=O)N[C@@H]4C[C@@H](C(F)(F)F)Oc5ccccc54)cc32)C(=N)N1 ; CC(C)[C@]1(C)CC(=O)N([C@@H]2CC(C)(C)Oc3ccc(C(=O)N[C@@H]4c5ccccc5CC4(C)O)cc32)C(=N)N1]

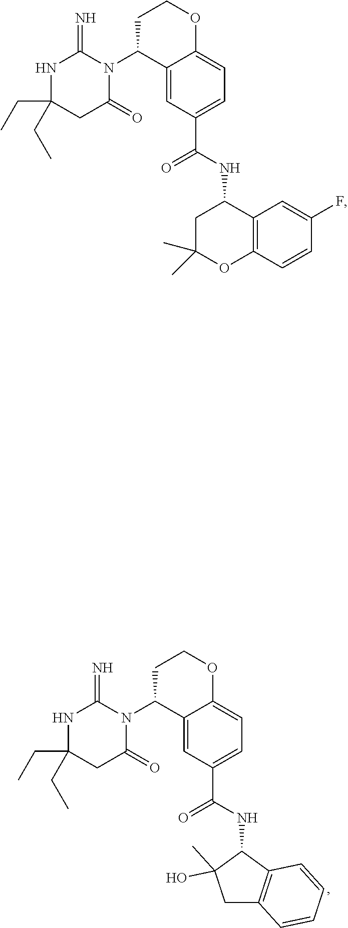 [SMILES: CCC1(CC)CC(=O)N([C@@H]2CCOc3ccc(C(=O)N[C@H]4CC(C)(C)Oc5ccc(F)cc54)cc32)C(=N)N1 ; CCC1(CC)CC(=O)N([C@@H]2CCOc3ccc(C(=O)N[C@@H]4c5ccccc5CC4(C)O)cc32)C(=N)N1]

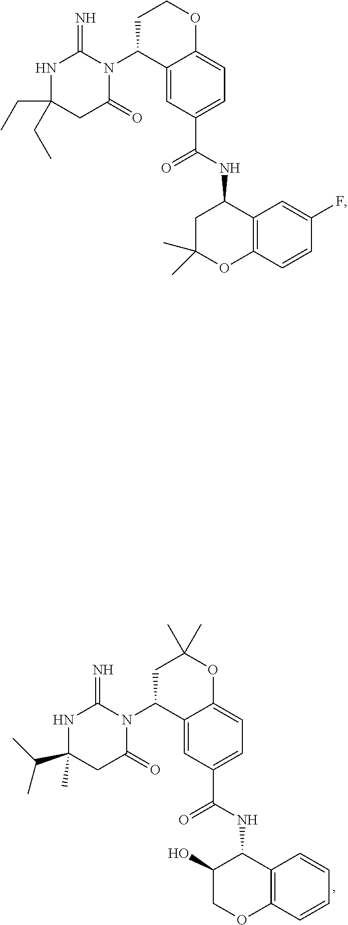 [SMILES: CCC1(CC)CC(=O)N([C@@H]2CCOc3ccc(C(=O)N[C@@H]4CC(C)(C)Oc5ccc(F)cc54)cc32)C(=N)N1 ; CC(C)[C@]1(C)CC(=O)N([C@@H]2CC(C)(C)Oc3ccc(C(=O)N[C@@H]4c5ccccc5OC[C@H]4O)cc32)C(=N)N1]

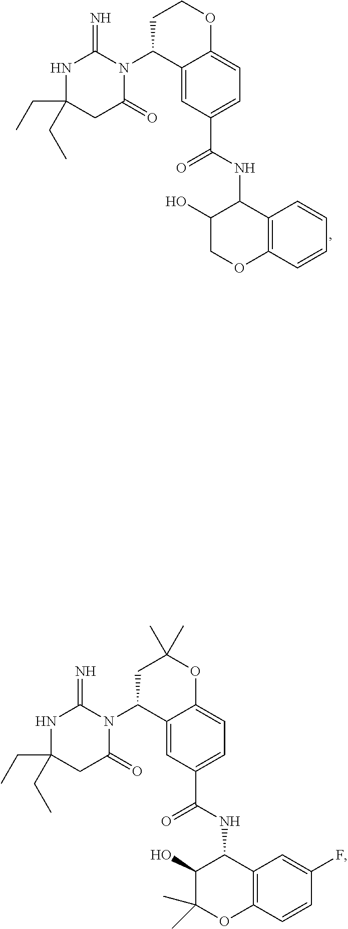 [SMILES: CCC1(CC)CC(=O)N([C@@H]2CCOc3ccc(C(=O)NC4c5ccccc5OCC4O)cc32)C(=N)N1 ; CCC1(CC)CC(=O)N([C@@H]2CC(C)(C)Oc3ccc(C(=O)N[C@@H]4c5cc(F)ccc5OC(C)(C)[C@H]4O)cc32)C(=N)N1]

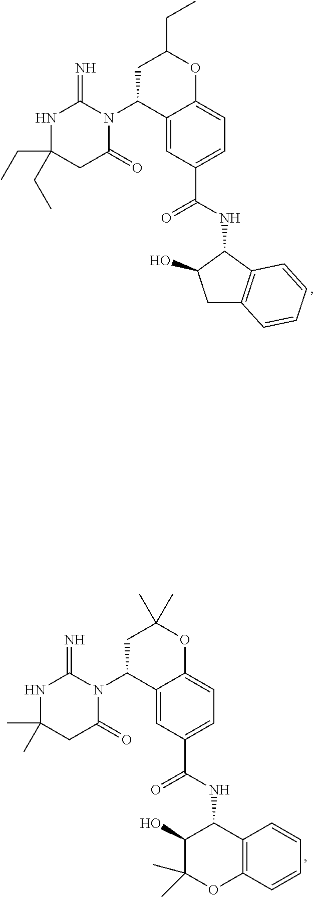

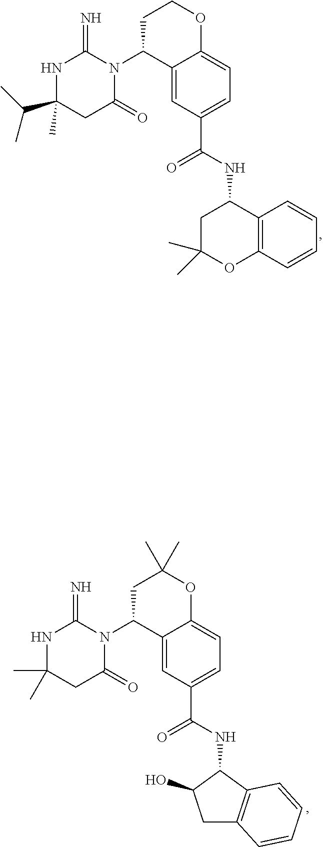 [SMILES: CC(C)[C@]1(C)CC(=O)N([C@@H]2CCOc3ccc(C(=O)N[C@H]4CC(C)(C)Oc5ccccc54)cc32)C(=N)N1 ; CC1(C)CC(=O)N([C@@H]2CC(C)(C)Oc3ccc(C(=O)N[C@@H]4c5ccccc5C[C@H]4O)cc32)C(=N)N1]

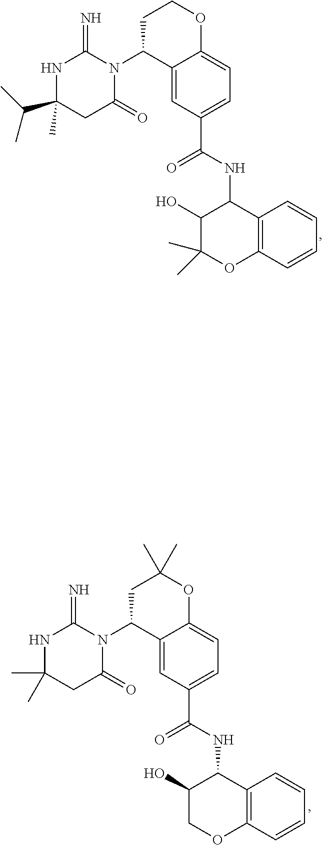 [SMILES: CC(C)[C@]1(C)CC(=O)N([C@@H]2CCOc3ccc(C(=O)NC4c5ccccc5OC(C)(C)C4O)cc32)C(=N)N1 ; CC1(C)CC(=O)N([C@@H]2CC(C)(C)Oc3ccc(C(=O)N[C@@H]4c5ccccc5OC[C@H]4O)cc32)C(=N)N1]

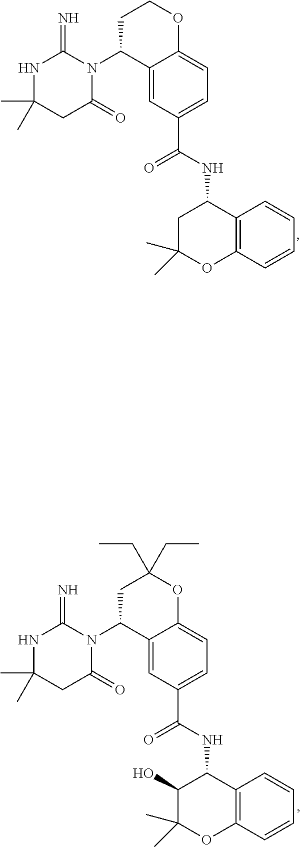 [SMILES: CC1(C)CC(=O)N([C@@H]2CCOc3ccc(C(=O)N[C@H]4CC(C)(C)Oc5ccccc54)cc32)C(=N)N1 ; CCC1(CC)C[C@@H](N2C(=N)NC(C)(C)CC2=O)c2cc(C(=O)N[C@@H]3c4ccccc4OC(C)(C)[C@H]3O)ccc2O1]

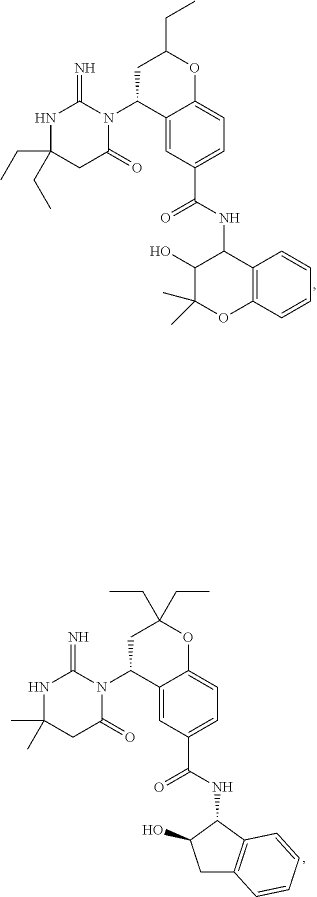

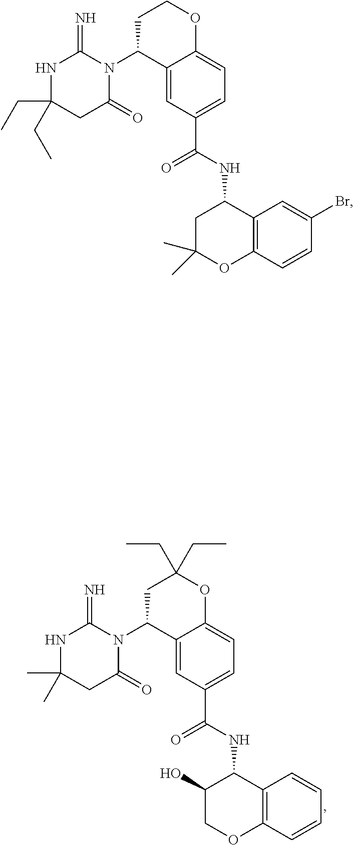 [SMILES: CCC1(CC)CC(=O)N([C@@H]2CCOc3ccc(C(=O)N[C@H]4CC(C)(C)Oc5ccc(Br)cc54)cc32)C(=N)N1 ; CCC1(CC)C[C@@H](N2C(=N)NC(C)(C)CC2=O)c2cc(C(=O)N[C@@H]3c4ccccc4OC[C@H]3O)ccc2O1]

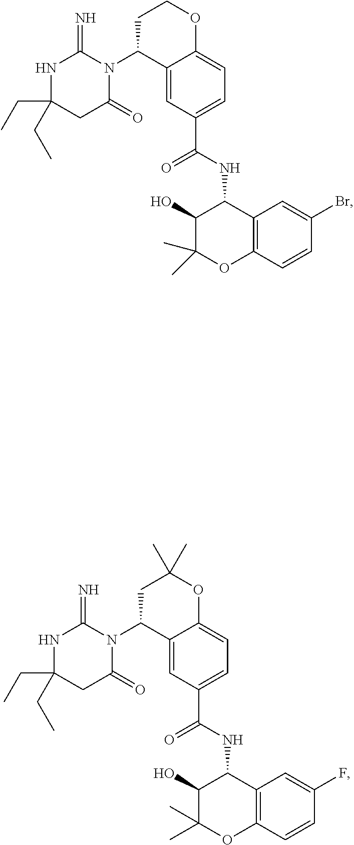 [SMILES: CCC1(CC)CC(=O)N([C@@H]2CCOc3ccc(C(=O)N[C@@H]4c5cc(Br)ccc5OC(C)(C)[C@H]4O)cc32)C(=N)N1 ; CCC1(CC)CC(=O)N([C@@H]2CC(C)(C)Oc3ccc(C(=O)N[C@@H]4c5cc(F)ccc5OC(C)(C)[C@H]4O)cc32)C(=N)N1]

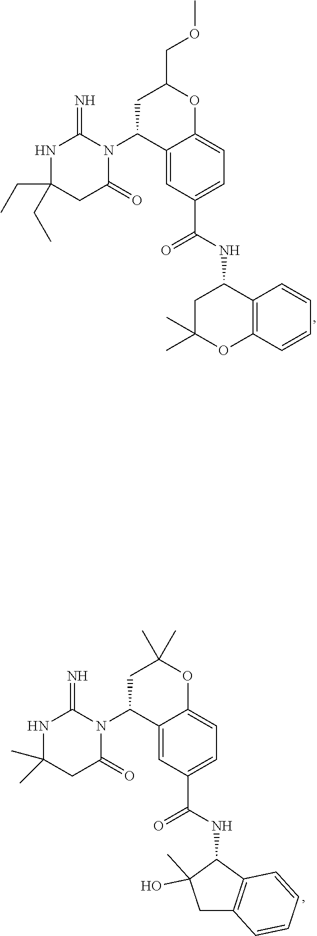

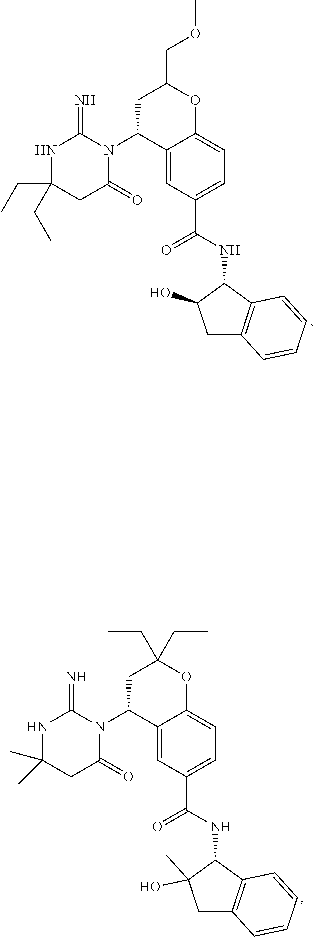

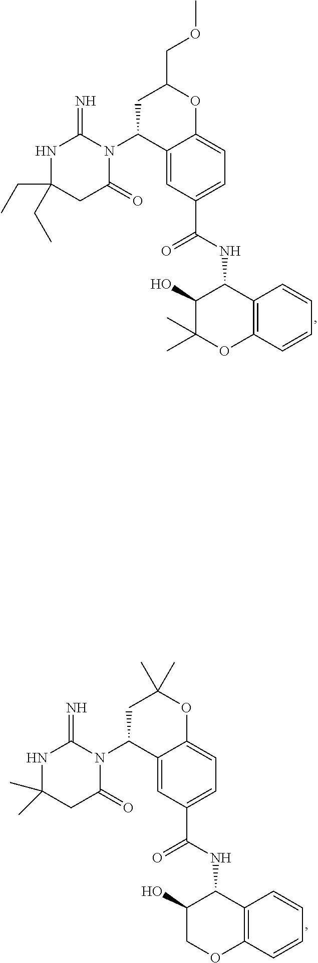

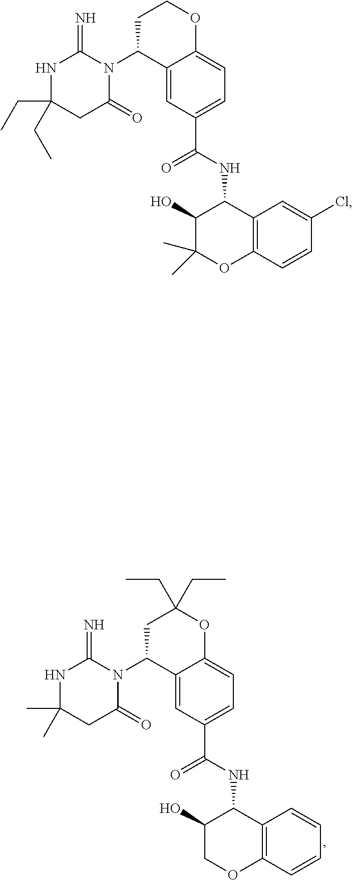 [SMILES: CCC1(CC)CC(=O)N([C@@H]2CCOc3ccc(C(=O)N[C@@H]4c5cc(Cl)ccc5OC(C)(C)[C@H]4O)cc32)C(=N)N1 ; CCC1(CC)C[C@@H](N2C(=N)NC(C)(C)CC2=O)c2cc(C(=O)N[C@@H]3c4ccccc4OC[C@H]3O)ccc2O1]

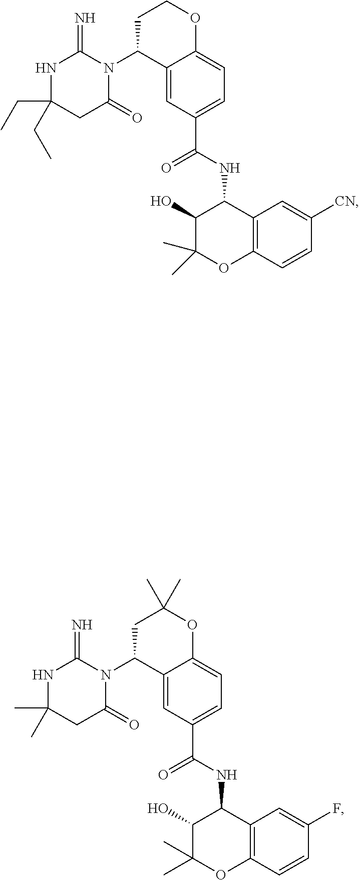 [SMILES: CCC1(CC)CC(=O)N([C@@H]2CCOc3ccc(C(=O)N[C@@H]4c5cc(C#N)ccc5OC(C)(C)[C@H]4O)cc32)C(=N)N1 ; CC1(C)CC(=O)N([C@@H]2CC(C)(C)Oc3ccc(C(=O)N[C@H]4c5cc(F)ccc5OC(C)(C)[C@@H]4O)cc32)C(=N)N1]

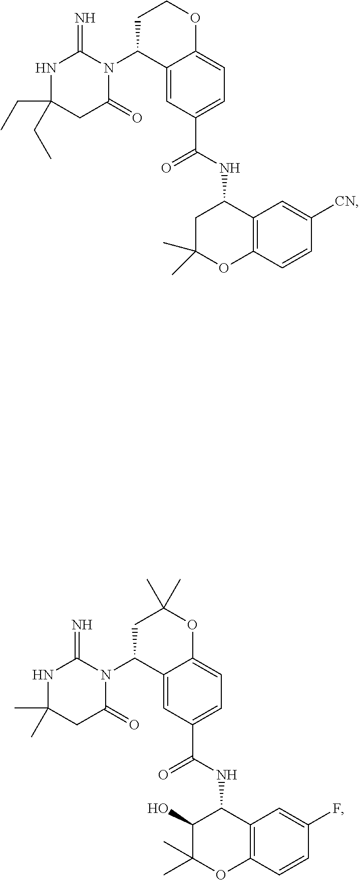 [SMILES: CCC1(CC)CC(=O)N([C@@H]2CCOc3ccc(C(=O)N[C@H]4CC(C)(C)Oc5ccc(C#N)cc54)cc32)C(=N)N1 ; CC1(C)CC(=O)N([C@@H]2CC(C)(C)Oc3ccc(C(=O)N[C@@H]4c5cc(F)ccc5OC(C)(C)[C@H]4O)cc32)C(=N)N1]

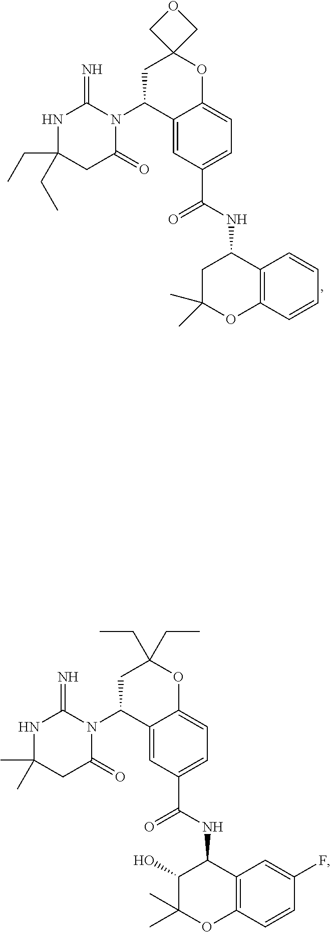 [SMILES: CCC1(CC)CC(=O)N([C@@H]2CC3(COC3)Oc3ccc(C(=O)N[C@H]4CC(C)(C)Oc5ccccc54)cc32)C(=N)N1 ; CCC1(CC)C[C@@H](N2C(=N)NC(C)(C)CC2=O)c2cc(C(=O)N[C@H]3c4cc(F)ccc4OC(C)(C)[C@@H]3O)ccc2O1]

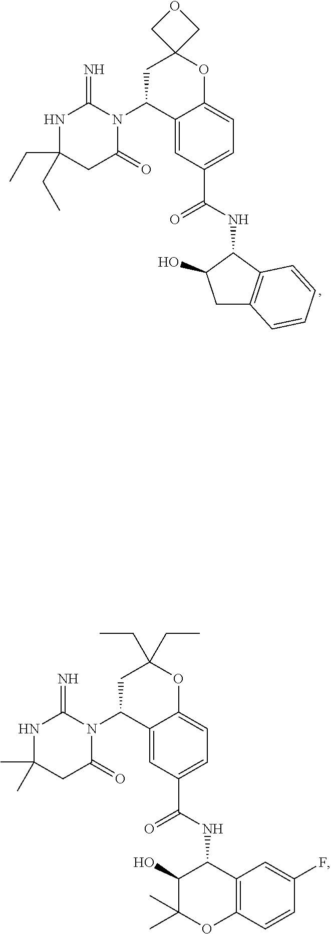 [SMILES: CCC1(CC)CC(=O)N([C@@H]2CC3(COC3)Oc3ccc(C(=O)N[C@@H]4c5ccccc5C[C@H]4O)cc32)C(=N)N1 ; CCC1(CC)C[C@@H](N2C(=N)NC(C)(C)CC2=O)c2cc(C(=O)N[C@@H]3c4cc(F)ccc4OC(C)(C)[C@H]3O)ccc2O1]

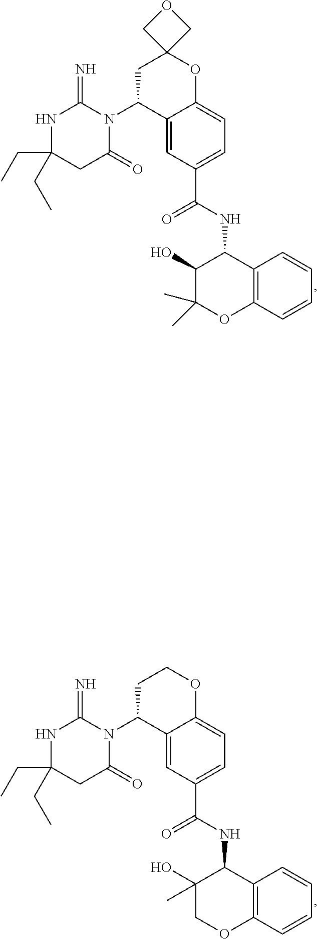 [SMILES: CCC1(CC)CC(=O)N([C@@H]2CC3(COC3)Oc3ccc(C(=O)N[C@@H]4c5ccccc5OC(C)(C)[C@H]4O)cc32)C(=N)N1 ; CCC1(CC)CC(=O)N([C@@H]2CCOc3ccc(C(=O)N[C@H]4c5ccccc5OCC4(C)O)cc32)C(=N)N1]

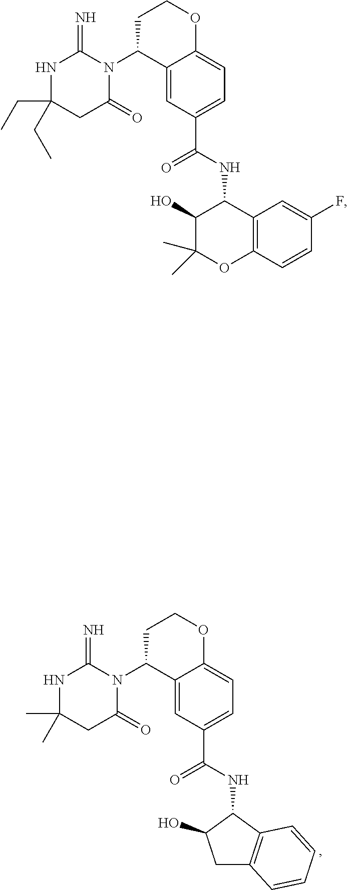 [SMILES: CCC1(CC)CC(=O)N([C@@H]2CCOc3ccc(C(=O)N[C@@H]4c5cc(F)ccc5OC(C)(C)[C@H]4O)cc32)C(=N)N1 ; CC1(C)CC(=O)N([C@@H]2CCOc3ccc(C(=O)N[C@@H]4c5ccccc5C[C@H]4O)cc32)C(=N)N1]

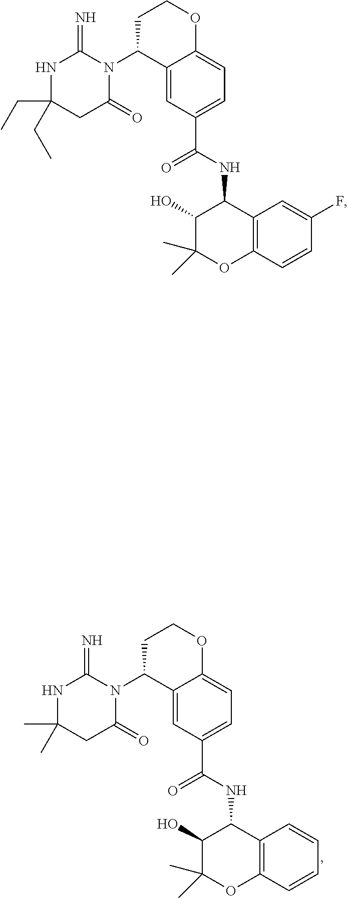 [SMILES: CCC1(CC)CC(=O)N([C@@H]2CCOc3ccc(C(=O)N[C@H]4c5cc(F)ccc5OC(C)(C)[C@@H]4O)cc32)C(=N)N1 ; CC1(C)CC(=O)N([C@@H]2CCOc3ccc(C(=O)N[C@@H]4c5ccccc5OC(C)(C)[C@H]4O)cc32)C(=N)N1]

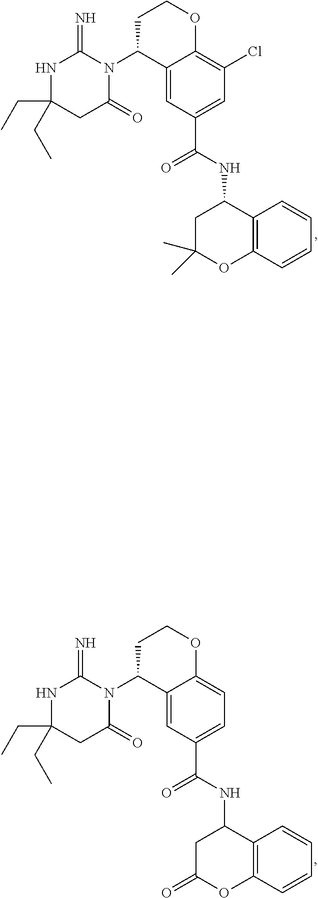 [SMILES: CCC1(CC)CC(=O)N([C@@H]2CCOc3c(Cl)cc(C(=O)N[C@H]4CC(C)(C)Oc5ccccc54)cc32)C(=N)N1 ; CCC1(CC)CC(=O)N([C@@H]2CCOc3ccc(C(=O)NC4CC(=O)Oc5ccccc54)cc32)C(=N)N1]

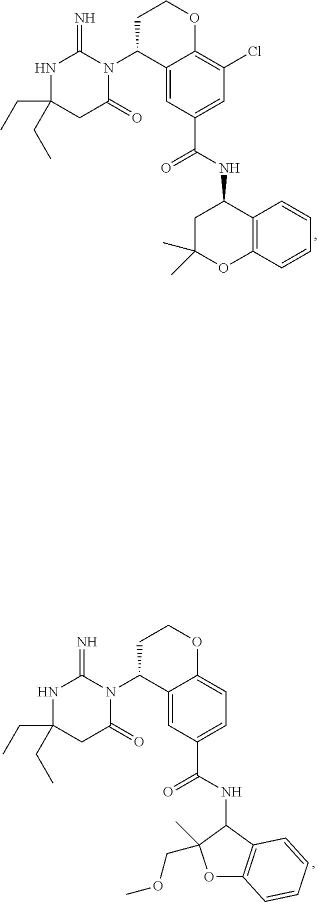 [SMILES: CCC1(CC)CC(=O)N([C@@H]2CCOc3c(Cl)cc(C(=O)N[C@@H]4CC(C)(C)Oc5ccccc54)cc32)C(=N)N1 ; CCC1(CC)CC(=O)N([C@@H]2CCOc3ccc(C(=O)NC4c5ccccc5OC4(C)COC)cc32)C(=N)N1]

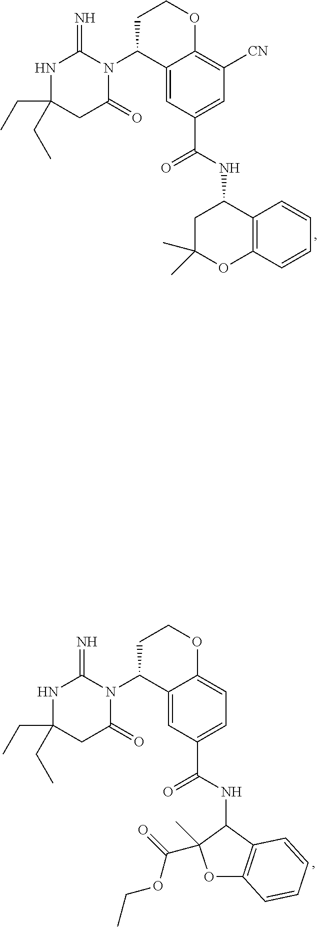 [SMILES: CCC1(CC)CC(=O)N([C@@H]2CCOc3c(C#N)cc(C(=O)N[C@H]4CC(C)(C)Oc5ccccc54)cc32)C(=N)N1 ; CCOC(=O)C1(C)Oc2ccccc2C1NC(=O)c1ccc2c(c1)[C@H](N1C(=N)NC(CC)(CC)CC1=O)CCO2]

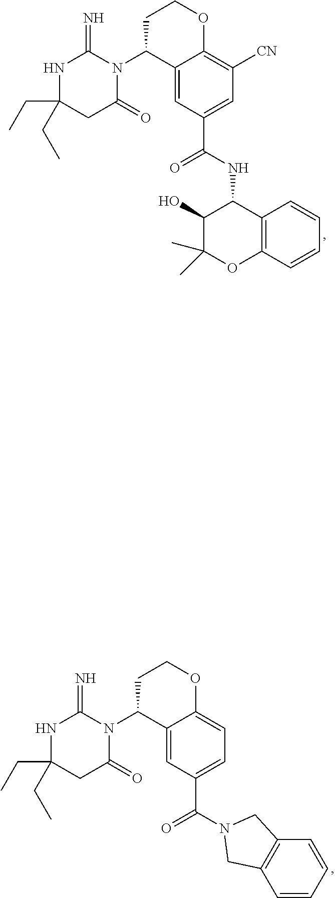 [SMILES: CCC1(CC)CC(=O)N([C@@H]2CCOc3c(C#N)cc(C(=O)N[C@@H]4c5ccccc5OC(C)(C)[C@H]4O)cc32)C(=N)N1 ; CCC1(CC)CC(=O)N([C@@H]2CCOc3ccc(C(=O)N4Cc5ccccc5C4)cc32)C(=N)N1]

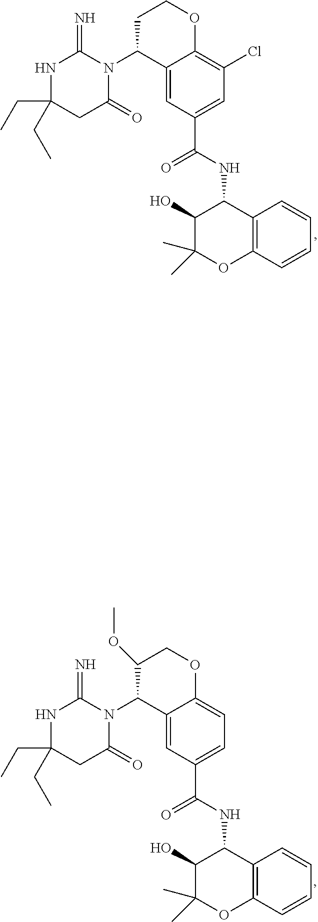

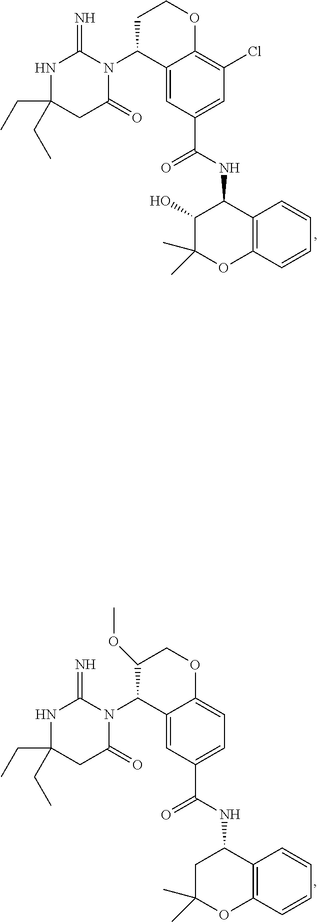

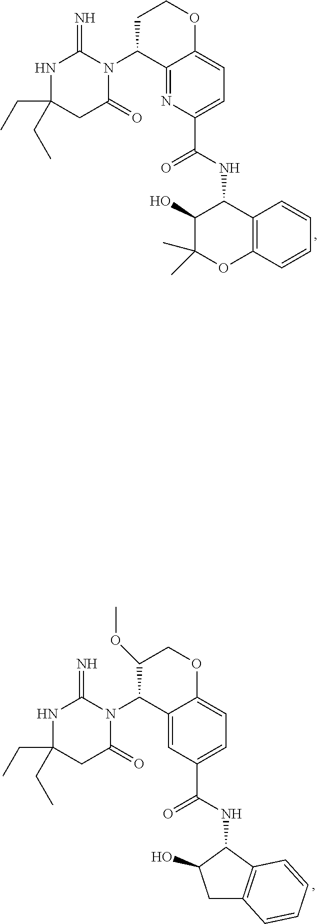

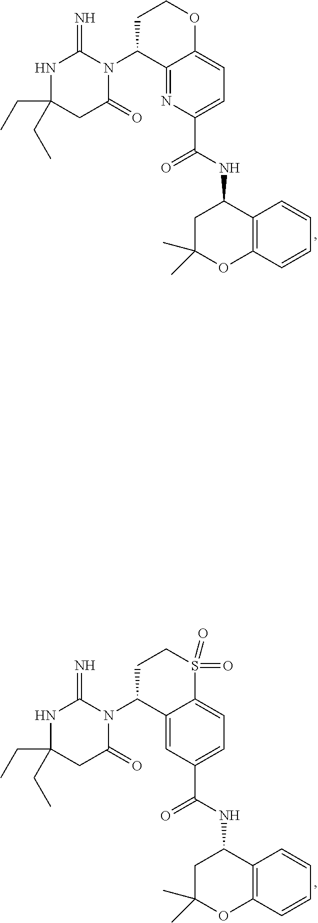 [SMILES: CCC1(CC)CC(=O)N([C@@H]2CCOc3ccc(C(=O)N[C@@H]4CC(C)(C)Oc5ccccc54)nc32)C(=N)N1 ; CCC1(CC)CC(=O)N([C@@H]2CCS(=O)(=O)c3ccc(C(=O)N[C@H]4CC(C)(C)Oc5ccccc54)cc32)C(=N)N1]

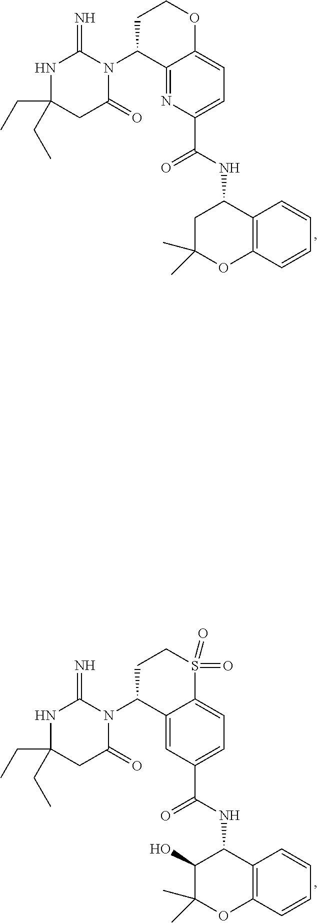 [SMILES: CCC1(CC)CC(=O)N([C@@H]2CCOc3ccc(C(=O)N[C@H]4CC(C)(C)Oc5ccccc54)nc32)C(=N)N1 ; CCC1(CC)CC(=O)N([C@@H]2CCS(=O)(=O)c3ccc(C(=O)N[C@@H]4c5ccccc5OC(C)(C)[C@H]4O)cc32)C(=N)N1]

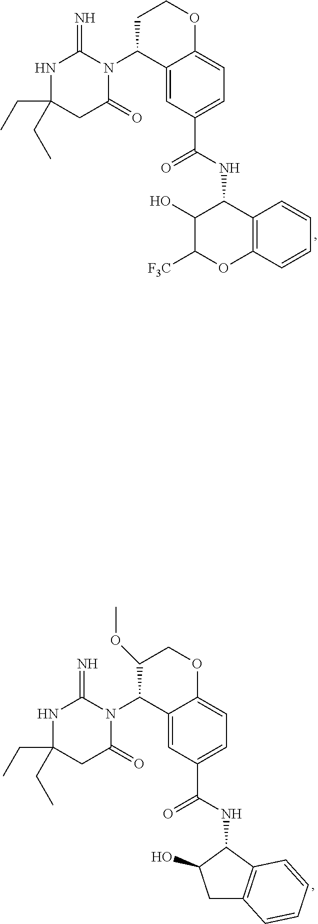

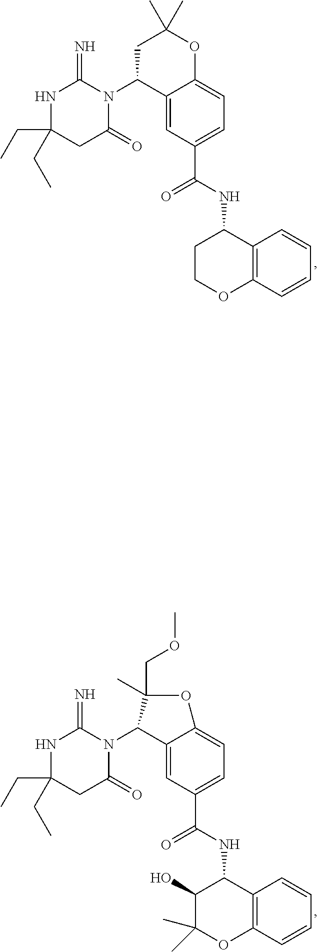

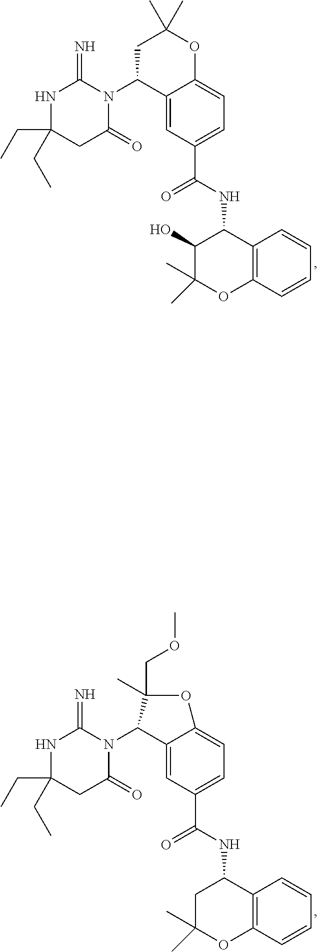

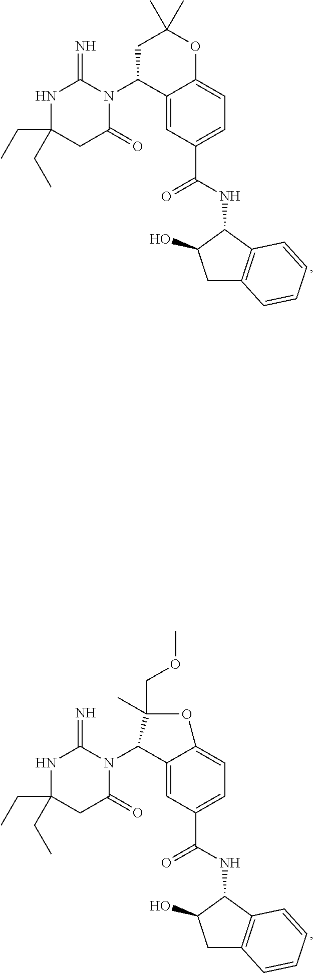

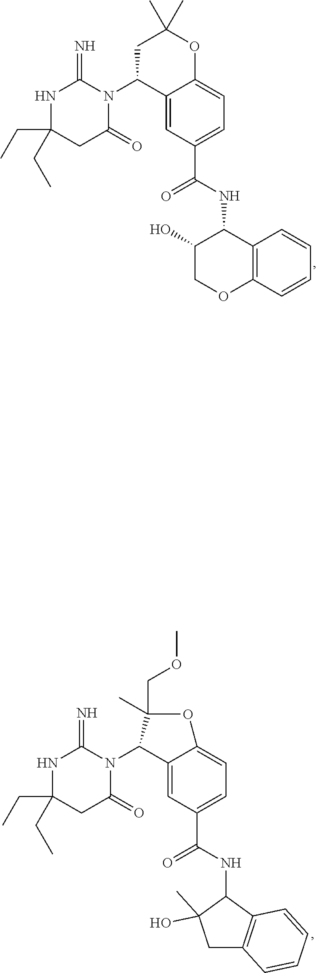

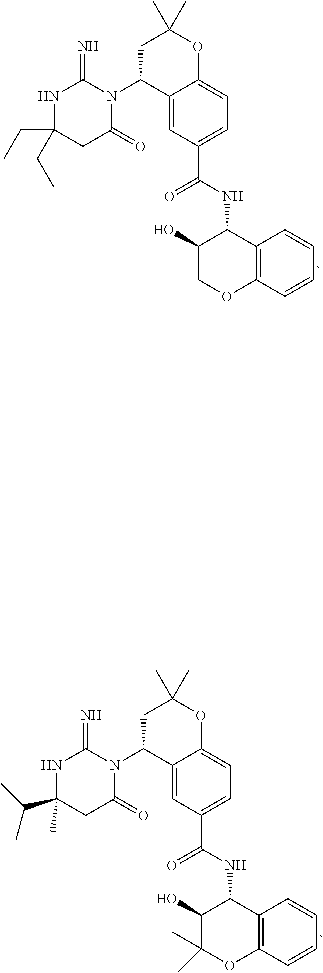 [SMILES: CCC1(CC)CC(=O)N([C@@H]2CC(C)(C)Oc3ccc(C(=O)N[C@@H]4c5ccccc5OC[C@H]4O)cc32)C(=N)N1 ; CC(C)[C@]1(C)CC(=O)N([C@@H]2CC(C)(C)Oc3ccc(C(=O)N[C@@H]4c5ccccc5OC(C)(C)[C@H]4O)cc32)C(=N)N1]

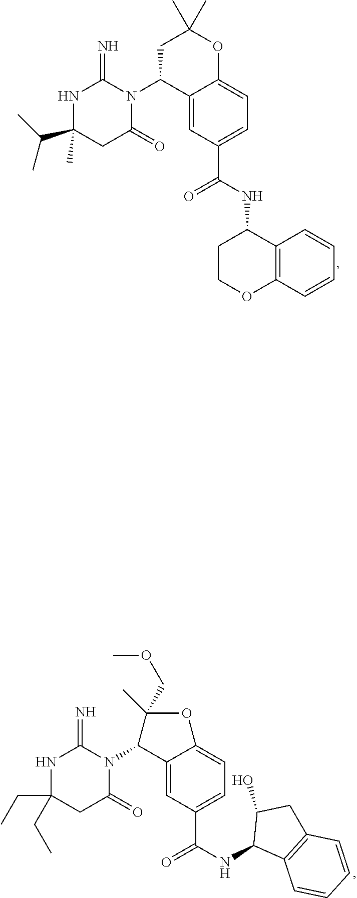 [SMILES: CC(C)[C@]1(C)CC(=O)N([C@@H]2CC(C)(C)Oc3ccc(C(=O)N[C@H]4CCOc5ccccc54)cc32)C(=N)N1 ; CCC1(CC)CC(=O)N([C@H]2c3cc(C(=O)N[C@@H]4c5ccccc5C[C@H]4O)ccc3O[C@@]2(C)COC)C(=N)N1]

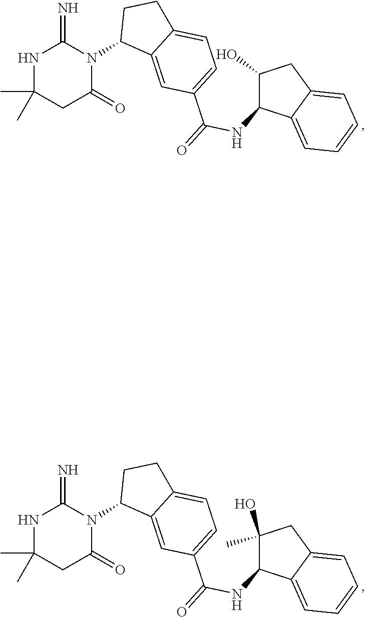 [SMILES: CC1(C)CC(=O)N([C@@H]2CCc3ccc(C(=O)N[C@@H]4c5ccccc5C[C@H]4O)cc32)C(=N)N1 ; CC1(C)CC(=O)N([C@@H]2CCc3ccc(C(=O)N[C@@H]4c5ccccc5C[C@]4(C)O)cc32)C(=N)N1]

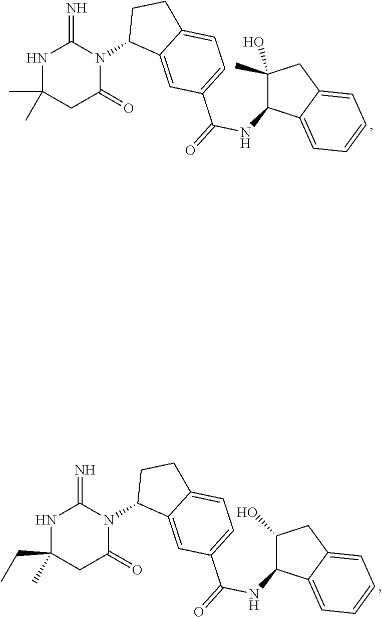 [SMILES: CC1(C)CC(=O)N([C@@H]2CCc3ccc(C(=O)N[C@@H]4c5ccccc5C[C@@]4(C)O)cc32)C(=N)N1 ; CC[C@]1(C)CC(=O)N([C@@H]2CCc3ccc(C(=O)N[C@@H]4c5ccccc5C[C@H]4O)cc32)C(=N)N1]

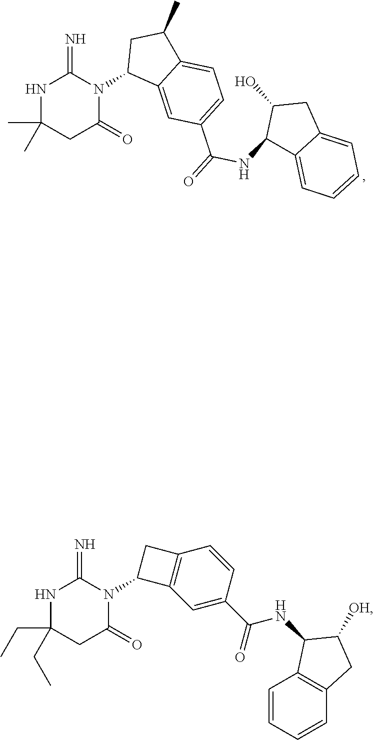 [SMILES: C[C@@H]1C[C@@H](N2C(=N)NC(C)(C)CC2=O)c2cc(C(=O)N[C@@H]3c4ccccc4C[C@H]3O)ccc21 ; CCC1(CC)CC(=O)N([C@@H]2Cc3ccc(C(=O)N[C@@H]4c5ccccc5C[C@H]4O)cc32)C(=N)N1]

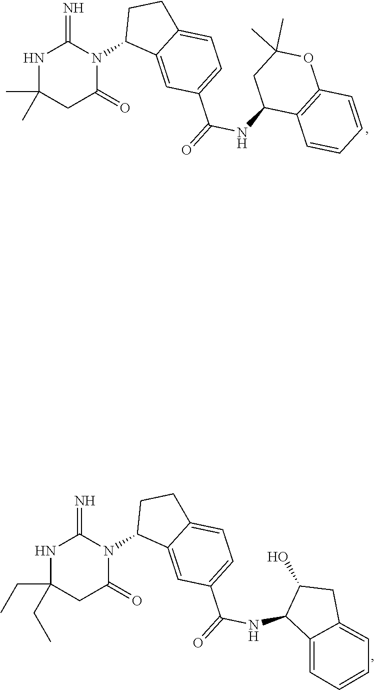 [SMILES: CC1(C)CC(=O)N([C@@H]2CCc3ccc(C(=O)N[C@H]4CC(C)(C)Oc5ccccc54)cc32)C(=N)N1 ; CCC1(CC)CC(=O)N([C@@H]2CCc3ccc(C(=O)N[C@@H]4c5ccccc5C[C@H]4O)cc32)C(=N)N1]

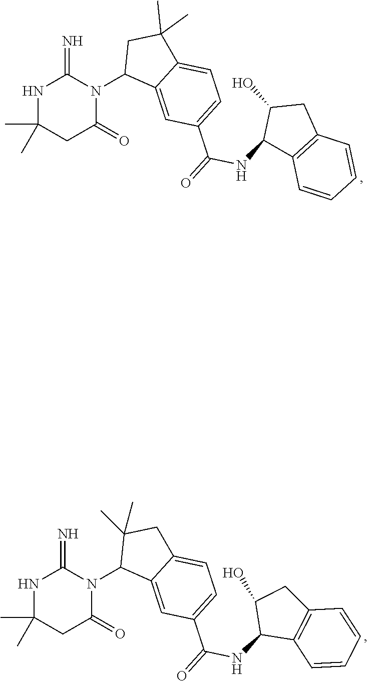 [SMILES: CC1(C)CC(=O)N(C2CC(C)(C)c3ccc(C(=O)N[C@@H]4c5ccccc5C[C@H]4O)cc32)C(=N)N1 ; CC1(C)CC(=O)N(C2c3cc(C(=O)N[C@@H]4c5ccccc5C[C@H]4O)ccc3CC2(C)C)C(=N)N1]

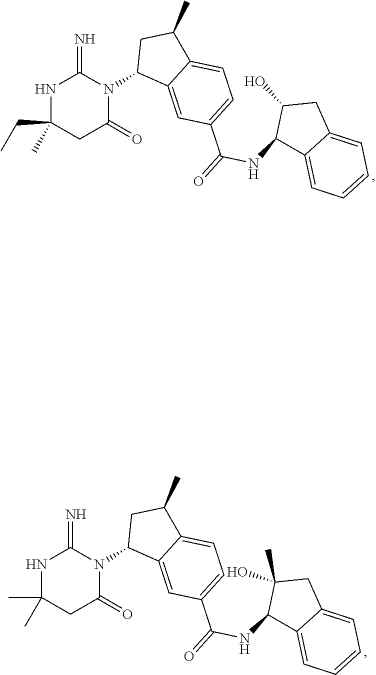 [SMILES: CC[C@]1(C)CC(=O)N([C@@H]2C[C@@H](C)c3ccc(C(=O)N[C@@H]4c5ccccc5C[C@H]4O)cc32)C(=N)N1 ; C[C@@H]1C[C@@H](N2C(=N)NC(C)(C)CC2=O)c2cc(C(=O)N[C@@H]3c4ccccc4C[C@@]3(C)O)ccc21]

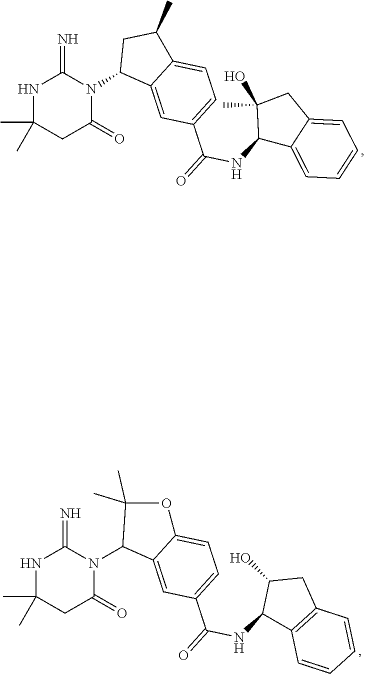 [SMILES: C[C@@H]1C[C@@H](N2C(=N)NC(C)(C)CC2=O)c2cc(C(=O)N[C@@H]3c4ccccc4C[C@]3(C)O)ccc21 ; CC1(C)CC(=O)N(C2c3cc(C(=O)N[C@@H]4c5ccccc5C[C@H]4O)ccc3OC2(C)C)C(=N)N1]

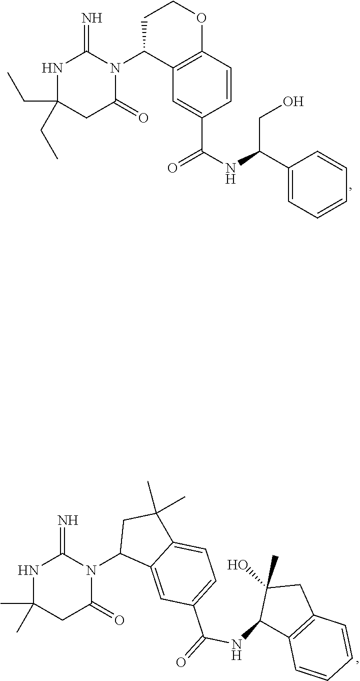 [SMILES: CCC1(CC)CC(=O)N([C@@H]2CCOc3ccc(C(=O)N[C@@H](CO)c4ccccc4)cc32)C(=N)N1 ; CC1(C)CC(=O)N(C2CC(C)(C)c3ccc(C(=O)N[C@@H]4c5ccccc5C[C@@]4(C)O)cc32)C(=N)N1]

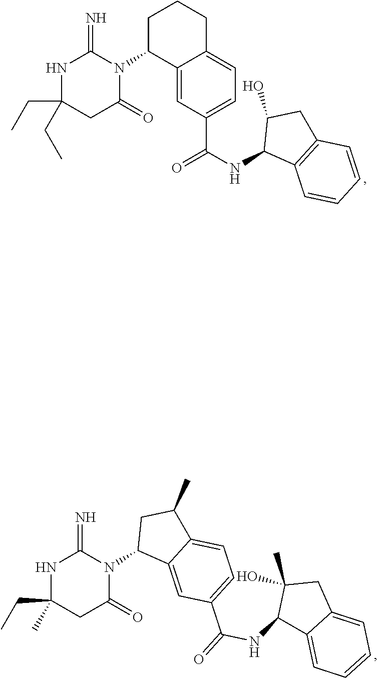 [SMILES: CCC1(CC)CC(=O)N([C@@H]2CCCc3ccc(C(=O)N[C@@H]4c5ccccc5C[C@H]4O)cc32)C(=N)N1 ; CC[C@]1(C)CC(=O)N([C@@H]2C[C@@H](C)c3ccc(C(=O)N[C@@H]4c5ccccc5C[C@@]4(C)O)cc32)C(=N)N1]

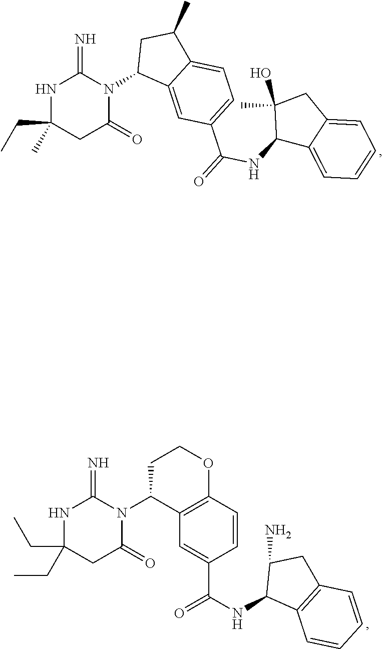 [SMILES: CC[C@]1(C)CC(=O)N([C@@H]2C[C@@H](C)c3ccc(C(=O)N[C@@H]4c5ccccc5C[C@]4(C)O)cc32)C(=N)N1 ; CCC1(CC)CC(=O)N([C@@H]2CCOc3ccc(C(=O)N[C@@H]4c5ccccc5C[C@H]4N)cc32)C(=N)N1]

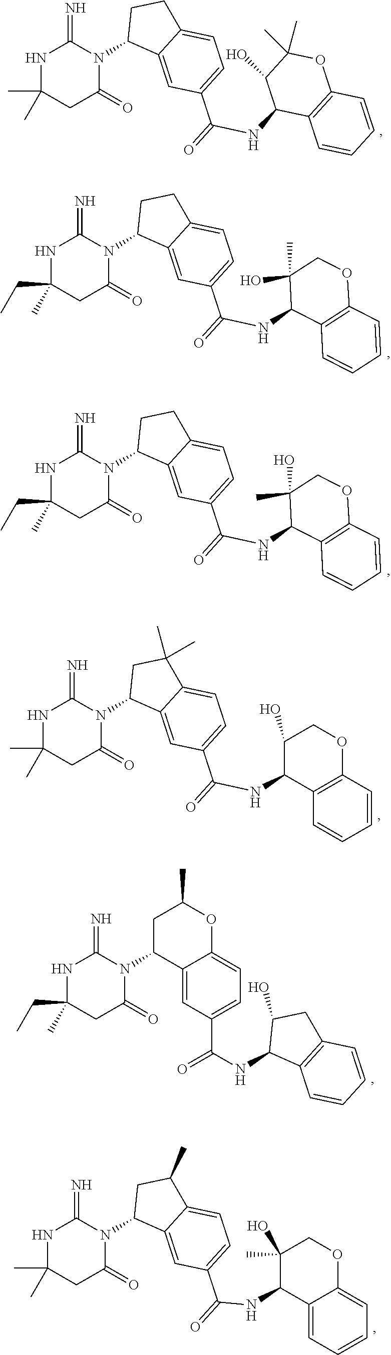 [SMILES: CC1(C)CC(=O)N([C@@H]2CCc3ccc(C(=O)N[C@@H]4c5ccccc5OC(C)(C)[C@H]4O)cc32)C(=N)N1 ; CC[C@]1(C)CC(=O)N([C@@H]2CCc3ccc(C(=O)N[C@@H]4c5ccccc5OC[C@]4(C)O)cc32)C(=N)N1 ; CC[C@]1(C)CC(=O)N([C@@H]2CCc3ccc(C(=O)N[C@@H]4c5ccccc5OC[C@@]4(C)O)cc32)C(=N)N1 ; CC1(C)CC(=O)N([C@@H]2CC(C)(C)c3ccc(C(=O)N[C@@H]4c5ccccc5OC[C@H]4O)cc32)C(=N)N1 ; CC[C@]1(C)CC(=O)N([C@@H]2C[C@@H](C)Oc3ccc(C(=O)N[C@@H]4c5ccccc5C[C@H]4O)cc32)C(=N)N1 ; C[C@@H]1C[C@@H](N2C(=N)NC(C)(C)CC2=O)c2cc(C(=O)N[C@@H]3c4ccccc4OC[C@]3(C)O)ccc21]

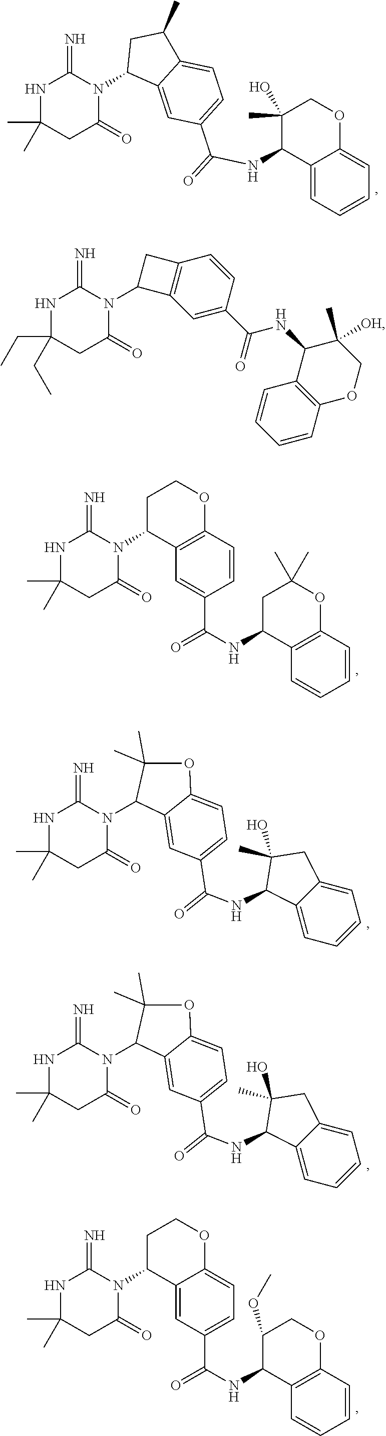 [SMILES: C[C@@H]1C[C@@H](N2C(=N)NC(C)(C)CC2=O)c2cc(C(=O)N[C@@H]3c4ccccc4OC[C@@]3(C)O)ccc21 ; CCC1(CC)CC(=O)N(C2Cc3ccc(C(=O)N[C@@H]4c5ccccc5OC[C@@]4(C)O)cc32)C(=N)N1 ; CC1(C)CC(=O)N([C@@H]2CCOc3ccc(C(=O)N[C@H]4CC(C)(C)Oc5ccccc54)cc32)C(=N)N1 ; CC1(C)CC(=O)N(C2c3cc(C(=O)N[C@@H]4c5ccccc5C[C@@]4(C)O)ccc3OC2(C)C)C(=N)N1 ; CC1(C)CC(=O)N(C2c3cc(C(=O)N[C@@H]4c5ccccc5C[C@]4(C)O)ccc3OC2(C)C)C(=N)N1 ; CO[C@@H]1COc2ccccc2[C@H]1NC(=O)c1ccc2c(c1)[C@H](N1C(=N)NC(C)(C)CC1=O)CCO2]

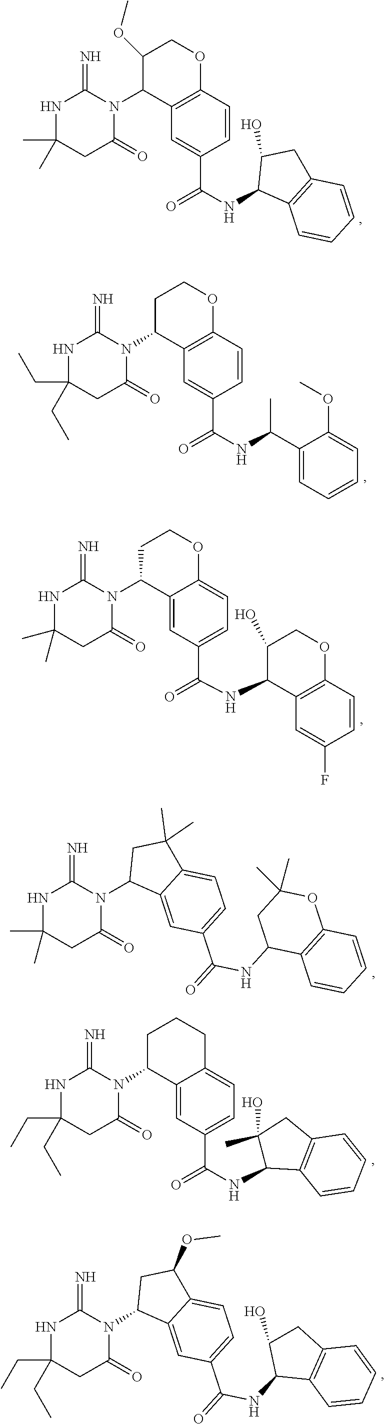

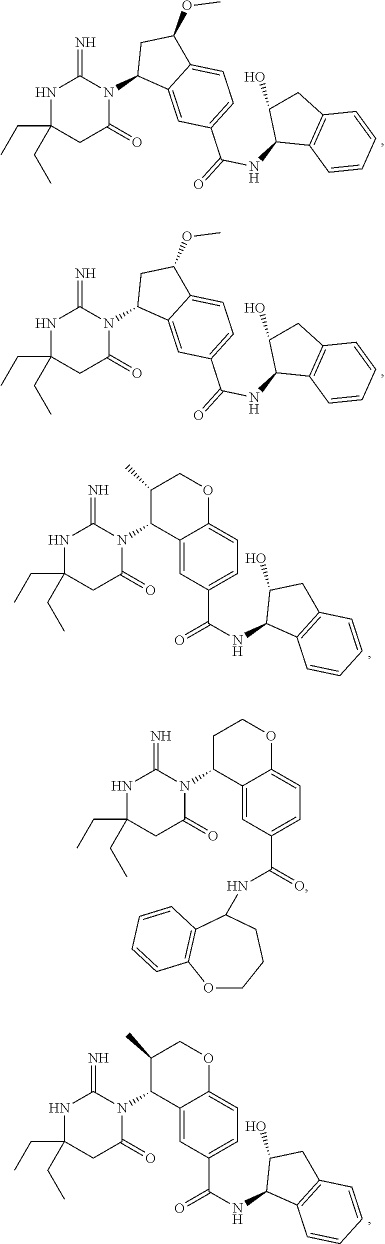 [SMILES: CCC1(CC)CC(=O)N([C@H]2C[C@@H](OC)c3ccc(C(=O)N[C@@H]4c5ccccc5C[C@H]4O)cc32)C(=N)N1 ; CCC1(CC)CC(=O)N([C@@H]2C[C@H](OC)c3ccc(C(=O)N[C@@H]4c5ccccc5C[C@H]4O)cc32)C(=N)N1 ; CCC1(CC)CC(=O)N([C@H]2c3cc(C(=O)N[C@@H]4c5ccccc5C[C@H]4O)ccc3OC[C@H]2C)C(=N)N1 ; CCC1(CC)CC(=O)N([C@@H]2CCOc3ccc(C(=O)NC4CCCOc5ccccc54)cc32)C(=N)N1 ; CCC1(CC)CC(=O)N([C@H]2c3cc(C(=O)N[C@@H]4c5ccccc5C[C@H]4O)ccc3OC[C@@H]2C)C(=N)N1]

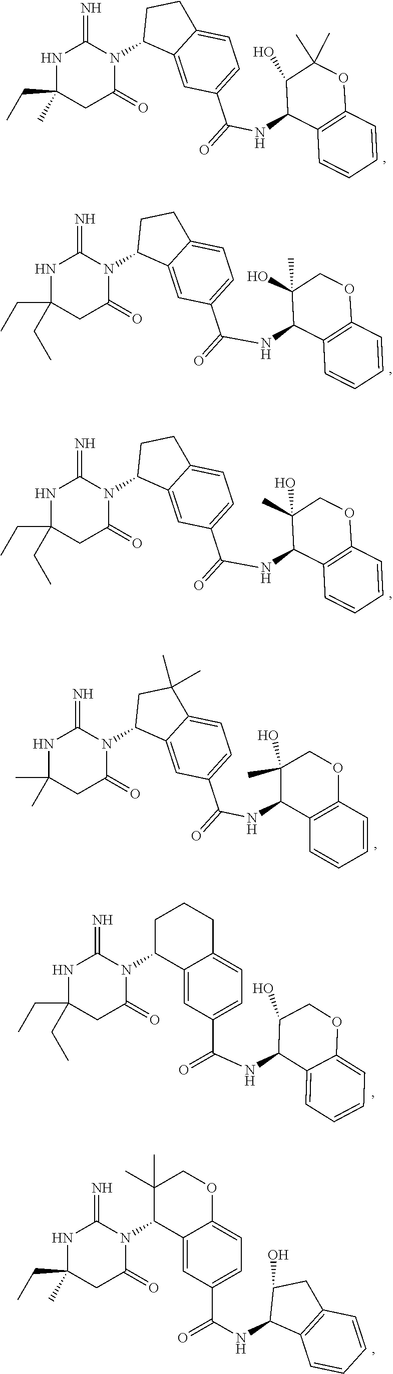 [SMILES: CC[C@]1(C)CC(=O)N([C@@H]2CCc3ccc(C(=O)N[C@@H]4c5ccccc5OC(C)(C)[C@H]4O)cc32)C(=N)N1 ; CCC1(CC)CC(=O)N([C@@H]2CCc3ccc(C(=O)N[C@@H]4c5ccccc5OC[C@]4(C)O)cc32)C(=N)N1 ; CCC1(CC)CC(=O)N([C@@H]2CCc3ccc(C(=O)N[C@@H]4c5ccccc5OC[C@@]4(C)O)cc32)C(=N)N1 ; CC1(C)CC(=O)N([C@@H]2CC(C)(C)c3ccc(C(=O)N[C@@H]4c5ccccc5OC[C@@]4(C)O)cc32)C(=N)N1 ; CCC1(CC)CC(=O)N([C@@H]2CCCc3ccc(C(=O)N[C@@H]4c5ccccc5OC[C@H]4O)cc32)C(=N)N1 ; CC[C@]1(C)CC(=O)N([C@H]2c3cc(C(=O)N[C@@H]4c5ccccc5C[C@H]4O)ccc3OCC2(C)C)C(=N)N1]

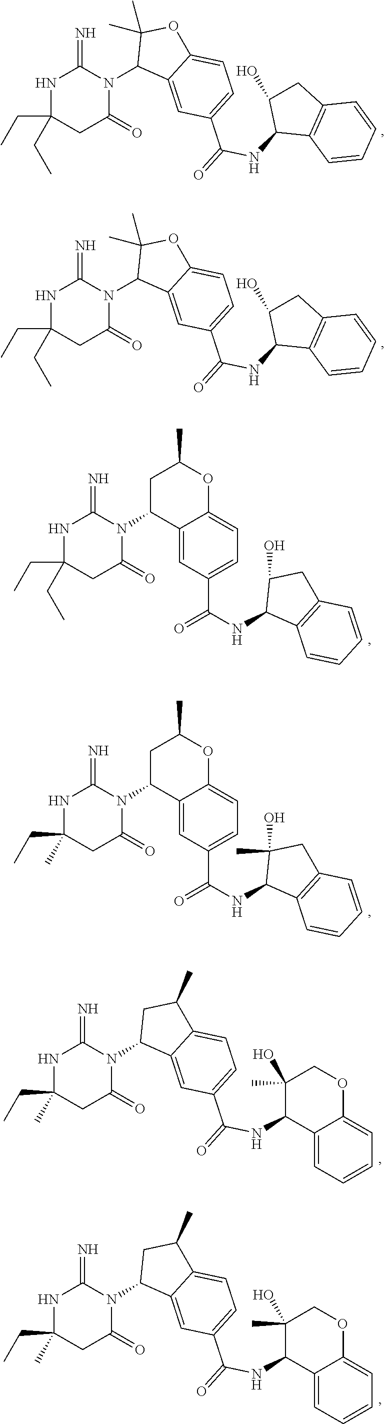

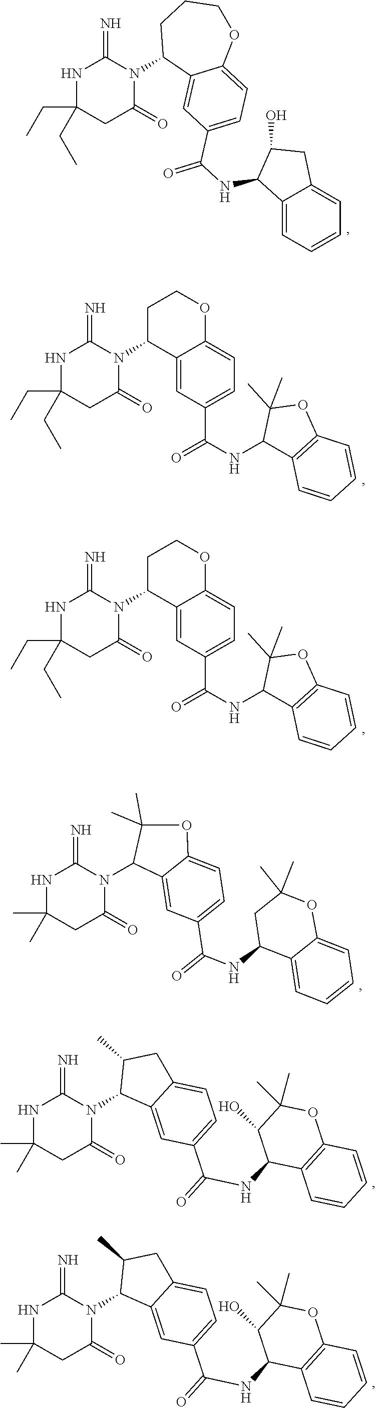 [SMILES: CCC1(CC)CC(=O)N([C@@H]2CCCOc3ccc(C(=O)N[C@@H]4c5ccccc5C[C@H]4O)cc32)C(=N)N1 ; CCC1(CC)CC(=O)N([C@@H]2CCOc3ccc(C(=O)NC4c5ccccc5OC4(C)C)cc32)C(=N)N1 ; CCC1(CC)CC(=O)N([C@@H]2CCOc3ccc(C(=O)NC4c5ccccc5OC4(C)C)cc32)C(=N)N1 ; CC1(C)CC(=O)N(C2c3cc(C(=O)N[C@H]4CC(C)(C)Oc5ccccc54)ccc3OC2(C)C)C(=N)N1 ; C[C@@H]1Cc2ccc(C(=O)N[C@@H]3c4ccccc4OC(C)(C)[C@H]3O)cc2[C@@H]1N1C(=N)NC(C)(C)CC1=O ; C[C@H]1Cc2ccc(C(=O)N[C@@H]3c4ccccc4OC(C)(C)[C@H]3O)cc2[C@@H]1N1C(=N)NC(C)(C)CC1=O]

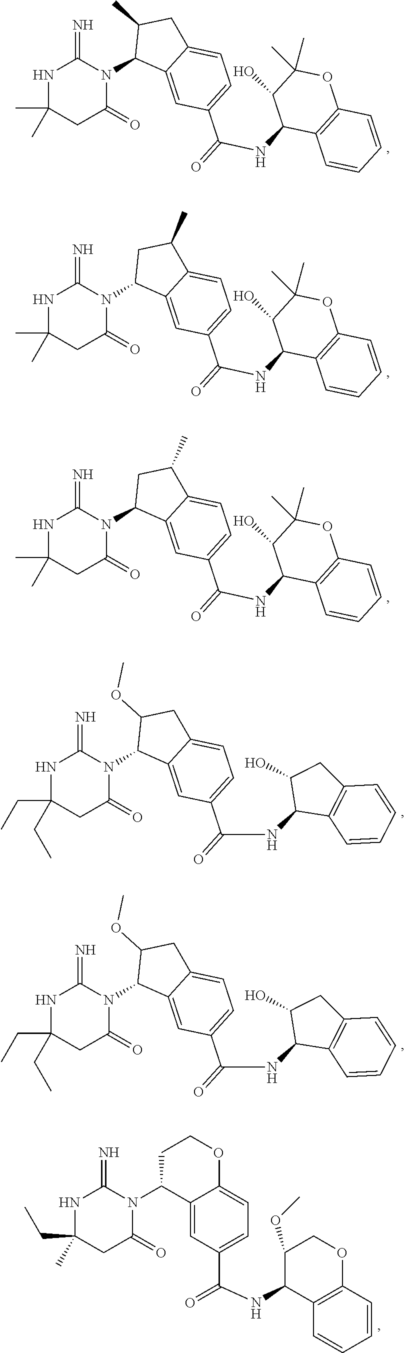

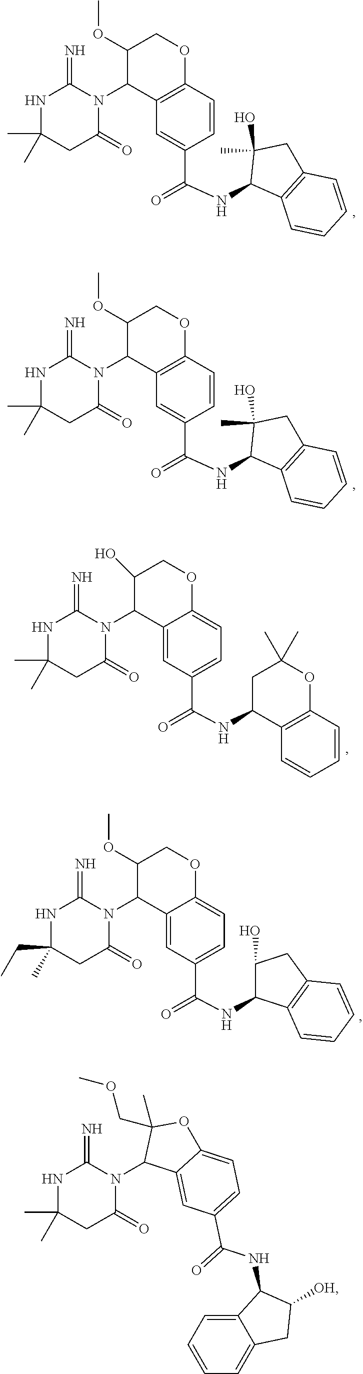 [SMILES: COC1COc2ccc(C(=O)N[C@@H]3c4ccccc4C[C@]3(C)O)cc2C1N1C(=N)NC(C)(C)CC1=O ; COC1COc2ccc(C(=O)N[C@@H]3c4ccccc4C[C@@]3(C)O)cc2C1N1C(=N)NC(C)(C)CC1=O ; CC1(C)CC(=O)N(C2c3cc(C(=O)N[C@H]4CC(C)(C)Oc5ccccc54)ccc3OCC2O)C(=N)N1 ; CC[C@]1(C)CC(=O)N(C2c3cc(C(=O)N[C@@H]4c5ccccc5C[C@H]4O)ccc3OCC2OC)C(=N)N1 ; COCC1(C)Oc2ccc(C(=O)N[C@@H]3c4ccccc4C[C@H]3O)cc2C1N1C(=N)NC(C)(C)CC1=O]

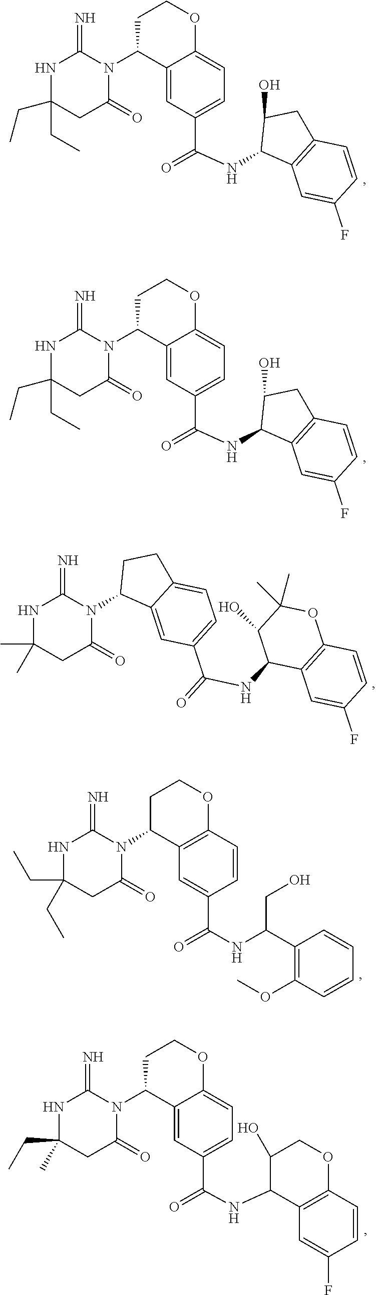 [SMILES: CCC1(CC)CC(=O)N([C@@H]2CCOc3ccc(C(=O)N[C@H]4c5cc(F)ccc5C[C@@H]4O)cc32)C(=N)N1 ; CCC1(CC)CC(=O)N([C@@H]2CCOc3ccc(C(=O)N[C@@H]4c5cc(F)ccc5C[C@H]4O)cc32)C(=N)N1 ; CC1(C)CC(=O)N([C@@H]2CCc3ccc(C(=O)N[C@@H]4c5cc(F)ccc5OC(C)(C)[C@H]4O)cc32)C(=N)N1 ; CCC1(CC)CC(=O)N([C@@H]2CCOc3ccc(C(=O)NC(CO)c4ccccc4OC)cc32)C(=N)N1 ; CC[C@]1(C)CC(=O)N([C@@H]2CCOc3ccc(C(=O)NC4c5cc(F)ccc5OCC4O)cc32)C(=N)N1]

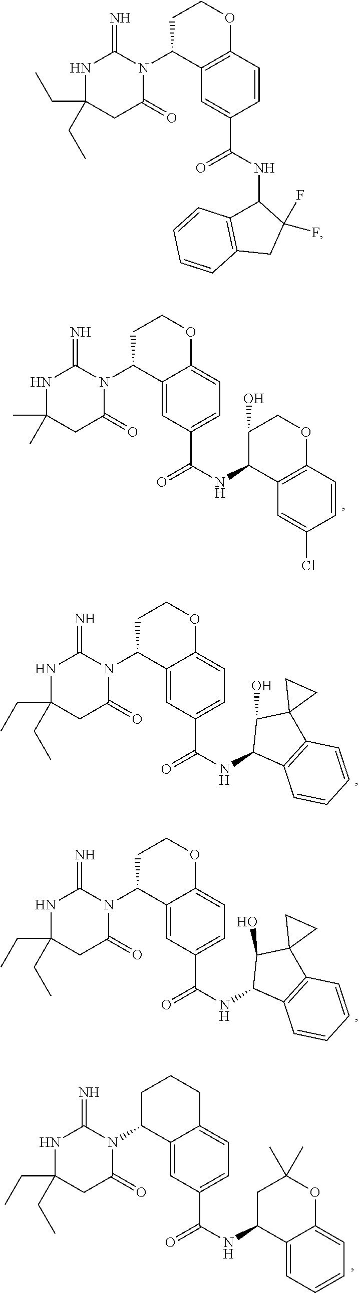 [SMILES: CCC1(CC)CC(=O)N([C@@H]2CCOc3ccc(C(=O)NC4c5ccccc5CC4(F)F)cc32)C(=N)N1 ; CC1(C)CC(=O)N([C@@H]2CCOc3ccc(C(=O)N[C@@H]4c5cc(Cl)ccc5OC[C@H]4O)cc32)C(=N)N1 ; CCC1(CC)CC(=O)N([C@@H]2CCOc3ccc(C(=O)N[C@@H]4c5ccccc5C5(CC5)[C@H]4O)cc32)C(=N)N1 ; CCC1(CC)CC(=O)N([C@@H]2CCOc3ccc(C(=O)N[C@H]4c5ccccc5C5(CC5)[C@@H]4O)cc32)C(=N)N1 ; CCC1(CC)CC(=O)N([C@@H]2CCCc3ccc(C(=O)N[C@H]4CC(C)(C)Oc5ccccc54)cc32)C(=N)N1]

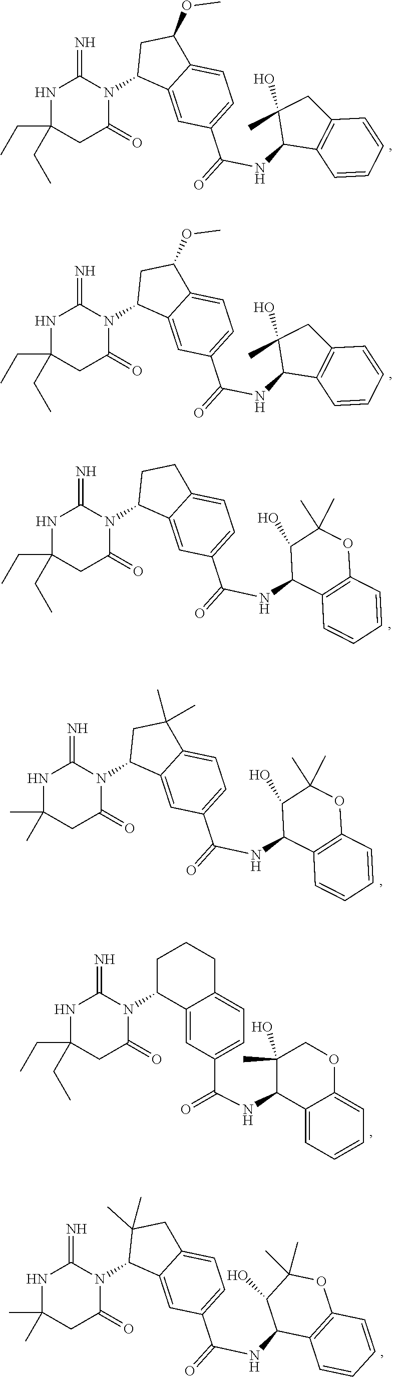

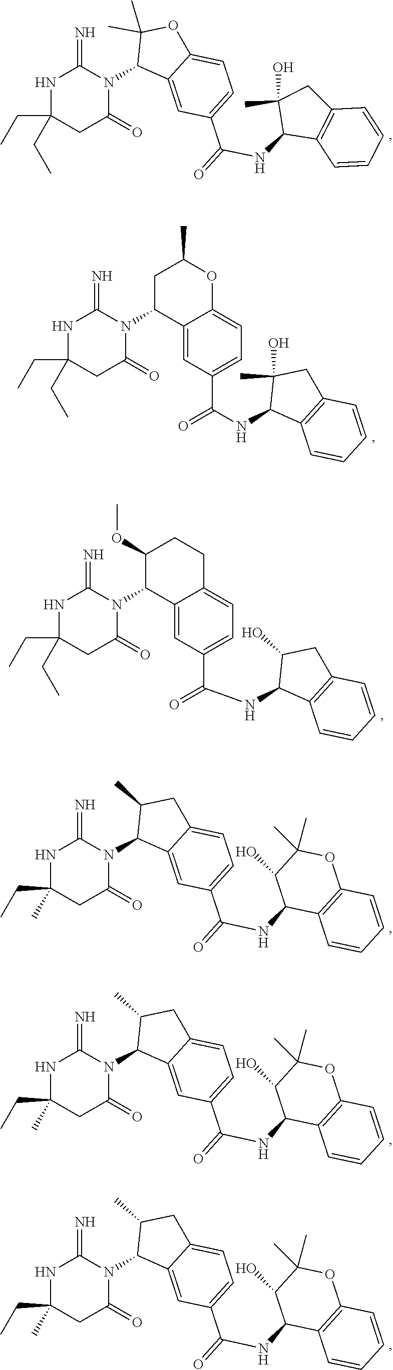

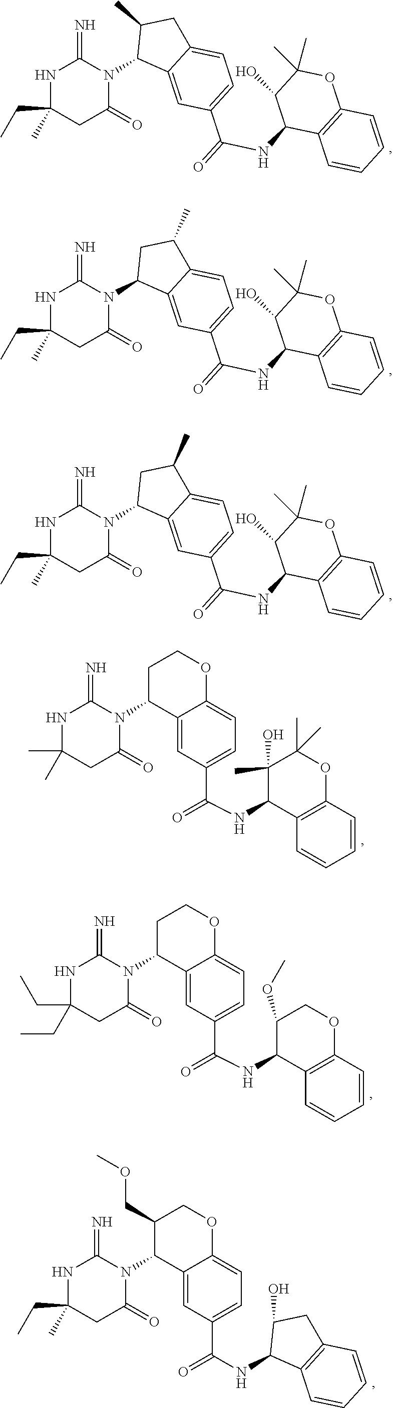

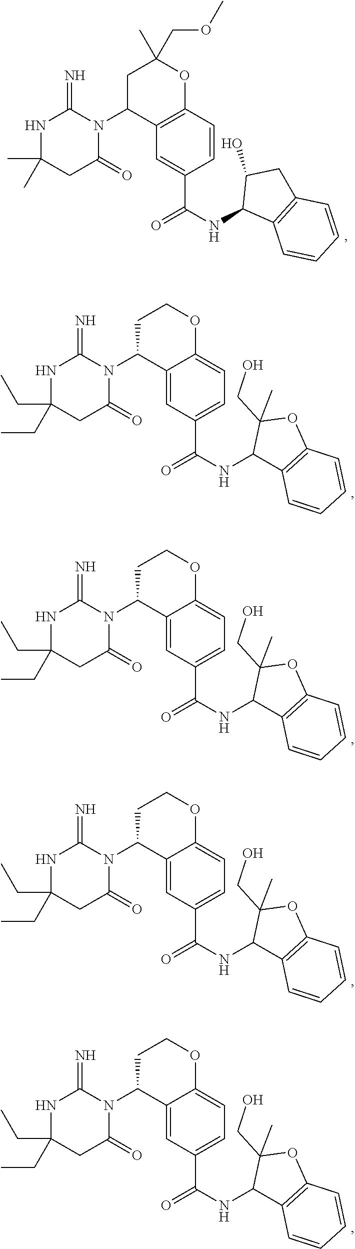 [SMILES: COCC1(C)CC(N2C(=N)NC(C)(C)CC2=O)c2cc(C(=O)N[C@@H]3c4ccccc4C[C@H]3O)ccc2O1 ; CCC1(CC)CC(=O)N([C@@H]2CCOc3ccc(C(=O)NC4c5ccccc5OC4(C)CO)cc32)C(=N)N1 ; CCC1(CC)CC(=O)N([C@@H]2CCOc3ccc(C(=O)NC4c5ccccc5OC4(C)CO)cc32)C(=N)N1 ; CCC1(CC)CC(=O)N([C@@H]2CCOc3ccc(C(=O)NC4c5ccccc5OC4(C)CO)cc32)C(=N)N1 ; CCC1(CC)CC(=O)N([C@@H]2CCOc3ccc(C(=O)NC4c5ccccc5OC4(C)CO)cc32)C(=N)N1]

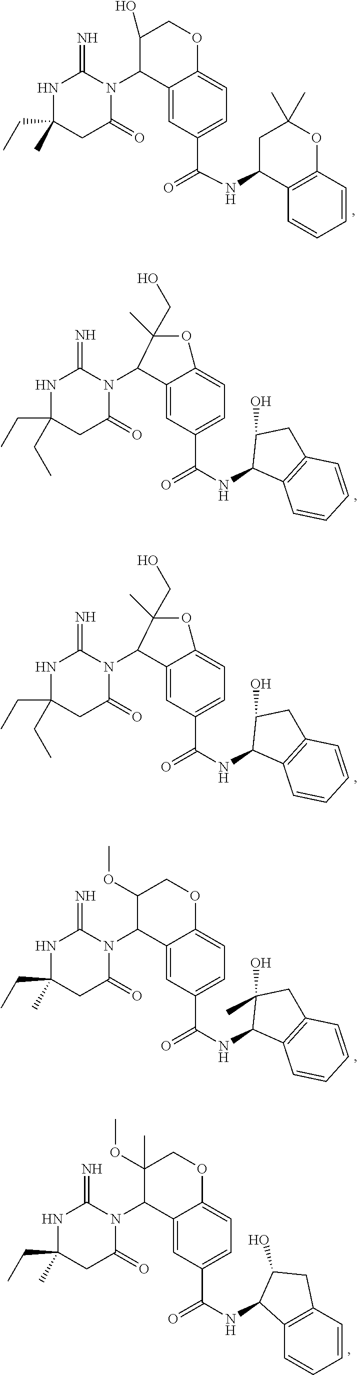 [SMILES: CC[C@@]1(C)CC(=O)N(C2c3cc(C(=O)N[C@H]4CC(C)(C)Oc5ccccc54)ccc3OCC2O)C(=N)N1 ; CCC1(CC)CC(=O)N(C2c3cc(C(=O)N[C@@H]4c5ccccc5C[C@H]4O)ccc3OC2(C)CO)C(=N)N1 ; CCC1(CC)CC(=O)N(C2c3cc(C(=O)N[C@@H]4c5ccccc5C[C@H]4O)ccc3OC2(C)CO)C(=N)N1 ; CC[C@]1(C)CC(=O)N(C2c3cc(C(=O)N[C@@H]4c5ccccc5C[C@@]4(C)O)ccc3OCC2OC)C(=N)N1 ; CC[C@]1(C)CC(=O)N(C2c3cc(C(=O)N[C@@H]4c5ccccc5C[C@H]4O)ccc3OCC2(C)OC)C(=N)N1]

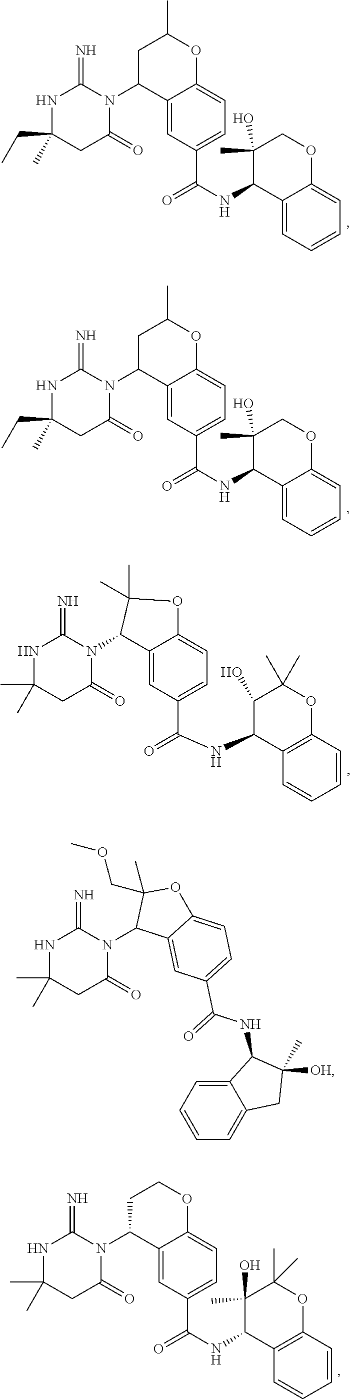 [SMILES: CC[C@]1(C)CC(=O)N(C2CC(C)Oc3ccc(C(=O)N[C@@H]4c5ccccc5OC[C@@]4(C)O)cc32)C(=N)N1 ; CC[C@]1(C)CC(=O)N(C2CC(C)Oc3ccc(C(=O)N[C@@H]4c5ccccc5OC[C@@]4(C)O)cc32)C(=N)N1 ; CC1(C)CC(=O)N([C@H]2c3cc(C(=O)N[C@@H]4c5ccccc5OC(C)(C)[C@H]4O)ccc3OC2(C)C)C(=N)N1 ; COCC1(C)Oc2ccc(C(=O)N[C@@H]3c4ccccc4C[C@]3(C)O)cc2C1N1C(=N)NC(C)(C)CC1=O ; CC1(C)CC(=O)N([C@@H]2CCOc3ccc(C(=O)N[C@H]4c5ccccc5OC(C)(C)[C@]4(C)O)cc32)C(=N)N1]

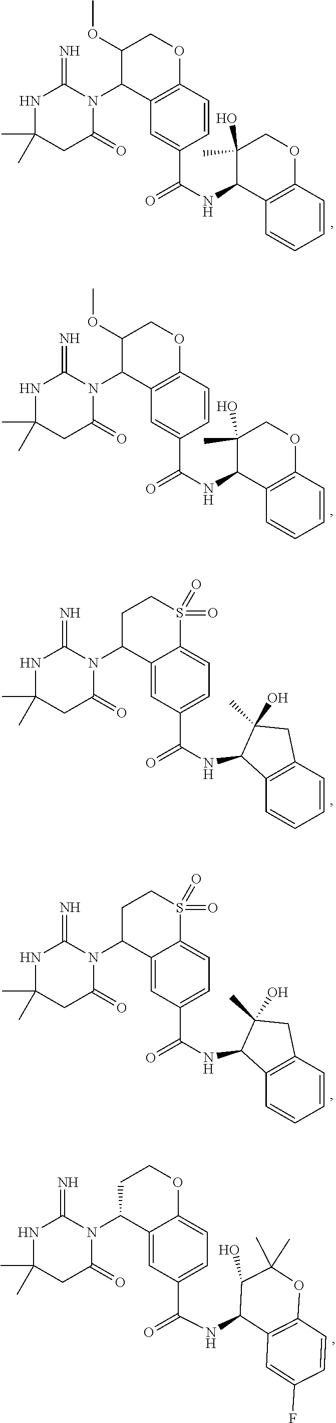 [SMILES: COC1COc2ccc(C(=O)N[C@@H]3c4ccccc4OC[C@]3(C)O)cc2C1N1C(=N)NC(C)(C)CC1=O ; COC1COc2ccc(C(=O)N[C@@H]3c4ccccc4OC[C@@]3(C)O)cc2C1N1C(=N)NC(C)(C)CC1=O ; CC1(C)CC(=O)N(C2CCS(=O)(=O)c3ccc(C(=O)N[C@@H]4c5ccccc5C[C@]4(C)O)cc32)C(=N)N1 ; CC1(C)CC(=O)N(C2CCS(=O)(=O)c3ccc(C(=O)N[C@@H]4c5ccccc5C[C@@]4(C)O)cc32)C(=N)N1 ; CC1(C)CC(=O)N([C@@H]2CCOc3ccc(C(=O)N[C@@H]4c5cc(F)ccc5OC(C)(C)[C@H]4O)cc32)C(=N)N1]

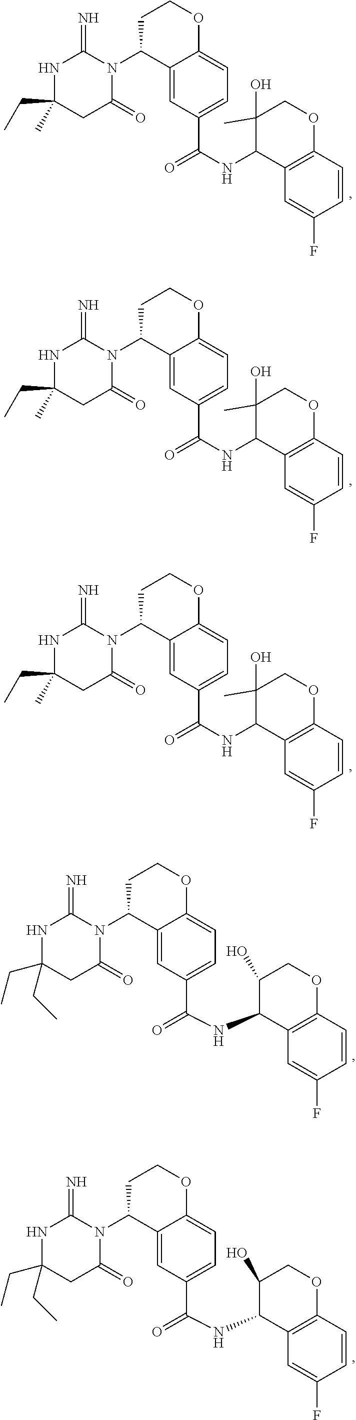 [SMILES: CC[C@]1(C)CC(=O)N([C@@H]2CCOc3ccc(C(=O)NC4c5cc(F)ccc5OCC4(C)O)cc32)C(=N)N1 ; CC[C@]1(C)CC(=O)N([C@@H]2CCOc3ccc(C(=O)NC4c5cc(F)ccc5OCC4(C)O)cc32)C(=N)N1 ; CC[C@]1(C)CC(=O)N([C@@H]2CCOc3ccc(C(=O)NC4c5cc(F)ccc5OCC4(C)O)cc32)C(=N)N1 ; CCC1(CC)CC(=O)N([C@@H]2CCOc3ccc(C(=O)N[C@@H]4c5cc(F)ccc5OC[C@H]4O)cc32)C(=N)N1 ; CCC1(CC)CC(=O)N([C@@H]2CCOc3ccc(C(=O)N[C@H]4c5cc(F)ccc5OC[C@@H]4O)cc32)C(=N)N1]

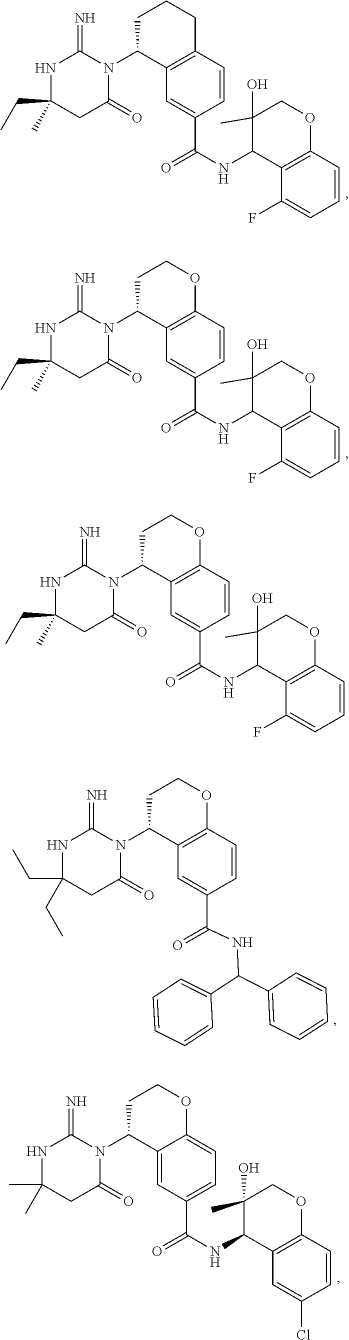 [SMILES: CC[C@]1(C)CC(=O)N([C@@H]2CCCc3ccc(C(=O)NC4c5c(F)cccc5OCC4(C)O)cc32)C(=N)N1 ; CC[C@]1(C)CC(=O)N([C@@H]2CCOc3ccc(C(=O)NC4c5c(F)cccc5OCC4(C)O)cc32)C(=N)N1 ; CC[C@]1(C)CC(=O)N([C@@H]2CCOc3ccc(C(=O)NC4c5c(F)cccc5OCC4(C)O)cc32)C(=N)N1 ; CCC1(CC)CC(=O)N([C@@H]2CCOc3ccc(C(=O)NC(c4ccccc4)c4ccccc4)cc32)C(=N)N1 ; CC1(C)CC(=O)N([C@@H]2CCOc3ccc(C(=O)N[C@@H]4c5cc(Cl)ccc5OC[C@@]4(C)O)cc32)C(=N)N1]

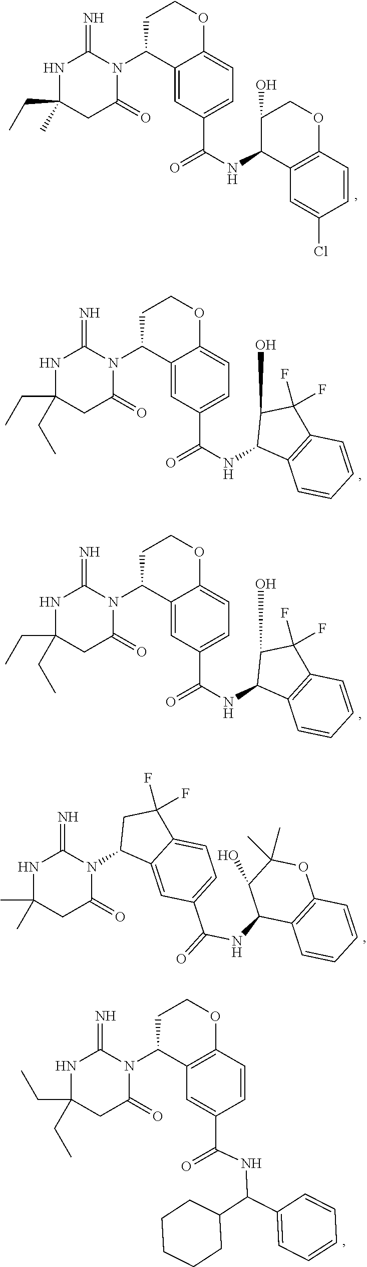

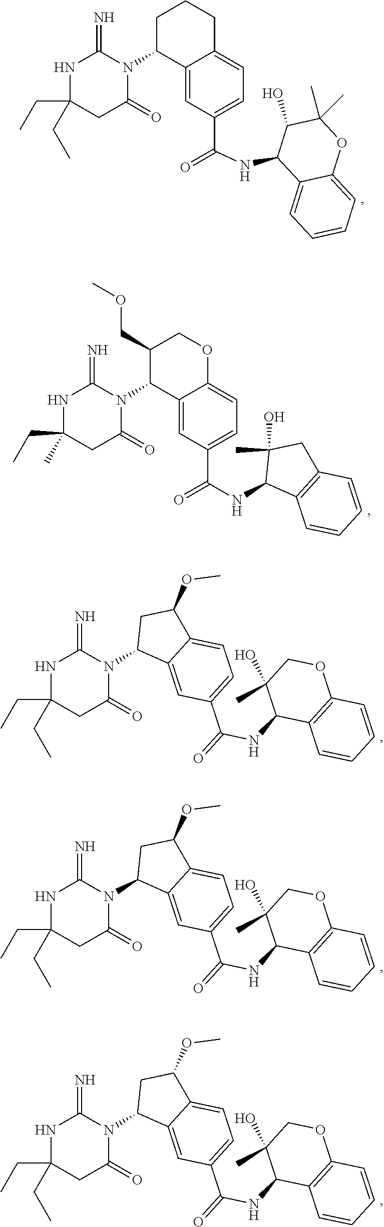 [SMILES: CCC1(CC)CC(=O)N([C@@H]2CCCc3ccc(C(=O)N[C@@H]4c5ccccc5OC(C)(C)[C@H]4O)cc32)C(=N)N1 ; CC[C@]1(C)CC(=O)N([C@H]2c3cc(C(=O)N[C@@H]4c5ccccc5C[C@@]4(C)O)ccc3OC[C@@H]2COC)C(=N)N1 ; CCC1(CC)CC(=O)N([C@@H]2C[C@@H](OC)c3ccc(C(=O)N[C@@H]4c5ccccc5OC[C@@]4(C)O)cc32)C(=N)N1 ; CCC1(CC)CC(=O)N([C@H]2C[C@@H](OC)c3ccc(C(=O)N[C@@H]4c5ccccc5OC[C@@]4(C)O)cc32)C(=N)N1 ; CCC1(CC)CC(=O)N([C@@H]2C[C@H](OC)c3ccc(C(=O)N[C@@H]4c5ccccc5OC[C@@]4(C)O)cc32)C(=N)N1]

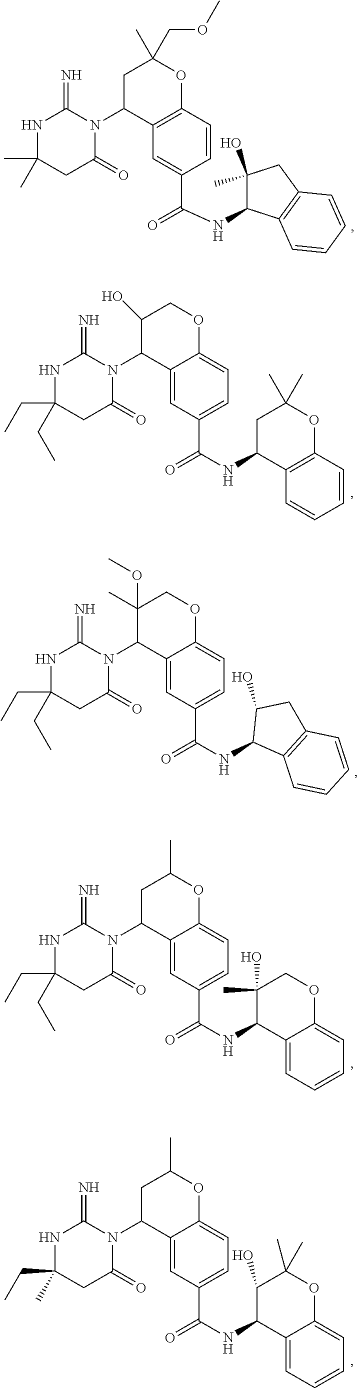 [SMILES: COCC1(C)CC(N2C(=N)NC(C)(C)CC2=O)c2cc(C(=O)N[C@@H]3c4ccccc4C[C@]3(C)O)ccc2O1 ; CCC1(CC)CC(=O)N(C2c3cc(C(=O)N[C@H]4CC(C)(C)Oc5ccccc54)ccc3OCC2O)C(=N)N1 ; CCC1(CC)CC(=O)N(C2c3cc(C(=O)N[C@@H]4c5ccccc5C[C@H]4O)ccc3OCC2(C)OC)C(=N)N1 ; CCC1(CC)CC(=O)N(C2CC(C)Oc3ccc(C(=O)N[C@@H]4c5ccccc5OC[C@@]4(C)O)cc32)C(=N)N1 ; CC[C@]1(C)CC(=O)N(C2CC(C)Oc3ccc(C(=O)N[C@@H]4c5ccccc5OC(C)(C)[C@H]4O)cc32)C(=N)N1]

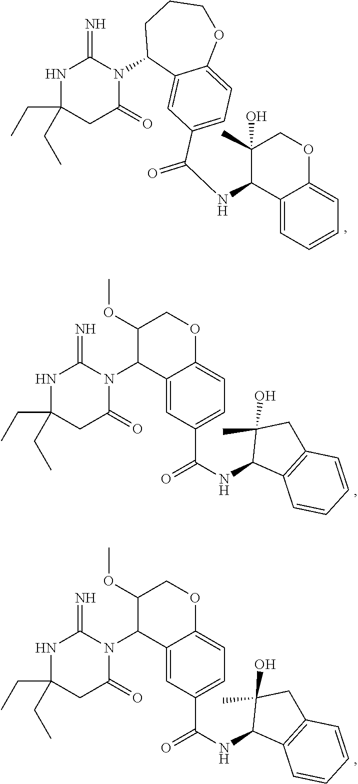 [SMILES: CCC1(CC)CC(=O)N([C@@H]2CCCOc3ccc(C(=O)N[C@@H]4c5ccccc5OC[C@@]4(C)O)cc32)C(=N)N1 ; CCC1(CC)CC(=O)N(C2c3cc(C(=O)N[C@@H]4c5ccccc5C[C@@]4(C)O)ccc3OCC2OC)C(=N)N1 ; CCC1(CC)CC(=O)N(C2c3cc(C(=O)N[C@@H]4c5ccccc5C[C@]4(C)O)ccc3OCC2OC)C(=N)N1]

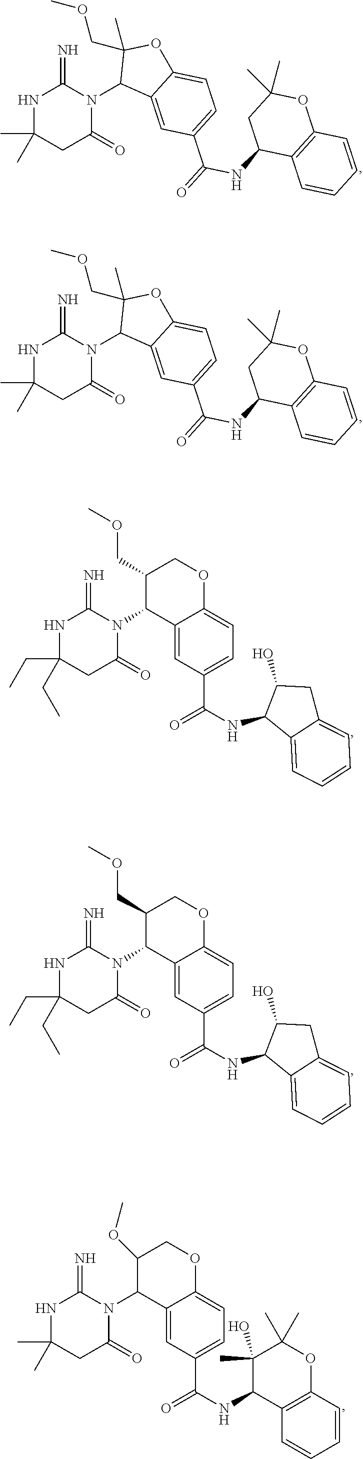 [SMILES: COCC1(C)Oc2ccc(C(=O)N[C@H]3CC(C)(C)Oc4ccccc43)cc2C1N1C(=N)NC(C)(C)CC1=O ; COCC1(C)Oc2ccc(C(=O)N[C@H]3CC(C)(C)Oc4ccccc43)cc2C1N1C(=N)NC(C)(C)CC1=O ; CCC1(CC)CC(=O)N([C@H]2c3cc(C(=O)N[C@@H]4c5ccccc5C[C@H]4O)ccc3OC[C@H]2COC)C(=N)N1 ; CCC1(CC)CC(=O)N([C@H]2c3cc(C(=O)N[C@@H]4c5ccccc5C[C@H]4O)ccc3OC[C@@H]2COC)C(=N)N1 ; COC1COc2ccc(C(=O)N[C@@H]3c4ccccc4OC(C)(C)[C@@]3(C)O)cc2C1N1C(=N)NC(C)(C)CC1=O]

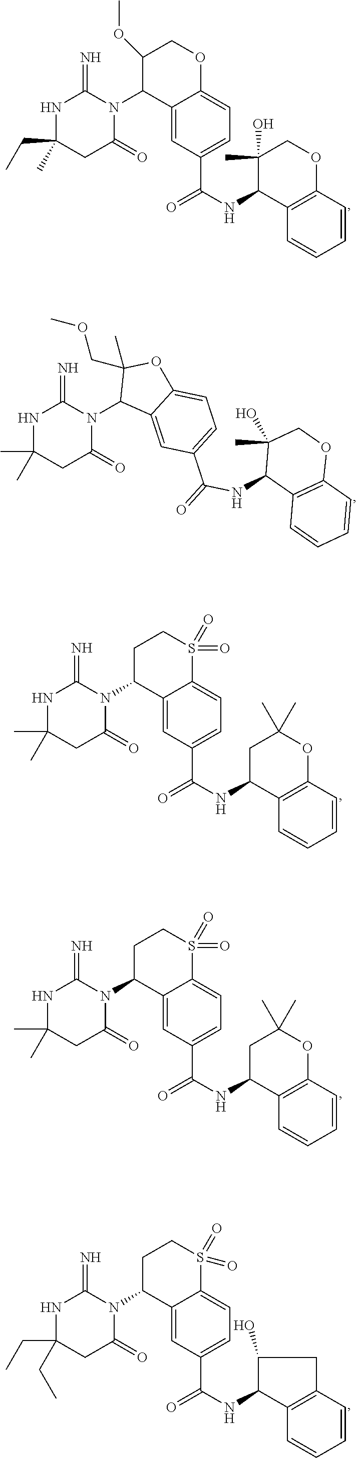 [SMILES: CC[C@]1(C)CC(=O)N(C2c3cc(C(=O)N[C@@H]4c5ccccc5OC[C@@]4(C)O)ccc3OCC2OC)C(=N)N1 ; COCC1(C)Oc2ccc(C(=O)N[C@@H]3c4ccccc4OC[C@@]3(C)O)cc2C1N1C(=N)NC(C)(C)CC1=O ; CC1(C)CC(=O)N([C@@H]2CCS(=O)(=O)c3ccc(C(=O)N[C@H]4CC(C)(C)Oc5ccccc54)cc32)C(=N)N1 ; CC1(C)CC(=O)N([C@H]2CCS(=O)(=O)c3ccc(C(=O)N[C@H]4CC(C)(C)Oc5ccccc54)cc32)C(=N)N1 ; CCC1(CC)CC(=O)N([C@@H]2CCS(=O)(=O)c3ccc(C(=O)N[C@@H]4c5ccccc5C[C@H]4O)cc32)C(=N)N1]

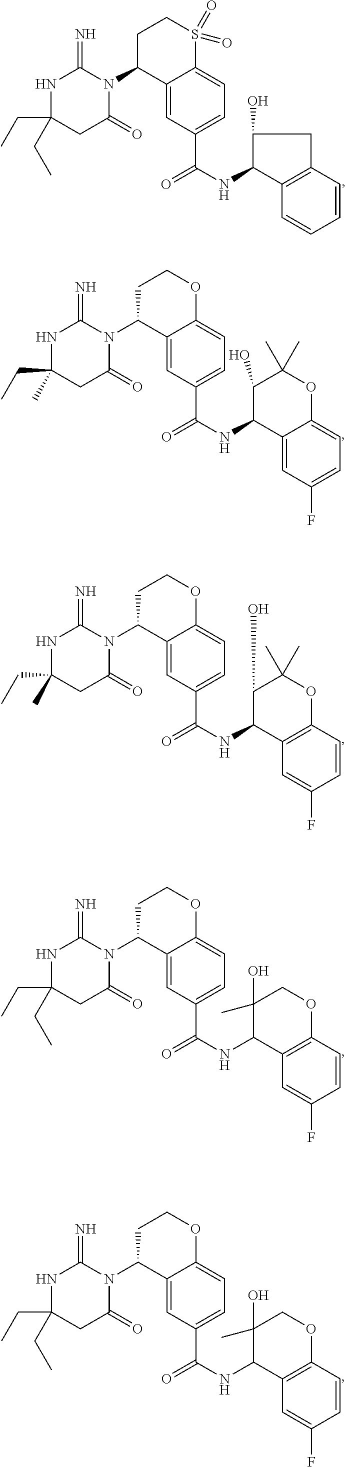 [SMILES: CCC1(CC)CC(=O)N([C@H]2CCS(=O)(=O)c3ccc(C(=O)N[C@@H]4c5ccccc5C[C@H]4O)cc32)C(=N)N1 ; CC[C@]1(C)CC(=O)N([C@@H]2CCOc3ccc(C(=O)N[C@@H]4c5cc(F)ccc5OC(C)(C)[C@H]4O)cc32)C(=N)N1 ; CC[C@@]1(C)CC(=O)N([C@@H]2CCOc3ccc(C(=O)N[C@@H]4c5cc(F)ccc5OC(C)(C)[C@H]4O)cc32)C(=N)N1 ; CCC1(CC)CC(=O)N([C@@H]2CCOc3ccc(C(=O)NC4c5cc(F)ccc5OCC4(C)O)cc32)C(=N)N1 ; CCC1(CC)CC(=O)N([C@@H]2CCOc3ccc(C(=O)NC4c5cc(F)ccc5OCC4(C)O)cc32)C(=N)N1]

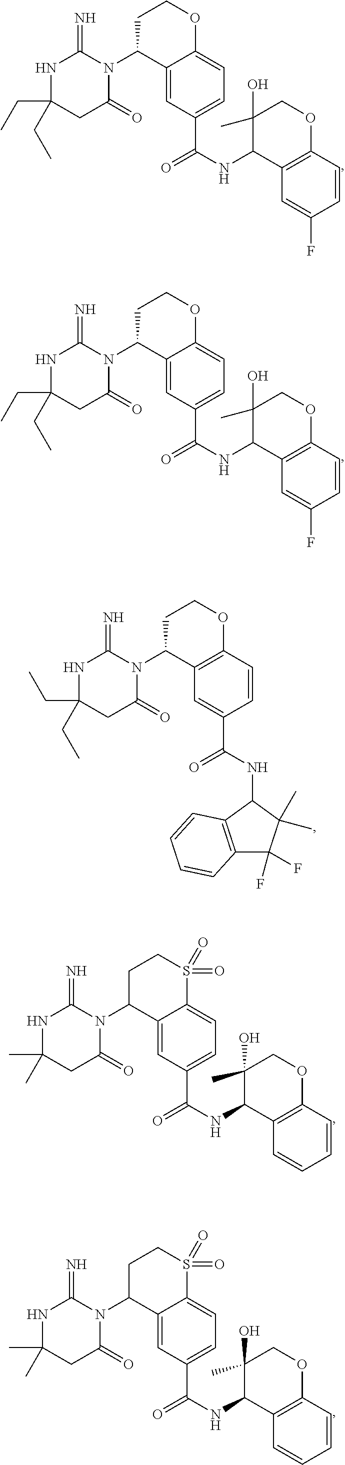

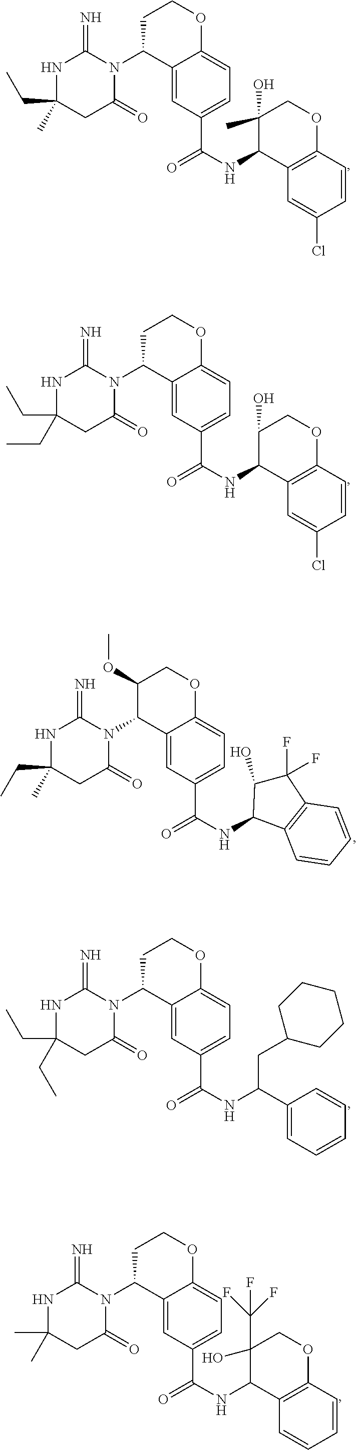 [SMILES: CC[C@]1(C)CC(=O)N([C@@H]2CCOc3ccc(C(=O)N[C@@H]4c5cc(Cl)ccc5OC[C@@]4(C)O)cc32)C(=N)N1 ; CCC1(CC)CC(=O)N([C@@H]2CCOc3ccc(C(=O)N[C@@H]4c5cc(Cl)ccc5OC[C@H]4O)cc32)C(=N)N1 ; CC[C@]1(C)CC(=O)N([C@H]2c3cc(C(=O)N[C@@H]4c5ccccc5C(F)(F)[C@H]4O)ccc3OC[C@@H]2OC)C(=N)N1 ; CCC1(CC)CC(=O)N([C@@H]2CCOc3ccc(C(=O)NC(CC4CCCCC4)c4ccccc4)cc32)C(=N)N1 ; CC1(C)CC(=O)N([C@@H]2CCOc3ccc(C(=O)NC4c5ccccc5OCC4(O)C(F)(F)F)cc32)C(=N)N1]

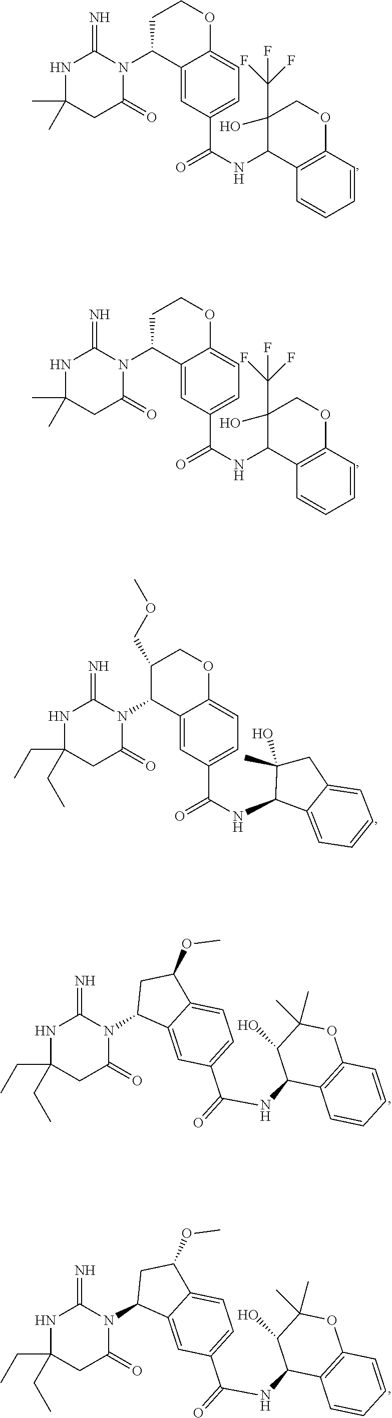 [SMILES: CC1(C)CC(=O)N([C@@H]2CCOc3ccc(C(=O)NC4c5ccccc5OCC4(O)C(F)(F)F)cc32)C(=N)N1 ; CC1(C)CC(=O)N([C@@H]2CCOc3ccc(C(=O)NC4c5ccccc5OCC4(O)C(F)(F)F)cc32)C(=N)N1 ; CCC1(CC)CC(=O)N([C@H]2c3cc(C(=O)N[C@@H]4c5ccccc5C[C@@]4(C)O)ccc3OC[C@H]2COC)C(=N)N1 ; CCC1(CC)CC(=O)N([C@@H]2C[C@@H](OC)c3ccc(C(=O)N[C@@H]4c5ccccc5OC(C)(C)[C@H]4O)cc32)C(=N)N1 ; CCC1(CC)CC(=O)N([C@H]2C[C@H](OC)c3ccc(C(=O)N[C@@H]4c5ccccc5OC(C)(C)[C@H]4O)cc32)C(=N)N1]

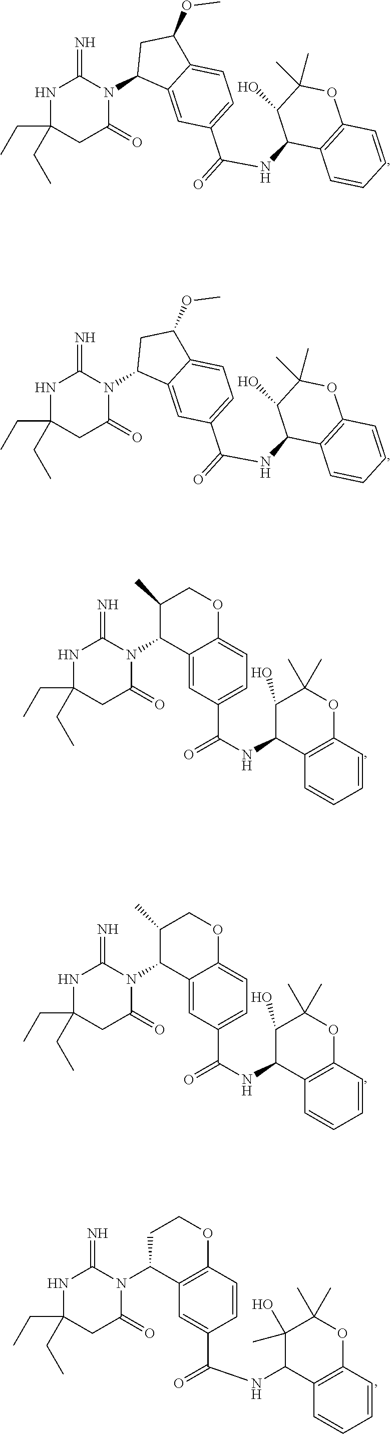 [SMILES: CCC1(CC)CC(=O)N([C@H]2C[C@@H](OC)c3ccc(C(=O)N[C@@H]4c5ccccc5OC(C)(C)[C@H]4O)cc32)C(=N)N1 ; CCC1(CC)CC(=O)N([C@@H]2C[C@H](OC)c3ccc(C(=O)N[C@@H]4c5ccccc5OC(C)(C)[C@H]4O)cc32)C(=N)N1 ; CCC1(CC)CC(=O)N([C@H]2c3cc(C(=O)N[C@@H]4c5ccccc5OC(C)(C)[C@H]4O)ccc3OC[C@@H]2C)C(=N)N1 ; CCC1(CC)CC(=O)N([C@H]2c3cc(C(=O)N[C@@H]4c5ccccc5OC(C)(C)[C@H]4O)ccc3OC[C@H]2C)C(=N)N1 ; CCC1(CC)CC(=O)N([C@@H]2CCOc3ccc(C(=O)NC4c5ccccc5OC(C)(C)C4(C)O)cc32)C(=N)N1]

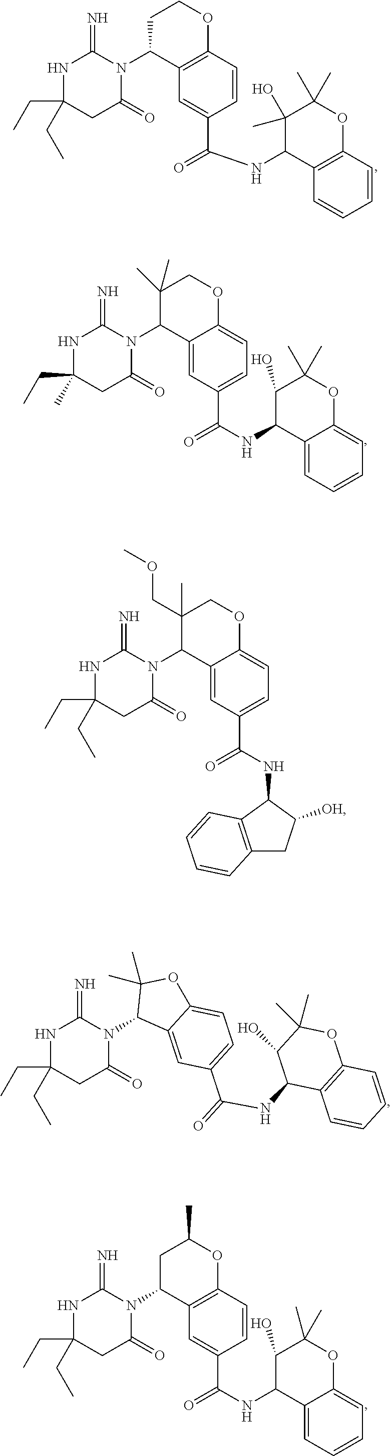 [SMILES: CCC1(CC)CC(=O)N([C@@H]2CCOc3ccc(C(=O)NC4c5ccccc5OC(C)(C)C4(C)O)cc32)C(=N)N1 ; CC[C@]1(C)CC(=O)N(C2c3cc(C(=O)N[C@@H]4c5ccccc5OC(C)(C)[C@H]4O)ccc3OCC2(C)C)C(=N)N1 ; CCC1(CC)CC(=O)N(C2c3cc(C(=O)N[C@@H]4c5ccccc5C[C@H]4O)ccc3OCC2(C)COC)C(=N)N1 ; CCC1(CC)CC(=O)N([C@H]2c3cc(C(=O)N[C@@H]4c5ccccc5OC(C)(C)[C@H]4O)ccc3OC2(C)C)C(=N)N1 ; CCC1(CC)CC(=O)N([C@@H]2C[C@@H](C)Oc3ccc(C(=O)NC4c5ccccc5OC(C)(C)[C@H]4O)cc32)C(=N)N1]

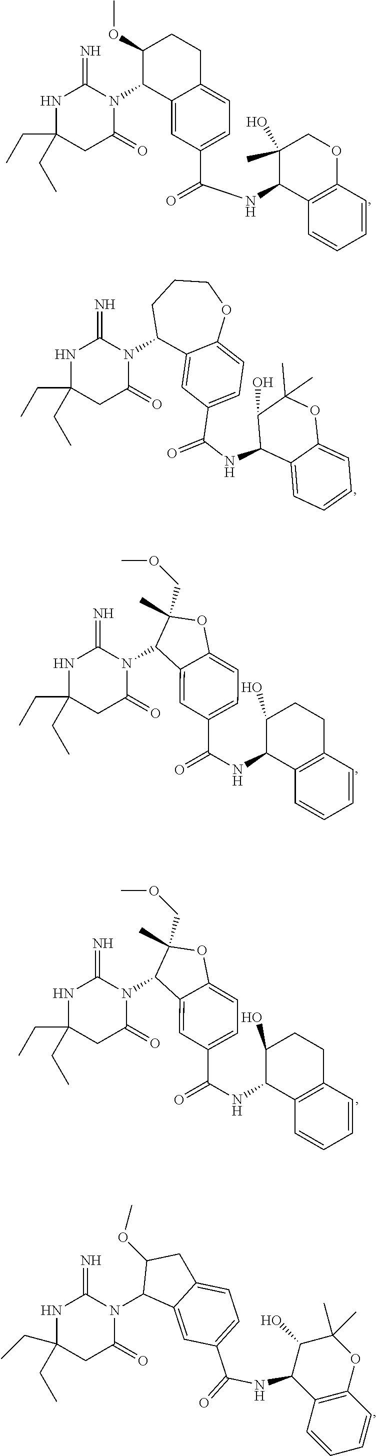

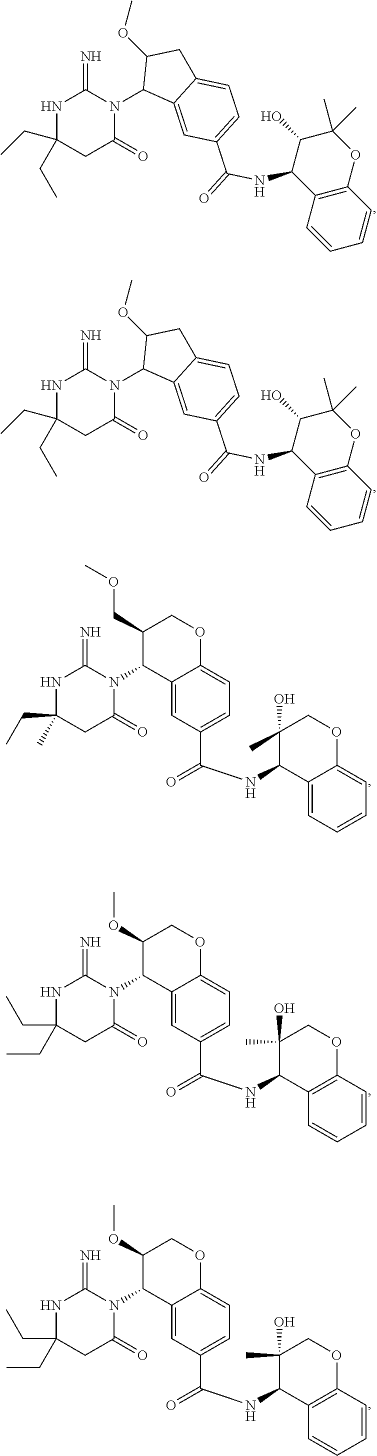 [SMILES: CCC1(CC)CC(=O)N(C2c3cc(C(=O)N[C@@H]4c5ccccc5OC(C)(C)[C@H]4O)ccc3CC2OC)C(=N)N1 ; CCC1(CC)CC(=O)N(C2c3cc(C(=O)N[C@@H]4c5ccccc5OC(C)(C)[C@H]4O)ccc3CC2OC)C(=N)N1 ; CC[C@]1(C)CC(=O)N([C@H]2c3cc(C(=O)N[C@@H]4c5ccccc5OC[C@@]4(C)O)ccc3OC[C@@H]2COC)C(=N)N1 ; CCC1(CC)CC(=O)N([C@H]2c3cc(C(=O)N[C@@H]4c5ccccc5OC[C@]4(C)O)ccc3OC[C@@H]2OC)C(=N)N1 ; CCC1(CC)CC(=O)N([C@H]2c3cc(C(=O)N[C@@H]4c5ccccc5OC[C@@]4(C)O)ccc3OC[C@@H]2OC)C(=N)N1]

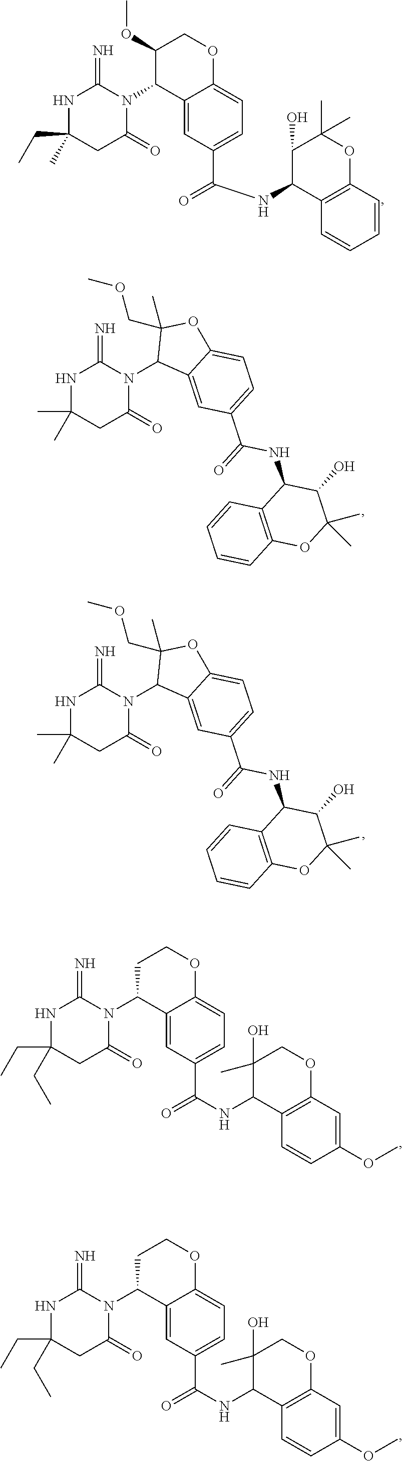

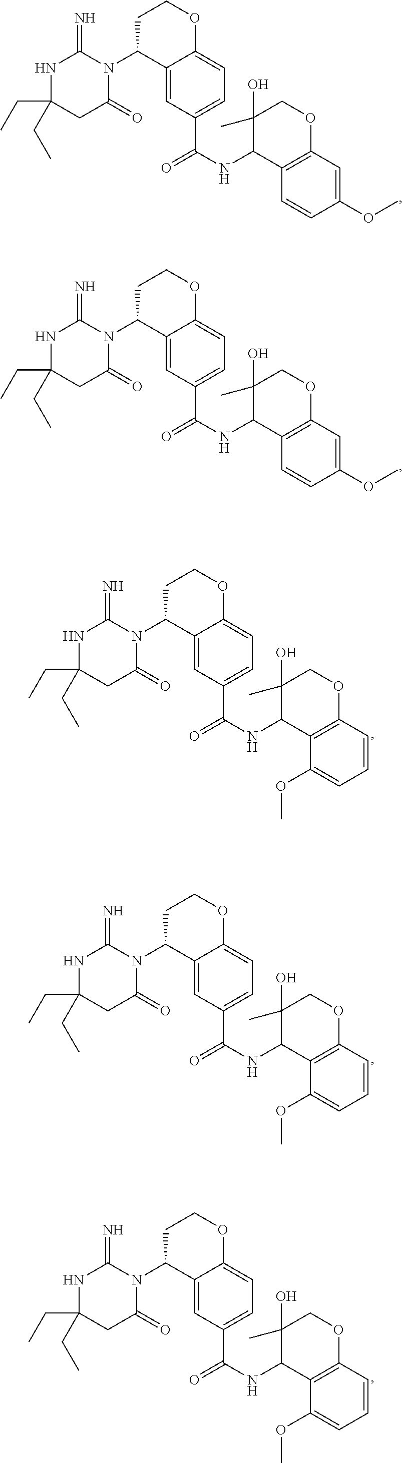

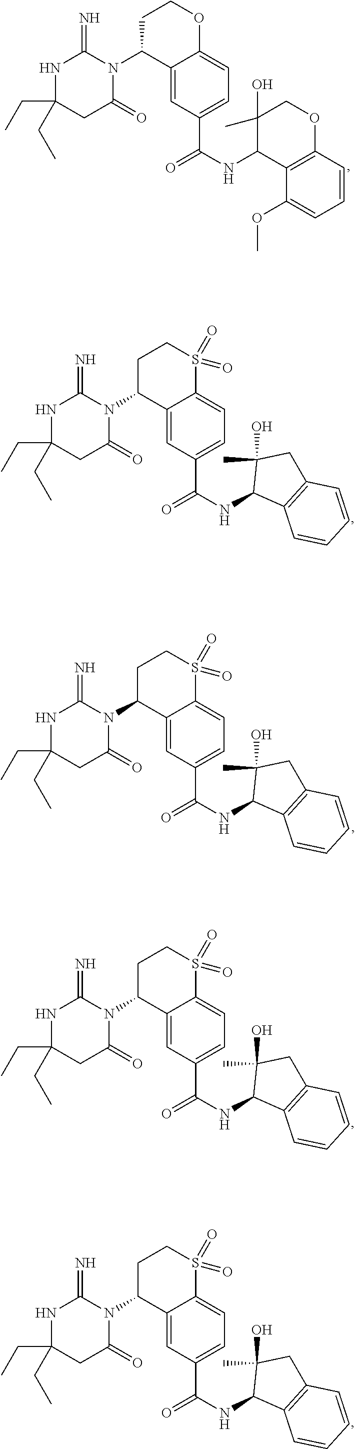 [SMILES: CCC1(CC)CC(=O)N([C@@H]2CCOc3ccc(C(=O)NC4c5c(OC)cccc5OCC4(C)O)cc32)C(=N)N1 ; CCC1(CC)CC(=O)N([C@@H]2CCS(=O)(=O)c3ccc(C(=O)N[C@@H]4c5ccccc5C[C@@]4(C)O)cc32)C(=N)N1 ; CCC1(CC)CC(=O)N([C@H]2CCS(=O)(=O)c3ccc(C(=O)N[C@@H]4c5ccccc5C[C@@]4(C)O)cc32)C(=N)N1 ; CCC1(CC)CC(=O)N([C@@H]2CCS(=O)(=O)c3ccc(C(=O)N[C@@H]4c5ccccc5C[C@]4(C)O)cc32)C(=N)N1 ; CCC1(CC)CC(=O)N([C@@H]2CCS(=O)(=O)c3ccc(C(=O)N[C@@H]4c5ccccc5C[C@]4(C)O)cc32)C(=N)N1]

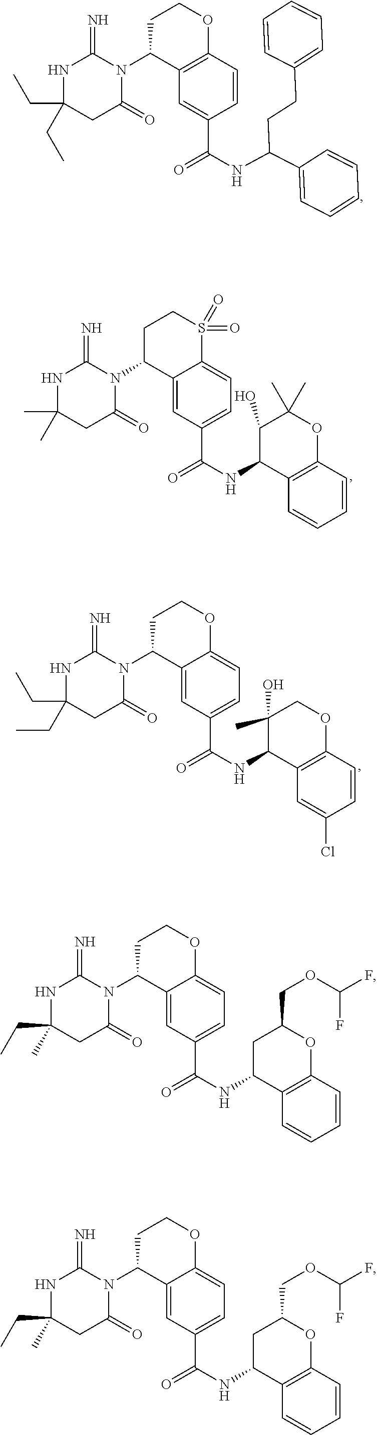 [SMILES: CCC1(CC)CC(=O)N([C@@H]2CCOc3ccc(C(=O)NC(CCc4ccccc4)c4ccccc4)cc32)C(=N)N1 ; CC1(C)CC(=O)N([C@@H]2CCS(=O)(=O)c3ccc(C(=O)N[C@@H]4c5ccccc5OC(C)(C)[C@H]4O)cc32)C(=N)N1 ; CCC1(CC)CC(=O)N([C@@H]2CCOc3ccc(C(=O)N[C@@H]4c5cc(Cl)ccc5OC[C@@]4(C)O)cc32)C(=N)N1 ; CC[C@]1(C)CC(=O)N([C@@H]2CCOc3ccc(C(=O)N[C@@H]4C[C@@H](COC(F)F)Oc5ccccc54)cc32)C(=N)N1 ; CC[C@]1(C)CC(=O)N([C@@H]2CCOc3ccc(C(=O)N[C@@H]4C[C@H](COC(F)F)Oc5ccccc54)cc32)C(=N)N1]

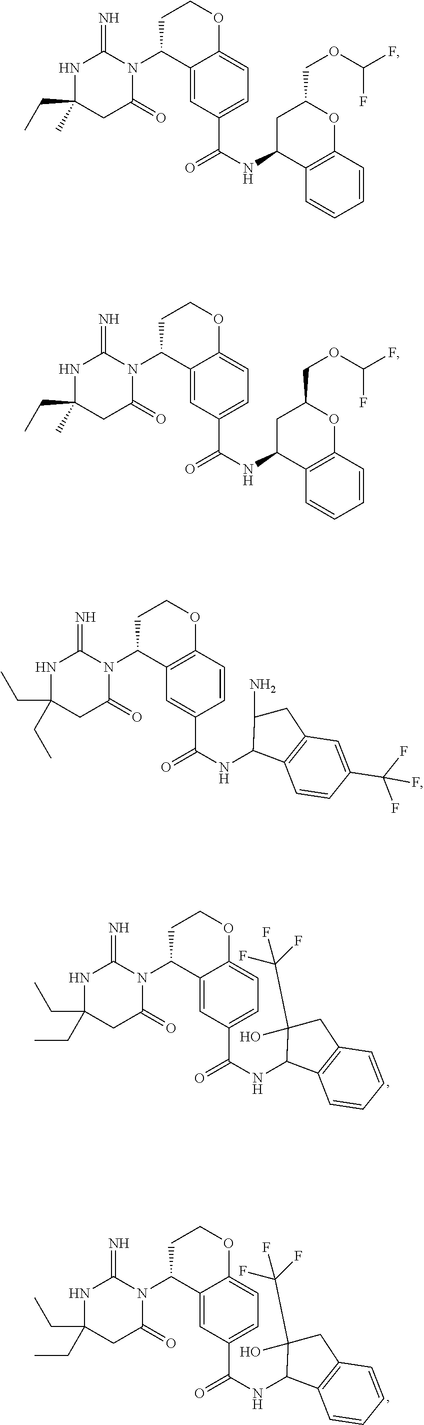 [SMILES: CC[C@]1(C)CC(=O)N([C@@H]2CCOc3ccc(C(=O)N[C@H]4C[C@H](COC(F)F)Oc5ccccc54)cc32)C(=N)N1 ; CC[C@]1(C)CC(=O)N([C@@H]2CCOc3ccc(C(=O)N[C@H]4C[C@@H](COC(F)F)Oc5ccccc54)cc32)C(=N)N1 ; CCC1(CC)CC(=O)N([C@@H]2CCOc3ccc(C(=O)NC4c5ccc(C(F)(F)F)cc5CC4N)cc32)C(=N)N1 ; CCC1(CC)CC(=O)N([C@@H]2CCOc3ccc(C(=O)NC4c5ccccc5CC4(O)C(F)(F)F)cc32)C(=N)N1 ; CCC1(CC)CC(=O)N([C@@H]2CCOc3ccc(C(=O)NC4c5ccccc5CC4(O)C(F)(F)F)cc32)C(=N)N1]

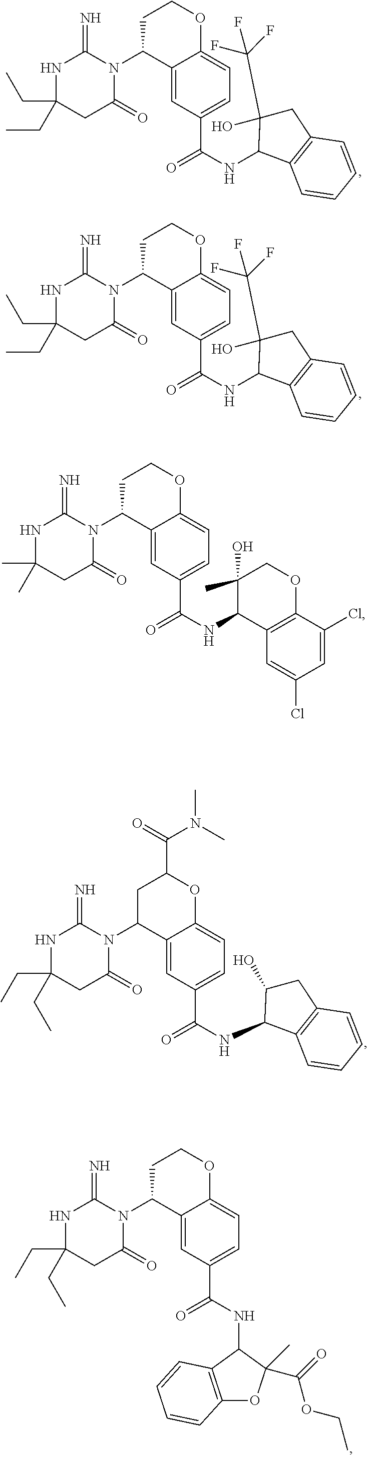 [SMILES: CCC1(CC)CC(=O)N([C@@H]2CCOc3ccc(C(=O)NC4c5ccccc5CC4(O)C(F)(F)F)cc32)C(=N)N1 ; CCC1(CC)CC(=O)N([C@@H]2CCOc3ccc(C(=O)NC4c5ccccc5CC4(O)C(F)(F)F)cc32)C(=N)N1 ; CC1(C)CC(=O)N([C@@H]2CCOc3ccc(C(=O)N[C@@H]4c5cc(Cl)cc(Cl)c5OC[C@@]4(C)O)cc32)C(=N)N1 ; CCC1(CC)CC(=O)N(C2CC(C(=O)N(C)C)Oc3ccc(C(=O)N[C@@H]4c5ccccc5C[C@H]4O)cc32)C(=N)N1 ; CCOC(=O)C1(C)Oc2ccccc2C1NC(=O)c1ccc2c(c1)[C@H](N1C(=N)NC(CC)(CC)CC1=O)CCO2]

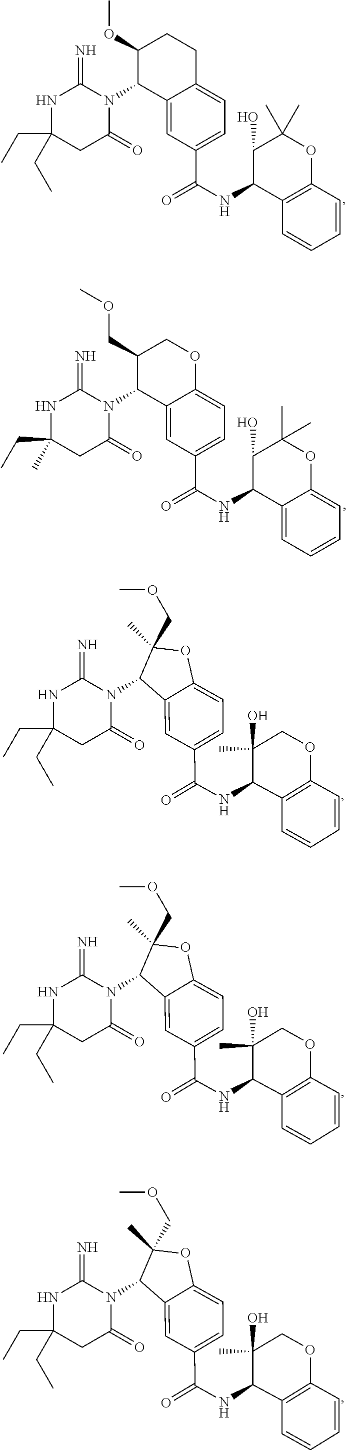 [SMILES: CCC1(CC)CC(=O)N([C@H]2c3cc(C(=O)N[C@@H]4c5ccccc5OC(C)(C)[C@H]4O)ccc3CC[C@@H]2OC)C(=N)N1 ; CC[C@]1(C)CC(=O)N([C@H]2c3cc(C(=O)N[C@@H]4c5ccccc5OC(C)(C)[C@H]4O)ccc3OC[C@@H]2COC)C(=N)N1 ; CCC1(CC)CC(=O)N([C@H]2c3cc(C(=O)N[C@@H]4c5ccccc5OC[C@]4(C)O)ccc3O[C@]2(C)COC)C(=N)N1 ; CCC1(CC)CC(=O)N([C@H]2c3cc(C(=O)N[C@@H]4c5ccccc5OC[C@@]4(C)O)ccc3O[C@]2(C)COC)C(=N)N1 ; CCC1(CC)CC(=O)N([C@H]2c3cc(C(=O)N[C@@H]4c5ccccc5OC[C@]4(C)O)ccc3O[C@@]2(C)COC)C(=N)N1]

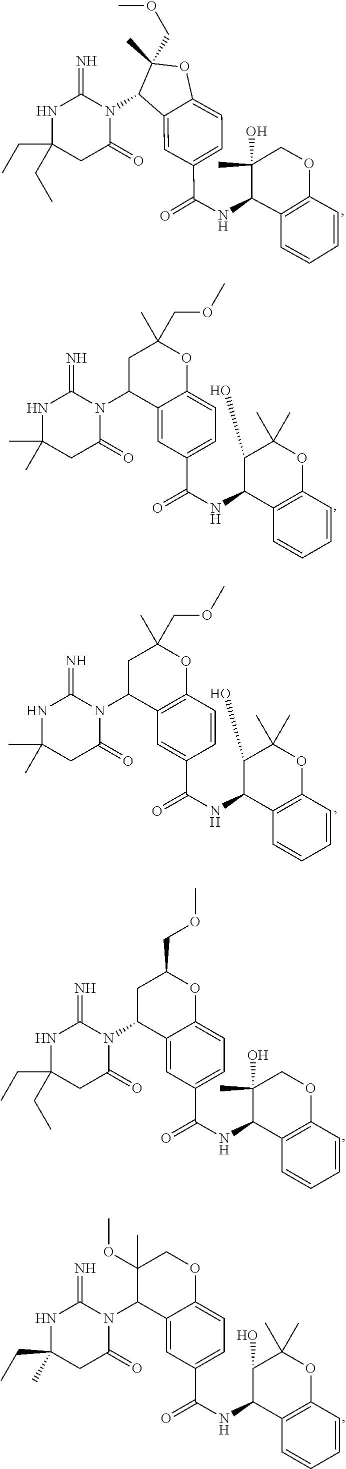 [SMILES: CCC1(CC)CC(=O)N([C@H]2c3cc(C(=O)N[C@@H]4c5ccccc5OC[C@@]4(C)O)ccc3O[C@@]2(C)COC)C(=N)N1 ; COCC1(C)CC(N2C(=N)NC(C)(C)CC2=O)c2cc(C(=O)N[C@@H]3c4ccccc4OC(C)(C)[C@H]3O)ccc2O1 ; COCC1(C)CC(N2C(=N)NC(C)(C)CC2=O)c2cc(C(=O)N[C@@H]3c4ccccc4OC(C)(C)[C@H]3O)ccc2O1 ; CCC1(CC)CC(=O)N([C@@H]2C[C@@H](COC)Oc3ccc(C(=O)N[C@@H]4c5ccccc5OC[C@@]4(C)O)cc32)C(=N)N1 ; CC[C@]1(C)CC(=O)N(C2c3cc(C(=O)N[C@@H]4c5ccccc5OC(C)(C)[C@H]4O)ccc3OCC2(C)OC)C(=N)N1]

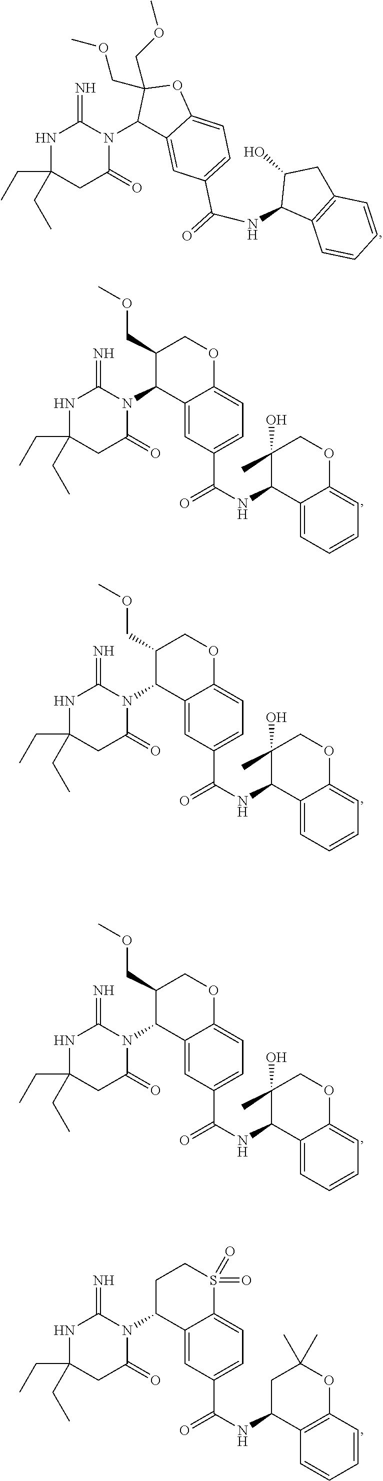 [SMILES: CCC1(CC)CC(=O)N(C2c3cc(C(=O)N[C@@H]4c5ccccc5C[C@H]4O)ccc3OC2(COC)COC)C(=N)N1 ; CCC1(CC)CC(=O)N([C@@H]2c3cc(C(=O)N[C@@H]4c5ccccc5OC[C@@]4(C)O)ccc3OC[C@@H]2COC)C(=N)N1 ; CCC1(CC)CC(=O)N([C@H]2c3cc(C(=O)N[C@@H]4c5ccccc5OC[C@@]4(C)O)ccc3OC[C@H]2COC)C(=N)N1 ; CCC1(CC)CC(=O)N([C@H]2c3cc(C(=O)N[C@@H]4c5ccccc5OC[C@@]4(C)O)ccc3OC[C@@H]2COC)C(=N)N1 ; CCC1(CC)CC(=O)N([C@@H]2CCS(=O)(=O)c3ccc(C(=O)N[C@H]4CC(C)(C)Oc5ccccc54)cc32)C(=N)N1]

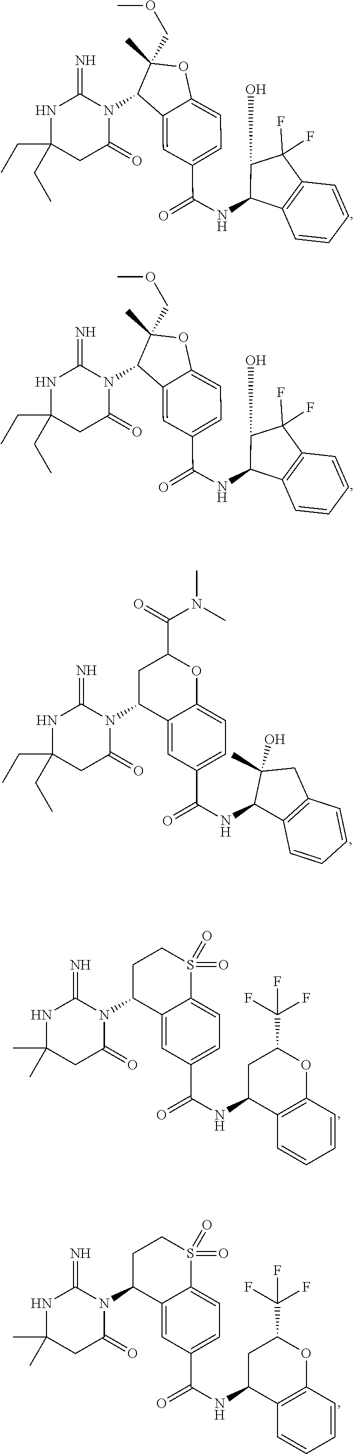 [SMILES: CCC1(CC)CC(=O)N([C@H]2c3cc(C(=O)N[C@@H]4c5ccccc5C(F)(F)[C@H]4O)ccc3O[C@@]2(C)COC)C(=N)N1 ; CCC1(CC)CC(=O)N([C@H]2c3cc(C(=O)N[C@@H]4c5ccccc5C(F)(F)[C@H]4O)ccc3O[C@@]2(C)COC)C(=N)N1 ; CCC1(CC)CC(=O)N([C@@H]2CC(C(=O)N(C)C)Oc3ccc(C(=O)N[C@@H]4c5ccccc5C[C@@]4(C)O)cc32)C(=N)N1 ; CC1(C)CC(=O)N([C@@H]2CCS(=O)(=O)c3ccc(C(=O)N[C@H]4C[C@H](C(F)(F)F)Oc5ccccc54)cc32)C(=N)N1 ; CC1(C)CC(=O)N([C@H]2CCS(=O)(=O)c3ccc(C(=O)N[C@H]4C[C@H](C(F)(F)F)Oc5ccccc54)cc32)C(=N)N1]

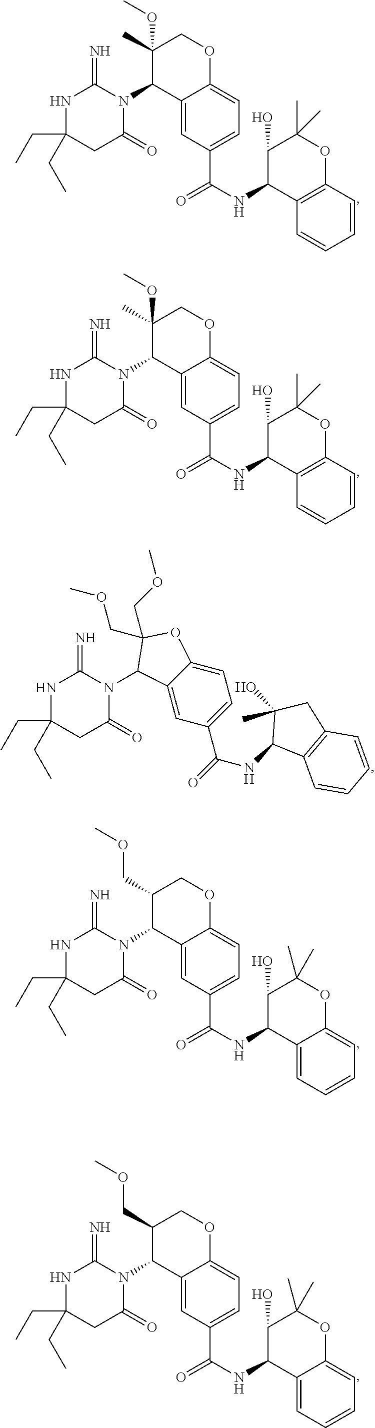 [SMILES: CCC1(CC)CC(=O)N([C@@H]2c3cc(C(=O)N[C@@H]4c5ccccc5OC(C)(C)[C@H]4O)ccc3OC[C@@]2(C)OC)C(=N)N1 ; CCC1(CC)CC(=O)N([C@H]2c3cc(C(=O)N[C@@H]4c5ccccc5OC(C)(C)[C@H]4O)ccc3OC[C@]2(C)OC)C(=N)N1 ; CCC1(CC)CC(=O)N(C2c3cc(C(=O)N[C@@H]4c5ccccc5C[C@@]4(C)O)ccc3OC2(COC)COC)C(=N)N1 ; CCC1(CC)CC(=O)N([C@H]2c3cc(C(=O)N[C@@H]4c5ccccc5OC(C)(C)[C@H]4O)ccc3OC[C@H]2COC)C(=N)N1 ; CCC1(CC)CC(=O)N([C@H]2c3cc(C(=O)N[C@@H]4c5ccccc5OC(C)(C)[C@H]4O)ccc3OC[C@@H]2COC)C(=N)N1]

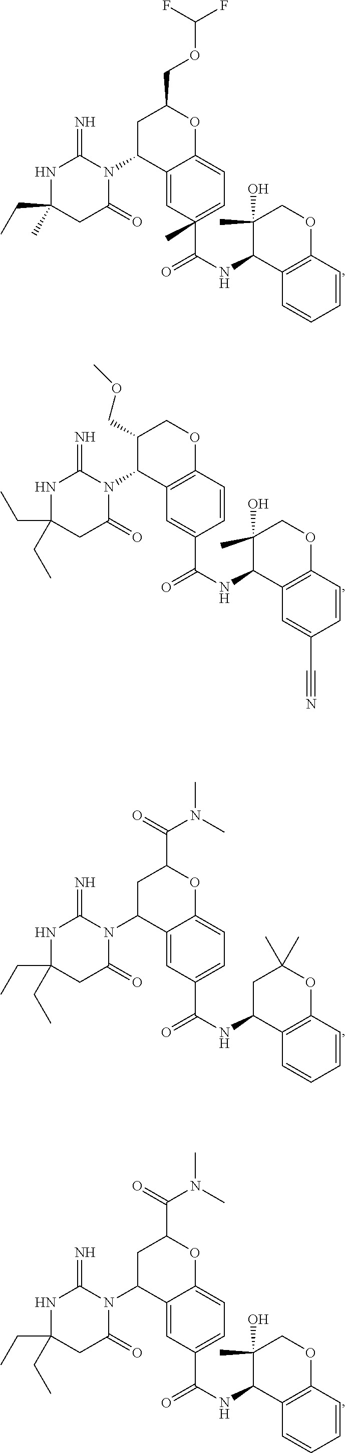

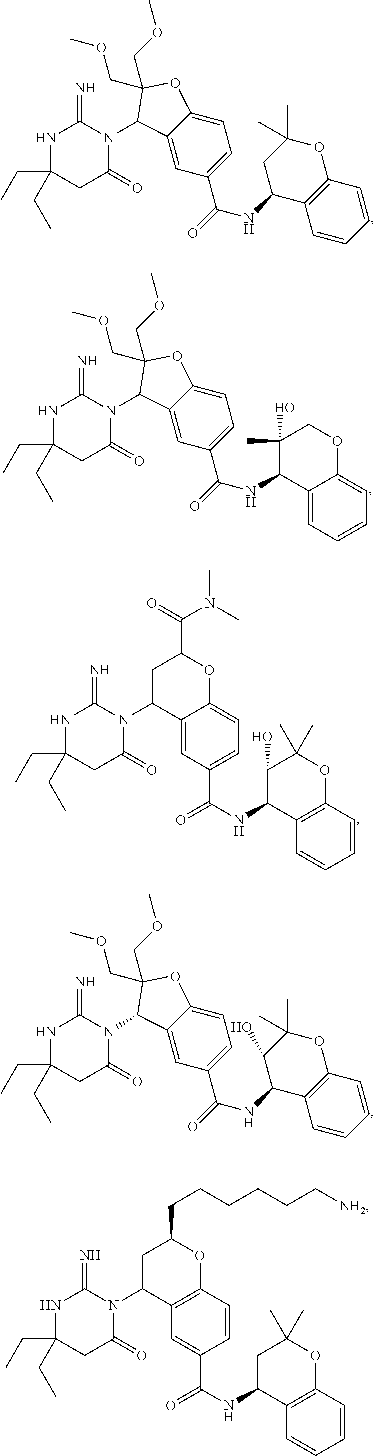 [SMILES: CCC1(CC)CC(=O)N(C2c3cc(C(=O)N[C@H]4CC(C)(C)Oc5ccccc54)ccc3OC2(COC)COC)C(=N)N1 ; CCC1(CC)CC(=O)N(C2c3cc(C(=O)N[C@@H]4c5ccccc5OC[C@@]4(C)O)ccc3OC2(COC)COC)C(=N)N1 ; CCC1(CC)CC(=O)N(C2CC(C(=O)N(C)C)Oc3ccc(C(=O)N[C@@H]4c5ccccc5OC(C)(C)[C@H]4O)cc32)C(=N)N1 ; CCC1(CC)CC(=O)N([C@H]2c3cc(C(=O)N[C@@H]4c5ccccc5OC(C)(C)[C@H]4O)ccc3OC2(COC)COC)C(=N)N1 ; CCC1(CC)CC(=O)N(C2C[C@@H](CCCCCCN)Oc3ccc(C(=O)N[C@H]4CC(C)(C)Oc5ccccc54)cc32)C(=N)N1]

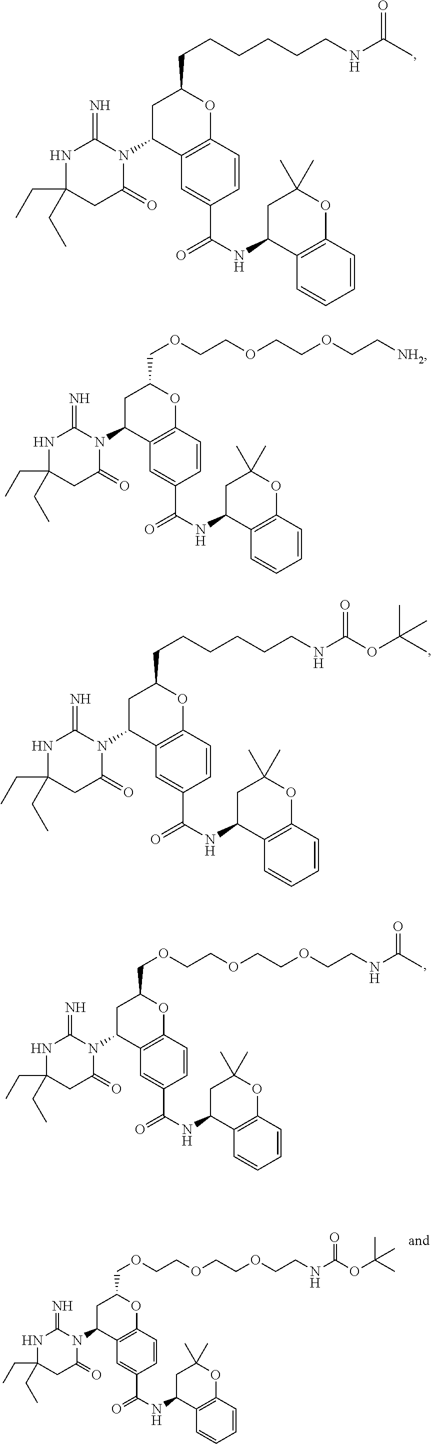

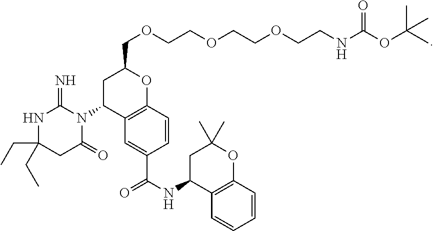 [SMILES: CCC1(CC)CC(=O)N([C@@H]2C[C@@H](COCCOCCOCCNC(=O)OC(C)(C)C)Oc3ccc(C(=O)N[C@H]4CC(C)(C)Oc5ccccc54)cc32)C(=N)N1]

In certain embodiments, the compound is selected from the group consisting of

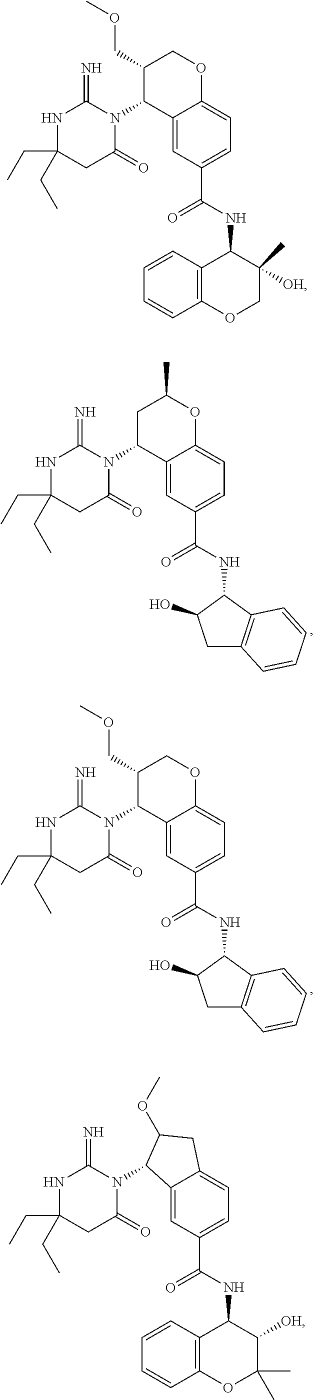

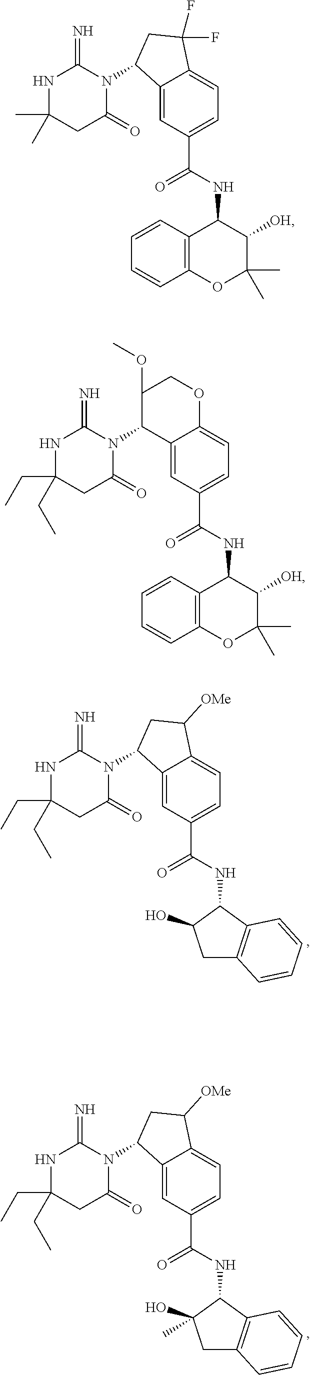

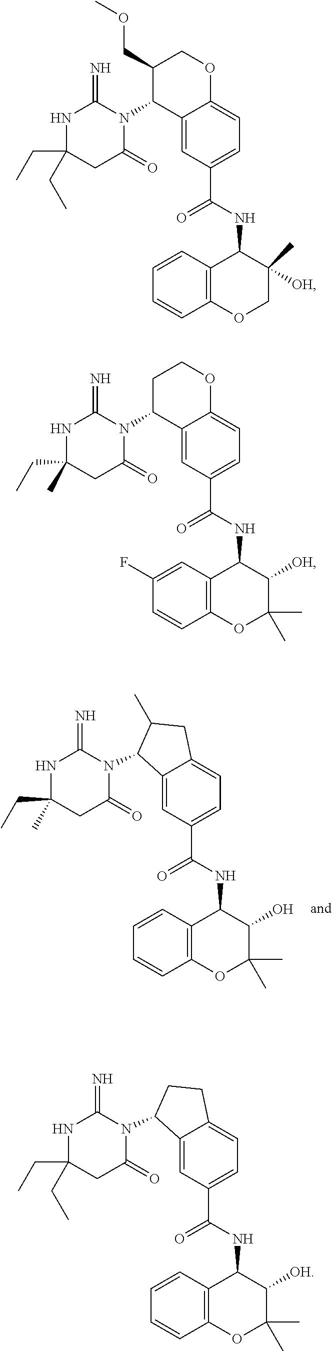

In other embodiments, the compound is selected from the group consisting of

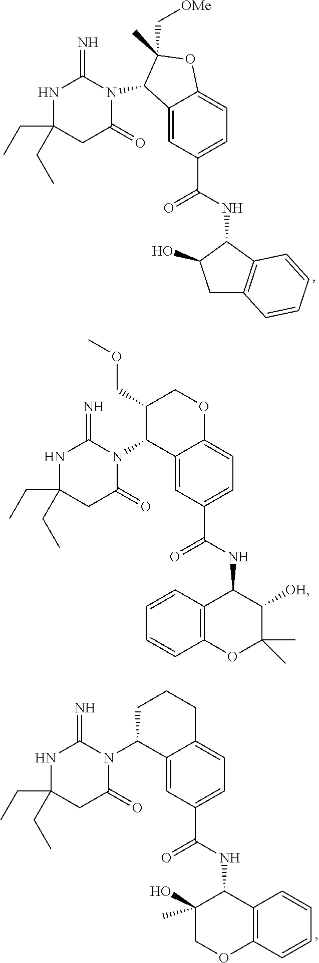

pharmaceutically acceptable salt thereof.

In certain embodiments described herein, the compound has the formula:

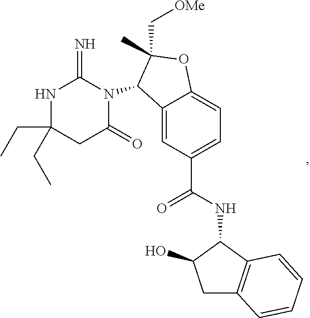 [SMILES: CCC1(CC)CC(=O)N([C@H]2c3cc(C(=O)N[C@@H]4c5ccccc5C[C@H]4O)ccc3O[C@@]2(C)COC)C(=N)N1]

or a pharmaceutically acceptable salt thereof.

In certain embodiments described herein, the compound has the formula:

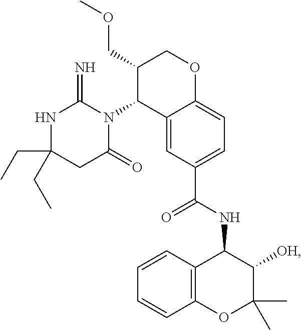 [SMILES: CCC1(CC)CC(=O)N([C@H]2c3cc(C(=O)N[C@@H]4c5ccccc5OC(C)(C)[C@H]4O)ccc3OC[C@H]2COC)C(=N)N1]

or a pharmaceutically acceptable salt thereof.

In certain embodiments described herein, the compound has the formula:

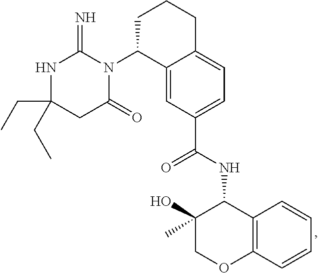 [SMILES: CCC1(CC)CC(=O)N([C@@H]2CCCc3ccc(C(=O)N[C@@H]4c5ccccc5OC[C@@]4(C)O)cc32)C(=N)N1]

or a pharmaceutically acceptable salt thereof.

The present invention is also directed to methods of chemovaccination against Plasmodium infections comprising administering to a patient, an effective amount of a dual inhibitor of plasmepsin IX and X of Formula (I′) or (I), or a pharmaceutically acceptable salt thereof. In certain embodiments, the compounds of Formula (I′) or (I), or a pharmaceutically acceptable salt thereof, are administered in the form of a pharmaceutical composition, further comprising a pharmaceutically acceptable carrier or excipient.

In certain embodiments described herein, the present invention is directed to methods of chemovaccination against malaria comprising administering to a patient, an effective amount of a dual inhibitor of plasmepsin IX and X, said compound having the structural Formula (I′) or (I) described herein. In some embodiments, the compounds of Formula (I′) or (I), or pharmaceutically acceptable salts thereof, are administered with a pharmaceutically acceptable carrier, as a pharmaceutical composition. Also provided herein are various embodiments of these methods, as described, infra.

The invention also relates to the use of a compound of Formula (I′), (I), (IA), (IB), (IC), (ID) or (IE) or a pharmaceutically acceptable salt thereof for inhibiting plasmepsin IX and X activity, for chemovaccination against a Plasmodium infection, or for chemovaccination against malaria. The invention further relates to the use of a compound of Formula (I′), (I), (IA), (IB), (IC), (ID) or (IE) or a pharmaceutically acceptable salt thereof in the manufacture of a medicament for inhibiting plasmepsin IX and X activity, for chemovaccination against a Plasmodium infection, or for chemovaccination against malaria. The compounds of Formula (I′), (I), (IA), (IB), (IC), (ID) or (IE) or pharmaceutically acceptable salts thereof described in any of the embodiments of the invention herein are useful for any of the uses above.

Accordingly, another embodiment provides methods for chemovaccination against malaria or for chemovaccination against Plasmodium infection, comprising administration of combinations comprising an amount of at least one compound of Formula (I′), (I), (IA), (IB), (IC), (ID) or (IE), or a pharmaceutically acceptable salt, solvate, ester or prodrug thereof, and treating malaria by administering an effective amount of one or more additional agents described below. In certain embodiments, described herein are methods for chemovaccination against and treatment of malaria or for chemovaccination against Plasmodium infection and treatment of Plasmodium infection, comprising administration of combinations comprising an amount of at least one compound of Formula (I′), (I), (IA), (IB), (IC), (ID) or (IE), or a pharmaceutically acceptable salt, solvate, ester or prodrug thereof, and an effective amount of one or more additional anti-malarial agents. In certain embodiments, described herein are methods for chemovaccination against malaria by inhibition of plasmepsin IX and X and treating malaria via at least one other mechanism, comprising administration of combinations comprising an amount of at least one compound of Formula (I′), (I), (IA), (IB), (IC), (ID) or (IE), or a pharmaceutically acceptable salt, solvate, ester or prodrug thereof, and an effective amount of one or more additional anti-malarial agents, wherein the additional anti-malarial agents act through a different mechanism than inhibiting plasmepsin IX or plasmepsin X.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names and chemical structures may be used interchangeably to describe that same structure. These definitions apply regardless of whether a term is used by itself or in combination with other terms, unless otherwise indicated. Hence the definition of “alkyl” applies to “alkyl” as well as the “alkyl” portion of “hydroxyalkyl”, “haloalkyl”, arylalkyl-, alkylaryl-, “alkoxy” etc.

It shall be understood that, in the various embodiments of the invention described herein, any variable not explicitly defined in the context of the embodiment is as defined in Formula (I′).

In the various embodiments described herein, each variable is selected independently of the others unless otherwise indicated.

“Chemovaccination” means induction of adaptive immune responses to Plasmodium infection during anti-viral drug administration.

“Drug resistant” means, in connection with a Plasmodium parasite strain, a Plasmodium species which is no longer susceptible to at least one previously effective drug; which has developed the ability to withstand attack by at least one previously effective drug. A drug resistant strain may relay that ability to withstand to its progeny. Said resistance may be due to random genetic mutations in the bacterial cell that alters its sensitivity to a single drug or to different drugs.

“Patient” includes both human and non-human animals. Non-human animals include those research animals and companion animals such as mice, rats, primates, monkeys, chimpanzees, great apes, dogs, and house cats.

As used herein “patient” means any patient with a liver stage Plasmodium infection, e.g. of Plasmodium falciparum or Plasmodium vivax. Alternatively, a “patient” could mean a patient without Plasmodium parasite infection, that is administered a Plasmodium parasite inoculum, such as a wild-type Plasmodium parasite or a genetically modified Plasmodium parasite and dual inhibitor of plasmepsin IX and X.

“Pharmaceutical composition” (or “pharmaceutically acceptable composition”) means a composition suitable for administration to a patient. Such compositions may contain the neat compound (or compounds) of the invention or mixtures thereof, or salts, solvates, prodrugs, isomers, or tautomers thereof, and one or more pharmaceutically acceptable carriers or diluents. The term “pharmaceutical composition” is also intended to encompass both the bulk composition and individual dosage units comprised of one or more (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

“Halogen” and “halo” mean fluorine, chlorine, bromine, or iodine. Preferred are fluorine, chlorine and bromine.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. Non-limiting examples of suitable alkyl groups include methyl, ethyl, n-propyl, isopropyl and t-butyl.

“Haloalkyl” means an alkyl as defined above wherein one or more hydrogen atoms on the alkyl is replaced by a halo group defined above.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different and are as defined herein. Non-limiting examples of suitable aryl groups include phenyl and naphthyl. “Monocyclic aryl” means phenyl.

“Cycloalkyl” means a non-aromatic mono- or multicyclic ring system comprising about 3 to about 12 carbon atoms, preferably about 3 to about 10 carbon atoms. Preferred cycloalkyl rings contain about 5 to about 10 ring atoms. The cycloalkyl can be optionally substituted with one or more substituents, which may be the same or different, as described herein. Monocyclic cycloalkyl refers to monocyclic versions of the cycloalkyl moieties described herein. Non-limiting examples of suitable monocyclic cycloalkyls include cyclopropyl, cyclopentyl, cyclohexyl, cycloheptyl and the like. Multicyclic cycloalkyls refers to multicyclic, including bicyclic, rings that include a non-aromatic ring. Non-limiting examples of suitable multicyclic cycloalkyls include 1-decalinyl, norbornyl, adamantyl and the like. In certain embodiments, a non-aromatic ring is fused to an aromatic ring. Further non-limiting examples of cycloalkyl include the following:

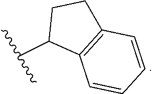 [SMILES: *C1CCc2ccccc21]

“Heterocycloalkyl” (or “heterocyclyl”) means a non-aromatic, saturated or partially saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more substituents, which may be the same or different, as described herein. The nitrogen or sulfur atom of the heterocyclyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. “Heterocyclyl” also includes rings wherein ═O replaces two available hydrogens on the same carbon atom (i.e., heterocyclyl includes rings having a carbonyl group in the ring). Such ═O groups may be referred to herein as “oxo.” An example of such a moiety is pyrrolidinone (or pyrrolidone):

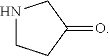

As used herein, the term “monocyclic heterocycloalkyl” refers monocyclic versions of the heterocycloalkyl moieties described herein and include a 4- to 7-membered monocyclic heterocycloalkyl groups comprising from 1 to 4 ring heteroatoms, said ring heteroatoms being independently selected from the group consisting of N, N-oxide, O, S, S-oxide, S(O), and S(O)₂. The point of attachment to the parent moiety is to any available ring carbon or ring heteroatom. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. A non-limiting example of a monocyclic heterocycloalkyl group include the moiety:

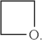

Non-limiting examples of multicyclic heterocycloalkyl groups include, bicyclic heterocycloalkyl groups. Specific examples include, but are not limited to,

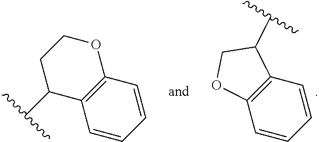 [SMILES: *C1CCOc2ccccc21 ; *C1COc2ccccc21]

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals, or moieties.

When a variable appears more than once in a group, e.g., R⁸ in —N(R⁸)₂, or a variable appears more than once in a structure presented herein, the variables can be the same or different.

A solid line [symbol] , as a bond generally indicates a mixture of, or either of, the possible isomers, e.g., containing (R)- and (S)-stereochemistry. For example:

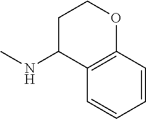

means containing either one of or both

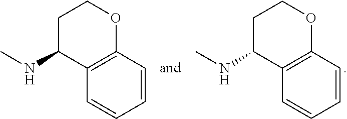

The wavy line [symbol] , as used herein shown crossing a line representing a chemical bond, indicates a point of attachment to the rest of the compound. Lines drawn into the ring systems, such as, for example

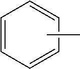

indicates that the indicated line (bond) may be attached to any of the substitutable ring atoms.

“Oxo” is defined as an oxygen atom that is double bonded to a ring carbon in a cycloalkyl, cycloalkenyl, heterocycloalkyl, heterocycloalkenyl, or another ring described herein, e.g.,

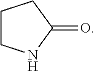

In this specification, where there are multiple oxygen and/or sulfur atoms in a ring system, there cannot be any adjacent oxygen and/or sulfur present in said ring system.

As well known in the art, a bond drawn from a particular atom wherein no moiety is depicted at the terminal end of the bond indicates a methyl group bound through that bond to the atom, unless stated otherwise. For example:

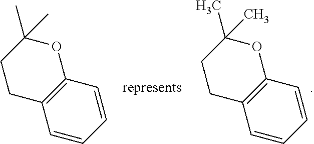

In another embodiment, the compounds useful in the methods of the invention, and/or compositions comprising them useful in said methods, are present in isolated and/or purified form. The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the

physical state of said compound (or a tautomer or stereoisomer thereof, or pharmaceutically acceptable salt or solvate of said compound, said stereoisomer, or said tautomer) after being obtained from a purification process or processes described herein or well known to the skilled artisan (e.g., chromatography, recrystallization and the like), in sufficient purity to be suitable for in vivo or medicinal use and/or characterizable by standard analytical techniques described herein or well known to the skilled artisan.

It shall be understood that any carbon as well as heteroatom with unsatisfied valences in the text, schemes, examples, and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

Another embodiment provides prodrugs and/or solvates of the compounds of the invention. A discussion of prodrugs is provided in T. Higuchi and V. Stella, Pro-dugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of the invention or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound or a pharmaceutically acceptable salt thereof, useful in the methods of the invention contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C1-C2)alkyl and piperidino-, pyrrolidino- or morpholino(C₂-C₃)alkyl, and the like.

Similarly, if a compound used in the methods of the invention contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxy carbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)O(C₁-C₆)alkyl)₂ or glycosyl (the radical resulting from the removal of a hydroxyl group of the hemiacetal form of a carbohydrate), and the like.

If a compound used in the methods of the invention incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y³ wherein Y⁴ is H or methyl and Y⁵ is mono-N— or di-N,N—(C₁-C₆)alkylamino morpholino, piperidin-1-yl or pyrrolidin-1-yl, and the like.

One or more compounds used in the methods of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of the invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds used in the methods of the invention may optionally be converted to a solvate. Preparation of solvates is generally known. Thus, for example M. Caira et al, J. Pharmaceutical Sci., 1993, 3, 601-611, describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 51), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition used in the methods of the present invention effective in inhibiting the above-noted diseases or enzyme activity and thus producing the desired therapeutic, ameliorative, inhibitory or preventative effect.

Another embodiment provides pharmaceutically acceptable salts of the compounds to be used in the methods of the invention. Thus, reference to a compound used in the methods of the invention herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds used in the methods of the invention may be formed, for example, by reacting a compound of the invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH S. Berge et al, Journal of Pharmaceutical Sciences (1977) 66(1) 1-19; P. Gould, International J. of Pharmaceutics (1986) 33201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Another embodiment provides pharmaceutically acceptable esters of the compounds used in the methods of the invention. Such esters include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

As mentioned herein, another embodiment provides tautomers of the compounds of the invention to be used in the methods herein, and salts, solvates, esters and prodrugs of said tautomers. It shall be understood that all tautomeric forms of such compounds are within the scope of the compounds used in the methods of the invention. For example, all keto-enol and imine-enamine forms of the compounds, when present, are included in the invention.

The compounds used in the methods of the invention may contain asymmetric or chiral centers, and, therefore, exist in different stereoisomeric forms. It is intended that all stereoisomeric forms of the compounds used in the methods of the invention as well as mixtures thereof, including racemic mixtures, form part of the present invention. In addition, the present invention embraces use of all geometric and positional isomers. For example, if a compound used in the methods of the invention incorporates a double bond or a fused ring, both the cis- and trans-forms, as well as mixtures, are embraced within the scope of the invention.

Another embodiment provides for diastereomeric mixtures and individual enantiomers of the compounds used in the methods of the invention. Diastereomeric mixtures can be separated into their individual diastereomers based on their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds used in the methods of the invention may be atropisomers (e.g., substituted biaryls) and are considered as part of this invention. Enantiomers can also be separated by use of chiral HPLC column.

All stereoisomers (for example, geometric isomers, optical isomers and the like) of the compounds used in the methods of the invention (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated as embodiments within the scope of this invention, as are positional isomers (such as, for example, 4-pyridyl and 3-pyridyl). (For example, if a compound of the invention incorporates a double bond or a fused ring, both the cis- and trans-forms, as well as mixtures, are embraced within the scope of the invention. Also, for example, all keto-enol and imine-enamine forms of the compounds are included in the methods of the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

Another embodiment provides isotopically-labelled compounds to be used in the methods the invention. Such compounds are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of the invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the invention can generally be prepared by following procedures analogous to those disclosed in the Schemes and/or in the Examples hereinbelow, by substituting an appropriate isotopically labelled reagent for a non-isotopically labelled reagent.

In the compounds used in the methods of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). The presence of deuterium in the compounds of the invention is indicated by “D”. Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of the invention can be prepared without undue experimentation by conventional techniques well known to those skilled in the art or by processes analogous to those described in the schemes and examples herein using appropriate isotopically-enriched reagents and/or intermediates.

Polymorphic forms of the compounds used in the methods of the invention, and of the salts, solvates, esters and prodrugs of the compounds of the invention, are intended to be included in the present invention.

As defined herein, an “adjuvant” is a substance that serves to enhance the immunogenicity of an immunogenic composition of the invention. An immune adjuvant may enhance an immune response to an antigen that is weakly immunogenic when administered alone. Thus, adjuvants are often given to boost the immune response and are well known to the skilled artisan. Suitable adjuvants to enhance effectiveness of the composition include, but are not limited to: (1) aluminum salts (alum), such as aluminum hydroxide, aluminum phosphate, aluminum sulfate, etc.; (2) oil-in-water emulsion formulations (with or without other specific immuno stimulating agents such as muramyl peptides (defined below) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP either micro fluidized into a submicron emulsion or vortexed to generate a larger particle size emulsion, (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094, trehalose dimycolate (TDM), and cell wall skeleton (CWS), preferably MPL+CWS (Detox™); and (d) a Montanide ISA; (3) saponin adjuvants, such as Quil A or STIMULON™ QS-21 (Antigenics, Framingham, Mass.) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix® (having essentially the same structure as an ISCOM but without the protein); (4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetrade-canoylaminojethyl 2-Deoxy-4-O-phosphono-3-O—[(R)-3-tetradecanoyloxytetradecanoyl]-2-[(R)-3-tetradecanoyloxy-tetradecanoylamino]-b-D-glucopyranoside, which is also known as 529 (formerly known as RC529), which is formulated as an aqueous form or as a stable emulsion (5) synthetic polynucleotides such as oligonucleotides containing CpG motif(s) (U.S. Pat. No. 6,207,646); and (6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.; and (7) complement, such as a trimer of complement component C3d.

### Dosage and Administration

Another embodiment provides suitable dosages and dosage forms of the compounds used in the methods of the invention. Suitable doses for administering compounds used in the methods of the invention to patients may readily be determined by those skilled in the art, e.g., by an attending physician, pharmacist, or other skilled worker, and may vary according to patient health, age, weight, frequency of administration, use with other active ingredients, and/or indication for which the compounds are administered. Doses may range from about 0.001 to 500 mg/kg of body weight/day of the compound of the invention. In one embodiment, the dosage is from about 0.01 to about 25 mg/kg of body weight/day of a compound of the invention, or a pharmaceutically acceptable salt or solvate of said compound. In another embodiment, the quantity of active compound in a unit dose of preparation may be varied or adjusted from about 1 mg to about 100 mg, in specific embodiments from about 1 mg to about 50 mg, in specific embodiments from about 1 mg to about 25 mg, according to the particular application. In another embodiment, a typical recommended daily dosage regimen for oral administration can range from about 1 mg/day to about 500 mg/day, in specific embodiments 1 mg/day to 200 mg/day, in two to four divided doses.

As discussed above, the amount and frequency of administration of the compounds of the invention and/or the pharmaceutically acceptable salts thereof will be regulated according to the judgment of the attending clinician considering such factors as age, condition and size of the patient as well as severity of the symptoms being treated.

Liquid form preparations include solutions, suspensions, and emulsions. As an example, may be the addition of the compounds described herein and water or water-propylene glycol solutions for parenteral injection or the addition of sweeteners and opacifiers for oral solutions, suspensions, and emulsions. Liquid form preparations may also include solutions for intranasal administration. Liquid preparations can also include an adjuvant.

Aerosol preparations suitable for inhalation may include solutions and solids in powder form, which may be in combination with a pharmaceutically acceptable carrier, such as an inert compressed gas, e.g. nitrogen.

In certain embodiments described herein, the preparation can be a long-acting injectable formulation. In certain embodiments of the methods described herein, a dual plasmepsin TX/X inhibitor is formulated as a long-acting injectable. In certain embodiments of the methods described herein, a compound of Formula (I′), (I), (IA), (IB), (IC), (ID) or (IE), or a pharmaceutically acceptable salt thereof is formulated as a long-acting injectable.

In certain embodiments, the present invention is also directed to methods of chemovaccination against Plasmodium infections comprising administering to a patient, wherein the patient does not have a Plasmodium parasite infection, a long-acting injectable formulation comprising an effective amount of a dual inhibitor of plasmepsin IX and X, or a pharmaceutically acceptable salt thereof, wherein the patient is eventually exposed to a Plasmodium parasite. In certain embodiments, the exposure to the Plasmodium parasite is through a mosquito bite.

Also included are solid form preparations that are intended to be converted, shortly before use, to liquid form preparations for either oral or parenteral administration. Such liquid forms include solutions, suspensions, and emulsions.

Another embodiment provides for use of compositions comprising a compound of Formula (I′), (I), (IA), (IB), (IC), (ID) or (IE), or a pharmaceutically acceptable salt thereof formulated for transdermal delivery. The transdermal compositions can take the form of creams, lotions, aerosols and/or emulsions and can be included in a transdermal patch of the matrix or reservoir type as are conventional in the art for this purpose.

Another embodiment provides for use of compositions comprising a compound of Formula (I′), (I), (IA), (IB), (IC), (ID) or (IE), or a pharmaceutically acceptable salt thereof formulated for subcutaneous delivery. Another embodiment provides for use of compositions suitable for oral delivery. In some embodiments, it may be advantageous for the pharmaceutical preparation comprising one or more compounds of Formula (I′), (I), (IA), (IB), (IC), (ID) or (IE), or a pharmaceutically acceptable salt thereof to be prepared in a unit dosage form. In such forms, the preparation is subdivided into suitably sized unit doses containing appropriate quantities of the active component, e.g., an effective amount to achieve the desired purpose. Each of the foregoing alternatives is considered as included in the various embodiments of the invention.

When used in combination with one or more additional therapeutic agents (“combination therapy”), the compounds used in the methods of this invention, i.e. the compounds of Formula (I′), (I), (IA), (IB), (IC), (ID) or (IE), may be administered together or sequentially. When administered sequentially, compounds of the invention may be administered before or after the one or more additional therapeutic agents, as determined by those skilled in the art or patient preference.

If formulated as a fixed dose, such combination products employ the compounds of Formula (I′), (I), (IA), (IB), (IC), (ID) or (IE), or a pharmaceutically acceptable salt thereof within the dosage range described herein and the other pharmaceutically active agent or treatment within its dosage range.

### Combination Therapy

Another embodiment provides for possible methods for chemovaccination using pharmaceutically acceptable compositions comprising a compound of the invention, either as the neat chemical or optionally further comprising additional ingredients. Such compositions are contemplated for preparation and use alone or in combination therapy. For preparing pharmaceutical compositions from the compounds of the invention, inert, pharmaceutically acceptable carriers can be either solid or liquid. Solid form preparations include powders, tablets, dispersible granules, capsules, cachets, and suppositories. The powders and tablets may be comprised of from about 5 to about 95 percent active ingredient. Suitable solid carriers are known in the art, e.g., magnesium carbonate, magnesium stearate, talc, sugar, or lactose. Tablets, powders, cachets, and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pennsylvania.

Non-limiting examples of additional drugs and active agents useful in combination therapies for chemovaccination against malaria, include the following: Coartem® (Novartis International AG, Basel, Switzerland; artemether+lumefantrine), Eurartesim® (Sigma-Tau Pharmaceuticals, Inc., Rome, Italy; dihydroartemisinin-piperaquine), Pyramax® (Shin Poong Pharmaceutical Co., Ltd., Seoul, Korea; pyronaridine-artesunate), ASAQ Winthrop® (Sanofi SA (Gentilly, France)/DNDi (Geneva, Switzerland); artesunate+amodiaquine), ASMQ (Cipla Limited (Mumbai, India)/DNDi, artesunate+mefloquine), SPAQ-CO™ (Guilin Pharmaceutical Co., Ltd. (Shanghai), amodiaquine+sulfadoxine, pyrimethamine), Artesun® (Guilin Pharmaceutical, artesunate), artemether, artesunate, dihydroartemisinin, lumefantrine, amodiaquine, mefloquine, piperaquine, quinine, chloroquine, atovaquone and proguanil and sulfadoxine-pyrimethamine, Tafenoquine (Glaxosmithkline), OZ439/PQP (Sanofi), OZ439/FQ (Sanofi), KAE609 (Novartis), KAF156 (Novartis), DSM265 (NIHITakeda), and MK-4815 (Merck & Co., Inc., Powles et al., Antimicrobial Agents and Chemotherapy 56(5): 2414-2419(2012)). Selection of such additional active ingredients will be according to the diseases or disorders present for which treatment is desired, as determined by the attending physician or other health care provider.

Thus, the invention also provides methods of using the compounds of Formula (I′), (I), (IA), (IB), (IC), (ID) or (IE), or a pharmaceutically acceptable salt thereof to inhibit plasmepsin X, plasmepsin IX or plasmepsin X and IX, and for chemovaccination against Plasmodium infection or chemovaccination against malaria wherein the method further comprises administering to a subject, one or more additional anti-malarial agents. In some embodiments, the one or more additional anti-malarial agents are selected from the group consisting of: artemether, lumefantrine, dihydroartemisinin, piperaquine, pyronaridine, artesunate, amodiaquine, mefloquine, sulfadoxine, pyrimethamine, lumefantrine, quinine, chloroquine, atovaquone, and proguanil.

### Processes of Making Plasmepsin IX and X Inhibitors

ACN=acetonitrile
AcOEt=ethylacetate
Bu₃P=Bis(tri-tert-butylphosphine)palladium(0)
DCM=dichloromethane
DIAD=Diisopropyl azodicarboxylate
DIEA=N, N-Diisopropylethylamine, or Hünig's base

### DMF=N,N-Dimethylformamide

DMP=Dess-Martin periodinane
DMSO=dimethyl sulfoxide
EDC=EDCI=1-ethyl-3-(3-dimethylaminopropyl)carbodiimide
EtOAc=ethyl acetate
h=hours

### HOBt=Hydroxybenzotriazole

LCMS=Liquid chromatography-mass spectrometry
LHMDS=LiHMDS=lithium bis(trimethylsilyl)amide
LiAlH₄=lithium aluminum hydride
min=minutes
Me=methyl
MeOH═CH₃OH=methanol
NaBH₄=sodium borohydride
Na₂SO₄=sodium sulfate
NH₄Cl=Ammonium chloride
Pd(dppf)Cl₂=[1,1′-Bis(diphenylphosphino)ferrocene]palladium(II) dichloride

### SFC=Supercritical Fluid Chromatography

TBAF=tetra-n-butylammonium fluoride
TEA=triethylamine
TFA=trifluoroacetic acid
THF=tetrahydrofuran

### TMS=Trimethylsilyl

CDCl₃=heavy chloroform
CD₃OD=heavy methanol
1 Standard atmosphere [atm]=101325 pascal [Pa]=14.6959488 psi

The meanings of the abbreviations in the nuclear magnetic resonance spectra are shown below: s=singlet, d=doublet, dd=double doublet, dt=double triplet, ddd=double double doublet, sept=septet, t=triplet, m=multiplet, br=broad, brs=broad singlet, q=quartet, J=coupling constant and Hz=hertz.

Several methods for preparing the compounds of this disclosure are described in the following Schemes and Examples. Starting materials and intermediates were purchased commercially from common catalog sources or were made using known procedures, or as otherwise illustrated. Some frequently applied routes to the compounds of Formula (I′) or (I) are described in in the Schemes that follow. In some cases, the order of carrying out the reaction steps in the schemes may be varied to facilitate the reaction or to avoid unwanted reaction products. An asterisk (*) may be used in a chemical structure drawing that indicates the location of a chiral center.

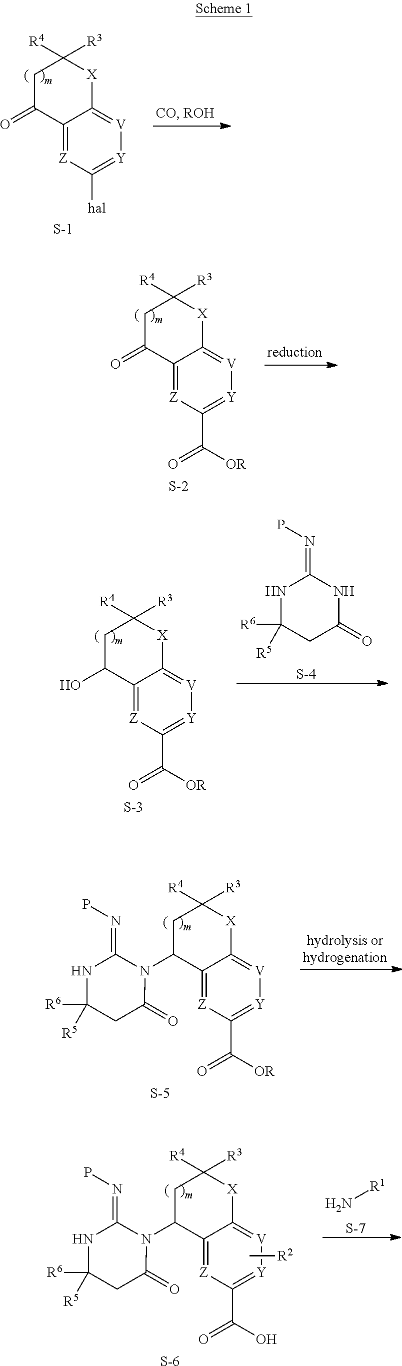

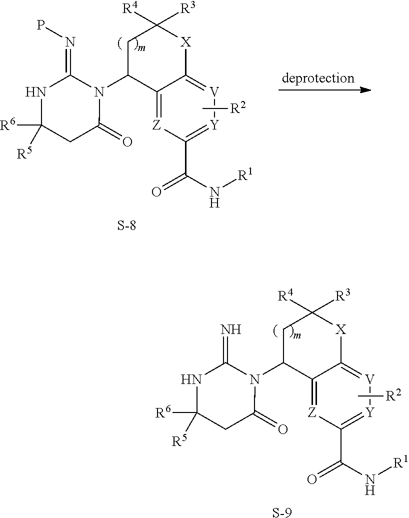

Intermediate compounds of Formula S-2, in which hal is a halogen such as Cl, Br and I, are prepared from S-1 after carbonylation in the presence of an alcohol. Ketone reduction in S-2 can be performed racemically using a hydride source such as NaBH₄ or LiAlH₄ or stereoselectivity using catalytic asymmetric hydrogenation or biocatalysis (ketoreductases) to yield alcohols S-3. Treatment of S-3 with N-protected iminopyrimidone S-4 (WO2017142825) under Mitsunobu conditions gives intermediates S-5. Alternatively, alcohol in intermediates S-3 could be transformed into a leaving group such as a mesylate, tosylate or halogen which can be displaced with iminopyrimidones S-4 to give intermediates S-5. Acid or base catalyzed hydrolysis or hydrogenation of the S-5 ester followed by coupling with amines S-7 provides intermediates S-8 which after protecting group removal yields the products of Formula S-9.

Reactions sensitive to moisture or air were performed inside a glove-box or under nitrogen or argon using anhydrous solvents and reagents. The progress of reactions was determined by either analytical thin layer chromatography (TLC) usually performed with pre-coated TLC plates or liquid chromatography-mass spectrometry (LC/MS).

Typically, the analytical LC-MS system used consisted of a Waters ZQ™ platform with electrospray ionization in positive ion detection mode with an Agilent 1100 series HPLC with autosampler. The column was commonly a Waters Xterra MS C18, 3.0×50 mm, 5 μm or a Waters Acquity UPLC® BEH C18 1.0×50 mm, 1.7 μm. The flow rate was 1 mL/min, and the injection volume was 10 μL. UV detection was in the range 210-400 nm. The mobile phase consisted of solvent A (water plus 0.05% TFA) and solvent B (MeCN plus 0.05% TFA) with a gradient of 100% solvent A for 0.7 min changing to 100% solvent B over 3.75 min, maintained for 1.1 min, then reverting to 100% solvent A over 0.2 min.

Preparative HPLC purifications were usually performed using either a mass spectrometry directed system or a non-mass guided system. Usually they were performed on a Waters Chromatography Workstation configured with LC-MS System consisting of: Waters ZQ™ single quad MS system with Electrospray Ionization, Waters 2525 Gradient Pump, Waters 2767 Injecto/Collector, Waters 996 PDA Detector, the MS Conditions of: 150-750 amu, Positive Electrospray, Collection Triggered by MS, and a Waters SUNFIRE® C-18 5-micron, 30 mm (id)×100 mm column. The mobile phases consisted of mixtures of acetonitrile (10-100%) in water containing 0.1% TFA. Flow rates were maintained at 50 mL/min, the injection volume was 1800 μL, and the UV detection range was 210-400 nm. An alternate preparative HPLC system used was a Gilson Workstation consisting of: Gilson GX-281 Injector/Collector, Gilson UV/VIS-155 Detector, Gilson 333 and 334 Pumps, and either a Phenomenex Gemini-NX C-18 5-micron, 50 mm (id)×250 mm column or a Waters XBridge™ C-18 5-micron OBD™, 30 mm (id)×250 mm column. The mobile phases consisted of mixtures of acetonitrile (0-75%) in water containing 5 mmol (NH₄)HCO₃. Flow rates were maintained at 50 mL/min for the Waters Xbridge™ column and 90 mL/min for the Phenomenex Gemini column. The injection volume ranged from 1000-8000 μL, and the UV detection range was 210-400 nm. Mobile phase gradients were optimized for the individual compounds. Reactions performed using microwave irradiation were normally carried out using an Emrys Optimizer manufactured by Personal Chemistry, or an Initiator manufactured by Biotage. Concentration of solutions was carried out on a rotary evaporator under reduced pressure. Flash chromatography was usually performed using either a Biotage® Flash Chromatography apparatus (Dyax Corp.), an ISCO CombiFlash® Rf apparatus, or an ISCO CombiFlash® Companion XL on silica gel (32-63 μM, 60 Å pore size) in pre-packed cartridges of the size noted. ¹H NMR spectra were acquired at 500 MHz spectrometers in CDCl₃ solutions unless otherwise noted. Chemical shifts were reported in parts per million (ppm). Tetramethylsilane (TMS) was used as internal reference in CDCl₃ solutions, and residual CH₃OH peak or TMS was used as internal reference in CD₃OD solutions. Coupling constants (J) were reported in hertz (Hz). Chiral analytical chromatography was most commonly performed on one of CHIRALPAK® AS, CHIRALPAK® AD, CHIRALCEL® OD, CHIRALCEL® IA, or CHIRALCEL® OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of CHIRALPAK AS, of CHIRALPAK AD, CHIRALCEL® OD, CHIRALCEL® IA, CHIRALCEL® OJ columns (20×250 mm) (Daicel Chemical Industries, Ltd.) with desired isocratic solvent systems identified on chiral analytical chromatography or by supercritical fluid (SFC) conditions.

It is understood that a chiral center in a compound may exist in the “S” or “R” stereo-configuration, or as a mixture of both. Within a molecule, each bond drawn as a straight line from a chiral center includes both the (R) and (S) stereoisomers as well as mixtures thereof.

### Example 1A

### 4-(4,4-diethyl-2-imino-6-oxotetrahydropyrimidin-1(2H)-yl)-N—((S)-2,2-dimethylchroman-4-yl)chroman-6-carboxamide

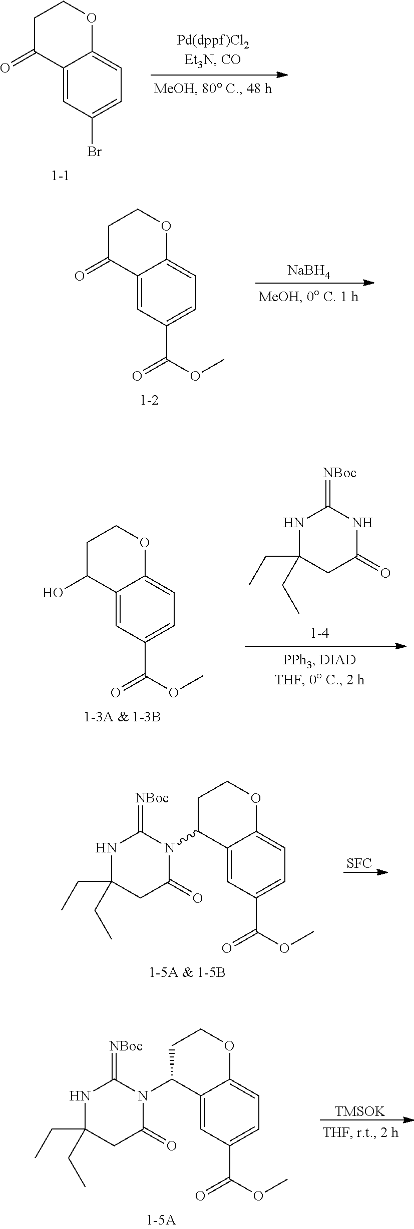

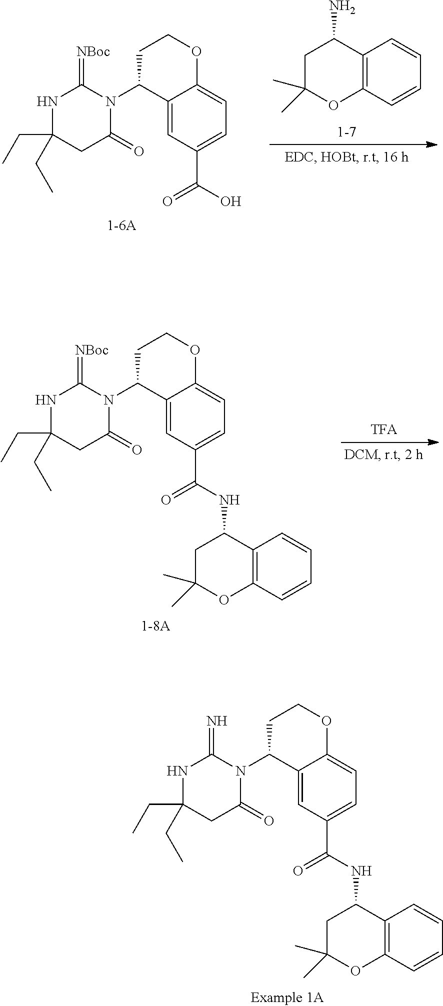

### Step A: methyl 4-oxochroman-6-carboxylate 1-2

Pd(dppf)Cl₂ (11.28 g, 15.41 mmol) and triethylamine (64.5 mL, 462 mmol) were added to a solution of 6-bromochroman-4-one 1-1 (35 g, 154 mmol) in MeOH (120 mL) at 25° C. The solution was stirred at 80° C. for 48 h under CO atmosphere (50 psi). The reaction mixture was cooled to room temperature, then concentrated under reduced pressure to give a residue. The residue was purified by flash silica gel chromatography (ISCO®; Agela® Flash Column Silica-CS (120 g), Eluent of 10% Ethyl acetate/petroleum ether gradient @55 mUmin) to give methyl 4-oxochroman-6-carboxylate 1-2.

MS (ESI) m/z: 248.1 (M+41+H⁺)

¹H NMR (400 MHz, CD₃OD) δ 8.49 (d, J=2.0 Hz, 1H), 8.12 (dd, J=8.8, 2.0 Hz, 1H), 7.08 (d, J=8.8 Hz, 1H), 4.60-4.65 (m, 2H), 3.89 (s, 3H), 2.85 (t, J=6.4 Hz, 2H).

### Step B: methyl 4-hydroxychroman-6-carboxylate 1-3A & 1-3B

Sodium borohydride (3.03 g, 80 mmol) at 0° C. was added in portions to a solution of methyl 4-oxochroman-6-carboxylate 1-2 (15 g, 72.7 mmol) in MeOH (100 mL). Then the mixture was stirred at 0° C. for 1 h. Then the mixture was quenched by addition of saturated NH₄Cl (50 mL), then concentrated under reduced pressure to give a residue, added water (100 mL), extracted with EtOAc (50 mL*2). The combined organic extracts were washed with brine (50 mL), dried over Na₂SO₄, filtered and concentrated under reduced pressure, which was purified by flash silica gel chromatography (ISCO®; Agela® Flash Column Silica-CS (40 g), Eluent of 0-22% Ethyl acetate/petroleum ether gradient @50 mL/min) to give methyl 4-hydroxychroman-6-carboxylate 1-3A & 1-3B.

MS (ESI) m/z: 209.0 (M+H⁺).

¹H NMR (400 MHz, chloroform-d) δ 8.03 (d, J=2.0 Hz, 1H), 7.86 (dd, J=2.0, 8.8 Hz, 1H), 6.85 (d, J=8.8 Hz, 1H), 4.82 (s, 1H), 4.41-4.24 (m, 2H), 3.87 (s, 3H), 2.20-2.05 (m, 3H)

### Step C: (E)-methyl 4-(2-((tert-butoxycarbonyl)imino)-4,4-diethyl-6-oxotetrahydropyrimidin-1(2H)-yl)chroman-6-carboxylate 1-5A & 1-5B

DIAD (18.86 mL, 96 mmol) was added dropwise to a solution of (Z)-tert-butyl (4,4-diethyl-6-oxotetrahydropyrimidin-2(1H)-ylidene)carbamate 1-4 (12.94 g, 48.0 mmol), methyl 4-hydroxychroman-6-carboxylate 1-3A & 1-3B (10 g, 48.0 mmol) and triphenylphosphine (25.2 g, 96 mmol) in THF (50 mL) at 0° C. under N₂ atmosphere. The mixture was stirred at 0° C. for 2 h. The reaction was quenched by water (60 mL) and extracted with ethyl acetate (50 mL*2). The combined organic extracts were washed with brine (50 mL), dried over Na₂SO₄, filtered and concentrated under reduced pressure to give a residue, which was purified by flash silica gel chromatography (ISCO®; Agela® Flash Column Silica-CS (40 g), Eluent of 0-25% Ethyl acetate/petroleum ether gradient @50 mL/min) to give (E)-methyl 4-(2-((tert-butoxycarbonyl)imino)-4,4-diethyl-6-oxotetrahydropyrimidin-1(2H)-yl)chroman-6-carboxylate 1-5A & 1-5B.

MS (ESI) m/z: 460.2 (M+H⁺).

¹H NMR (500 MHz, chloroform-d) δ 7.75 (dd, J=2.0, 8.5 Hz, 1H), 7.60 (s, 1H), 6.82 (d, J=8.5 Hz, 1H), 6.37 (dd, J=7.0, 10.1 Hz, 1H), 4.43 (m, 1H), 4.22 (m, 1H), 3.85-3.78 (m, 3H), 2.84-2.70 (m, 1H), 2.62-2.44 (m, 2H), 2.11-2.03 (m, 1H), 1.79-1.59 (m, 5H), 1.50 (s, 9H), 1.02-0.89 (m, 6H)

### Step D: of methyl 4-(2-((tert-butoxycarbonyl)imino)-4,4-diethyl-6-oxotetrahydropyrimidin-1(2H)-yl)chroman-6-carboxylate 1-5A

Methyl 4-(2-((tert-butoxycarbonyl)imino)-4,4-diethyl-6-oxotetrahydropyrimidin-1(2H)-yl)chroman-6-carboxylate 1-5A (6.5 g, 14.14 mmol) was purified by SFC on (Instrument SFC 5, Method Column DAICEL CHIRALCEL OD (250 mm*50 mm, 10 um), Condition 0.1% aqNH₃ MeOH, begin B 40%, end B 40%, FlowRate (mL/min) 200, Injections 150) to afford methyl 4-(2-((tert-butoxycarbonyl)imino)-4,4-diethyl-6-oxotetrahydropyrimidin-1(2H)-yl)chroman-6-carboxylate 1-5A (Rt=6.460 min).

MS (ESI) m/z: 460.2 (M+H⁺).

1-5A: ¹H NMR (500 MHz, chloroform-d) δ 10.10 (s, 1H), 7.75 (dd, J=2.0, 8.5 Hz, 1H), 7.61 (s, 1H), 6.83 (d, J=8.5 Hz, 1H), 6.45-6.31 (m, 1H), 4.51-4.39 (m, 1H), 4.25-4.20 (m, 1H), 3.82 (s, 3H), 2.82-2.70 (m, 1H), 2.61-2.48 (m, 2H), 2.11-2.03 (m, 1H), 1.79-1.61 (m, 4H), 1.51 (s, 9H), 1.03-0.90 (m, 6H)

### Step E: 4-(2-((tert-butoxycarbonyl)imino)-4,4-diethyl-6-oxotetrahydropyrimidin-1(2H)-yl)chroman-6-carboxylic acid 1-6A

Potassium trimethylsilanolate (1.842 g, 14.36 mmol) was added to a solution of methyl 4-(2-((tert-butoxycarbonyl)imino)-4,4-diethyl-6-oxotetrahydropyrimidin-1(2H)-yl)chroman-6-carboxylate 1-5A (2.2 g, 4.79 mmol) in THF (35 mL). The reaction was stirred at 22° C. for 1 h. The solution of 4-(2-((tert-butoxycarbonyl)imino)-4,4-diethyl-6-oxotetrahydropyrimidin-1(2H)-yl)chroman-6-carboxylic acid 1-6A was used for next step directly without any further manipulation or purification.

MS (ESI) m/z: 446.1 (M+H⁺)

### Step F: tert-butyl (1-(6-(((S)-2,2-dimethylchroman-4-yl)carbamoyl)chroman-4-yl)-4,4-diethyl-6-oxotetrahydropyrimidin-2(1H)-ylidene)carbamate 1-8A

DIEA (3.14 mL, 17.96 mmol) was added to a solution of 4-(2-((tert-butoxycarbonyl)imino)-4,4-diethyl-6-oxotetrahydropyrimidin-1(2H)-yl)chroman-6-carboxylic acid 1-6A (2.0 g, 4.49 mmol), EDC (1.721 g, 8.98 mmol), 1H-benzo[d][1,2,3]triazol-1-ol 7 (1.213 g, 8.98 mmol) and (S)-2,2-dimethylchroman-4-amine (1.591 g, 8.98 mmol) in THF (35 mL). The reaction was stirred at 22° C. for 5 h. The mixture was quenched with water (10 mL) and extracted with EtOAc (30 mL*3). The organic layers were washed with brine (30 mL), dried over Na₂SO₄, filtered, and concentrated in vacuo to afford crude product, which was purified by flash silica gel chromatography (ISCO®; Agela® Flash Column Silica-CS (40 g), Eluent of 0˜26% AcOEt/petroleum ether gradient @50 mL/min) to give tert-butyl (1-(6-(((S)-2,2-dimethylchroman-4-yl)carbamoyl)chroman-4-yl)-4,4-diethyl-6-oxotetrahydropyrimidin-2(1H)-ylidene)carbamate 1-8A.

MS (ESI) m/z: 605.4 (M+H⁺)

¹H NMR (400 MHz, chloroform-d) δ 10.10 (s, 1H), 7.51 (s, 1H), 7.39 (d, J=8.4 Hz, 1H), 7.25-7.22 (m, 1H), 7.16 (t, J=7.6 Hz, 1H), 6.92-6.75 (m, 3H), 6.48-6.34 (m, 1H), 6.07 (d, J=8.8 Hz, 1H), 5.53-5.39 (m, 1H), 4.44 (d, J=11.2 Hz, 1H), 4.20 (t, J=11.2 Hz, 1H), 2.83-2.67 (m, 1H), 2.58-2.46 (m, 2H), 2.27 (m, 1H), 2.07 (m, 1H), 1.84-1.56 (m, 7H), 1.50 (s, 9H), 1.43 (s, 3H), 1.35 (s, 3H), 0.92 (m, 6H)

### Step G: 4-(4,4-diethyl-2-imino-6-oxotetrahydropyrimidin-1(2H)-yl)-N—((S)-2,2-dimethylchroman-4-yl)chroman-6-carboxamide Example 1A

A solution of tert-butyl (1-(6-(((S)-2,2-dimethylchroman-4-yl)carbamoyl)chroman-4-yl)-4,4-diethyl-6-oxotetrahydropyrimidin-2(1H)-ylidene)carbamate 1-8A (2.4 g, 3.97 mmol) in DCM (15 mL) and TFA (3 mL) was stirred at 20° C. for 2 h. The mixture was concentrated in vacuo to give the crude product which was purified by Prep-HPLC (0.1% TFA) (Instrument EG Method Column Waters XSELECT C18 150*30 mm*5 um Condition water (0.1% TFA)-ACN Begin B 22 End B 55 Gradient Time (min) 11 100% B Hold Time (min) 2 FlowRate (mL/min) 25 Injections 10) to afford 4-(4,4-diethyl-2-imino-6-oxotetrahydropyrimidin-1(2H)-yl)-N—((S)-2,2-dimethylchroman-4-yl)chroman-6-carboxamide Example 1A.

MS (ESI) m/z: 505.3 (M+H⁺)

¹H NMR (400 MHz, methanol-d₄) δ 8.54 (d, J=8.8 Hz, 1H), 7.70 (d, J=8.0 Hz, 1H), 7.64 (s, 1H), 7.18-7.08 (m, 2H), 6.90 (d, J=8.4 Hz, 1H), 6.83 (t, J=7.2 Hz, 1H), 6.74 (d, J=8.0 Hz, 1H), 5.54-5.38 (m, 1H), 4.57-4.44 (m, 1H), 4.22 (t, J=10.8 Hz, 1H), 2.93-2.67 (m, 3H), 2.27-2.17 (m, 1H), 2.12-2.10 (m, 1H), 2.10-2.00 (m, 1H), 1.83-1.63 (m, 4H), 1.44 (s, 3H), 1.34 (s, 3H), 0.98-0.93 (m, 6H)

### Example 2A

### 4-(4,4-diethyl-2-imino-6-oxotetrahydropyrimidin-1(2H)-yl)-N-((3S,4R)-3-hydroxy-2,2-dimethylchroman-4-yl)chroman-6-carboxamide

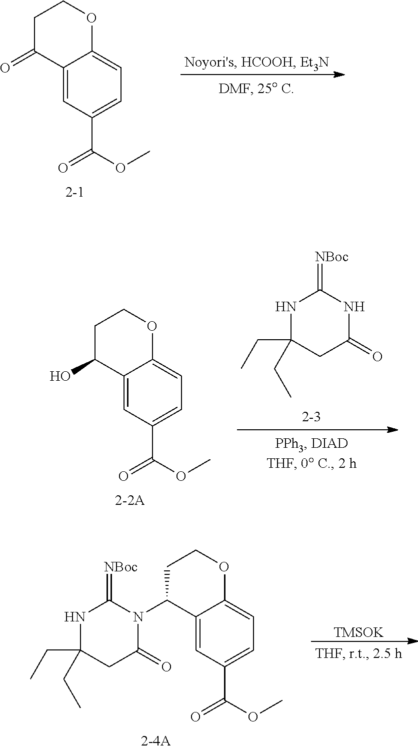

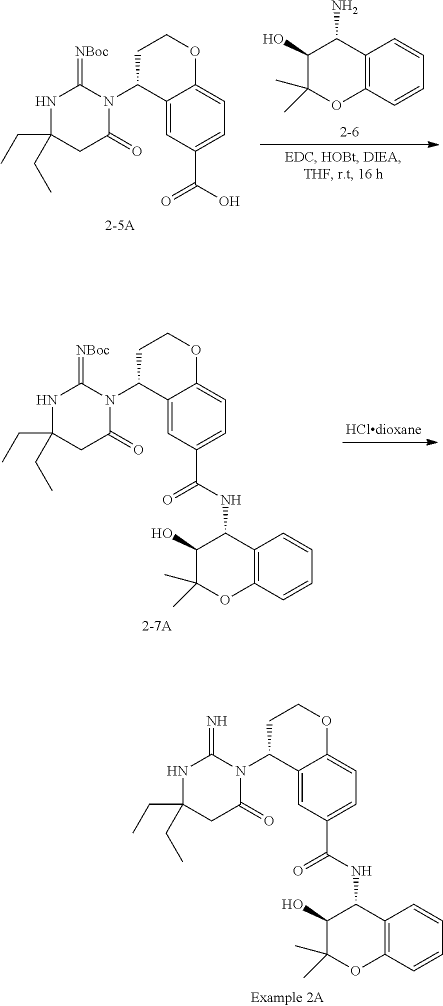

### Step A: (S)-methyl 4-hydroxychroman-6-carboxylate 2-2A

A solution of formic acid (30 g, 652 mmol) and triethylamine (120 g, 1186 mmol) in DMF (320 mL) was stirred for 15 min, then methyl 4-oxochroman-6-carboxylate 2-1 (40 g, 194 mmol) and (S,S)—N-(p-toluenesulfonyl)-1,2-diphenylethanediamine(chlorox[-cymene)ruthenium(II) (1.234 g, 1.940 mmol) were added, and the mixture was stirred for 10 h at 25° C. The mixture was diluted with water (300 mL) and extracted with EtOAc (250 mL*3). The organic layers were washed with brine (300 mL), dried over anhydrous Na₂SO₄, filtered and concentrated. The residue was purified by flash silica gel chromatography (ISCO®; Agela® Flash Column Silica-CS (150 g), Eluent of 0˜30% Ethyl acetate/petroleum ether gradient @65 mL/min) to afford (S)-methyl 4-hydroxychroman-6-carboxylate 2-2A.

MS (ESI) m/z: 209.0 (M+H⁺)

¹H NMR (500 MHz, chloroform-d) δ 8.02 (s, 1H), 7.84 (d, J=8.50 Hz, 1H), 6.83 (d, J=8.0 Hz, 1H), 4.81 (q, J=4.0 Hz, 1H), 4.26-4.39 (m, 2H), 3.85 (s, 3H), 2.59 (d, J=4.0 Hz, 1H), 2.02-2.15 (m, 2H).

### Step B: methyl 4-(2-((tert-butoxycarbonyl)imino)-4,4-diethyl-6-oxotetrahydropyrimidin-1(2H)-yl)chroman-6-carboxylate 2-4A

(E)-diisopropyl diazene-1,2-dicarboxylate (34.0 mL, 173 mmol) was added dropwise to a solution of (Z)-tert-butyl (4,4-diethyl-6-oxotetrahydropyrimidin-2(1H)-ylidene)carbamate 2-3 (40 g, 149 mmol), methyl 4-hydroxychroman-6-carboxylate 2-2A (30 g, 144 mmol) and triphenylphosphine (48 g, 183 mmol) in THF (500 mL) at 0° C. under N₂ atmosphere. Then the mixture was stirred at 25° C. for 3 h. The mixture was concentrated in vacuo, then purified by flash silica gel chromatography (ISCO®; Agela® Flash Column Silica-CS (150 g), Eluent of 0-5% AcOEt/DCM gradient @65 mL/min) to give methyl 4-(2-((tert-butoxycarbonyl)imino)-4,4-diethyl-6-oxotetrahydropyrimidin-1(2H)-yl)chroman-6-carboxylate 2-4A.

MS (ESI) m/z: 460.2 (M+H⁺).

¹H NMR (400 MHz, chloroform-d) δ 10.48-10.83 (m, 1H), 7.78 (d, J=8.8 Hz, 1H), 7.60 (s, 1H), 6.85 (d, J=8.8 Hz, 1H), 5.93-6.04 (m, 1H), 4.43 (d, J=11.2 Hz, 1H), 4.18 (t, J=11.2 Hz, 1H), 3.82 (s, 3H), 2.72-2.77 (m, 1H), 2.51-2.56 (m, 2H), 2.04-2.08 (m, 1H), 1.60-1.70 (m, 4H), 1.49 (s, 9H), 0.90-0.99 (m, 6H).

### Step C: (R)-4-(2-((tert-butoxycarbonyl)imino)-4,4-diethyl-6-oxotetrahydropyrimidin-1(2H)-yl)chroman-6-carboxylic acid 2-5A

Potassium trimethylsilanolate (7.54 g, 58.8 mmol) was added to a solution of (R)-methyl 4-(2-((tert-butoxycarbonyl)imino)-4,4-diethyl-6-oxotetrahydropyrimidin-1(2H)-yl)chroman-6-carboxylate 2-4A (9.0 g, 19.59 mmol) in THF (300 mL). The reaction was stirred at 22° C. for 2.5 h. The solution of (R)-4-(2-((tert-butoxycarbonyl)imino)-4,4-diethyl-6-oxotetrahydropyrimidin-1(2H)-yl)chroman-6-carboxylic acid 2-5A was used for next step directly without any further manipulation or purification.

MS (ESI) m/z: 446.0 (M+H⁺)

### Step D: tert-butyl (4,4-diethyl-1-((R)-6-(((3S,4R)-3-hydroxy-2,2-dimethylchroman-4-yl)carbamoyl)chroman-4-yl)-6-oxotetrahydropyrimidin-2(1H)-ylidene)carbamate 2-7A

DIEA (13.69 mL, 78 mmol) was added to a solution of (R)-4-(2-((tert-butoxycarbonyl)imino)-4,4-diethyl-6-oxotetrahydropyrimidin-1(2H)-yl)chroman-6-carboxylic acid 2-5A (8.73 g, 19.60 mmol), EDC (9.39 g, 49.0 mmol), 1H-benzo[d][1,2,3]triazol-1-ol (5.30 g, 39.2 mmol) and (3S,4R)-4-amino-2,2-dimethylchroman-3-ol 2-6 (4.54 g, 23.51 mmol) in THF (300 mL). The reaction was stirred at 22° C. for 16 h. The mixture was quenched with water (80 mL) and extracted with EtOAc (80 mL×3). The organic layers were washed with brine (100 mL), dried over Na₂SO₄, filtered, and concentrated in vacuo to afford crude product as an oil, which was purified by flash silica gel chromatography (ISCO®; Agela® Flash Column Silica-CS (150 g), Eluent of 0˜30% Ethyl acetate/petroleum ether gradient @65 mL/min) to give tert-butyl (4,4-diethyl-1-((R)-6-(((3S,4R)-3-hydroxy-2,2-dimethylchroman-4-yl)carbamoyl)chroman-4-yl)-6-oxotetrahydropyrimidin-2(1H)-ylidene)carbamate 2-7A.

MS (ESI) m/z: 621.3 (M+H⁺)

¹H NMR (500 MHz, chloroform-d) δ 10.12 (br, 1H), 7.55 (s, 1H), 7.36-7.43 (m, 1H), 7.25-7.27 (m, 1H), 7.18-7.23 (m, 1H), 6.94 (dt, J=1.0, 7.5 Hz, 1H), 6.80-6.87 (m, 2H), 6.34-6.43 (m, 2H), 5.11-5.12 (m, 1H), 4.84 (s, 1H), 4.39-4.49 (m, 1H), 4.23 (dt, J=2.0, 11.5 Hz, 1H), 3.73 (d, J=8.0 Hz, 1H), 2.68-2.83 (m, 1H), 2.45-2.56 (m, 2H), 2.05-2.09 (m, 1H), 1.60-1.74 (m, 4H), 1.49-1.50 (m, 12H), 1.28 (s, 3H), 0.90-0.98 (m, 6H).

### Step E: 4-(4,4-diethyl-2-imino-6-oxotetrahydropyrimidin-1(2H)-yl)-N-((3S,4R)-3-hydroxy-2,2-dimethylchroman-4-yl)chroman-6-carboxamide Example 2A

A solution of tert-butyl (4,4-diethyl-1-(6-(((3S,4R)-3-hydroxy-2,2-dimethylchroman-4-yl)carbamoyl)chroman-4-yl)-6-oxotetrahydropyrimidin-2(1H)-ylidene)carbamate 2-7A (14 g, 22.55 mmol) in HCl-dioxane (4M) (200 mL) was stirred at 25° C. for 10 h. The mixture was concentrated in vacuo. The crude was purified by Prep-HPLC (Instrument ACSSH-PrepL-K2 Method Column YMC-Triart Prep C18 250*50 mm*10 um Condition water (0.1% TFA)-ACN Begin B 20 End B 50 Gradient Time (min) 25 100% B Hold Time (min) 3 FlowRate (mL/min) 120) to afford 4-(4,4-diethyl-2-imino-6-oxotetrahydropyrimidin-1(2H)-yl)-N-((3S,4R)-3-hydroxy-2,2-dimethylchroman-4-yl)chroman-6-carboxamide Example 2A.

MS (ESI) m/z 521.3 (M+H⁺)

¹H NMR (400 MHz, methanol-d₄) δ 8.56 (d, J=8.8 Hz, 1H), 7.72 (d, J=8.4 Hz, 1H), 7.66 (s, 1H), 7.06-7.14 (m, 2H), 6.89 (d, J=8.4 Hz, 1H), 6.80-6.85 (m, 1H), 6.74 (d, J=8.0 Hz, 1H), 5.30-5.88 (m, 1H), 5.22 (t, J=8.8 Hz, 1H), 4.49 (td, J=3.6, 11.6 Hz, 1H), 4.21 (t, J=10.8 Hz, 1H), 3.75 (d, J=9.6 Hz, 1H), 2.65-2.87 (m, 3H), 2.16-2.24 (m, 1H), 1.62-1.81 (m, 4H), 1.46 (s, 3H), 1.24 (s, 3H), 0.91-0.98 (m, 6H).

The compounds in Table 1-4 were prepared in an analogous fashion to that described in Scheme 1 and the experimentals described herein. The isomers were separated by preparative HPLC or/and preparative chiral SFC.

TABLE 1

| | | LC/MS | |
|---|---|---|---|
| Example | Structure | (M + 1)⁺ | Name |
| 3A | 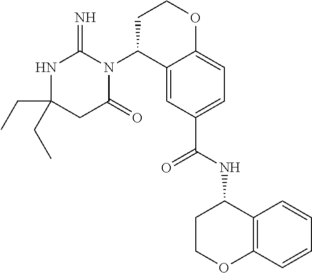 [SMILES: CCC1(CC)CC(=O)N([C@@H]2CCOc3ccc(C(=O)N[C@H]4CCOc5ccccc54)cc32)C(=N)N1] | 477.5 | (R)-N-((S)-chroman-4-yl)-4- (4,4-diethyl-2-imino-6- oxotetrahydropyrimidin-1(2H)- yl)chromane-6-carboxamide |
| 4A | 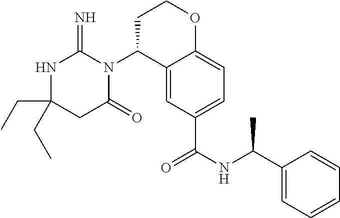 [SMILES: CCC1(CC)CC(=O)N([C@@H]2CCOc3ccc(C(=O)N[C@@H](C)c4ccccc4)cc32)C(=N)N1] | 449.2 | (R)-4-(4,4-diethyl-2-imino-6- oxotetrahydropyrimidin-1(2H)- yl)-N-((S)-1- phenylethyl)chromane-6- carboxamide |
| 5A | 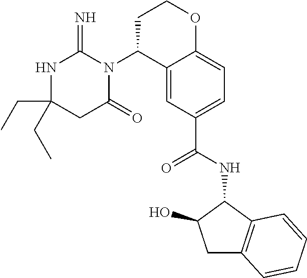 [SMILES: CCC1(CC)CC(=O)N([C@@H]2CCOc3ccc(C(=O)N[C@@H]4c5ccccc5C[C@H]4O)cc32)C(=N)N1] | 477.3 | (R)-4-(4,4-diethyl-2-imino-6- oxotetrahydropyrimidin-1(2H)- yl)-N-((1R,2R)-2-hydroxy-2,3- dihydro-1H-inden-1- yl)chromane-6-carboxamide |
| 6A | 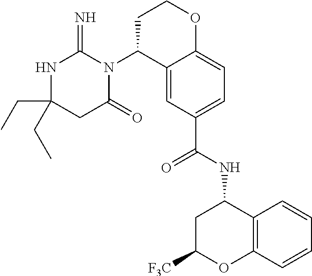 [SMILES: CCC1(CC)CC(=O)N([C@@H]2CCOc3ccc(C(=O)N[C@H]4C[C@H](C(F)(F)F)Oc5ccccc54)cc32)C(=N)N1] | 545.2 | (R)-4-(4,4-diethyl-2-imino-6- oxotetrahydropyrimidin-1(2H)- yl)-N-((2R,4S)-2- (trifluoromethyl)chroman-4- yl)chromane-6-carboxamide |
| 6B | 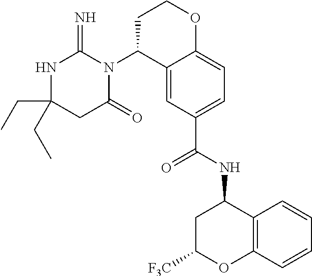 [SMILES: CCC1(CC)CC(=O)N([C@@H]2CCOc3ccc(C(=O)N[C@@H]4C[C@@H](C(F)(F)F)Oc5ccccc54)cc32)C(=N)N1] | 545.2 | (R)-4-(4,4-diethyl-2-imino-6- oxotetrahydropyrimidin-1(2H)- yl)-N-((2S,4R)-2- (trifluoromethyl)chroman-4- yl)chromane-6-carboxamide |
| 7A | 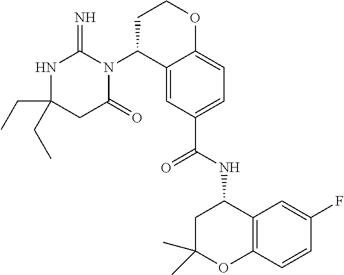 [SMILES: CCC1(CC)CC(=O)N([C@@H]2CCOc3ccc(C(=O)N[C@H]4CC(C)(C)Oc5ccc(F)cc54)cc32)C(=N)N1] | 523.2 | (R)-4-(4,4-diethyl-2-imino-6- oxotetrahydropyrimidin-1(2H)- yl)-N-((S)-6-fluoro-2,2- dimethylchroman-4- yl)chromane-6-carboxamide |
| 7B | 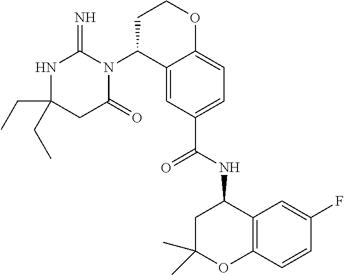 [SMILES: CCC1(CC)CC(=O)N([C@@H]2CCOc3ccc(C(=O)N[C@@H]4CC(C)(C)Oc5ccc(F)cc54)cc32)C(=N)N1] | 523.2 | (R)-4-(4,4-diethyl-2-imino-6- oxotetrahydropyrimidin-1(2H)- yl)-N-((R)-6-fluoro-2,2- dimethylchroman-4- yl)chromane-6-carboxamide |
| 8A | 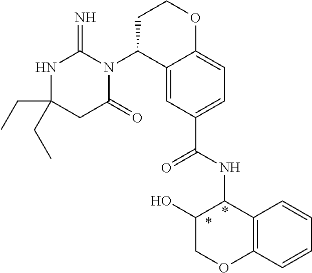 [SMILES: CCC1(CC)CC(=O)N([C@@H]2CCOc3ccc(C(=O)NC4c5ccccc5OCC4O)cc32)C(=N)N1] | 493.3 | (4R)-4-(4,4-diethyl-2-imino-6- oxotetrahydropyrimidin-1(2H)- yl)-N-(3-hydroxychroman-4- yl)chromane-6-carboxamide |
| 8B | 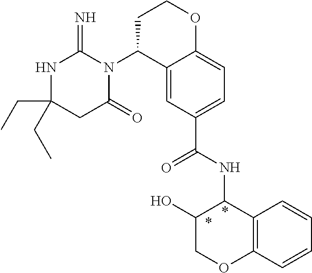 [SMILES: CCC1(CC)CC(=O)N([C@@H]2CCOc3ccc(C(=O)NC4c5ccccc5OCC4O)cc32)C(=N)N1] | 493.3 | (4R)-4-(4,4-diethyl-2-imino-6- oxotetrahydropyrimidin-1(2H)- yl)-N-(3-hydroxychroman-4- yl)chromane-6-carboxamide |
| 8C | 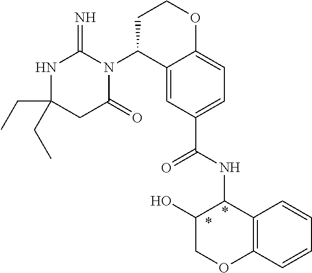 [SMILES: CCC1(CC)CC(=O)N([C@@H]2CCOc3ccc(C(=O)NC4c5ccccc5OCC4O)cc32)C(=N)N1] | 493.1 | (4R)-4-(4,4-diethyl-2-imino-6- oxotetrahydropyrimidin-1(2H)- yl)-N-(3-hydroxychroman-4- yl)chromane-6-carboxamide |
| 8D | 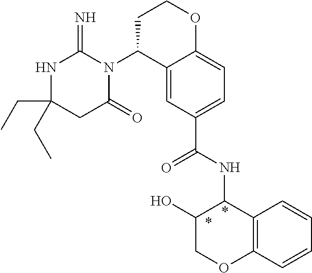 [SMILES: CCC1(CC)CC(=O)N([C@@H]2CCOc3ccc(C(=O)NC4c5ccccc5OCC4O)cc32)C(=N)N1] | 493.3 | (4R)-4-(4,4-diethyl-2-imino-6- oxotetrahydropyrimidin-1(2H)- yl)-N-(3-hydroxychroman-4- yl)chromane-6-carboxamide |
| 9A | 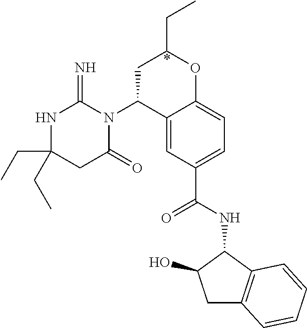 | 505.3 | (4R)-4-(4,4-diethyl-2-imino-6- oxotetrahydropyrimidin-1(2H)- yl)-2-ethyl-N-((1R,2R)-2- hydroxy-2,3-dihydro-1H- inden-1-yl)chromane-6- carboxamide |
| 9B | 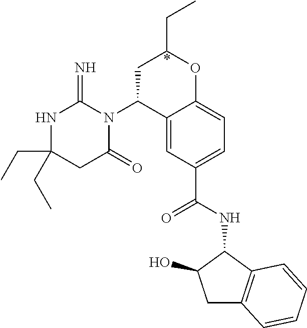 | 505.3 | (4R)-4-(4,4-diethyl-2-imino-6- oxotetrahydropyrimidin-1(2H)- yl)-2-ethyl-N-((1R,2R)-2- hydroxy-2,3-dihydro-1H- inden-1-yl)chromane-6- carboxamide |
| 10A | 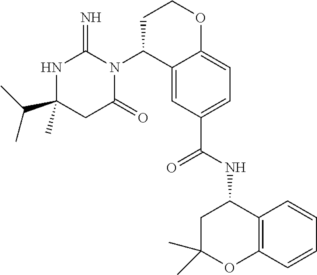 [SMILES: CC(C)[C@]1(C)CC(=O)N([C@@H]2CCOc3ccc(C(=O)N[C@H]4CC(C)(C)Oc5ccccc54)cc32)C(=N)N1] | 505.3 | (R)-N-((S)-2,2- dimethylchroman-4-yl)-4-((S)- 2-imino-4-isopropyl-4-methyl- 6-oxotetrahydropyrimidin- 1(2H)-yl)chromane-6- carboxamide |
| 11A | 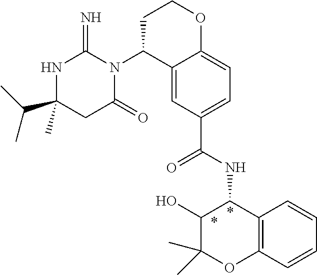 [SMILES: CC(C)[C@]1(C)CC(=O)N([C@@H]2CCOc3ccc(C(=O)N[C@@H]4c5ccccc5OC(C)(C)C4O)cc32)C(=N)N1] | 521.4 | (4R)-N-(3-hydroxy-2,2- dimethylchroman-4-yl)-4-((S)- 2-imino-4-isopropyl-4-methyl- 6-oxotetrahydropyrimidin- 1(2H)-yl)chromane-6- carboxamide |
| 11B | 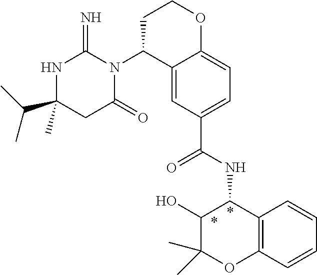 [SMILES: CC(C)[C@]1(C)CC(=O)N([C@@H]2CCOc3ccc(C(=O)N[C@@H]4c5ccccc5OC(C)(C)C4O)cc32)C(=N)N1] | 521.3 | (4R)-N-(3-hydroxy-2,2- dimethylchroman-4-yl)-4-((S)- 2-imino-4-isopropyl-4-methyl- 6-oxotetrahydropyrimidin- 1(2H)-yl)chromane-6- carboxamide |
| 12A | 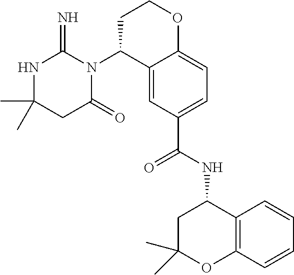 [SMILES: CC1(C)CC(=O)N([C@@H]2CCOc3ccc(C(=O)N[C@H]4CC(C)(C)Oc5ccccc54)cc32)C(=N)N1] | 477.3 | (R)-N-((S)-2,2- dimethylchroman-4-yl)-4-(2- imino-4,4-dimethyl-6- oxotetrahydropyrimidin-1(2H)- yl)chromane-6-carboxamide |
| 13A | 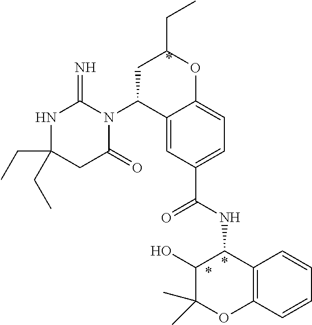 | 549.3 | (4R)-4-(4,4-diethyl-2-imino-6- oxotetrahydropyrimidin-1(2H)- yl)-2-ethyl-N-(3-hydroxy-2,2- dimethylchroman-4- yl)chromane-6-carboxamide |
| 13B | 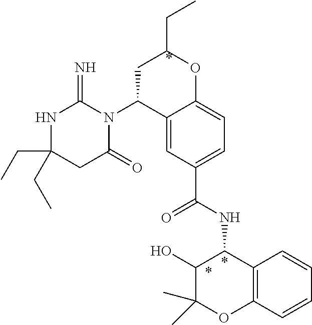 | 549.3 | (4R)-4-(4,4-diethyl-2-imino-6- oxotetrahydropyrimidin-1(2H)- yl)-2-ethyl-N-(3-hydroxy-2,2- dimethylchroman-4- yl)chromane-6-carboxamide |
| 14A | 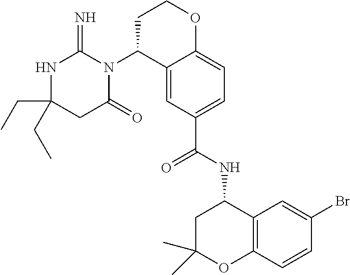 [SMILES: CCC1(CC)CC(=O)N([C@@H]2CCOc3ccc(C(=O)N[C@H]4CC(C)(C)Oc5ccc(Br)cc54)cc32)C(=N)N1] | 583.1, 585.2 | (R)-N-((S)-6-bromo-2,2- dimethylchroman-4-yl)-4-(4,4- diethyl-2-imino-6- oxotetrahydropyrimidin-1(2H)- yl)chromane-6-carboxamide |
| 15A | 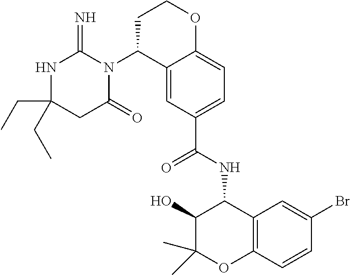 [SMILES: CCC1(CC)CC(=O)N([C@@H]2CCOc3ccc(C(=O)N[C@@H]4c5cc(Br)ccc5OC(C)(C)[C@H]4O)cc32)C(=N)N1] | 599.2,601.3 | (R)-N-((3S,4R)-6-bromo-3- hydroxy-2,2-dimethylchroman- 4-yl)-4-(4,4-diethyl-2-imino-6- oxotetrahydropyrimidin-1(2H)- yl)chromane-6-carboxamide |
| 16A | 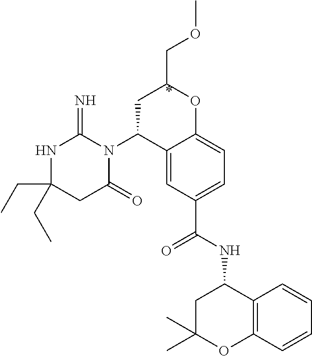 | 549.3 | (4R)-4-(4,4-diethyl-2-imino-6- oxotetrahydropyrimidin-1(2H)- yl)-N-((S)-2,2- dimethylchroman-4-yl)-2- (methoxymethyl)chromane-6- carboxamide |
| 16B | 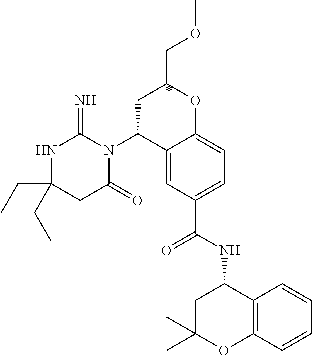 | 549.3 | (4R)-4-(4,4-diethyl-2-imino-6- oxotetrahydropyrimidin-1(2H)- yl)-N-((S)-2,2- dimethylchroman-4-yl)-2- (methoxymethyl)chromane-6- carboxamide |
| 17A | 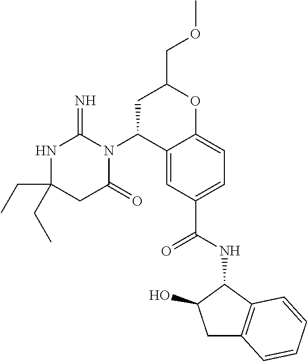 | 521.3 | (4R)-4-(4,4-diethyl-2-imino-6- oxotetrahydropyrimidin-1(2H)- yl)-N-((1R,2R)-2-hydroxy-2,3- dihydro-1H-inden-1-yl)-2- (methoxymethyl)chromane-6- carboxamide |
| 18A | 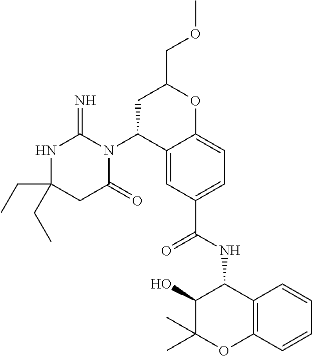 | 565.3 | (4R)-4-(4,4-diethyl-2-imino-6- oxotetrahydropyrimidin-1(2H)- yl)-N-((3S,4R)-3-hydroxy-2,2- dimethylchroman-4-yl)-2- (methoxymethyl)chromane-6- carboxamide |
| 19A | 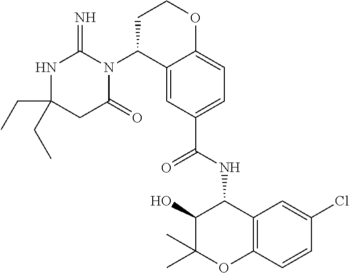 [SMILES: CCC1(CC)CC(=O)N([C@@H]2CCOc3ccc(C(=O)N[C@@H]4c5cc(Cl)ccc5OC(C)(C)[C@H]4O)cc32)C(=N)N1] | 555.2, 557.3 | (R)-N-((3S,4R)-6-chloro-3- hydroxy-2,2-dimethylchroman- 4-yl)-4-(4,4-diethyl-2-imino-6- oxotetrahydropyrimidin-1(2H)- yl)chromane-6-carboxamide |
| 20A | 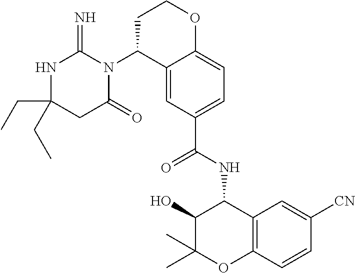 [SMILES: CCC1(CC)CC(=O)N([C@@H]2CCOc3ccc(C(=O)N[C@@H]4c5cc(C#N)ccc5OC(C)(C)[C@H]4O)cc32)C(=N)N1] | 546.3 | (R)-N-((3S,4R)-6-cyano-3- hydroxy-2,2-dimethylchroman- 4-yl)-4-(4,4-diethyl-2-imino-6- oxotetrahydropyrimidin-1(2H)- yl)chromane-6-carboxamide |
| 21A | 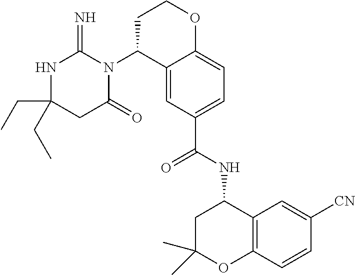 [SMILES: CCC1(CC)CC(=O)N([C@@H]2CCOc3ccc(C(=O)N[C@H]4CC(C)(C)Oc5ccc(C#N)cc54)cc32)C(=N)N1] | 530.3 | (R)-N-((S)-6-cyano-2,2- dimethylchroman-4-yl)-4-(4,4- diethyl-2-imino-6- oxotetrahydropyrimidin-1(2H)- yl)chromane-6-carboxamide |
| 22A | 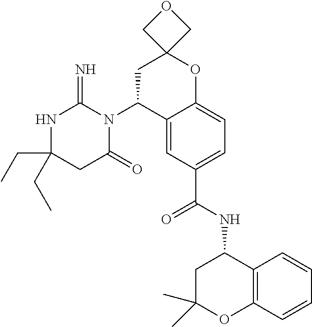 [SMILES: CCC1(CC)CC(=O)N([C@@H]2CC3(COC3)Oc3ccc(C(=O)N[C@H]4CC(C)(C)Oc5ccccc54)cc32)C(=N)N1] | 547.4 | (R)-4-(4,4-diethyl-2-imino-6- oxotetrahydropyrimidin-1(2H)- yl)-N-((S)-2,2- dimethylchroman-4- yl)spiro[chromane-2,3′- oxetane]-6-carboxamide |
| 23A | 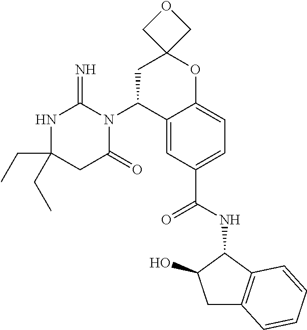 [SMILES: CCC1(CC)CC(=O)N([C@@H]2CC3(COC3)Oc3ccc(C(=O)N[C@@H]4c5ccccc5C[C@H]4O)cc32)C(=N)N1] | 519.2 | (R)-4-(4,4-diethyl-2-imino-6- oxotetrahydropyrimidin-1(2H)- yl)-N-((1R,2R)-2-hydroxy-2,3- dihydro-1H-inden-1- yl)spiro[chromane-2,3′- oxetane]-6-carboxamide |
| 24A | 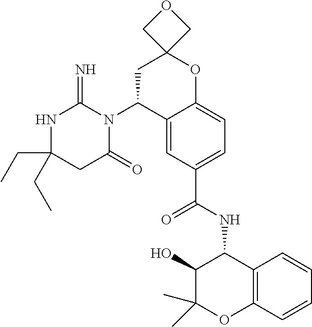 [SMILES: CCC1(CC)CC(=O)N([C@@H]2CC3(COC3)Oc3ccc(C(=O)N[C@@H]4c5ccccc5OC(C)(C)[C@H]4O)cc32)C(=N)N1] | 563.3 | (R)-4-(4,4-diethyl-2-imino-6- oxotetrahydropyrimidin-1(2H)- yl)-N-((3S,4R)-3-hydroxy-2,2- dimethylchroman-4- yl)spiro[chromane-2,3′- oxetane]-6-carboxamide |
| 25A | 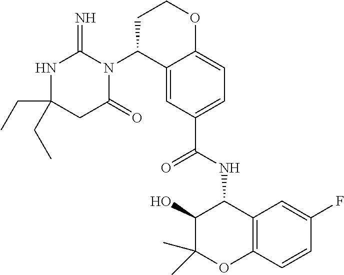 [SMILES: CCC1(CC)CC(=O)N([C@@H]2CCOc3ccc(C(=O)N[C@@H]4c5cc(F)ccc5OC(C)(C)[C@H]4O)cc32)C(=N)N1] | 539.3 | (R)-4-(4,4-diethyl-2-imino-6- oxotetrahydropyrimidin-1(2H)- yl)-N-((3S,4R)-6-fluoro-3- hydroxy-2,2-dimethylchroman- 4-yl)chromane-6-carboxamide |
| 25B | 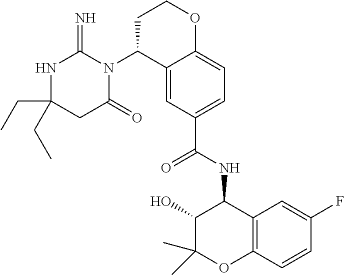 [SMILES: CCC1(CC)CC(=O)N([C@@H]2CCOc3ccc(C(=O)N[C@H]4c5cc(F)ccc5OC(C)(C)[C@@H]4O)cc32)C(=N)N1] | 539.3 | (R)-4-(4,4-diethyl-2-imino-6- oxotetrahydropyrimidin-1(2H)- yl)-N-((3R,4S)-6-fluoro-3- hydroxy-2,2-dimethylchroman- 4-yl)chromane-6-carboxamide |
| 26A | 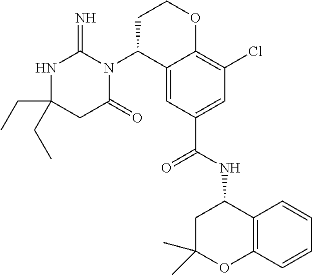 [SMILES: CCC1(CC)CC(=O)N([C@@H]2CCOc3c(Cl)cc(C(=O)N[C@H]4CC(C)(C)Oc5ccccc54)cc32)C(=N)N1] | 539.3, 541.3 | (R)-8-chloro-4-(4,4-diethyl-2- imino-6- oxotetrahydropyrimidin-1(2H)- yl)-N-((S)-2,2- dimethylchroman-4- yl)chromane-6-carboxamide |
| 26B | 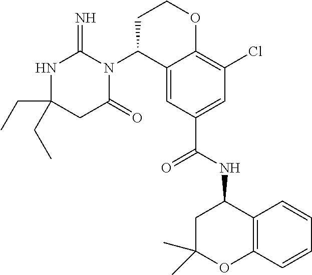 [SMILES: CCC1(CC)CC(=O)N([C@@H]2CCOc3c(Cl)cc(C(=O)N[C@@H]4CC(C)(C)Oc5ccccc54)cc32)C(=N)N1] | 539.2, 541.3 | (R)-8-chloro-4-(4,4-diethyl-2- imino-6- oxotetrahydropyrimidin-1(2H)- yl)-N-((R)-2,2- dimethylchroman-4- yl)chromane-6-carboxamide |
| 27A | 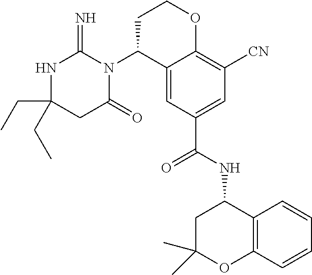 [SMILES: CCC1(CC)CC(=O)N([C@@H]2CCOc3c(C#N)cc(C(=O)N[C@H]4CC(C)(C)Oc5ccccc54)cc32)C(=N)N1] | 530.3 | (R)-8-cyano-4-(4,4-diethyl-2- imino-6- oxotetrahydropyrimidin-1(2H)- yl)-N-((S)-2,2- dimethylchroman-4- yl)chromane-6-carboxamide |
| 28A | 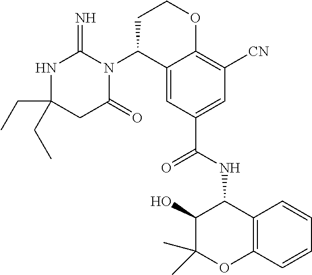 [SMILES: CCC1(CC)CC(=O)N([C@@H]2CCOc3c(C#N)cc(C(=O)N[C@@H]4c5ccccc5OC(C)(C)[C@H]4O)cc32)C(=N)N1] | 546.3 | (R)-8-cyano-4-(4,4-diethyl-2- imino-6- oxotetrahydropyrimidin-1(2H)- yl)-N-((3S,4R)-3-hydroxy-2,2- dimethylchroman-4- yl)chromane-6-carboxamide |
| 29A | 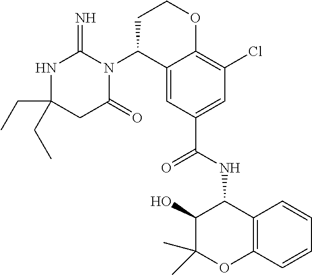 [SMILES: CCC1(CC)CC(=O)N([C@@H]2CCOc3c(Cl)cc(C(=O)N[C@@H]4c5ccccc5OC(C)(C)[C@H]4O)cc32)C(=N)N1] | 555.3,557.3 | (R)-8-chloro-4-(4,4-diethyl-2- imino-6- oxotetrahydropyrimidin-1(2H)- yl)-N-((3S,4R)-3-hydroxy-2,2- dimethylchroman-4- yl)chromane-6-carboxamide |
| 29B | 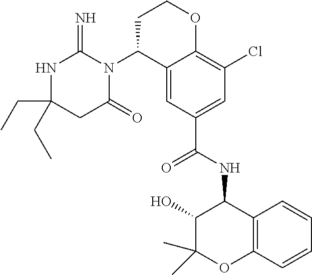 [SMILES: CCC1(CC)CC(=O)N([C@@H]2CCOc3c(Cl)cc(C(=O)N[C@H]4c5ccccc5OC(C)(C)[C@@H]4O)cc32)C(=N)N1] | 555.3, 557.3 | (R)-8-chloro-4-(4,4-diethyl-2- imino-6- oxotetrahydropyrimidin-1(2H)- yl)-N-((3R,4S)-3-hydroxy-2,2- dimethylchroman-4- yl)chromane-6-carboxamide |
| 30A | 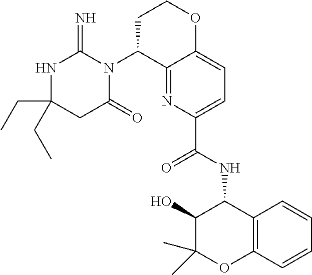 [SMILES: CCC1(CC)CC(=O)N([C@@H]2CCOc3ccc(C(=O)N[C@@H]4c5ccccc5OC(C)(C)[C@H]4O)nc32)C(=N)N1] | 522.3 | (R)-4-(4,4-diethyl-2-imino-6- oxotetrahydropyrimidin-1(2H)- yl)-N-((3S,4R)-3-hydroxy-2,2- dimethylchroman-4-yl)-3,4- dihydro-2H-pyrano[3,2- b]pyridine-6-carboxamide |
| 31A | 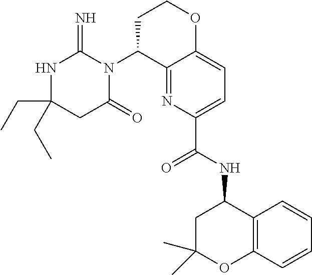 [SMILES: CCC1(CC)CC(=O)N([C@@H]2CCOc3ccc(C(=O)N[C@@H]4CC(C)(C)Oc5ccccc54)nc32)C(=N)N1] | 506.3 | (R)-4-(4,4-diethyl-2-imino-6- oxotetrahydropyrimidin-1(2H)- yl)-N-((R)-2,2- dimethylchroman-4-yl)-3,4- dihydro-2H-pyrano[3,2- b]pyridine-6-carboxamide |
| 31B | 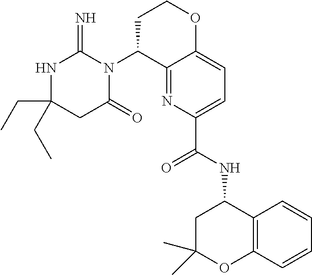 [SMILES: CCC1(CC)CC(=O)N([C@@H]2CCOc3ccc(C(=O)N[C@H]4CC(C)(C)Oc5ccccc54)nc32)C(=N)N1] | 506.3 | (R)-4-(4,4-diethyl-2-imino-6- oxotetrahydropyrimidin-1(2H)- yl)-N-((S)-2,2- dimethylchroman-4-yl)-3,4- dihydro-2H-pyrano[3,2- b]pyridine-6-carboxamide |
| 32A | 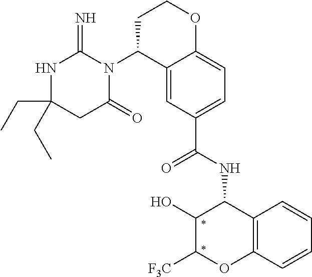 [SMILES: CCC1(CC)CC(=O)N([C@@H]2CCOc3ccc(C(=O)N[C@@H]4c5ccccc5OC(C(F)(F)F)C4O)cc32)C(=N)N1] | 561.3 | (4R)-4-(4,4-diethyl-2-imino-6- oxotetrahydropyrimidin-1(2H)- yl)-N-((4R)-3-hydroxy-2- (trifluoromethyl)chroman-4- yl)chromane-6-carboxamide |
| 32B | 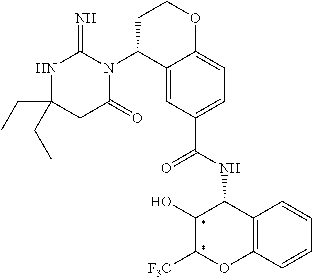 [SMILES: CCC1(CC)CC(=O)N([C@@H]2CCOc3ccc(C(=O)N[C@@H]4c5ccccc5OC(C(F)(F)F)C4O)cc32)C(=N)N1] | 561.3 | (4R)-4-(4,4-diethyl-2-imino-6- oxotetrahydropyrimidin-1(2H)- yl)-N-((4R)-3-hydroxy-2- (trifluoromethyl)chroman-4- yl)chromane-6-carboxamide |
| 33A | 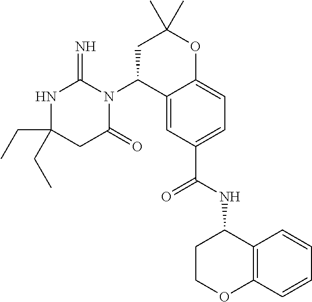 [SMILES: CCC1(CC)CC(=O)N([C@@H]2CC(C)(C)Oc3ccc(C(=O)N[C@H]4CCOc5ccccc54)cc32)C(=N)N1] | 505.3 | (R)-N-((S)-chroman-4-yl)-4- (4,4-diethyl-2-imino-6- oxotetrahydropyrimidin-1(2H)- yl)-2,2-dimethylchromane-6- carboxamide |
| 34A | 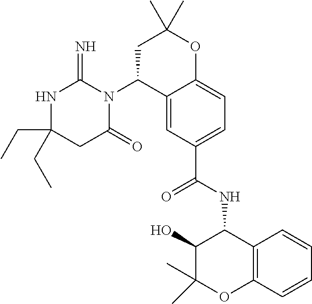 [SMILES: CCC1(CC)CC(=O)N([C@@H]2CC(C)(C)Oc3ccc(C(=O)N[C@@H]4c5ccccc5OC(C)(C)[C@H]4O)cc32)C(=N)N1] | 549.3 | (R)-4-(4,4-diethyl-2-imino-6- oxotetrahydropyrimidin-1(2H)- yl)-N-((3S,4R)-3-hydroxy-2,2- dimethylchroman-4-yl)-2,2- dimethylchromane-6- carboxamide |
| 35A | 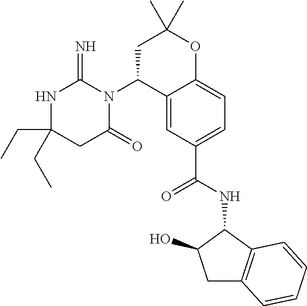 [SMILES: CCC1(CC)CC(=O)N([C@@H]2CC(C)(C)Oc3ccc(C(=O)N[C@@H]4c5ccccc5C[C@H]4O)cc32)C(=N)N1] | 505.3 | (R)-4-(4,4-diethyl-2-imino-6- oxotetrahydropyrimidin-1(2H)- yl)-N-((1R,2R)-2-hydroxy-2,3- dihydro-1H-inden-1-yl)-2,2- dimethylchromane-6- carboxamide |
| 36A | 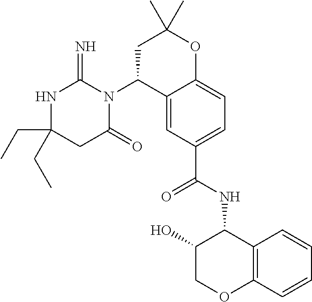 [SMILES: CCC1(CC)CC(=O)N([C@@H]2CC(C)(C)Oc3ccc(C(=O)N[C@@H]4c5ccccc5OC[C@@H]4O)cc32)C(=N)N1] | 521.3 | (R)-4-(4,4-diethyl-2-imino-6- oxotetrahydropyrimidin-1(2H)- yl)-N-((3R,4R)-3- hydroxychroman-4-yl)-2,2- dimethylchromane-6- carboxamide |
| 36B | 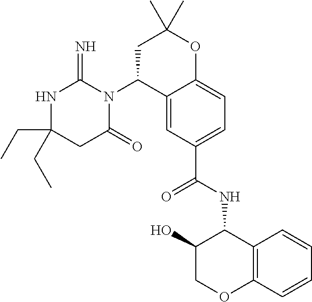 [SMILES: CCC1(CC)CC(=O)N([C@@H]2CC(C)(C)Oc3ccc(C(=O)N[C@@H]4c5ccccc5OC[C@H]4O)cc32)C(=N)N1] | 521.6 | (R)-4-(4,4-diethyl-2-imino-6- oxotetrahydropyrimidin-1(2H)- yl)-N-((3S,4R)-3- hydroxychroman-4-yl)-2,2- dimethylchromane-6- carboxamide |
| 37A | 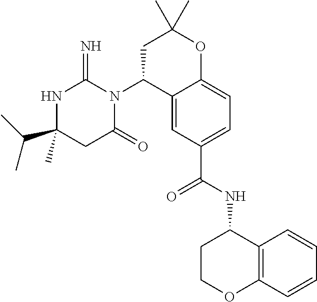 [SMILES: CC(C)[C@]1(C)CC(=O)N([C@@H]2CC(C)(C)Oc3ccc(C(=O)N[C@H]4CCOc5ccccc54)cc32)C(=N)N1] | 521.2 | (R)-N-((S)-chroman-4-yl)-4- ((S)-2-imino-4-isopropyl-4- methyl-6- oxotetrahydropyrimidin-1(2H)- yl)-2,2-dimethylchromane-6- carboxamide |
| 38A | 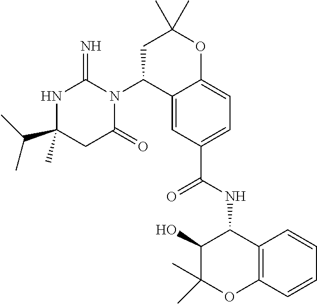 [SMILES: CC(C)[C@]1(C)CC(=O)N([C@@H]2CC(C)(C)Oc3ccc(C(=O)N[C@@H]4c5ccccc5OC(C)(C)[C@H]4O)cc32)C(=N)N1] | 549.3 | (R)-N-((3S,4R)-3-hydroxy-2,2- dimethylchroman-4-yl)-4-((S)- 2-imino-4-isopropyl-4-methyl- 6-oxotetrahydropyrimidin- 1(2H)-yl)-2,2- dimethylchromane-6- carboxamide |
| 39A | 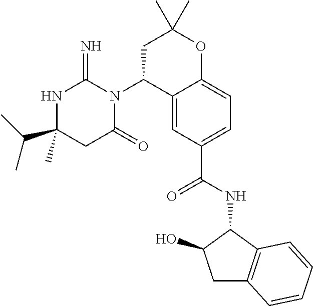 [SMILES: CC(C)[C@]1(C)CC(=O)N([C@@H]2CC(C)(C)Oc3ccc(C(=O)N[C@@H]4c5ccccc5C[C@H]4O)cc32)C(=N)N1] | 505.3 | (R)-N-((1R,2R)-2-hydroxy- 2,3-dihydro-1H-inden-1-yl)-4- ((S)-2-imino-4-isopropyl-4- methyl-6- oxotetrahydropyrimidin-1(2H)- yl)-2,2-dimethylchromane-6- carboxamide |
| 40A | 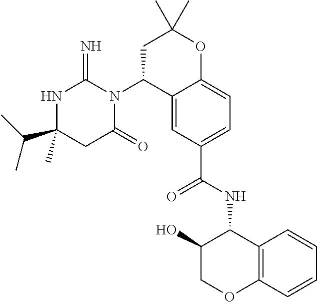 [SMILES: CC(C)[C@]1(C)CC(=O)N([C@@H]2CC(C)(C)Oc3ccc(C(=O)N[C@@H]4c5ccccc5OC[C@H]4O)cc32)C(=N)N1] | 521.3 | (R)-N-((3S,4R)-3- hydroxychroman-4-yl)-4-((S)- 2-imino-4-isopropyl-4-methyl- 6-oxotetrahydropyrimidin- 1(2H)-yl)-2,2- dimethylchromane-6- carboxamide |
| 41A | 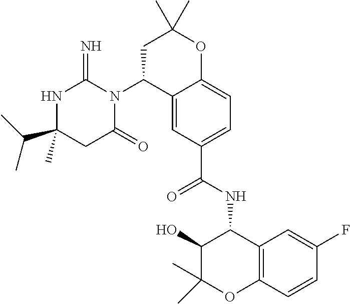 [SMILES: CC(C)[C@]1(C)CC(=O)N([C@@H]2CC(C)(C)Oc3ccc(C(=O)N[C@@H]4c5cc(F)ccc5OC(C)(C)[C@H]4O)cc32)C(=N)N1] | 567.3 | (R)-N-((3S,4R)-6-fluoro-3- hydroxy-2,2-dimethylchroman- 4-yl)-4-((S)-2-imino-4- isopropyl-4-methyl-6- oxotetrahydropyrimidin-1(2H)- yl)-2,2-dimethylchromane-6- carboxamide |
| 42A | 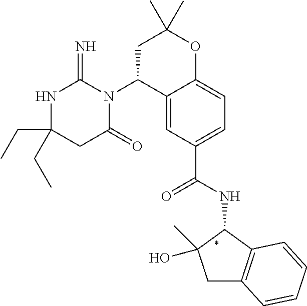 [SMILES: CCC1(CC)CC(=O)N([C@@H]2CC(C)(C)Oc3ccc(C(=O)N[C@@H]4c5ccccc5CC4(C)O)cc32)C(=N)N1] | 519.3 | (4R)-4-(4,4-diethyl-2-imino-6- oxotetrahydropyrimidin-1(2H)- yl)-N-((1R)-2-hydroxy-2- methyl-2,3-dihydro-1H-inden- 1-yl)-2,2-dimethylchromane-6- carboxamide |
| 42B | 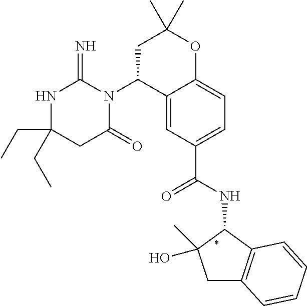 [SMILES: CCC1(CC)CC(=O)N([C@@H]2CC(C)(C)Oc3ccc(C(=O)N[C@@H]4c5ccccc5CC4(C)O)cc32)C(=N)N1] | 519.2 | (4R)-4-(4,4-diethyl-2-imino-6- oxotetrahydropyrimidin-1(2H)- yl)-N-((1R)-2-hydroxy-2- methyl-2,3-dihydro-1H-inden- 1-yl)-2,2-dimethylchromane-6- carboxamide |
| 43A | 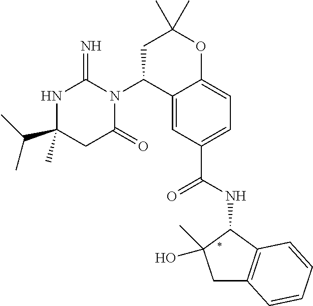 [SMILES: CC(C)[C@]1(C)CC(=O)N([C@@H]2CC(C)(C)Oc3ccc(C(=O)N[C@@H]4c5ccccc5CC4(C)O)cc32)C(=N)N1] | 519.2 | (4R)-N-((1R)-2-hydroxy-2- methyl-2,3-dihydro-1H-inden- 1-yl)-4-((S)-2-imino-4- isopropyl-4-methyl-6- oxotetrahydropyrimidin-1(2H)- yl)-2,2-dimethylchromane-6- carboxamide |
| 43B | 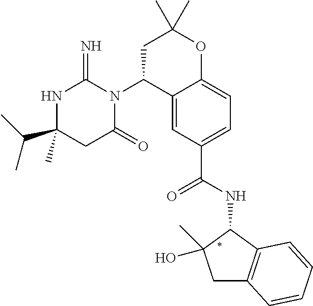 [SMILES: CC(C)[C@]1(C)CC(=O)N([C@@H]2CC(C)(C)Oc3ccc(C(=O)N[C@@H]4c5ccccc5CC4(C)O)cc32)C(=N)N1] | 519.2 | (4R)-N-((1R)-2-hydroxy-2- methyl-2,3-dihydro-1H-inden- 1-yl)-4-((S)-2-imino-4- isopropyl-4-methyl-6- oxotetrahydropyrimidin-1(2H)- yl)-2,2-dimethylchromane-6- carboxamide |
| 44A | 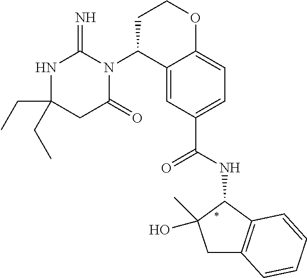 [SMILES: CCC1(CC)CC(=O)N([C@@H]2CCOc3ccc(C(=O)N[C@@H]4c5ccccc5CC4(C)O)cc32)C(=N)N1] | 491.2 | (4R)-4-(4,4-diethyl-2-imino-6- oxotetrahydropyrimidin-1(2H)- yl)-N-((1R)-2-hydroxy-2- methyl-2,3-dihydro-1H-inden- 1-yl)chromane-6-carboxamide |
| 44B | 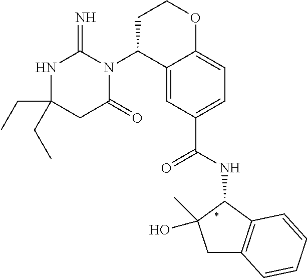 [SMILES: CCC1(CC)CC(=O)N([C@@H]2CCOc3ccc(C(=O)N[C@@H]4c5ccccc5CC4(C)O)cc32)C(=N)N1] | 491.2 | (4R)-4-(4,4-diethyl-2-imino-6- oxotetrahydropyrimidin-1(2H)- yl)-N-((1R)-2-hydroxy-2- methyl-2,3-dihydro-1H-inden- 1-yl)chromane-6-carboxamide |
| 45A | 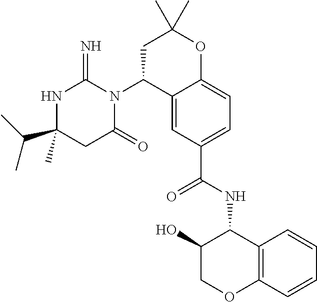 [SMILES: CC(C)[C@]1(C)CC(=O)N([C@@H]2CC(C)(C)Oc3ccc(C(=O)N[C@@H]4c5ccccc5OC[C@H]4O)cc32)C(=N)N1] | 521.2 | (R)-N-((3S,4R)-3- hydroxychroman-4-yl)-4-((S)- 2-imino-4-isopropyl-4-methyl- 6-oxotetrahydropyrimidin- 1(2H)-yl)-2,2- dimethylchromane-6- carboxamide |
| 46A | 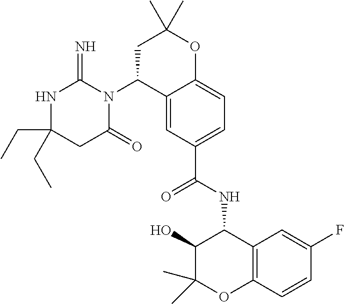 [SMILES: CCC1(CC)CC(=O)N([C@@H]2CC(C)(C)Oc3ccc(C(=O)N[C@@H]4c5cc(F)ccc5OC(C)(C)[C@H]4O)cc32)C(=N)N1] | 567.3 | (R)-4-(4,4-diethyl-2-imino-6- oxotetrahydropyrimidin-1(2H)- yl)-N-((3S,4R)-6-fluoro-3- hydroxy-2,2-dimethylchroman- 4-yl)-2,2-dimethylchromane-6- carboxamide |
| 47A | 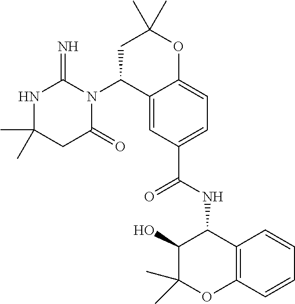 [SMILES: CC1(C)CC(=O)N([C@@H]2CC(C)(C)Oc3ccc(C(=O)N[C@@H]4c5ccccc5OC(C)(C)[C@H]4O)cc32)C(=N)N1] | 521.3 | (R)-N-((3S,4R)-3-hydroxy-2,2- dimethylchroman-4-yl)-4-(2- imino-4,4-dimethyl-6- oxotetrahydropyrimidin-1(2H)- yl)-2,2-dimethylchromane-6- carboxamide |
| 48A | 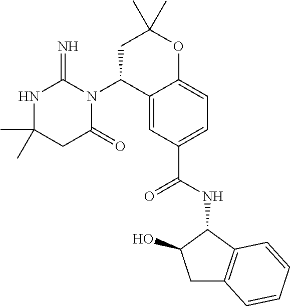 [SMILES: CC1(C)CC(=O)N([C@@H]2CC(C)(C)Oc3ccc(C(=O)N[C@@H]4c5ccccc5C[C@H]4O)cc32)C(=N)N1] | 477.3 | (R)-N-((1R,2R)-2-hydroxy- 2,3-dihydro-1H-inden-1-yl)-4- (2-imino-4,4-dimethyl-6- oxotetrahydropyrimidin-1(2H)- yl)-2,2-dimethylchromane-6- carboxamide |
| 49A | 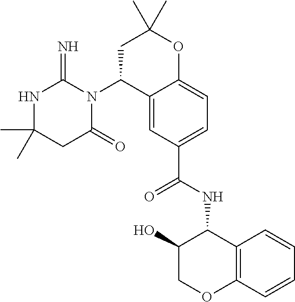 [SMILES: CC1(C)CC(=O)N([C@@H]2CC(C)(C)Oc3ccc(C(=O)N[C@@H]4c5ccccc5OC[C@H]4O)cc32)C(=N)N1] | 493.3 | (R)-N-((3S,4R)-3- hydroxychroman-4-yl)-4-(2- imino-4,4-dimethyl-6- oxotetrahydropyrimidin-1(2H)- yl)-2,2-dimethylchromane-6- carboxamide |
| 50A | 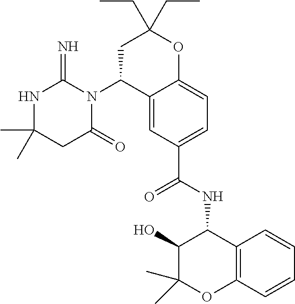 [SMILES: CCC1(CC)C[C@@H](N2C(=N)NC(C)(C)CC2=O)c2cc(C(=O)N[C@@H]3c4ccccc4OC(C)(C)[C@H]3O)ccc2O1] | 549.3 | (R)-2,2-diethyl-N-((3S,4R)-3- hydroxy-2,2-dimethylchroman- 4-yl)-4-(2-imino-4,4-dimethyl- 6-oxotetrahydropyrimidin- 1(2H)-yl)chromane-6- carboxamide |
| 51A | 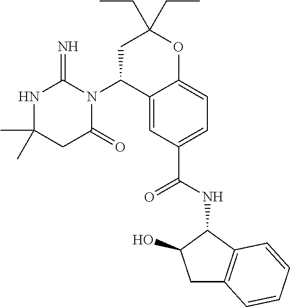 [SMILES: CCC1(CC)C[C@@H](N2C(=N)NC(C)(C)CC2=O)c2cc(C(=O)N[C@@H]3c4ccccc4C[C@H]3O)ccc2O1] | 505.3 | (R)-2,2-diethyl-N-((1R,2R)-2- hydroxy-2,3-dihydro-1H- inden-1-yl)-4-(2-imino-4,4- dimethyl-6- oxotetrahydropyrimidin-1(2H)- yl)chromane-6-carboxamide |
| 52A | 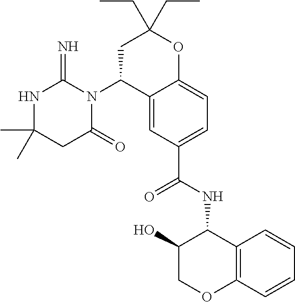 [SMILES: CCC1(CC)C[C@@H](N2C(=N)NC(C)(C)CC2=O)c2cc(C(=O)N[C@@H]3c4ccccc4OC[C@H]3O)ccc2O1] | 521.3 | (R)-2,2-diethyl-N-((3S,4R)-3- hydroxychroman-4-yl)-4-(2- imino-4,4-dimethyl-6- oxotetrahydropyrimidin-1(2H)- yl)chromane-6-carboxamide |
| 53A | 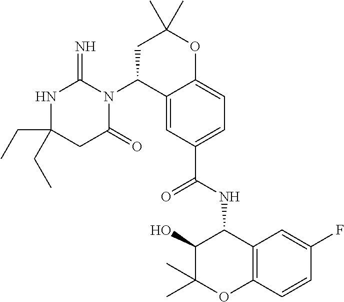 [SMILES: CCC1(CC)CC(=O)N([C@@H]2CC(C)(C)Oc3ccc(C(=O)N[C@@H]4c5cc(F)ccc5OC(C)(C)[C@H]4O)cc32)C(=N)N1] | 567.4 | (R)-4-(4,4-diethyl-2-imino-6- oxotetrahydropyrimidin-1(2H)- yl)-N-((3S,4R)-6-fluoro-3- hydroxy-2,2-dimethylchroman- 4-yl)-2,2-dimethylchromane-6- carboxamide |
| 54A | 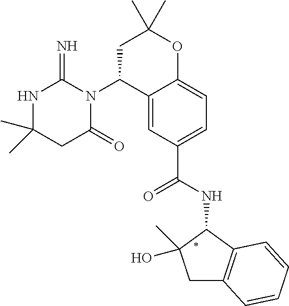 [SMILES: CC1(C)CC(=O)N([C@@H]2CC(C)(C)Oc3ccc(C(=O)N[C@@H]4c5ccccc5CC4(C)O)cc32)C(=N)N1] | 491.3 | (4R)-N-((1R)-2-hydroxy-2- methyl-2,3-dihydro-1H-inden- 1-yl)-4-(2-imino-4,4-dimethyl- 6-oxotetrahydropyrimidin- 1(2H)-yl)-2,2- dimethylchromane-6- carboxamide |
| 54B | 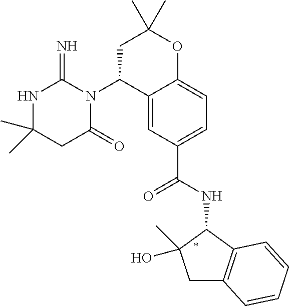 [SMILES: CC1(C)CC(=O)N([C@@H]2CC(C)(C)Oc3ccc(C(=O)N[C@@H]4c5ccccc5CC4(C)O)cc32)C(=N)N1] | 491.6 | (4R)-N-((1R)-2-hydroxy-2- methyl-2,3-dihydro-1H-inden- 1-yl)-4-(2-imino-4,4-dimethyl- 6-oxotetrahydropyrimidin- 1(2H)-yl)-2,2- dimethylchromane-6- carboxamide |
| 55A | 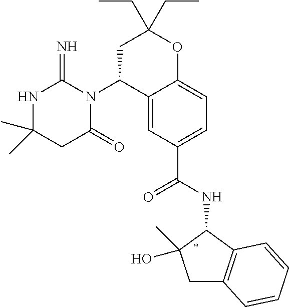 [SMILES: CCC1(CC)C[C@@H](N2C(=N)NC(C)(C)CC2=O)c2cc(C(=O)N[C@@H]3c4ccccc4CC3(C)O)ccc2O1] | 519.3 | (4R)-2,2-diethyl-N-((1R)-2- hydroxy-2-methyl-2,3-dihydro- 1H-inden-1-yl)-4-(2-imino-4,4- dimethyl-6- oxotetrahydropyrimidin-1(2H)- yl)chromane-6-carboxamide |
| 55B | 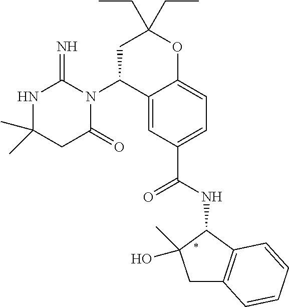 [SMILES: CCC1(CC)C[C@@H](N2C(=N)NC(C)(C)CC2=O)c2cc(C(=O)N[C@@H]3c4ccccc4CC3(C)O)ccc2O1] | 519.4 | (4R)-2,2-diethyl-N-((1R)-2- hydroxy-2-methyl-2,3-dihydro- 1H-inden-1-yl)-4-(2-imino-4,4- dimethyl-6- oxotetrahydropyrimidin-1(2H)- yl)chromane-6-carboxamide |
| 56A | 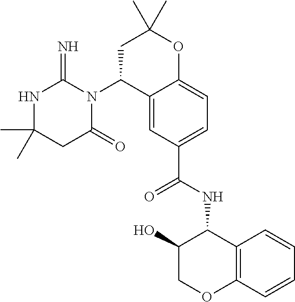 [SMILES: CC1(C)CC(=O)N([C@@H]2CC(C)(C)Oc3ccc(C(=O)N[C@@H]4c5ccccc5OC[C@H]4O)cc32)C(=N)N1] | 493.2 | (R)-N-((3S,4R)-3- hydroxychroman-4-yl)-4-(2- imino-4,4-dimethyl-6- oxotetrahydropyrimidin-1(2H)- yl)-2,2-dimethylchromane-6- carboxamide |
| 57A | 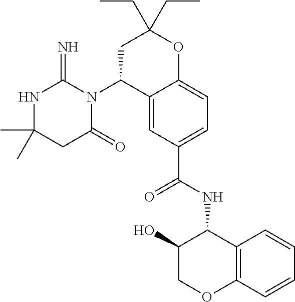 [SMILES: CCC1(CC)C[C@@H](N2C(=N)NC(C)(C)CC2=O)c2cc(C(=O)N[C@@H]3c4ccccc4OC[C@H]3O)ccc2O1] | 521.3 | (R)-2,2-diethyl-N-((3S,4R)-3- hydroxychroman-4-yl)-4-(2- imino-4,4-dimethyl-6- oxotetrahydropyrimidin-1(2H)- yl)chromane-6-carboxamide |
| 58A | 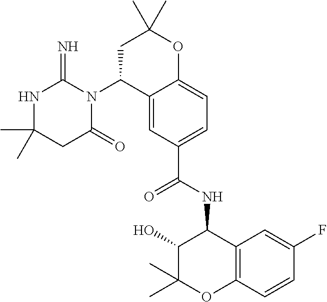 [SMILES: CC1(C)CC(=O)N([C@@H]2CC(C)(C)Oc3ccc(C(=O)N[C@H]4c5cc(F)ccc5OC(C)(C)[C@@H]4O)cc32)C(=N)N1] | 539.3 | (R)-N-((3R,4S)-6-fluoro-3- hydroxy-2,2-dimethylchroman- 4-yl)-4-(2-imino-4,4-dimethyl- 6-oxotetrahydropyrimidin- 1(2H)-yl)-2,2- dimethylchromane-6- carboxamide |
| 58B | 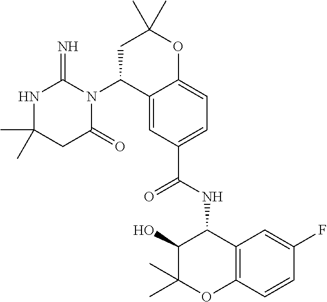 [SMILES: CC1(C)CC(=O)N([C@@H]2CC(C)(C)Oc3ccc(C(=O)N[C@@H]4c5cc(F)ccc5OC(C)(C)[C@H]4O)cc32)C(=N)N1] | 539.3 | (R)-N-((3S,4R)-6-fluoro-3- hydroxy-2,2-dimethylchroman- 4-yl)-4-(2-imino-4,4-dimethyl- 6-oxotetrahydropyrimidin- 1(2H)-yl)-2,2- dimethylchromane-6- carboxamide |
| 59A | 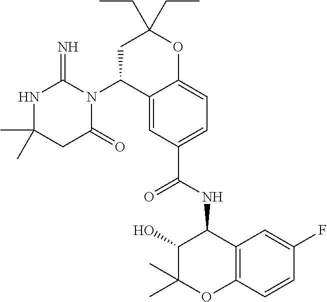 [SMILES: CCC1(CC)C[C@@H](N2C(=N)NC(C)(C)CC2=O)c2cc(C(=O)N[C@H]3c4cc(F)ccc4OC(C)(C)[C@@H]3O)ccc2O1] | 567.3 | (R)-2,2-diethyl-N-((3R,4S)-6- fluoro-3-hydroxy-2,2- dimethylchroman-4-yl)-4-(2- imino-4,4-dimethyl-6- oxotetrahydropyrimidin-1(2H)- yl)chromane-6-carboxamide |
| 59B | 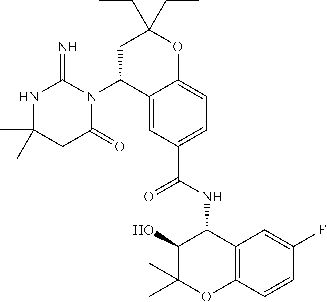 [SMILES: CCC1(CC)C[C@@H](N2C(=N)NC(C)(C)CC2=O)c2cc(C(=O)N[C@@H]3c4cc(F)ccc4OC(C)(C)[C@H]3O)ccc2O1] | 567.3 | (R)-2,2-diethyl-N-((3S,4R)-6- fluoro-3-hydroxy-2,2- dimethylchroman-4-yl)-4-(2- imino-4,4-dimethyl-6- oxotetrahydropyrimidin-1(2H)- yl)chromane-6-carboxamide |
| 60A | 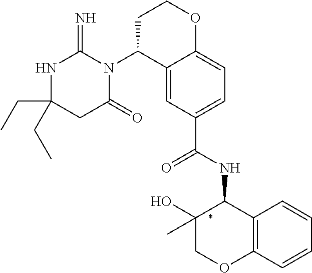 [SMILES: CCC1(CC)CC(=O)N([C@@H]2CCOc3ccc(C(=O)N[C@H]4c5ccccc5OCC4(C)O)cc32)C(=N)N1] | 507.2 | (4R)-4-(4,4-diethyl-2-imino-6- oxotetrahydropyrimidin-1(2H)- yl)-N-((4S)-3-hydroxy-3- methylchroman-4- yl)chromane-6-carboxamide |
| 60B | 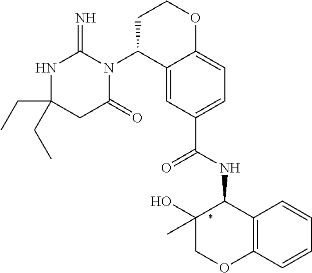 [SMILES: CCC1(CC)CC(=O)N([C@@H]2CCOc3ccc(C(=O)N[C@H]4c5ccccc5OCC4(C)O)cc32)C(=N)N1] | 507.3 | (4R)-4-(4,4-diethyl-2-imino-6- oxotetrahydropyrimidin-1(2H)- yl)-N-((4S)-3-hydroxy-3- methylchroman-4- yl)chromane-6-carboxamide |
| 61A | 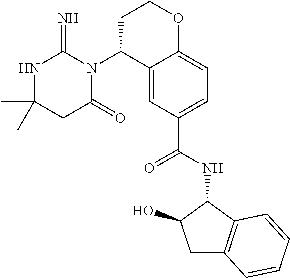 [SMILES: CC1(C)CC(=O)N([C@@H]2CCOc3ccc(C(=O)N[C@@H]4c5ccccc5C[C@H]4O)cc32)C(=N)N1] | 449.3 | (R)-N-((1R,2R)-2-hydroxy- 2,3-dihydro-1H-inden-1-yl)-4- (2-imino-4,4-dimethyl-6- oxotetrahydropyrimidin-1(2H)- yl)chromane-6-carboxamide |
| 62A | 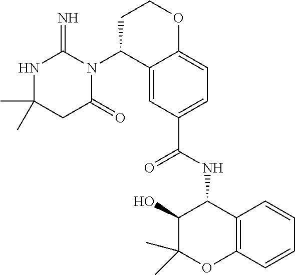 [SMILES: CC1(C)CC(=O)N([C@@H]2CCOc3ccc(C(=O)N[C@@H]4c5ccccc5OC(C)(C)[C@H]4O)cc32)C(=N)N1] | 493.3 | (R)-N-((3S,4R)-3-hydroxy-2,2- dimethylchroman-4-yl)-4-(2- imino-4,4-dimethyl-6- oxotetrahydropyrimidin-1(2H)- yl)chromane-6-carboxamide |
| 63A | 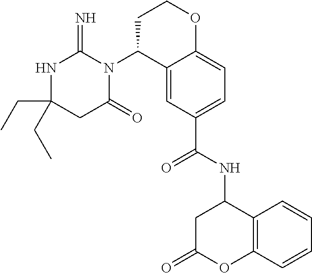 [SMILES: CCC1(CC)CC(=O)N([C@@H]2CCOc3ccc(C(=O)NC4CC(=O)Oc5ccccc54)cc32)C(=N)N1] | 491.3 | (4R)-4-(4,4-diethyl-2-imino-6- oxotetrahydropyrimidin-1(2H)- yl)-N-(2-oxochroman-4- yl)chromane-6-carboxamide |
| 64A | 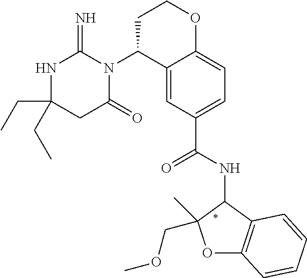 [SMILES: CCC1(CC)CC(=O)N([C@@H]2CCOc3ccc(C(=O)NC4c5ccccc5OC4(C)COC)cc32)C(=N)N1] | 521.3 | (4R)-4-(4,4-diethyl-2-imino-6- oxotetrahydropyrimidin-1(2H)- yl)-N-(2-(methoxymethyl)-2- methyl-2,3-dihydrobenzofuran- 3-yl)chromane-6-carboxamide |
| 64B | 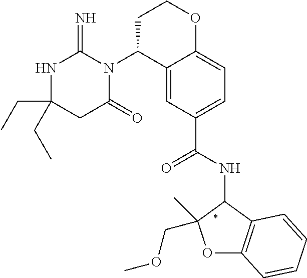 [SMILES: CCC1(CC)CC(=O)N([C@@H]2CCOc3ccc(C(=O)NC4c5ccccc5OC4(C)COC)cc32)C(=N)N1] | 521.3 | (4R)-4-(4,4-diethyl-2-imino-6- oxotetrahydropyrimidin-1(2H)- yl)-N-(2-(methoxymethyl)-2- methyl-2,3-dihydrobenzofuran- 3-yl)chromane-6-carboxamide |
| 64C | 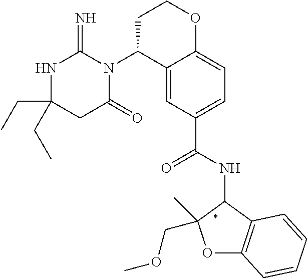 [SMILES: CCC1(CC)CC(=O)N([C@@H]2CCOc3ccc(C(=O)NC4c5ccccc5OC4(C)COC)cc32)C(=N)N1] | 521.3 | (4R)-4-(4,4-diethyl-2-imino-6- oxotetrahydropyrimidin-1(2H)- yl)-N-(2-(methoxymethyl)-2- methyl-2,3-dihydrobenzofuran- 3-yl)chromane-6-carboxamide |
| 64D | 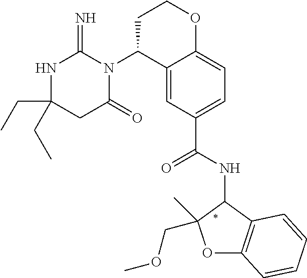 [SMILES: CCC1(CC)CC(=O)N([C@@H]2CCOc3ccc(C(=O)NC4c5ccccc5OC4(C)COC)cc32)C(=N)N1] | 521.3 | (4R)-4-(4,4-diethyl-2-imino-6- oxotetrahydropyrimidin-1(2H)- yl)-N-(2-(methoxymethyl)-2- methyl-2,3-dihydrobenzofuran- 3-yl)chromane-6-carboxamide |
| 65A | 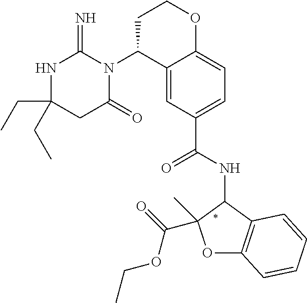 [SMILES: CCOC(=O)C1(C)Oc2ccccc2C1NC(=O)c1ccc2c(c1)[C@H](N1C(=N)NC(CC)(CC)CC1=O)CCO2] | 549.3 | ethyl 3-((R)-4-(4,4-diethyl-2- imino-6- oxotetrahydropyrimidin-1(2H)- yl)chromane-6-carboxamido)- 2-methyl-2,3- dihydrobenzofuran-2- carboxylate |
| 65B | 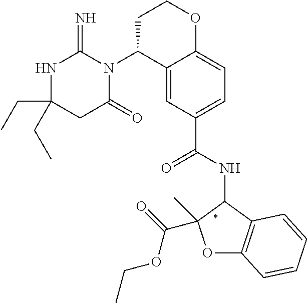 [SMILES: CCOC(=O)C1(C)Oc2ccccc2C1NC(=O)c1ccc2c(c1)[C@H](N1C(=N)NC(CC)(CC)CC1=O)CCO2] | 549.3 | ethyl 3-((R)-4-(4,4-diethyl-2- imino-6- oxotetrahydropyrimidin-1(2H)- yl)chromane-6-carboxamido)- 2-methyl-2,3- dihydrobenzofuran-2- carboxylate |
| 65C | 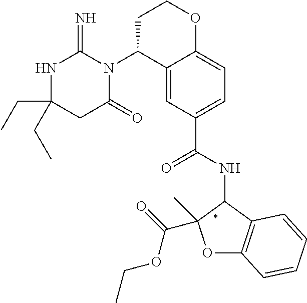 [SMILES: CCOC(=O)C1(C)Oc2ccccc2C1NC(=O)c1ccc2c(c1)[C@H](N1C(=N)NC(CC)(CC)CC1=O)CCO2] | 549.3 | ethyl 3-((R)-4-(4,4-diethyl-2- imino-6- oxotetrahydropyrimidin-1(2H)- yl)chromane-6-carboxamido)- 2-methyl-2,3- dihydrobenzofuran-2- carboxylate |
| 66A | 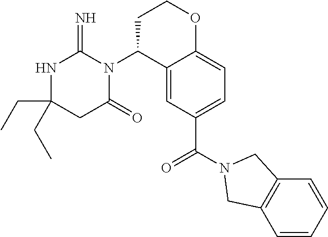 [SMILES: CCC1(CC)CC(=O)N([C@@H]2CCOc3ccc(C(=O)N4Cc5ccccc5C4)cc32)C(=N)N1] | 447.3 | (R)-6,6-diethyl-2-imino-3-(6- (isoindoline-2- carbonyl)chroman-4- yl)tetrahydropyrimidin-4(1H)- one |
| 67A | 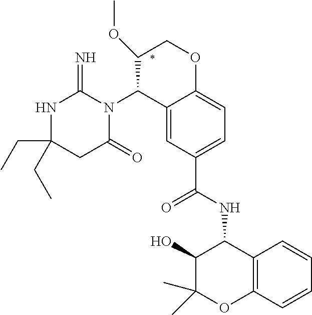 | 551.3 | (3R,4S)-4-(4,4-diethyl-2- imino-6- oxotetrahydropyrimidin-1(2H)- yl)-N-((3S,4R)-3-hydroxy-2,2- dimethylchroman-4-yl)-3- methoxychromane-6- carboxamide |
| 67B | 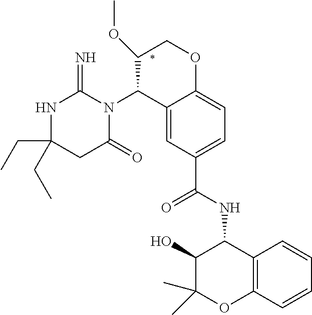 | 551.3 | (3R,4S)-4-(4,4-diethyl-2- imino-6- oxotetrahydropyrimidin-1(2H)- yl)-N-((3S,4R)-3-hydroxy-2,2- dimethylchroman-4-yl)-3- methoxychromane-6- carboxamide |
| 68A | 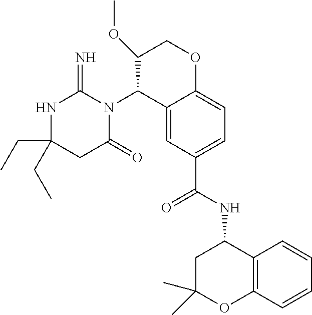 | 535.3 | (3R,4S)-4-(4,4-diethyl-2- imino-6- oxotetrahydropyrimidin-1(2H)- yl)-N-((S)-2,2- dimethylchroman-4-yl)-3- methoxychromane-6- carboxamide |
| 69A | 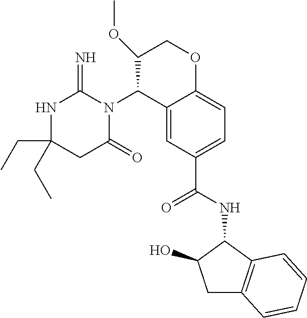 | 507.3 | (3R,4S)-4-(4,4-diethyl-2- imino-6- oxotetrahydropyrimidin-1(2H)- yl)-N-((1R,2R)-2-hydroxy-2,3- dihydro-1H-inden-1-yl)-3- methoxychromane-6- carboxamide |
| 70A | 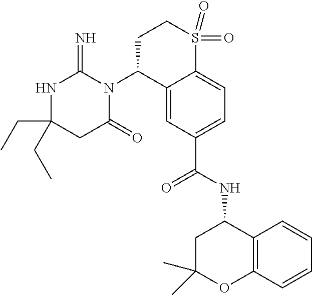 [SMILES: CCC1(CC)CC(=O)N([C@@H]2CCS(=O)(=O)c3ccc(C(=O)N[C@H]4CC(C)(C)Oc5ccccc54)cc32)C(=N)N1] | 553.3 | (R)-4-(4,4-diethyl-2-imino-6- oxotetrahydropyrimidin-1(2H)- yl)-N-((S)-2,2- dimethylchroman-4- yl)thiochromane-6- carboxamide 1,1-dioxide |
| 71A | 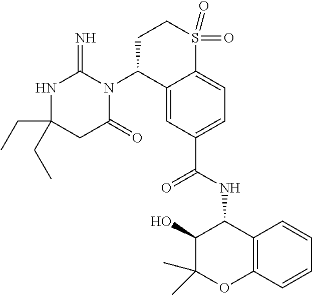 [SMILES: CCC1(CC)CC(=O)N([C@@H]2CCS(=O)(=O)c3ccc(C(=O)N[C@@H]4c5ccccc5OC(C)(C)[C@H]4O)cc32)C(=N)N1] | 569.3 | (R)-4-(4,4-diethyl-2-imino-6- oxotetrahydropyrimidin-1(2H)- yl)-N-((3S,4R)-3-hydroxy-2,2- dimethylchroman-4- yl)thiochromane-6- carboxamide 1,1-dioxide |
| 72A | 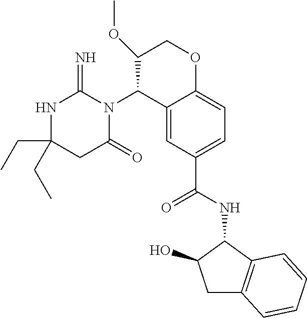 | 507.3 | (3R,4S)-4-(4,4-diethyl-2- imino-6- oxotetrahydropyrimidin-1(2H)- yl)-N-((1R,2R)-2-hydroxy-2,3- dihydro-1H-inden-1-yl)-3- methoxychromane-6- carboxamide |
| 73A | 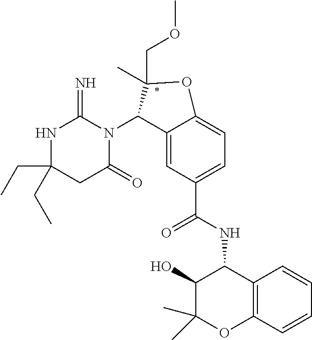 | 565.3 | (2S,3S)-3-(4,4-diethyl-2- imino-6- oxotetrahydropyrimidin-1(2H)- yl)-N-((3S,4R)-3-hydroxy-2,2- dimethylchroman-4-yl)-2- (methoxymethyl)-2-methyl- 2,3-dihydrobenzofuran-5- carboxamide |
| 73B | 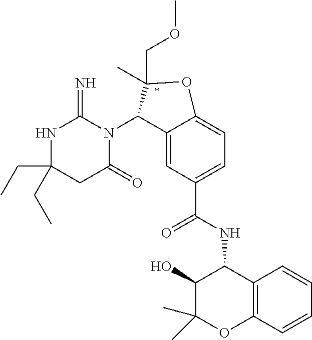 | 565.2 | (2S,3S)-3-(4,4-diethyl-2- imino-6- oxotetrahydropyrimidin-1(2H)- yl)-N-((3S,4R)-3-hydroxy-2,2- dimethylchroman-4-yl)-2- (methoxymethyl)-2-methyl- 2,3-dihydrobenzofuran-5- carboxamide |
| 74A | 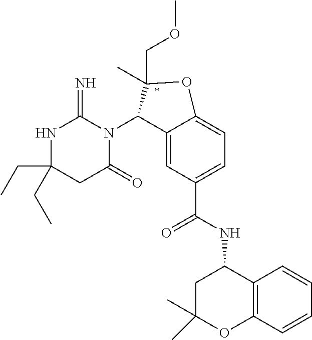 | 549.2 | (2S,3S)-3-(4,4-diethyl-2- imino-6- oxotetrahydropyrimidin-1(2H)- yl)-N-((S)-2,2- dimethylchroman-4-yl)-2- (methoxymethyl)-2-methyl- 2,3-dihydrobenzofuran-5- carboxamide |
| 74B | 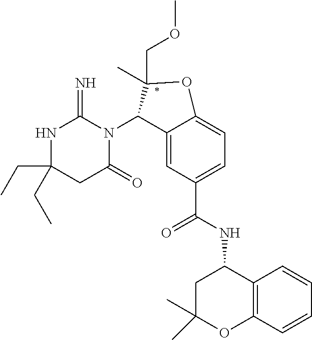 | 549.2 | (2S,3S)-3-(4,4-diethyl-2- imino-6- oxotetrahydropyrimidin-1(2H)- yl)-N-((S)-2,2- dimethylchroman-4-yl)-2- (methoxymethyl)-2-methyl- 2,3-dihydrobenzofuran-5- carboxamide |

### Example 75A

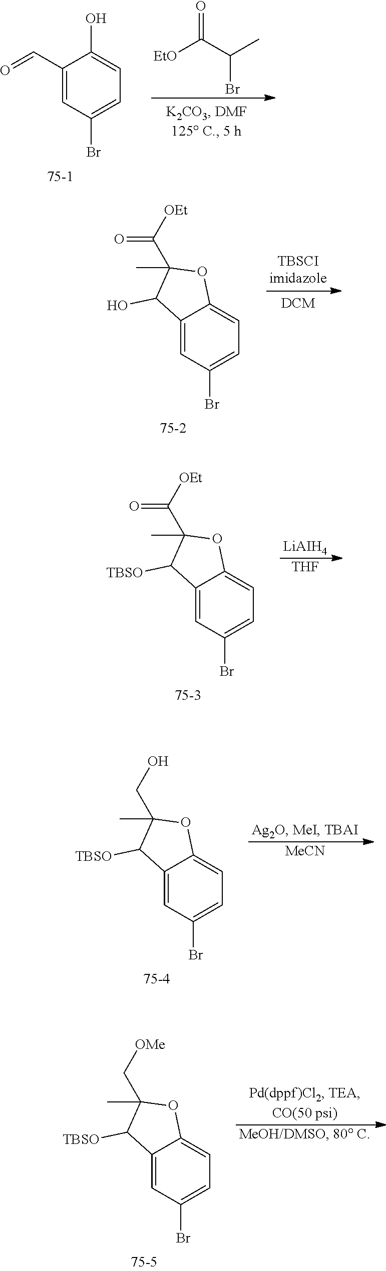

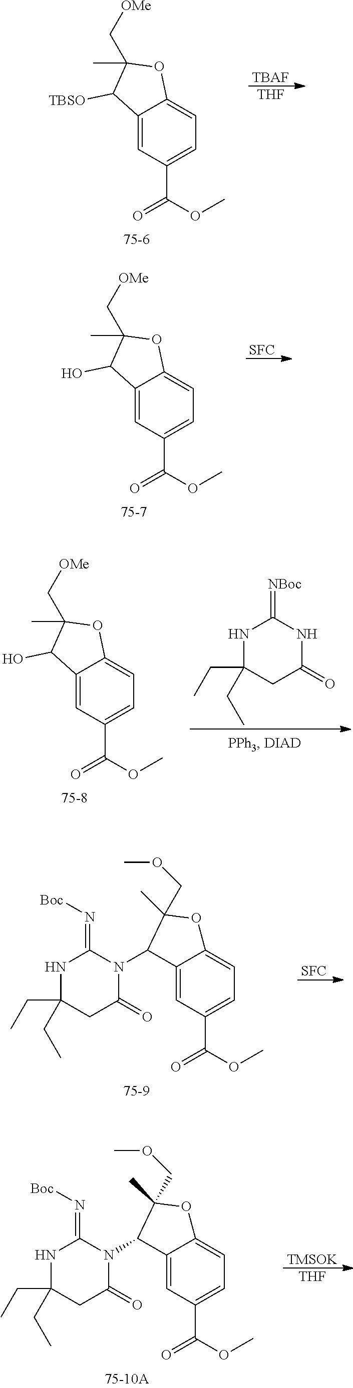

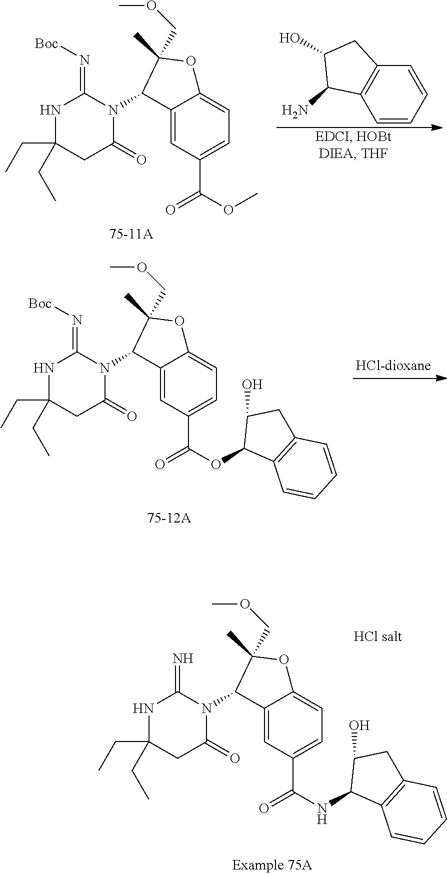

### Step A: ethyl 5-bromo-3-hydroxy-2-methyl-2,3-dihydrobenzofuran-2-carboxylate 75-2

Ethyl 2-bromopropanoate (270 g, 1492 mmol) and K₂CO₃ (413 g, 2985 mmol) were added to a solution of 5-bromo-2-hydroxybenzaldehyde 75-1 (300 g, 1492 mmol) in DMF (3 L). The mixture was stirred at 125° C. for 5 h. The mixture was diluted with water (5000 mL) and extracted with EtOAc (2000 mL*3) The organic layers were washed with brine (2000 mL), dried over anhydrous Na₂SO₄, filtered and concentrated. The residue was purified by column chromatography (SiO₂, petroleum ether:EtOAc=20:1-5:1) to afford product ethyl 5-bromo-3-hydroxy-2-methyl-2,3-dihydrobenzofuran-2-carboxylate 75-2.

¹H NMR (400 MHz, chloroform-d) δ 7.45 (d, J=1.6 Hz, 1H), 7.34 (dd, J=2.0, 8.4 Hz, 1H), 6.79 (d, J=8.4 Hz, 1H), 5.27 (s, 1H), 4.12-4.20 (m, 2H), 2.42-2.44 (m, 1H), 1.64 (s, 3H), 1.22 (t, J=7.2 Hz, 3H).

### Step B: ethyl 5-bromo-3-((tert-butyldimethylsilyl)oxy)-2-methyl-2,3-dihydrobenzofiran-2-carboxylate 75-3

Ethyl 5-bromo-3-hydroxy-2-methyl-2,3-dihydrobenzofuran-2-carboxylate 75-2 (300 g, 996 mmol) in DCM (3000 mL) was added to chlorotrimethylsilane (70 g, 644 mmol) and 1H-imidazole (60 g, 881 mmol). The mixture was stirred at 25° C. for 10 h under N₂ atmosphere. Water (3000 mL) was added to the mixture, and the mixture was extracted with DCM (500 mL*2). The organic layers were washed with brine (1000 mL), dried over Na₂SO₄, filtered and concentrated in vacuo, then purified by flash column (petroleum ether/EtOAc=50:1) to give the product ethyl 5-bromo-3-((tert-butyldimethylsilyl)oxy)-2-methyl-2,3-dihydrobenzofuran-2-carboxylate 75-3.

¹H NMR (400 MHz, chloroform-d) δ 7.26-7.35 (m, 2H), 6.78 (d, J=8.4 Hz, 1H), 5.35 (s, 1H), 4.14-4.23 (m, 2H), 1.60 (s, 3H), 1.22-1.27 (m, 3H), 0.93 (s, 9H), 0.18 (s, 6H)

### Step C: (5-bromo-3-((tert-butyldimethylsilyl)oxy)-2-methyl-2,3-dihydrobenzofuran-2-yl)methanol 75-4

LiAlH₄ (64.0 g, 1685 mmol) was added to a solution of ethyl 5-bromo-3-((tert-butyldimethylsilyl)oxy)-2-methyl-2,3-dihydrobenzofuran-2-carboxylate 75-3 (350 g, 843 mmol) in THF (3000 mL), in portions at 0° C. for 30 min. Then the mixture was stirred at 27° C. for another 30 min. The mixture was quenched with water (100 mL), diluted with EtOAc (5000 mL), dried over anhydrous Na₂SO₄ and MgSO₄, filtered and concentrated to afford product (5-bromo-3-((tert-butyldimethylsilyl)oxy)-2-methyl-2,3-dihydrobenzofuran-2-yl)methanol 75-4. The product was used for the next step without purification.

¹H NMR (400 MHz, chloroform-d) δ 7.27-7.32 (m, 2H), 6.60-6.70 (m, 1H), 5.23 (s, 1H), 3.55-3.66 (m, 2H), 1.77-1.86 (m, 1H), 1.35 (s, 3H), 0.93 (s, 9H), 0.21 (s, 3H), 0.16 (s, 3H).

### Step D: ((5-bromo-2-(methoxymethyl)-2-methyl-2,3-dihydrobenzofuran-3-yl)oxy)(tert-butyl)dimethylsilane 75-5

Iodomethane (968.150 g, 6821 mmol) and TBAI (20 g, 54.1 mmol) was added to a solution of (5-bromo-3-((tert-butyldimethylsilyl)oxy)-2-methyl-2,3-dihydrobenzofuran-2-yl)methanol 75-4 (270 g, 723 mmol) and monosilver(I) monosilver(III) monooxide (335 g, 1446 mmol) in MCCN (2.0 L mL) at 27° C. The mixture was stirred at 50° C. for 15 h under N₂ atmosphere. The mixture was filtered and concentrated. The residue was purified by flash column (SiO₂, petroleum ether/EtOAc=100:1) to afford product ((5-bromo-2-(methoxymethyl)-2-methyl-2,3-dihydrobenzofuran-3-yl)oxy)(tert-butyl)dimethylsilane 75-5.

¹H NMR (500 MHz, chloroform-d) δ 7.20-7.29 (m, 2H), 6.63 (d, J=8.5 Hz, 1H), 5.14 (s, 1H), 3.14-3.46 (m, 5H), 1.34 (s, 3H), 0.89 (s, 9H), 0.07-0.19 (m, 6H).

### Step E: methyl 3-((tert-butyldimethylsilyl)oxy)-2-(methoxymethyl)-2-methyl-2,3-dihydrobenzofuran-5-carboxylate 75-6

Pd(dppf)Cl₂ (18.9 g, 25.8 mmol) and triethylamine (131 g, 1291 mmol) was added to a solution of ((5-bromo-2-(methoxymethyl)-2-methyl-2,3-dihydrobenzofuran-3-yl)oxy)(tert-butyl)dimethylsilane 75-5 (100 g, 258 mmol) in MeOH (1000 mL) and DMSO (500 mL). The mixture was stirred at 80° C. for 12 h under 50 psi CO atmosphere. The mixture was filtered and concentrated. The residue was added water (500 mL) and extracted with EtOAc (300 mL×2). The organic layers were washed with brine (300 mL), dried over Na₂SO₄, filtered and concentrated in vacuo, then purified by flash column (petroleum ether/EtOAc=100:0 to 10:1) to afford product methyl 3-((tert-butyldimethylsilyl)oxy)-2-(methoxymethyl)-2-methyl-2,3-dihydrobenzofuran-5-carboxylate 75-6.

¹H NMR (500 MHz, chloroform-d) δ 7.91-7.99 (m, 2H), 6.79 (d, J=9.5 Hz, 1H), 5.21 (s, 1H), 3.87 (s, 3H), 3.41-3.46 (m, 1H), 3.36 (s, 3H), 3.32-3.35 (m, 1H), 1.41 (s, 3H), 0.92 (s, 9H), 0.22 (s, 3H), 0.17 (s, 3H).

### Step F: methyl 3-hydroxy-2-(methoxymethyl)-2-methyl-2,3-dihydrobenzofuran-5-carboxylate 75-7

TBAF (355 mL, 355 mmol) was added to a solution of methyl 3-((tert-butyldimethylsilyl)oxy)-2-(methoxymethyl)-2-methyl-2,3-dihydrobenzofuran-5-carboxylate 75-6 (65 g, 177 mmol) in THF (100 mL). The mixture was stirred at 27° C. for 0.5 hours. The mixture was diluted with water (200 mL) and extracted with EtOAc (100 mL*3). The organic layers were washed with brine (100 mL), dried over anhydrous Na₂SO₄, filtered and concentrated. The residue was purified by column chromatography (SiO₂, petroleum ether:EtOAc=10:1 to 3:1) to afford product methyl 3-hydroxy-2-(methoxymethyl)-2-methyl-2,3-dihydrobenzofuran-5-carboxylate 75-7.

¹H NMR (400 MHz, chloroform-d) δ 8.04 (d, J=2.0 Hz, 1H), 7.90 (dd, J=2.0, 8.5 Hz, 1H), 6.78 (d, J=8.5 Hz, 1H), 5.08 (s, 1H), 3.85 (s, 3H), 3.30-3.47 (m, 5H), 2.61 (br, 1H), 1.49 (s, 3H).

### Step G: methyl (2S,3R)-3-hydroxy-2-(methoxymethyl)-2-methyl-2,3-dihydrobenzofuran-5-carboxylate 75-8 (P1) and methyl (2R,3S)-3-hydroxy-2-(methoxymethyl)-2-methyl-2,3-dihydrobenzofuran-5-carboxylate 75-8 (P2)

The methyl 3-hydroxy-2-(methoxymethyl)-2-methyl-2,3-dihydrobenzofuran-5-carboxylate 75-7 (40 g, 159 mmol) was purified by SFC (SFC-7 Method Column DAICEL CHIRALPAK AD (250 mm×50 mm, 10 um). Condition 0.1% NH₃H₂O IPA Begin B 25% End B 25%. Gradient time (min) 100% B Hold Time (min) FlowRate (ml/min) 200) to give methyl (2S,3R)-3-hydroxy-2-(methoxymethyl)-2-methyl-2,3-dihydrobenzofiran-5-carboxylate 75-8 (P1) (Rt=3.450) and methyl (2R,3S)-3-hydroxy-2-(methoxymethyl)-2-methyl-2,3-dihydrobenzofuran-5-carboxylate 75-8 (P2) (Rt=3.951).

75-8 (P1): MS (ESI) m/z 253.1 (M+H⁺)

¹H NMR (400 MHz, chloroform-d) δ 8.04 (s, 1H), 7.91 (dd, J=2.0, 8.5 Hz, 1H), 6.78 (d, J=8.5 Hz, 1H), 5.07 (d, J=4.5 Hz, 1H), 3.84 (s, 3H), 3.27-3.46 (m, 5H), 2.47 (br, J=7.5 Hz, 1H), 1.47 (s, 3H).

75-8 (P2): MS (ESI) m/z 253.1 (M+H⁺)

¹H NMR (400 MHz, chloroform-d) δ 8.01 (d, T=1.2 Hz, 1H), 7.87 (dd, J=1.6, 8.4 Hz, 1H), 6.76 (d, J=8.4 Hz, 1H), 5.05 (s, 1H), 3.82 (s, 3H), 3.39-3.46 (m, 1H), 3.28-3.33 (m, 4H), 2.74 (br, 1H), 1.46 (s, 3H).

### Step H: methyl 3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylate 75-9

DIAD (10.58 mL, 54.4 mmol) was added dropwise to a solution of tert-butyl (4,4-diethyl-6-oxotetrahydropyrimidin-2(1H)-ylidene)carbamate (11 g, 40.8 mmol), methyl (2S,3R)-3-hydroxy-2-(methoxymethyl)-2-methyl-2,3-dihydrobenzofuran-5-carboxylate 75-8 (10 g, 39.6 mmol) and triphenylphosphine (14 g, 53.4 mmol) in THF (150 mL), at 25° C. under N₂ atmosphere. Then the mixture was stirred at 27° C. for 2 h. The mixture was concentrated and added EtOAc (100 mL) to dissolved and added pet. ether slowly to solid appear. The mixture filtered and concentrated in vacuo. The residue was purified by column chromatography (SiO₂, petroleum ether:EtOAc:DCM=100:10:1-100:10:10) to afford product methyl 3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylate 75-9.

MS (ESI) m/z: 504.2 (M+H⁺)

¹H NMR (400 MHz, chloroform-d) δ 9.78-10.08 (m, 1H), 7.87-7.99 (m, 1H), 7.68-7.79 (m, 1H), 6.79-6.89 (m, 1H), 6.44-6.47 (m, 1H), 3.83-3.84 (m, 3H), 3.52-3.58 (m, 2H), 3.40 (s, 3H), 2.45-2.49 (m, 2H), 1.57-1.68 (m, 4H), 1.35-1.54 (m, 11H), 0.89-0.98 (m, 6H).

### Step I: (2R,3S)-methyl 3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylate 75-10A and methyl 3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylate 75-10B

The methyl 3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylate 75-9 (25.5 g, 50.6 mmol) was purified by SFC (Instrument SFC-17 Method Column DAICEL CHIRALPAK AD-H (250 mm×30 mm, 5 um), Condition 0.1% NH₃H₂O IPA Begin B 30% End B 30% Gradient Time (min) 100% B Hold Time (min) FlowRate (mL/min) 60 Injections 100) to give (2R,3S)-methyl 3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylate 75-10A (P1) (9.0 g, 17.87 mmol, 35.3% yield) (Rt=1.869) as an oil and methyl 3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylate 75-10B (P2).

75-10A (P1): MS (ESI) m/z 504.3 (M+H⁺)

¹H NMR (500 MHz, chloroform-d) δ 10.00 (s, 1H), 7.93 (dd, J=1.5, 8.5 Hz, 1H), 7.76 (s, 1H), 6.84 (d, J=8.5 Hz, 1H), 6.44 (s, 1H), 3.84 (s, 3H), 3.54 (s, 2H), 3.39 (s, 3H), 2.45 (s, 2H), 1.58-1.64 (m, 4H), 1.50-1.54 (m, 12H), 0.91-0.94 (m, 6H).

75-10B (P2): MS (ESI) m/z 504.3 (M+H⁺)

¹H NMR (500 MHz, chloroform-d) δ 9.91 (s, 1H), 7.91 (dd, J=1.5, 8.5 Hz, 1H), 7.71 (s, 1H), 6.83 (d, J=8.5 Hz, 1H), 6.47 (s, 1H), 3.83 (s, 3H), 3.53-3.59 (m, 2H), 3.40 (s, 3H), 2.50 (s, 2H), 1.60-1.66 (m, 4H), 1.51 (s, 9H), 1.37 (s, 3H), 0.90-0.97 (m, 6H).

### Step J: (2R,3S)-3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylic acid 75-11A

Potassium trimethylsilanolate (5.60 g, 43.7 mmol) was added to a solution of (2R,3S)-methyl 3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylate 75-10A (5.5 g, 10.92 mmol) in THF (100 mL). The reaction was stirred at 27° C. for 1 h under N₂ atmosphere. The solution of (2R,3S)-3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylic acid 75-11A was used for next step directly without any further manipulation or purification.

MS (ESI) m/z: 490.1 (M+H⁺)

### Step K: tert-butyl (4,4-diethyl-1-((2R,3S)-5-(((1R,2R)-2-hydroxy-2,3-dihydro-1H-inden-1-yl)carbamoyl)-2-(methoxymethyl)-2-methyl-2,3-dihydrobenzofuran-3-yl)-6-oxotetrahydropyrimidin-2(1H)-ylidene)carbamate 75-12A

N-ethyl-N-isopropylpropan-2-amine (7.06 g, 54.6 mmol) was added to a solution of (2R,3S)-3-(2-((tert-butoxycarbonyl)imino)-4,4-diethyl-6-oxotetrahydropyrimidin-1(2H)-yl)-2-(methoxymethyl)-2-methyl-2,3-dihydrobenzofuran-5-carboxylic acid 75-11A (5.35 g, 10.93 mmol), N1-((ethylimino)methylene)-N3,N3-dimethylpropane-1,3-diamine hydrochloride (4.19 g, 21.86 mmol), (1R,2R)-1-amino-2,3-dihydro-1H-inden-2-ol (1.956 g, 13.11 mmol) and 1H-benzo[d][1,2,3]triazol-1-ol (2.95 g, 21.86 mmol) in THF (100 mL). The reaction was stirred at 27° C. for 3 h under N₂ atmosphere. The mixture was quenched with water (100 mL) and extracted with EtOAc (100 mL×2). The organic layers were washed with brine (100 mL), dried over Na₂SO₄, filtered, and concentrated in vacuo. The residue was purified by column chromatography (SiO₂, petroleum ether:EtOAc:DCM=100:10:10-20:10:10) to give tert-butyl (4,4-diethyl-1-((2R,3S)-5-(((1R,2R)-2-hydroxy-2,3-dihydro-1H-inden-1-yl)carbamoyl)-2-(methoxymethyl)-2-methyl-2,3-dihydrobenzofuran-3-yl)-6-oxotetrahydropyrimidin-2(1H)-ylidene)carbamate 75-12A.

MS (ESI) m/z: 621.5 (M+H⁺)

¹H NMR (500 MHz, chloroform-d) δ 10.00 (s, 1H), 7.68 (s, 1H), 7.61 (dd, J=1.5, 8.5 Hz, 1H), 7.26-7.32 (m, 3H), 6.85 (d, J=8.5 Hz, 1H), 6.51 (s, 1H), 6.44 (d, J=6.0 Hz, 1H), 5.28-5.29 (m, 1H), 4.82 (s, 1H), 4.47 (q, J=7.5 Hz, 1H), 3.55 (d, J=1.5 Hz, 2H), 3.39 (s, 3H), 3.30-3.37 (m, 1H), 2.98 (dd, J=8.0, 15.5 Hz, 1H), 2.45 (d, J=2.5 Hz, 2H), 1.58-1.66 (m, 4H), 1.49-1.56 (m, 12H), 0.94 (q, J=7.5 Hz, 6H).

### Step L: (2R,3S)-3-(4,4-diethyl-2-imino-6-oxotetrahydropyrimidin-1(2H)-yl)-N-((1R,2R)-2-hydroxy-2,3-dihydro-1H-inden-1-yl)-2-(methoxymethyl)-2-methyl-2,3-dihydrobenzofuran-5-carboxamide Example 75A

A solution of tert-butyl (4,4-diethyl-1-((2R,3S)-5-(((1R,2R)-2-hydroxy-2,3-dihydro-1H-inden-1-yl)carbamoyl)-2-(methoxymethyl)-2-methyl-2,3-dihydrobenzofuran-3-yl)-6-oxotetrahydropyrimidin-2(1H)-ylidene)carbamate 75-12A (5.5 g, 8.86 mmol) in 4N HCl-dioxane (100 mL) was stirred at 27° C. for 1 h. The mixture was concentrated and purified by HPLC (Instrument ACSSH-prepL-K3 Method Column YMC-Triart Prep C18 250*50 mm*10 um Condition water (0.05% ammonia hydroxide v/v)-ACN Begin B 35 End B 55 Gradient Time (min) 15 100% B Hold Time (min) 5 FlowRate (mL/min) 110) then freeze-drying to give free base of desired product. The free base product was dissolved in MeCN (50 mL) and conc HCl (2 mL) in water (150 mL) and freeze-drying to afford product (2R,3S)-3-(4,4-diethyl-2-imino-6-oxotetrahydropyrimidin-1(2H)-yl)-N-((1R,2R)-2-hydroxy-2,3-dihydro-1H-inden-1-yl)-2-(methoxymethyl)-2-methyl-2,3-dihydrobenzofuran-5-carboxamide Example 75A.

MS (ESI) m/z: 521.2 (M+H⁺)

¹H NMR (500 MHz, methanol-d₄) δ 7.81-8.05 (m, 2H), 7.15-7.30 (m, 4H), 7.06 (d, J=8.5 Hz, 0.38H), 6.89 (d, J=9.0 Hz, 0.6H), 6.38 (s, 0.4H), 5.40-5.47 (m, 1.6H), 4.47-4.53 (m, 1H), 3.66-3.79 (m, 1.4H), 3.53 (d, J=9.5 Hz, 0.6H), 3.40-3.46 (m, 3H), 3.27-3.31 (m, 1H), 2.84-3.09 (m, 2H), 2.56-2.81 (m, 1H), 1.64-1.94 (m, 4H), 1.55 (d, J=14.5 Hz, 3H), 0.93-1.04 (m, 6H).

TABLE 2

| | | LC/MS | |
|---|---|---|---|
| Example | Structure | (M + 1)⁺ | Name |
| 75B | 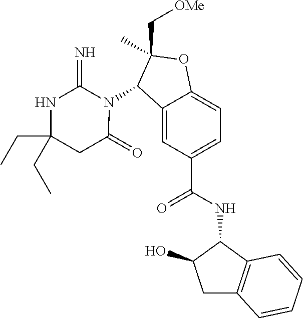 [SMILES: CCC1(CC)CC(=O)N([C@H]2c3cc(C(=O)N[C@@H]4c5ccccc5C[C@H]4O)ccc3O[C@]2(C)COC)C(=N)N1] | 521.2 | (2S,3S)-3-(4,4-diethyl-2-imino- 6-oxotetrahydropyrimidin- 1(2H)-yl)-N-((1R,2R)-2- hydroxy-2,3-dihydro-1H-inden- 1-yl)-2-(methoxymethyl)-2- methyl-2,3-dihydrobenzofuran- 5-carboxamide |
| 76A | 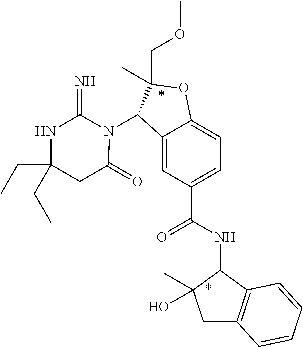 | 535.3 | (2S,3S)-3-(4,4-diethyl-2-imino- 6-oxotetrahydropyrimidin- 1(2H)-yl)-N-(2-hydroxy-2- methyl-2,3-dihydro-1H-inden-1- yl)-2-(methoxymethyl)-2- methyl-2,3-dihydrobenzofuran- 5-carboxamide |
| 76B | 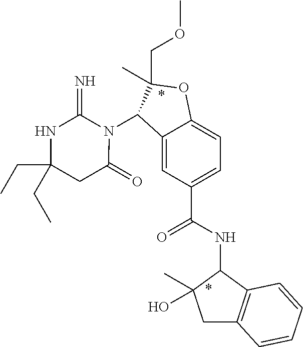 | 535.4 | (2S,3S)-3-(4,4-diethyl-2-imino- 6-oxotetrahydropyrimidin- 1(2H)-yl)-N-(2-hydroxy-2- methyl-2,3-dihydro-1H-inden-1- yl)-2-(methoxymethyl)-2- methyl-2,3-dihydrobenzofuran- 5-carboxamide |
| 77A | 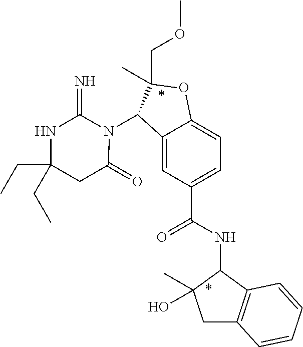 | 535.3 | (2S,3S)-3-(4,4-diethyl-2-imino- 6-oxotetrahydropyrimidin- 1(2H)-yl)-N-(2-hydroxy-2- methyl-2,3-dihydro-1H-inden-1- yl)-2-(methoxymethyl)-2- methyl-2,3-dihydrobenzofuran- 5-carboxamide |
| 77B | 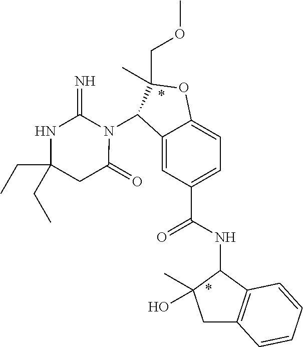 | 535.3 | (2S,3S)-3-(4,4-diethyl-2-imino- 6-oxotetrahydropyrimidin- 1(2H)-yl)-N-(2-hydroxy-2- methyl-2,3-dihydro-1H-inden-1- yl)-2-(methoxymethyl)-2- methyl-2,3-dihydrobenzofuran- 5-carboxamide |
| 78 | 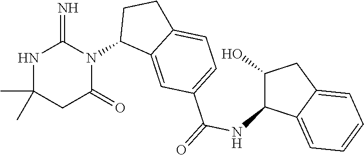 [SMILES: CC1(C)CC(=O)N([C@@H]2CCc3ccc(C(=O)N[C@@H]4c5ccccc5C[C@H]4O)cc32)C(=N)N1] | 433.0 | (3R)-N-[(1R,2R)-2- hydroxyindan-1-yl]-3-(2-imino- 4,4-dimethyl-6-oxo- hexahydropyrimidin-1- yl)indane-5-carboxamide |
| 79 | 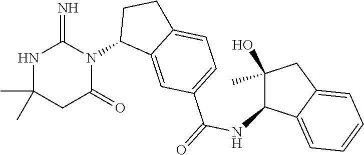 [SMILES: CC1(C)CC(=O)N([C@@H]2CCc3ccc(C(=O)N[C@@H]4c5ccccc5C[C@]4(C)O)cc32)C(=N)N1] | 447.3 | (3R)-N-[(1R,2S)-2-hydroxy-2- methyl-indan-1-yl]-3-(2-imino- 4,4-dimethyl-6-oxo- hexahydropyrimidin-1- yl)indane-5-carboxamide |
| 80 | 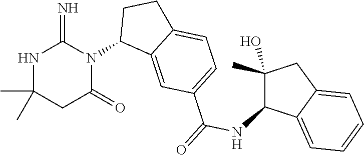 [SMILES: CC1(C)CC(=O)N([C@@H]2CCc3ccc(C(=O)N[C@@H]4c5ccccc5C[C@@]4(C)O)cc32)C(=N)N1] | 447.0 | (3R)-N-[(1R,2R)-2-hydroxy-2- methyl-indan-1-yl]-3-(2-imino- 4,4-dimethyl-6-oxo- hexahydropyrimidin-1- yl)indane-5-carboxamide |
| 81 | 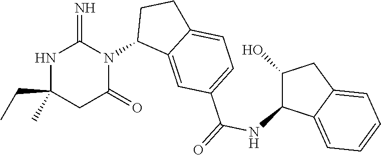 [SMILES: CC[C@]1(C)CC(=O)N([C@@H]2CCc3ccc(C(=O)N[C@@H]4c5ccccc5C[C@H]4O)cc32)C(=N)N1] | 447.3 | (3R)-3-[(4R)-4-ethyl-2-imino-4- methyl-6-oxo- hexahydropyrimidin-1-yl]-N- [(1R,2R)-2-hydroxyindan-1- yl]indane-5-carboxamide |
| 82 | 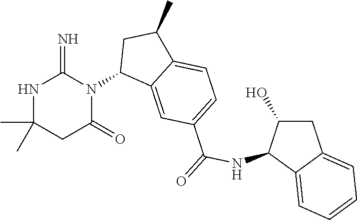 [SMILES: C[C@@H]1C[C@@H](N2C(=N)NC(C)(C)CC2=O)c2cc(C(=O)N[C@@H]3c4ccccc4C[C@H]3O)ccc21] | 447.3 | (1R,3R)-N-[(1R,2R)-2- hydroxyindan-1-yl]-3-(2-imino- 4,4-dimethyl-6-oxo- hexahydropyrimidin-1-yl)-1- methyl-indane-5-carboxamide |
| 83 | 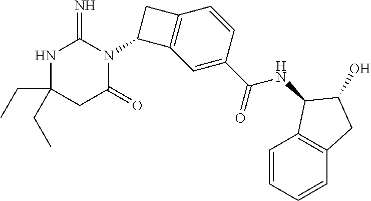 [SMILES: CCC1(CC)CC(=O)N([C@@H]2Cc3ccc(C(=O)N[C@@H]4c5ccccc5C[C@H]4O)cc32)C(=N)N1] | 447.1 | (8R)-8-(4,4-diethyl-2-imino-6- oxo-hexahydropyrimidin-1-yl)- N-[(1R,2R)-2-hydroxyindan-1- yl]bicyclo[4.2.0]octa-1(6),2,4- triene-3-carboxamide |
| 84 | 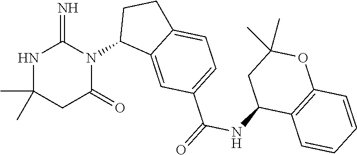 [SMILES: CC1(C)CC(=O)N([C@@H]2CCc3ccc(C(=O)N[C@H]4CC(C)(C)Oc5ccccc54)cc32)C(=N)N1] | 461.1 | (3R)-N-[(4S)-2,2- dimethylchroman-4-yl]-3-(2- imino-4,4-dimethyl-6-oxo- hexahydropyrimidin-1- yl)indane-5-carboxamide |
| 85 | 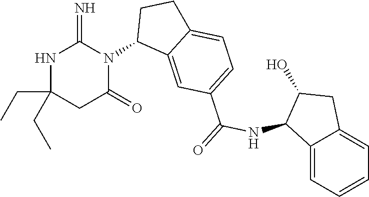 [SMILES: CCC1(CC)CC(=O)N([C@@H]2CCc3ccc(C(=O)N[C@@H]4c5ccccc5C[C@H]4O)cc32)C(=N)N1] | 461.3 | (3R)-3-(4,4-diethyl-2-imino-6- oxo-hexahydropyrimidin-1-yl)- N-[(1R,2R)-2-hydroxyindan-1- yl]indane-5-carboxamide |
| 86 | 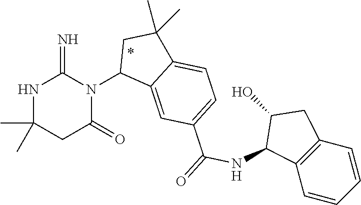 [SMILES: CC1(C)CC(=O)N(C2CC(C)(C)c3ccc(C(=O)N[C@@H]4c5ccccc5C[C@H]4O)cc32)C(=N)N1] | 461.3 | N-[(1R,2R)-2-hydroxyindan-1- yl]-3-(2-imino-4,4-dimethyl-6- oxo-hexahydropyrimidin-1-yl)- 1,1-dimethyl-indane-5- carboxamide |
| 87 | 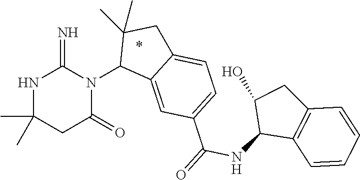 [SMILES: CC1(C)CC(=O)N(C2c3cc(C(=O)N[C@@H]4c5ccccc5C[C@H]4O)ccc3CC2(C)C)C(=N)N1] | 461.3 | N-[(1R,2R)-2-hydroxyindan-1- yl]-3-(2-imino-4,4-dimethyl-6- oxo-hexahydropyrimidin-1-yl)- 2,2-dimethyl-indane-5- carboxamide |
| 88 | 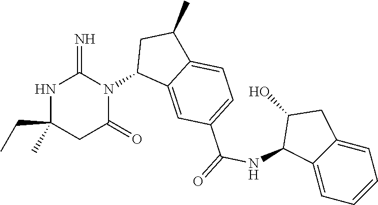 [SMILES: CC[C@]1(C)CC(=O)N([C@@H]2C[C@@H](C)c3ccc(C(=O)N[C@@H]4c5ccccc5C[C@H]4O)cc32)C(=N)N1] | 461.3 | (1R,3R)-3-[(4R)-4-ethyl-2- imino-4-methyl-6-oxo- hexahydropyrimidin-1-yl]-N- [(1R,2R)-2-hydroxyindan-1-yl]- 1-methyl-indane-5-carboxamide |
| 89 | 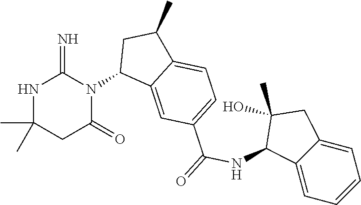 [SMILES: C[C@@H]1C[C@@H](N2C(=N)NC(C)(C)CC2=O)c2cc(C(=O)N[C@@H]3c4ccccc4C[C@@]3(C)O)ccc21] | 461.3 | (1R,3R)-N-[(1R,2R)-2-hydroxy- 2-methyl-indan-1-yl]-3-(2- imino-4,4-dimethyl-6-oxo- hexahydropyrimidin-1-yl)-1- methyl-indane-5-carboxamide |
| 90 | 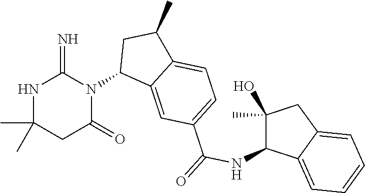 [SMILES: C[C@@H]1C[C@@H](N2C(=N)NC(C)(C)CC2=O)c2cc(C(=O)N[C@@H]3c4ccccc4C[C@]3(C)O)ccc21] | 461.3 | (1R,3R)-N-[(1R,2S)-2-hydroxy- 2-methyl-indan-1-yl]-3-(2- imino-4,4-dimethyl-6-oxo- hexahydropyrimidin-1-yl)-1- methyl-indane-5-carboxamide |
| 91 | 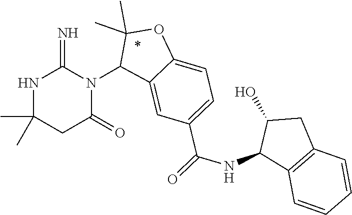 [SMILES: CC1(C)CC(=O)N(C2c3cc(C(=O)N[C@@H]4c5ccccc5C[C@H]4O)ccc3OC2(C)C)C(=N)N1] | 463.1 | N-[(1R,2R)-2-hydroxyindan-1- yl]-3-(2-imino-4,4-dimethyl-6- oxo-hexahydropyrimidin-1-yl)- 2,2-dimethyl-3H-benzofuran-5- carboxamide |
| 92 | 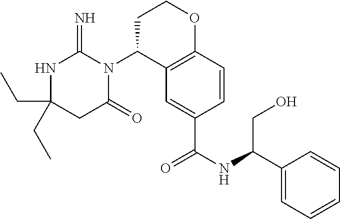 [SMILES: CCC1(CC)CC(=O)N([C@@H]2CCOc3ccc(C(=O)N[C@@H](CO)c4ccccc4)cc32)C(=N)N1] | 465.3 | (4R)-4-(4,4-diethyl-2-imino-6- oxo-hexahydropyrimidin-1-yl)- N-[(1R)-2-hydroxy-1-phenyl- ethyl]chromane-6-carboxamide |
| 93 | 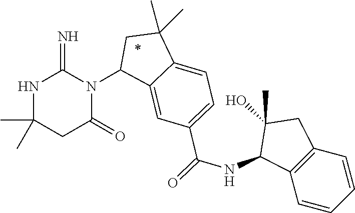 [SMILES: CC1(C)CC(=O)N(C2CC(C)(C)c3ccc(C(=O)N[C@@H]4c5ccccc5C[C@@]4(C)O)cc32)C(=N)N1] | 475.3 | N-[(1R,2R)-2-hydroxy-2- methyl-indan-1-yl]-3-(2-imino- 4,4-dimethyl-6-oxo- hexahydropyrimidin-1-yl)-1,1- dimethyl-indane-5-carboxamide |
| 94 | 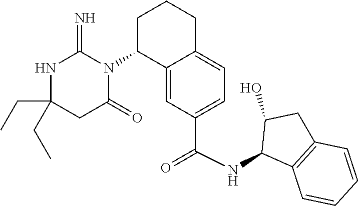 [SMILES: CCC1(CC)CC(=O)N([C@@H]2CCCc3ccc(C(=O)N[C@@H]4c5ccccc5C[C@H]4O)cc32)C(=N)N1] | 475.3 | (4R)-4-(4,4-diethyl-2-imino-6- oxo-hexahydropyrimidin-1-yl)- N-[(1R,2R)-2-hydroxyindan-1- yl]tetralin-6-carboxamide |
| 95 | 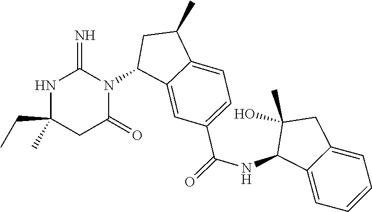 [SMILES: CC[C@]1(C)CC(=O)N([C@@H]2C[C@@H](C)c3ccc(C(=O)N[C@@H]4c5ccccc5C[C@@]4(C)O)cc32)C(=N)N1] | 475.3 | (1R,3R)-3-[(4R)-4-ethyl-2- imino-4-methyl-6-oxo- hexahydropyrimidin-1-yl]-N- [(1R,2R)-2-hydroxy-2-methyl- indan-1-yl]-1-methyl-indane-5- carboxamide |
| 96 | 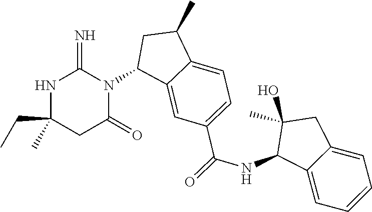 [SMILES: CC[C@]1(C)CC(=O)N([C@@H]2C[C@@H](C)c3ccc(C(=O)N[C@@H]4c5ccccc5C[C@]4(C)O)cc32)C(=N)N1] | 475.3 | (1R,3R)-3-[(4R)-4-ethyl-2- imino-4-methyl-6-oxo- hexahydropyrimidin-1-yl]-N- [(1R,2S)-2-hydroxy-2-methyl- indan-1-yl]-1-methyl-indane-5- carboxamide |
| 97 | 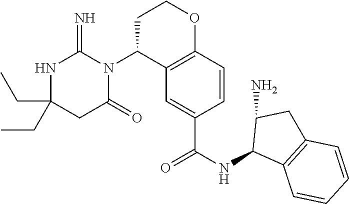 [SMILES: CCC1(CC)CC(=O)N([C@@H]2CCOc3ccc(C(=O)N[C@@H]4c5ccccc5C[C@H]4N)cc32)C(=N)N1] | 476.2 | (4R)-N-[(1R,2R)-2-aminoindan- 1-yl]-4-(4,4-diethyl-2-imino-6- oxo-hexahydropyrimidin-1- yl)chromane-6-carboxamide |
| 98 | 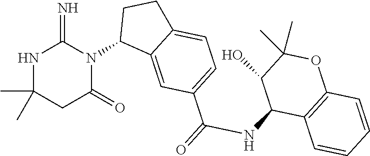 [SMILES: CC1(C)CC(=O)N([C@@H]2CCc3ccc(C(=O)N[C@@H]4c5ccccc5OC(C)(C)[C@H]4O)cc32)C(=N)N1] | 477.1 | (3R)-N-[(3S,4R)-3-hydroxy-2,2- dimethyl-chroman-4-yl]-3-(2- imino-4,4-dimethyl-6-oxo- hexahydropyrimidin-1- yl)indane-5-carboxamide |
| 99 | 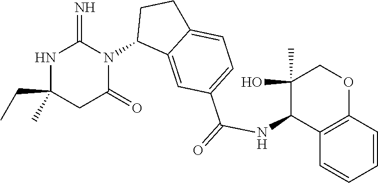 [SMILES: CC[C@]1(C)CC(=O)N([C@@H]2CCc3ccc(C(=O)N[C@@H]4c5ccccc5OC[C@]4(C)O)cc32)C(=N)N1] | 477.4 | (3R)-3-[(4R)-4-ethyl-2-imino-4- methyl-6-oxo- hexahydropyrimidin-1-yl]-N- [(3R,4R)-3-hydroxy-3-methyl- chroman-4-yl]indane-5- carboxamide |
| 100 | 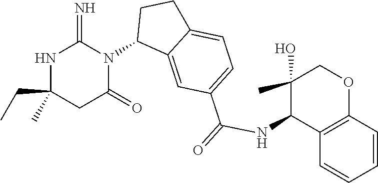 [SMILES: CC[C@]1(C)CC(=O)N([C@@H]2CCc3ccc(C(=O)N[C@@H]4c5ccccc5OC[C@@]4(C)O)cc32)C(=N)N1] | 477.3 | (3R)-3-[(4R)-4-ethyl-2-imino-4- methyl-6-oxo- hexahydropyrimidin-1-yl]-N- [(3S,4R)-3-hydroxy-3-methyl- chroman-4-yl]indane-5- carboxamide |
| 101 | 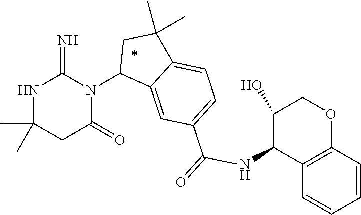 [SMILES: CC1(C)CC(=O)N(C2CC(C)(C)c3ccc(C(=O)N[C@@H]4c5ccccc5OC[C@H]4O)cc32)C(=N)N1] | 477.3 | N-[(3S,4R)-3-hydroxychroman- 4-yl]-3-(2-imino-4,4-dimethyl- 6-oxo-hexahydropyrimidin-1- yl)-1,1-dimethyl-indane-5- carboxamide |
| 102 | 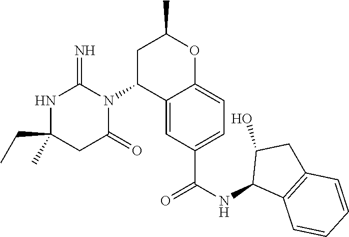 [SMILES: CC[C@]1(C)CC(=O)N([C@@H]2C[C@@H](C)Oc3ccc(C(=O)N[C@@H]4c5ccccc5C[C@H]4O)cc32)C(=N)N1] | 477.3 | (2R,4R)-4-[(4R)-4-ethyl-2- imino-4-methyl-6-oxo- hexahydropyrimidin-1-yl]-N- [(1R,2R)-2-hydroxyindan-1-yl]- 2-methyl-chromane-6- carboxamide |
| 103 | 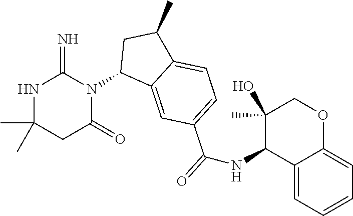 [SMILES: C[C@@H]1C[C@@H](N2C(=N)NC(C)(C)CC2=O)c2cc(C(=O)N[C@@H]3c4ccccc4OC[C@]3(C)O)ccc21] | 477.3 | (1R,3R)-N-[(3R,4R)-3-hydroxy- 3-methyl-chroman-4-yl]-3-(2- imino-4,4-dimethyl-6-oxo- hexahydropyrimidin-1-yl)-1- methyl-indane-5-carboxamide |
| 104 | 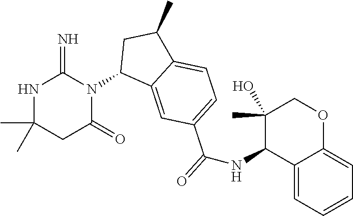 [SMILES: C[C@@H]1C[C@@H](N2C(=N)NC(C)(C)CC2=O)c2cc(C(=O)N[C@@H]3c4ccccc4OC[C@@]3(C)O)ccc21] | 477.2 | (1R,3R)-N-[(3S,4R)-3-hydroxy- 3-methyl-chroman-4-yl]-3-(2- imino-4,4-dimethyl-6-oxo- hexahydropyrimidin-1-yl)-1- methyl-indane-5-carboxamide |
| 105 | 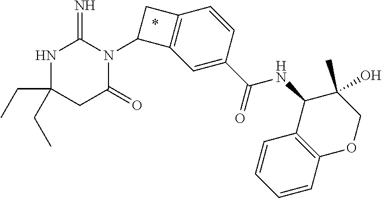 [SMILES: CCC1(CC)CC(=O)N(C2Cc3ccc(C(=O)N[C@@H]4c5ccccc5OC[C@@]4(C)O)cc32)C(=N)N1] | 477.1 | 8-(4,4-diethyl-2-imino-6-oxo- hexahydropyrimidin-1-yl)-N- [(3S,4R)-3-hydroxy-3-methyl- chroman-4- yl]bicyclo[4.2.0]octa-1(6),2,4- triene-3-carboxamide |
| 106 | 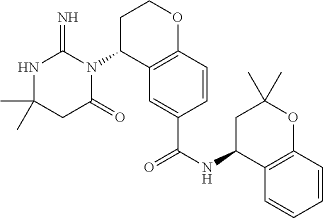 [SMILES: CC1(C)CC(=O)N([C@@H]2CCOc3ccc(C(=O)N[C@H]4CC(C)(C)Oc5ccccc54)cc32)C(=N)N1] | 477.3 | (4R)-N-[(4S)-2,2- dimethylchroman-4-yl]-4-(2- imino-4,4-dimethyl-6-oxo- hexahydropyrimidin-1- yl)chromane-6-carboxamide |
| 107 | 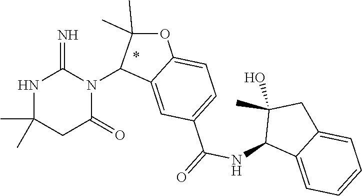 [SMILES: CC1(C)CC(=O)N(C2c3cc(C(=O)N[C@@H]4c5ccccc5C[C@@]4(C)O)ccc3OC2(C)C)C(=N)N1] | 477.3 | N-[(1R,2R)-2-hydroxy-2- methyl-indan-1-yl]-3-(2-imino- 4,4-dimethyl-6-oxo- hexahydropyrimidin-1-yl)-2,2- dimethyl-3H-benzofuran-5- carboxamide |
| 108 | 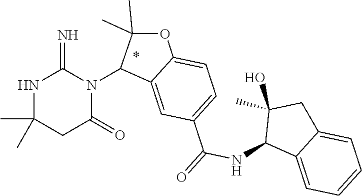 [SMILES: CC1(C)CC(=O)N(C2c3cc(C(=O)N[C@@H]4c5ccccc5C[C@]4(C)O)ccc3OC2(C)C)C(=N)N1] | 477.3 | N-[(1R,2S)-2-hydroxy-2- methyl-indan-1-yl]-3-(2-imino- 4,4-dimethyl-6-oxo- hexahydropyrimidin-1-yl)-2,2- dimethyl-3H-benzofuran-5- carboxamide |
| 109 | 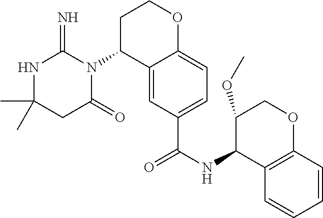 [SMILES: CO[C@@H]1COc2ccccc2[C@H]1NC(=O)c1ccc2c(c1)[C@H](N1C(=N)NC(C)(C)CC1=O)CCO2] | 479.2 | (4R)-4-(2-imino-4,4-dimethyl-6- oxo-hexahydropyrimidin-1-yl)- N-[(3S,4R)-3-methoxychroman- 4-yl]chromane-6-carboxamide |
| 110 | 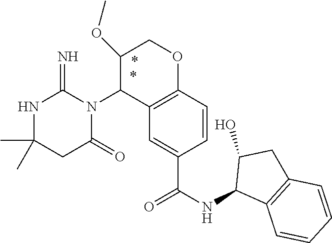 [SMILES: COC1COc2ccc(C(=O)N[C@@H]3c4ccccc4C[C@H]3O)cc2C1N1C(=N)NC(C)(C)CC1=O] | 479.2 | N-[(1R,2R)-2-hydroxyindan-1- yl]-4-(2-imino-4,4-dimethyl-6- oxo-hexahydropyrimidin-1-yl)- 3-methoxy-chromane-6- carboxamide |
| 111 | 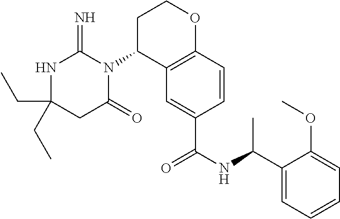 [SMILES: CCC1(CC)CC(=O)N([C@@H]2CCOc3ccc(C(=O)N[C@@H](C)c4ccccc4OC)cc32)C(=N)N1] | 479.3 | (4R)-4-(4,4-diethyl-2-imino-6- oxo-hexahydropyrimidin-1-yl)- N-[(1S)-1-(2- methoxyphenyl)ethyl]chromane- 6-carboxamide |
| 112 | 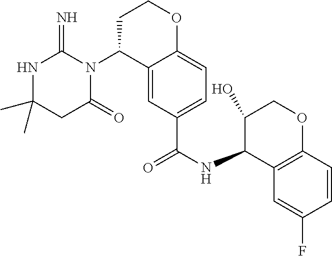 [SMILES: CC1(C)CC(=O)N([C@@H]2CCOc3ccc(C(=O)N[C@@H]4c5cc(F)ccc5OC[C@H]4O)cc32)C(=N)N1] | 483.3 | (4R)-N-[(3S,4R)-6-fluoro-3- hydroxy-chroman-4-yl]-4-(2- imino-4,4-dimethyl-6-oxo- hexahydropyrimidin-1- yl)chromane-6-carboxamide |
| 113 | 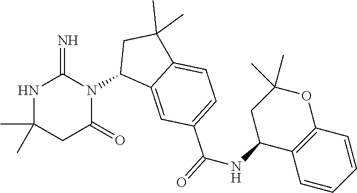 [SMILES: CC1(C)CC(=O)N([C@@H]2CC(C)(C)c3ccc(C(=O)N[C@H]4CC(C)(C)Oc5ccccc54)cc32)C(=N)N1] | 489.3 | (3R)-N-[(4S)-2,2- dimethylchroman-4-yl]-3-(2- imino-4,4-dimethyl-6-oxo- hexahydropyrimidin-1-yl)-1,1- dimethyl-indane-5-carboxamide |
| 114 | 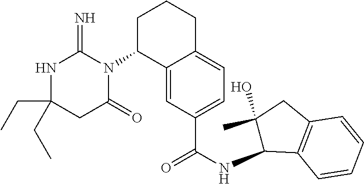 [SMILES: CCC1(CC)CC(=O)N([C@@H]2CCCc3ccc(C(=O)N[C@@H]4c5ccccc5C[C@@]4(C)O)cc32)C(=N)N1] | 489.3 | (4R)-4-(4,4-diethyl-2-imino-6- oxo-hexahydropyrimidin-1-yl)- N-[(1R,2R)-2-hydroxy-2- methyl-indan-1-yl]tetralin-6- carboxamide |
| 115 | 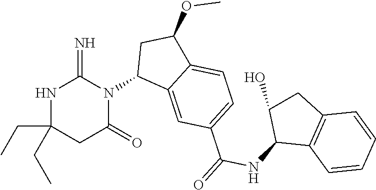 [SMILES: CCC1(CC)CC(=O)N([C@@H]2C[C@@H](OC)c3ccc(C(=O)N[C@@H]4c5ccccc5C[C@H]4O)cc32)C(=N)N1] | 491.3 | (1R,3R)-3-(4,4-diethyl-2-imino- 6-oxo-hexahydropyrimidin-1- yl)-N-[(1R,2R)-2-hydroxyindan- 1-yl]-1-methoxy-indane-5- carboxamide |
| 116 | 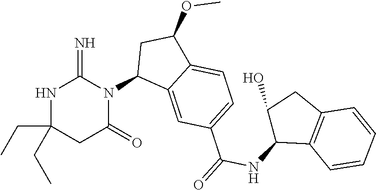 [SMILES: CCC1(CC)CC(=O)N([C@H]2C[C@@H](OC)c3ccc(C(=O)N[C@@H]4c5ccccc5C[C@H]4O)cc32)C(=N)N1] | 491.3 | (1R,3S)-3-(4,4-diethyl-2-imino- 6-oxo-hexahydropyrimidin-1- yl)-N-[(1R,2R)-2-hydroxyindan- 1-yl]-1-methoxy-indane-5- carboxamide |
| 117 | 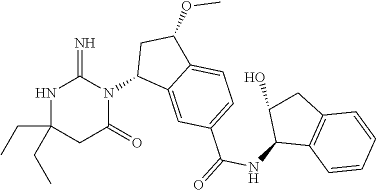 [SMILES: CCC1(CC)CC(=O)N([C@@H]2C[C@H](OC)c3ccc(C(=O)N[C@@H]4c5ccccc5C[C@H]4O)cc32)C(=N)N1] | 491.3 | (1S,3R)-3-(4,4-diethyl-2-imino- 6-oxo-hexahydropyrimidin-1- yl)-N-[(1R,2R)-2-hydroxyindan- 1-yl]-1-methoxy-indane-5- carboxamide |
| 118 | 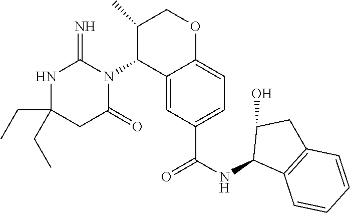 [SMILES: CCC1(CC)CC(=O)N([C@H]2c3cc(C(=O)N[C@@H]4c5ccccc5C[C@H]4O)ccc3OC[C@H]2C)C(=N)N1] | 491.3 | (3S,4R)-4-(4,4-diethyl-2-imino- 6-oxo-hexahydropyrimidin-1- yl)-N-[(1R,2R)-2-hydroxyindan- 1-yl]-3-methyl-chromane-6- carboxamide |
| 119 | 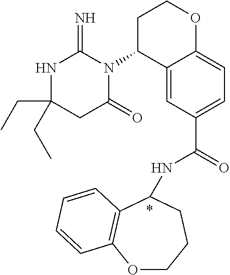 [SMILES: CCC1(CC)CC(=O)N([C@@H]2CCOc3ccc(C(=O)NC4CCCOc5ccccc54)cc32)C(=N)N1] | 491.3 | (4R)-4-(4,4-diethyl-2-imino-6- oxo-hexahydropyrimidin-1-yl)- N-(2,3,4,5-tetrahydro-1- benzoxepin-5-yl)chromane-6- carboxamide |
| 120 | 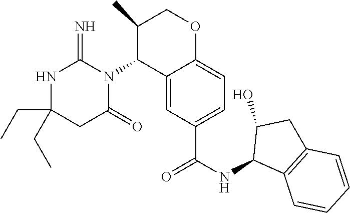 [SMILES: CCC1(CC)CC(=O)N([C@H]2c3cc(C(=O)N[C@@H]4c5ccccc5C[C@H]4O)ccc3OC[C@@H]2C)C(=N)N1] | 491.3 | (3R,4R)-4-(4,4-diethyl-2-imino- 6-oxo-hexahydropyrimidin-1- yl)-N-[(1R,2R)-2-hydroxyindan- 1-yl]-3-methyl-chromane-6- carboxamide |
| 121 | 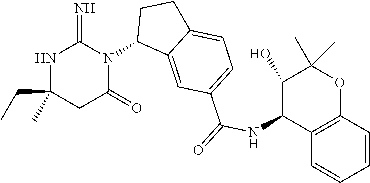 [SMILES: CC[C@]1(C)CC(=O)N([C@@H]2CCc3ccc(C(=O)N[C@@H]4c5ccccc5OC(C)(C)[C@H]4O)cc32)C(=N)N1] | 491.3 | (3R)-3-[(4R)-4-ethyl-2-imino-4- methyl-6-oxo- hexahydropyrimidin-1-yl]-N- [(3S,4R)-3-hydroxy-2,2- dimethyl-chroman-4-yl]indane- 5-carboxamide |
| 122 | 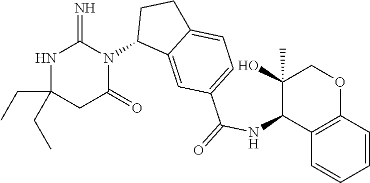 [SMILES: CCC1(CC)CC(=O)N([C@@H]2CCc3ccc(C(=O)N[C@@H]4c5ccccc5OC[C@]4(C)O)cc32)C(=N)N1] | 491.3 | (3R)-3-(4,4-diethyl-2-imino-6- oxo-hexahydropyrimidin-1-yl)- N-[(3R,4R)-3-hydroxy-3- methyl-chroman-4-yl]indane-5- carboxamide |
| 123 | 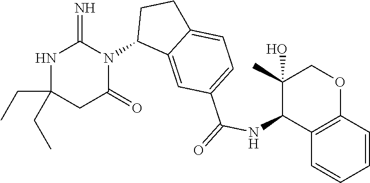 [SMILES: CCC1(CC)CC(=O)N([C@@H]2CCc3ccc(C(=O)N[C@@H]4c5ccccc5OC[C@@]4(C)O)cc32)C(=N)N1] | 491.3 | (3R)-3-(4,4-diethyl-2-imino-6- oxo-hexahydropyrimidin-1-yl)- N-[(3S,4R)-3-hydroxy-3- methyl-chroman-4-yl]indane-5- carboxamide |
| 124 | 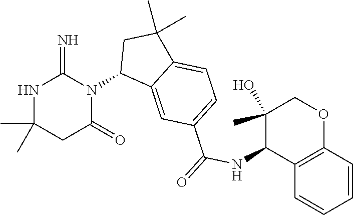 [SMILES: CC1(C)CC(=O)N([C@@H]2CC(C)(C)c3ccc(C(=O)N[C@@H]4c5ccccc5OC[C@@]4(C)O)cc32)C(=N)N1] | 491.3 | (3R)-N-[(3S,4R)-3-hydroxy-3- methyl-chroman-4-yl]-3-(2- imino-4,4-dimethyl-6-oxo- hexahydropyrimidin-1-yl)-1,1- dimethyl-indane-5-carboxamide |
| 125 | 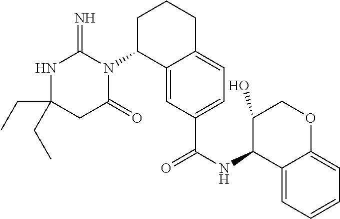 [SMILES: CCC1(CC)CC(=O)N([C@@H]2CCCc3ccc(C(=O)N[C@@H]4c5ccccc5OC[C@H]4O)cc32)C(=N)N1] | 491.3 | (4R)-4-(4,4-diethyl-2-imino-6- oxo-hexahydropyrimidin-1-yl)- N-[(3S,4R)-3-hydroxychroman- 4-yl]tetralin-6-carboxamide |
| 126 | 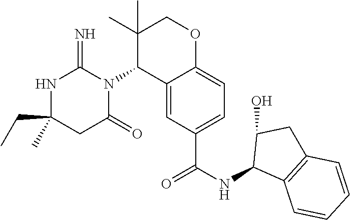 [SMILES: CC[C@]1(C)CC(=O)N([C@H]2c3cc(C(=O)N[C@@H]4c5ccccc5C[C@H]4O)ccc3OCC2(C)C)C(=N)N1] | 491.3 | (4R)-4-[(4R)-4-ethyl-2-imino-4- methyl-6-oxo- hexahydropyrimidin-1-yl]-N- [(1R,2R)-2-hydroxyindan-1-yl]- 3,3-dimethyl-chromane-6- carboxamide |
| 127A | 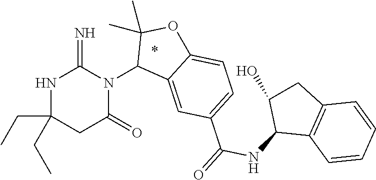 [SMILES: CCC1(CC)CC(=O)N(C2c3cc(C(=O)N[C@@H]4c5ccccc5C[C@H]4O)ccc3OC2(C)C)C(=N)N1] | 491.3 | 3-(4,4-diethyl-2-imino-6-oxo- hexahydropyrimidin-1-yl)-N- [(1R,2R)-2-hydroxyindan-1-yl]- 2,2-dimethyl-3H-benzofuran-5- carboxamide |
| 127B | 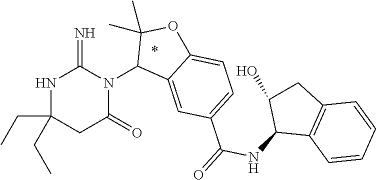 [SMILES: CCC1(CC)CC(=O)N(C2c3cc(C(=O)N[C@@H]4c5ccccc5C[C@H]4O)ccc3OC2(C)C)C(=N)N1] | 491.3 | 3-(4,4-diethyl-2-imino-6-oxo- hexahydropyrimidin-1-yl)-N- [(1R,2R)-2-hydroxyindan-1-yl]- 2,2-dimethyl-3H-benzofuran-5- carboxamide |
| 128 | 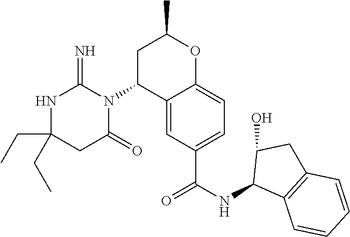 [SMILES: CCC1(CC)CC(=O)N([C@@H]2C[C@@H](C)Oc3ccc(C(=O)N[C@@H]4c5ccccc5C[C@H]4O)cc32)C(=N)N1] | 491.3 | (2R,4R)-4-(4,4-diethyl-2-imino- 6-oxo-hexahydropyrimidin-1- yl)-N-[(1R,2R)-2-hydroxyindan- 1-yl]-2-methyl-chromane-6- carboxamide |
| 129 | 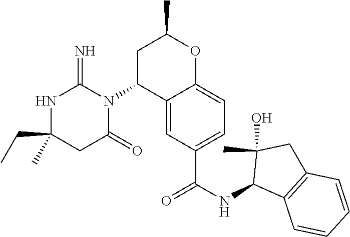 [SMILES: CC[C@]1(C)CC(=O)N([C@@H]2C[C@@H](C)Oc3ccc(C(=O)N[C@@H]4c5ccccc5C[C@@]4(C)O)cc32)C(=N)N1] | 491.3 | (2R,4R)-4-[(4R)-4-ethyl-2- imino-4-methyl-6-oxo- hexahydropyrimidin-1-yl]-N- [(1R,2R)-2-hydroxy-2-methyl- indan-1-yl]-2-methyl-chromane- 6-carboxamide |
| 130 | 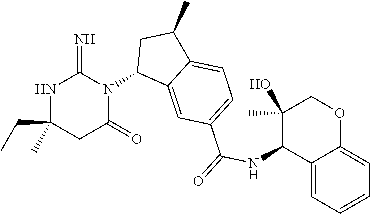 [SMILES: CC[C@]1(C)CC(=O)N([C@@H]2C[C@@H](C)c3ccc(C(=O)N[C@@H]4c5ccccc5OC[C@]4(C)O)cc32)C(=N)N1] | 491.3 | (1R,3R)-3-[(4R)-4-ethyl-2- imino-4-methyl-6-oxo- hexahydropyrimidin-1-yl]-N- [(3R,4R)-3-hydroxy-3-methyl- chroman-4-yl]-1-methyl-indane- 5-carboxamide |
| 131 | 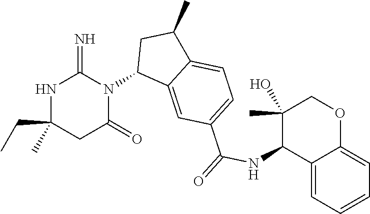 [SMILES: CC[C@]1(C)CC(=O)N([C@@H]2C[C@@H](C)c3ccc(C(=O)N[C@@H]4c5ccccc5OC[C@@]4(C)O)cc32)C(=N)N1] | 491.2 | (1R,3R)-3-[(4R)-4-ethyl-2- imino-4-methyl-6-oxo- hexahydropyrimidin-1-yl]-N- [(3S,4R)-3-hydroxy-3-methyl- chroman-4-yl]-1-methyl-indane- 5-carboxamide |
| 132 | 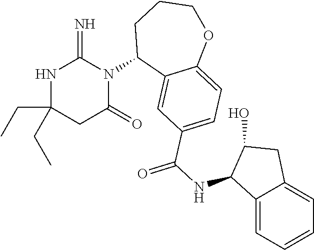 [SMILES: CCC1(CC)CC(=O)N([C@@H]2CCCOc3ccc(C(=O)N[C@@H]4c5ccccc5C[C@H]4O)cc32)C(=N)N1] | 491.1 | (5R)-5-(4,4-diethyl-2-imino-6- oxo-hexahydropyrimidin-1-yl)- N-[(1R,2R)-2-hydroxyindan-1- yl]-2,3,4,5-tetrahydro-1- benzoxepine-7-carboxamide |
| 133A | 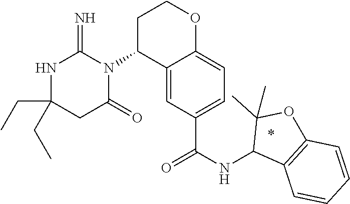 [SMILES: CCC1(CC)CC(=O)N([C@@H]2CCOc3ccc(C(=O)NC4c5ccccc5OC4(C)C)cc32)C(=N)N1] | 491.3 | (4R)-4-(4,4-diethyl-2-imino-6- oxo-hexahydropyrimidin-1-yl)- N-(2,2-dimethyl-3H- benzofuran-3-yl)chromane-6- carboxamide |
| 133B | 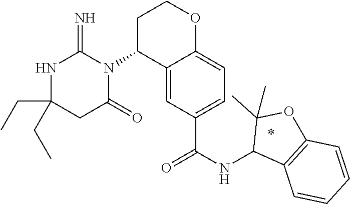 [SMILES: CCC1(CC)CC(=O)N([C@@H]2CCOc3ccc(C(=O)NC4c5ccccc5OC4(C)C)cc32)C(=N)N1] | 491.3 | (4R)-4-(4,4-diethyl-2-imino-6- oxo-hexahydropyrimidin-1-yl)- N-(2,2-dimethyl-3H- benzofuran-3-yl)chromane-6- carboxamide |
| 134 | 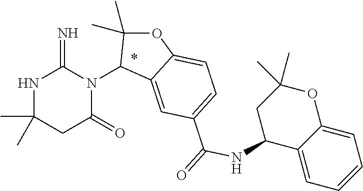 [SMILES: CC1(C)CC(=O)N(C2c3cc(C(=O)N[C@H]4CC(C)(C)Oc5ccccc54)ccc3OC2(C)C)C(=N)N1] | 491.3 | N-[(4S)-2,2-dimethylchroman- 4-yl]-3-(2-imino-4,4-dimethyl- 6-oxo-hexahydropyrimidin-1- yl)-2,2-dimethyl-3H- benzofuran-5-carboxamide |
| 135 | 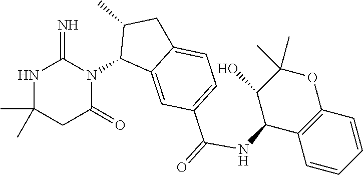 [SMILES: C[C@@H]1Cc2ccc(C(=O)N[C@@H]3c4ccccc4OC(C)(C)[C@H]3O)cc2[C@@H]1N1C(=N)NC(C)(C)CC1=O] | 491.3 | (2R,3R)-N-[(3S,4R)-3-hydroxy- 2,2-dimethyl-chroman-4-yl]-3- (2-imino-4,4-dimethyl-6-oxo- hexahydropyrimidin-1-yl)-2- methyl-indane-5-carboxamide |
| 136 | 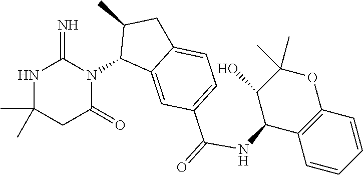 [SMILES: C[C@H]1Cc2ccc(C(=O)N[C@@H]3c4ccccc4OC(C)(C)[C@H]3O)cc2[C@@H]1N1C(=N)NC(C)(C)CC1=O] | 491.3 | (2S,3R)-N-[(3S,4R)-3-hydroxy- 2,2-dimethyl-chroman-4-yl]-3- (2-imino-4,4-dimethyl-6-oxo- hexahydropyrimidin-1-yl)-2- methyl-indane-5-carboxamide |
| 137 | 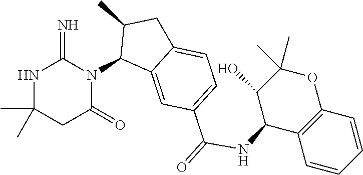 [SMILES: C[C@H]1Cc2ccc(C(=O)N[C@@H]3c4ccccc4OC(C)(C)[C@H]3O)cc2[C@H]1N1C(=N)NC(C)(C)CC1=O] | 491.3 | (2S,3S)-N-[(3S,4R)-3-hydroxy- 2,2-dimethyl-chroman-4-yl]-3- (2-imino-4,4-dimethyl-6-oxo- hexahydropyrimidin-1-yl)-2- methyl-indane-5-carboxamide |
| 138 | 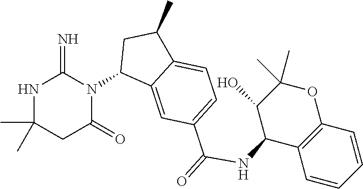 [SMILES: C[C@@H]1C[C@@H](N2C(=N)NC(C)(C)CC2=O)c2cc(C(=O)N[C@@H]3c4ccccc4OC(C)(C)[C@H]3O)ccc21] | 491.3 | (1R,3R)-N-[(3S,4R)-3-hydroxy- 2,2-dimethyl-chroman-4-yl]-3- (2-imino-4,4-dimethyl-6-oxo- hexahydropyrimidin-1-yl)-1- methyl-indane-5-carboxamide |
| 139 | 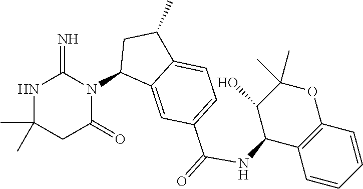 [SMILES: C[C@H]1C[C@H](N2C(=N)NC(C)(C)CC2=O)c2cc(C(=O)N[C@@H]3c4ccccc4OC(C)(C)[C@H]3O)ccc21] | 491.3 | (1S,3S)-N-[(3S,4R)-3-hydroxy- 2,2-dimethyl-chroman-4-yl]-3- (2-imino-4,4-dimethyl-6-oxo- hexahydropyrimidin-1-yl)-1- methyl-indane-5-carboxamide |
| 140A | 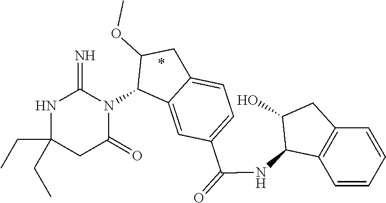 [SMILES: CCC1(CC)CC(=O)N([C@H]2c3cc(C(=O)N[C@@H]4c5ccccc5C[C@H]4O)ccc3CC2OC)C(=N)N1] | 491.3 | (3S)-3-(4,4-diethyl-2-imino-6- oxo-hexahydropyrimidin-1-yl)- N-[(1R,2R)-2-hydroxyindan-1- yl]-2-methoxy-indane-5- carboxamide |
| 140B | 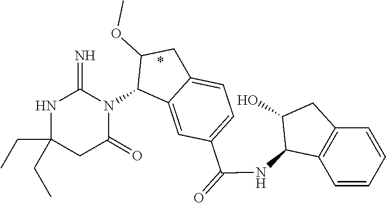 [SMILES: CCC1(CC)CC(=O)N([C@H]2c3cc(C(=O)N[C@@H]4c5ccccc5C[C@H]4O)ccc3CC2OC)C(=N)N1] | 491.3 | (3S)-3-(4,4-diethyl-2-imino-6- oxo-hexahydropyrimidin-1-yl)- N-[(1R,2R)-2-hydroxyindan-1- yl]-2-methoxy-indane-5- carboxamide |
| 141 | 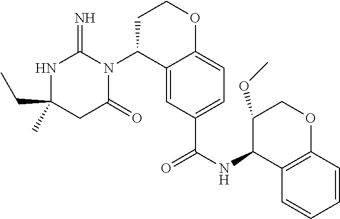 [SMILES: CC[C@]1(C)CC(=O)N([C@@H]2CCOc3ccc(C(=O)N[C@@H]4c5ccccc5OC[C@H]4OC)cc32)C(=N)N1] | 493.2 | (4R)-4-[(4R)-4-ethyl-2-imino-4- methyl-6-oxo- hexahydropyrimidin-1-yl]-N- [(3S,4R)-3-methoxychroman-4- yl]chromane-6-carboxamide |
| 142 | 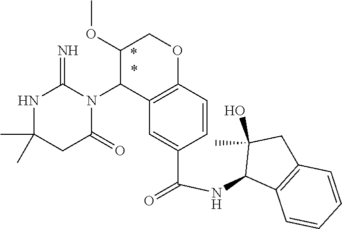 [SMILES: COC1COc2ccc(C(=O)N[C@@H]3c4ccccc4C[C@]3(C)O)cc2C1N1C(=N)NC(C)(C)CC1=O] | 493.2 | N-[(1R,2S)-2-hydroxy-2- methyl-indan-1-yl]-4-(2-imino- 4,4-dimethyl-6-oxo- hexahydropyrimidin-1-yl)-3- methoxy-chromane-6- carboxamide |
| 143 | 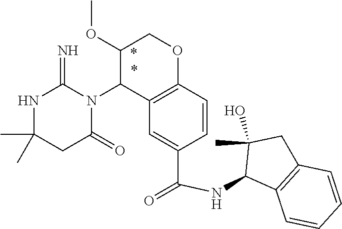 [SMILES: COC1COc2ccc(C(=O)N[C@@H]3c4ccccc4C[C@@]3(C)O)cc2C1N1C(=N)NC(C)(C)CC1=O] | 493.2 | N-[(1R,2R)-2-hydroxy-2- methyl-indan-1-yl]-4-(2-imino- 4,4-dimethyl-6-oxo- hexahydropyrimidin-1-yl)-3- methoxy-chromane-6- carboxamide |
| 144 | 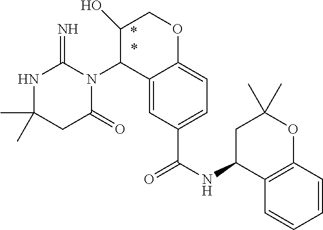 [SMILES: CC1(C)CC(=O)N(C2c3cc(C(=O)N[C@H]4CC(C)(C)Oc5ccccc54)ccc3OCC2O)C(=N)N1] | 493.3 | N-[(4S)-2,2-dimethylchroman- 4-yl]-3-hydroxy-4-(2-imino-4,4- dimethyl-6-oxo- hexahydropyrimidin-1- yl)chromane-6-carboxamide |
| 145 | 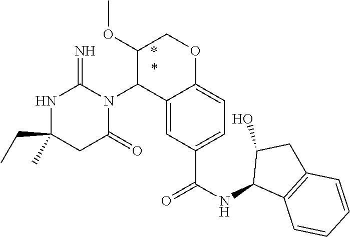 [SMILES: CC[C@]1(C)CC(=O)N(C2c3cc(C(=O)N[C@@H]4c5ccccc5C[C@H]4O)ccc3OCC2OC)C(=N)N1] | 493.2 | 4-[(4R)-4-ethyl-2-imino-4- methyl-6-oxo- hexahydropyrimidin-1-yl]-N- [(1R,2R)-2-hydroxyindan-1-yl]- 3-methoxy-chromane-6- carboxamide |
| 146 | 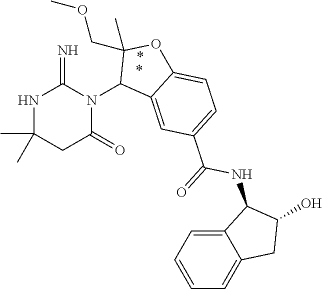 [SMILES: COCC1(C)Oc2ccc(C(=O)N[C@@H]3c4ccccc4C[C@H]3O)cc2C1N1C(=N)NC(C)(C)CC1=O] | 493.1 | N-[(1R,2R)-2-hydroxyindan-1- yl]-3-(2-imino-4,4-dimethyl-6- oxo-hexahydropyrimidin-1-yl)- 2-(methoxymethyl)-2-methyl- 3H-benzofuran-5-carboxamide |
| 147 | 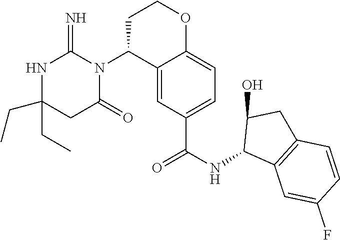 [SMILES: CCC1(CC)CC(=O)N([C@@H]2CCOc3ccc(C(=O)N[C@H]4c5cc(F)ccc5C[C@@H]4O)cc32)C(=N)N1] | 495.3 | (4R)-4-(4,4-diethyl-2-imino-6- oxo-hexahydropyrimidin-1-yl)- N-[(1S,2S)-6-fluoro-2-hydroxy- indan-1-yl]chromane-6- carboxamide |
| 148 | 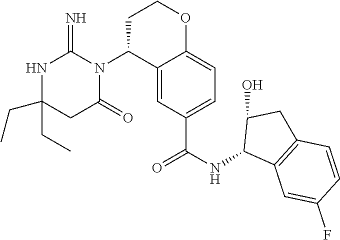 [SMILES: CCC1(CC)CC(=O)N([C@@H]2CCOc3ccc(C(=O)N[C@H]4c5cc(F)ccc5C[C@H]4O)cc32)C(=N)N1] | 495.3 | (4R)-4-(4,4-diethyl-2-imino-6- oxo-hexahydropyrimidin-1-yl)- N-[(1R,2R)-6-fluoro-2-hydroxy- indan-1-yl]chromane-6- carboxamide |
| 149 | 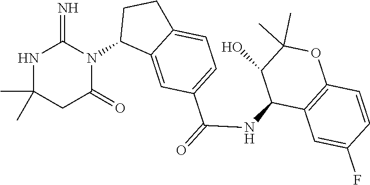 [SMILES: CC1(C)CC(=O)N([C@@H]2CCc3ccc(C(=O)N[C@@H]4c5cc(F)ccc5OC(C)(C)[C@H]4O)cc32)C(=N)N1] | 495.1 | (3R)-N-[(3S,4R)-6-fluoro-3- hydroxy-2,2-dimethyl-chroman- 4-yl]-3-(2-imino-4,4-dimethyl- 6-oxo-hexahydropyrimidin-1- yl)indane-5-carboxamide |
| 150 | 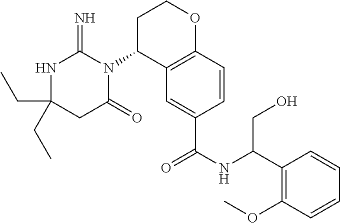 [SMILES: CCC1(CC)CC(=O)N([C@@H]2CCOc3ccc(C(=O)NC(CO)c4ccccc4OC)cc32)C(=N)N1] | 495.3 | (4R)-4-(4,4-diethyl-2-imino-6- oxo-hexahydropyrimidin-1-yl)- N-[2-hydroxy-1-(2- methoxyphenyl)ethyl]chromane- 6-carboxamide |
| 151 | 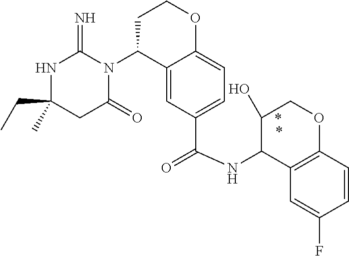 [SMILES: CC[C@]1(C)CC(=O)N([C@@H]2CCOc3ccc(C(=O)NC4c5cc(F)ccc5OCC4O)cc32)C(=N)N1] | 497.2 | (4R)-4-[(4R)-4-ethyl-2-imino-4- methyl-6-oxo- hexahydropyrimidin-1-yl]-N-(6- fluoro-3-hydroxy-chroman-4- yl)chromane-6-carboxamide |
| 152 | 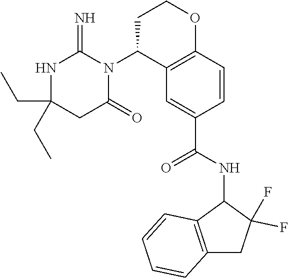 [SMILES: CCC1(CC)CC(=O)N([C@@H]2CCOc3ccc(C(=O)NC4c5ccccc5CC4(F)F)cc32)C(=N)N1] | 497.2 | (4R)-4-(4,4-diethyl-2-imino-6- oxo-hexahydropyrimidin-1-yl)- N-(2,2-difluoroindan-1- yl)chromane-6-carboxamide |
| 153 | 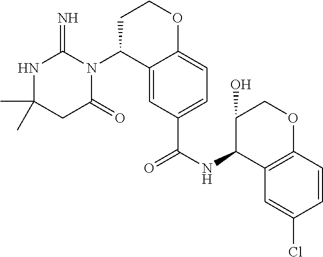 [SMILES: CC1(C)CC(=O)N([C@@H]2CCOc3ccc(C(=O)N[C@@H]4c5cc(Cl)ccc5OC[C@H]4O)cc32)C(=N)N1] | 499.1 | (4R)-N-[(3S,4R)-6-chloro-3- hydroxy-chroman-4-yl]-4-(2- imino-4,4-dimethyl-6-oxo- hexahydropyrimidin-1- yl)chromane-6-carboxamide |
| 154 | 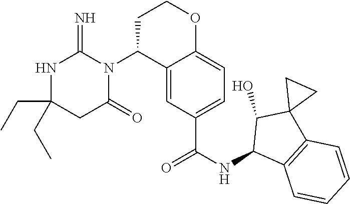 [SMILES: CCC1(CC)CC(=O)N([C@@H]2CCOc3ccc(C(=O)N[C@@H]4c5ccccc5C5(CC5)[C@H]4O)cc32)C(=N)N1] | 503.2 | (4R)-4-(4,4-diethyl-2-imino-6- oxo-hexahydropyrimidin-1-yl)- N-[(1′R,2′R)-2′- hydroxyspiro[cyclopropane- 1,3′-indane]-1′-yl]chromane-6- carboxamide |
| 155 | 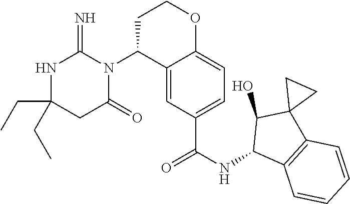 [SMILES: CCC1(CC)CC(=O)N([C@@H]2CCOc3ccc(C(=O)N[C@H]4c5ccccc5C5(CC5)[C@@H]4O)cc32)C(=N)N1] | 503.3 | (4R)-4-(4,4-diethyl-2-imino-6- oxo-hexahydropyrimidin-1-yl)- N-[(1′S,2′S)-2′- hydroxyspiro[cyclopropane- 1,3′-indane]-1′-yl]chromane-6- carboxamide |
| 156 | 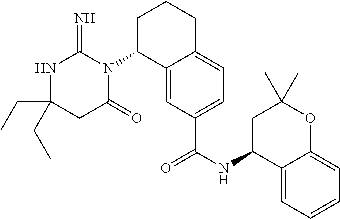 [SMILES: CCC1(CC)CC(=O)N([C@@H]2CCCc3ccc(C(=O)N[C@H]4CC(C)(C)Oc5ccccc54)cc32)C(=N)N1] | 503.3 | (4R)-4-(4,4-diethyl-2-imino-6- oxo-hexahydropyrimidin-1-yl)- N-[(4S)-2,2-dimethylchroman- 4-yl]tetralin-6-carboxamide |
| 157 | 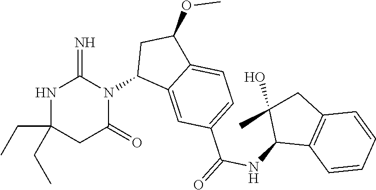 [SMILES: CCC1(CC)CC(=O)N([C@@H]2C[C@@H](OC)c3ccc(C(=O)N[C@@H]4c5ccccc5C[C@@]4(C)O)cc32)C(=N)N1] | 505.3 | (1R,3R)-3-(4,4-diethyl-2-imino- 6-oxo-hexahydropyrimidin-1- yl)-N-[(1R,2R)-2-hydroxy-2- methyl-indan-1-yl]-1-methoxy- indane-5-carboxamide |
| 158 | 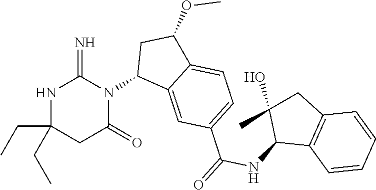 [SMILES: CCC1(CC)CC(=O)N([C@@H]2C[C@H](OC)c3ccc(C(=O)N[C@@H]4c5ccccc5C[C@@]4(C)O)cc32)C(=N)N1] | 505.3 | (1S,3R)-3-(4,4-diethyl-2-imino- 6-oxo-hexahydropyrimidin-1- yl)-N-[(1R,2R)-2-hydroxy-2- methyl-indan-1-yl]-1-methoxy- indane-5-carboxamide |
| 159 | 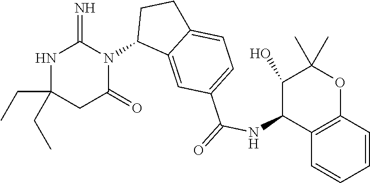 [SMILES: CCC1(CC)CC(=O)N([C@@H]2CCc3ccc(C(=O)N[C@@H]4c5ccccc5OC(C)(C)[C@H]4O)cc32)C(=N)N1] | 505.4 | (3R)-3-(4,4-diethyl-2-imino-6- oxo-hexahydropyrimidin-1-yl)- N-[(3S,4R)-3-hydroxy-2,2- dimethyl-chroman-4-yl]indane- 5-carboxamide |
| 160 | 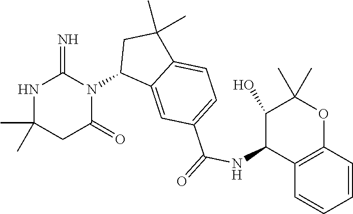 [SMILES: CC1(C)CC(=O)N([C@@H]2CC(C)(C)c3ccc(C(=O)N[C@@H]4c5ccccc5OC(C)(C)[C@H]4O)cc32)C(=N)N1] | 505.2 | (3R)-N-[(3S,4R)-3-hydroxy-2,2- dimethyl-chroman-4-yl]-3-(2- imino-4,4-dimethyl-6-oxo- hexahydropyrimidin-1-yl)-1,1- dimethyl-indane-5-carboxamide |

### Example 161

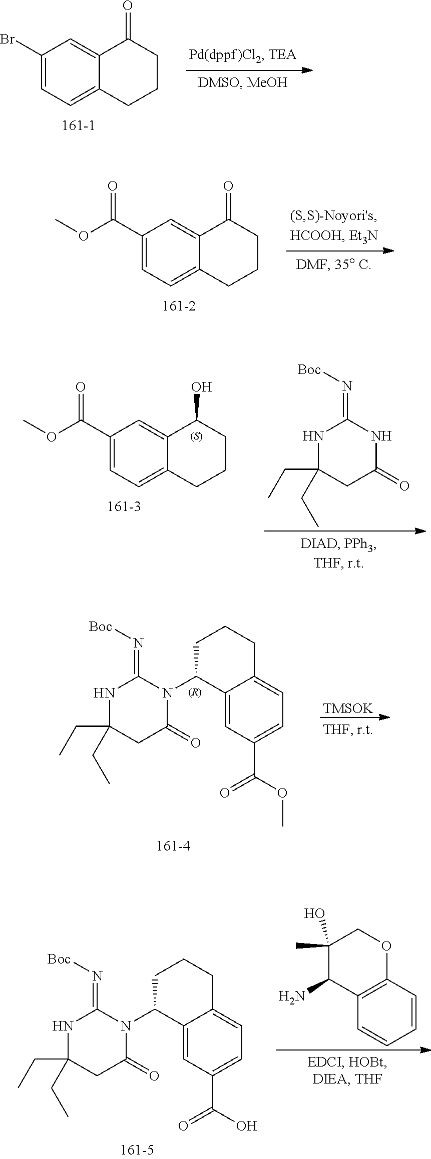

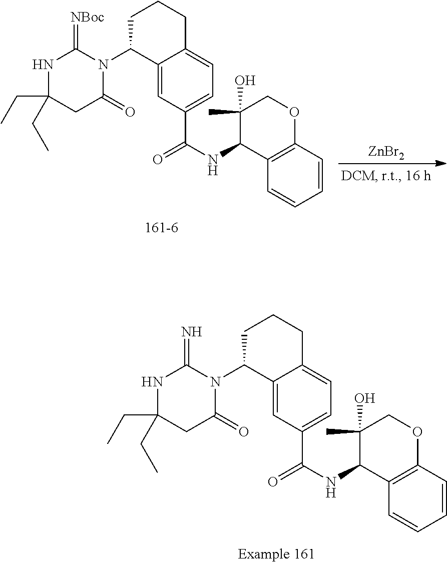

### Step A: methyl 8-oxo-5,6,7,8-tetrahydronaphthalene-2-carboxylate 161-2

PdCl₂(dppf) (11 g, 15.03 mmol) and triethylamine (108 mL, 777 mmol) was added to a solution of 7-bromo-1-tetralone 161-1 (35 g, 155 mmol) in MeOH (200 mL) and DMSO (100 mL). The mixture was stirred at 80° C. for 48 h under 50 psi CO atmosphere. After cooled, the mixture was concentrated in vacuo. The residue was diluted with water (200 mL) and extracted with EtOAc (200 mL*3). The combined organic layers were washed with water (400 mL) and brine (400 mL), dried over Na₂SO₄, filtered, and concentrated in vacuo. The crude was purified by column chromatography (SiO₂, petroleum ether:EtOAc=3:1) to afford methyl 8-oxo-5,6,7,8-tetrahydronaphthalene-2-carboxylate 161-2.

¹H NMR (400 MHz, chloroform-d) δ 8.63 (d, J=1.2 Hz, 1H), 8.08 (dd, J=2.0, 6.0 Hz, 1H), 7.31 (d, J=8.0 Hz, 1H), 3.90 (s, 3H), 2.99 (t, J=6.0 Hz, 2H), 2.66 (t, J=6.4 Hz, 2H), 2.10-2.17 (m, 2H)

### Step B: methyl (S)-8-hydroxy-5,6,7,8-tetrahydronaphthalene-2-carboxylate 161-3

A solution of formic acid (27.7 mL, 734 mmol) and TEA (205 mL, 1469 mmol) in DMF (400 mL) was stirred for 15 min, then methyl 8-oxo-5,6,7,8-tetrahydronaphthalene-2-carboxylate 161-2 (50 g, 245 mmol) and (S,S)—N-(p-toluenesulfonyl)-1,2-diphenylethanediamine(chloroxp-cymene)ruthenium(II) (6.23 g, 9.79 mmol) were added and the mixture was stirred at 35° C. for 16 h. The mixture was diluted with water (800 mL) and extracted with EtOAc (500 mL×3). The combined organic layers were washed with water (500 mL) and brine (500 mL), dried over anhydrous Na₂SO₄, filtered and concentrated. The residue which was purified by flash silica gel chromatography (ISCO®; Agela® Flash Column Silica-CS (330 g), Eluent of 0-30% Ethyl acetate/petroleum ether gradient @85 mL/min) to afford methyl (S)-8-hydroxy-5,6,7,8-tetrahydronaphthalene-2-carboxylate 161-3.

¹H NMR (400 MHz, chloroform-d) δ 8.13 (s, 1H), 7.84 (d, J=7.6 Hz, 1H), 7.16 (d, J=8.4 Hz, 1H), 4.82-4.83 (m, 1H), 3.90 (s, 3H), 2.85-2.92 (m, 1H), 2.73-2.80 (m, 1H), 2.00-2.05 (m, 2H), 1.89-1.91 (m, 1H), 1.76-1.84 (m, 1H)

### Step C: give methyl (R,E)-8-(2-((tert-butoxycarbonyl)imino)-4,4-diethyl-6-oxotetrahydropyrimidin-1(2H)-yl)-5,6,7,8-tetrahydronaphthalene-2-carboxylate 161-4

DIAD (36.8 mL, 189 mmol) was added dropwise to a solution of tert-butyl (E)-(4,4-diethyl-6-oxotetrahydropyrimidin-2(1H)-ylidene)carbamate (43.1 g, 160 mmol), methyl (S)-8-hydroxy-5,6,7,8-tetrahydronaphthalene-2-carboxylate 161-3 (30 g, 145 mmol) and triphenylphosphane (49.6 g, 189 mmol) in THF (500 mL) at 0° C. under N₂ atmosphere. The mixture was stirred at 27° C. for 2 h. The product was diluted with EtOAc (100 mL) and petroleum ether (500 mL) was slowly added while stirring. The mixture was filtered. The filtrate was concentrated in vacuo and purified by flash silica gel chromatography (ISCO®; Agela® Flash Column Silica-CS (330 g), Eluent of 10% Ethyl acetate/petroleum ether gradient @85 mL/min to give methyl (R,E)-8-(2-((tert-butoxycarbonyl)imino)-4,4-diethyl-6-oxotetrahydropyrimidin-1(2H)-yl)-5,6,7,8-tetrahydronaphthalene-2-carboxylate 161-4. MS (ESI) m/z: 458.3 (M+H⁺)

¹H NMR (400 MHz, chloroform-d) δ 10.11 (br s, 1H), 7.72 (d, J=8.0 Hz, 1H), 7.62 (s, 1H), 7.13 (d, J=8.4 Hz, 1H), 6.17-6.33 (m, 1H), 3.85 (s, 3H), 2.90-3.08 (m, 1H), 2.75-2.78 (m, 1H), 2.47-2.59 (m, 2H), 2.23-2.35 (m, 1H), 2.01-2.10 (m, 2H), 1.77-1.88 (m, 1H), 1.62-1.76 (m, 4H), 1.51 (br s, 9H), 0.92-1.01 (m, 6H).

### Step D: (E)-8-(2-((tert-butoxycarbonyl)imino)-4,4-diethyl-6-oxotetrahydropyrimidin-1(2H)-yl)-5,6,7,8-tetrahydronaphthalene-2-carboxylic acid 161-5

Potassium trimethylsilanolate (21.31 g, 166 mmol) was added to a solution of methyl (E)-8-(2-((tert-butoxycarbonyl)imino)-4,4-diethyl-6-oxotetrahydropyrimidin-1(2H)-yl)-5,6,7,8-tetrahydronaphthalene-2-carboxylate 161-4 (19 g, 41.5 mmol) in THF (450 mL). The reaction was stirred at 25° C. for 0.5 h. The solution of (E)-8-(2-((tert-butoxycarbonyl)imino)-4,4-diethyl-6-oxotetrahydropyrimidin-1(2H)-yl)-5,6,7,8-tetrahydronaphthalene-2-carboxylic acid 161-5 was used for next step directly without any further manipulation or purification.

MS (ESI) m/z 444.7 (M+H⁺)

### Step E: tert-butyl ((E)-4,4-diethyl-1-(7-(((3S,4R)-3-hydroxy-3-methylchroman-4-yl)carbamoyl)-1,2,3,4-tetrahydronaphthalen-1-yl)-6-oxotetrahydropyrimidin-2(1H)-ylidene)carbamate 161-6

DIEA (36.4 mL, 209 mmol) was added to a solution of (E)-8-(2-((tert-butoxycarbonyl)imino)-4,4-diethyl-6-oxotetrahydropyrimidin-1(2H)-yl)-5,6,7,8-tetrahydronaphthalene-2-carboxylic acid 161-5 (18.5 g, 41.7 mmol), EDCI (40.0 g, 209 mmol), HOBt (16.91 g, 125 mmol) and (3S,4R)-4-amino-3-methylchroman-3-ol (8.22 g, 45.9 mmol) in THF (450 mL). The reaction was stirred at 25° C. for 2.5 h. The mixture was quenched with water (400 mL) and extracted with EtOAc (500 mL*3). The combined organic layers were washed with brine (350 mL), dried over Na₂SO₄, filtered, and concentrated in vacuo. The crude was purified by flash silica gel chromatography (ISCO®; Agela® Flash Column Silica-CS (120 g), Eluent of 0˜30% Ethyl acetate/petroleum ether gradient @85 mL/min) to afford tert-butyl ((E)-4,4-diethyl-1-(7-(((3S,4R)-3-hydroxy-3-methylchroman-4-yl)carbamoyl)-1,2,3,4-tetrahydronaphthalen-1-yl)-6-oxotetrahydropyrimidin-2(1H)-ylidene)carbamate 161-6. MS (ESI) m/z 605.3 (M+H⁺)

¹H NMR (400 MHz, chloroform-d) δ 10.15 (br s, 1H), 7.56 (br s, 1H), 7.42 (d, J=8.0 Hz, 1H), 7.27-7.31 (m, 1H), 7.23 (d, J=7.6 Hz, 11H), 7.15 (d, J=8.0 Hz, 1H), 6.99 (t, J=7.6 Hz, 1H), 6.91 (d, J=8.0 Hz, 1H), 6.36 (d, J=7.2 Hz, 1H), 6.25-6.28 (m, 1H), 5.31 (d, J=7.2 Hz, 1H), 4.96 (s, 1H), 3.96-4.04 (m, 2H), 2.91-3.09 (m, 1H), 2.76-2.80 (m, 1H), 2.53 (br s, 2H), 2.28-2.32 (m, 1H), 2.00-2.11 (m, 2H), 1.73-1.88 (m, 1H), 1.62-1.67 (m, 4H), 1.50 (s, 9H), 1.24 (s, 3H), 0.88-0.97 (m, 6H).

### Step F: 8-(4,4-diethyl-2-imino-6-oxotetrahydropyrimidin-1(2H)-yl)-N-((3S,4R)-3-hydroxy-3-methylchroman-4-yl)-5,6,7,8-tetrahydronaphthalene-2-carboxamide Example 161

A solution of tert-butyl ((E)-4,4-diethyl-1-(7-(((3S,4R)-3-hydroxy-3-methylchroman-4-yl)carbamoyl)-1,2,3,4-tetrahydronaphthalen-1-yl)-6-oxotetrahydropyrimidin-2(1H)-ylidene)carbamate 161-6 (25 g, 41.3 mmol) and zinc(II) bromide (37.17 g, 165.2 mmol) in DCM (300 mL) at 25° C. under N₂ atmosphere was stirred at 25° C. for 16 h. The mixture was concentrated in vacuo at room temperature. MCCN (300 mL) was added and the mixture was stirred and then filtered. The filtrate was concentrated and purified by prep-HPLC (Instrument PREPL-X Method Column YMC-Triart Prep C18 250*50 mm*10 um Condition water (0.05% HCl)-ACN Begin B 10 End B 40 Gradient Time (min) 20 100% B Hold Time (min) 3 FlowRate (ml/min) 120 Injections 6) to afford 8-(4,4-diethyl-2-imino-6-oxotetrahydropyrimidin-1(2H)-yl)-N-((3S,4R)-3-hydroxy-3-methylchroman-4-yl)-5,6,7,8-tetrahydronaphthalene-2-carboxamide Example 161. MS (ESI) m/z 505.2 (M+H⁺)

¹H NMR (400 MHz, methanol-d₄) δ 7.63 (br s, 1H), 7.59 (s, 1H), 720-7.24 (m, 1H), 7.17 (d, J=8.0 Hz, 2H), 6.88-6.94 (m, 11H), 6.86 (d, J=8.00 Hz, 1H), 5.19-5.64 (m, 2H), 3.94-4.05 (m, 2H), 2.67-2.97 (m, 4H), 2.08-2.45 (m, 3H), 1.64-1.89 (m, 5H), 1.28 (s, 3H), 0.98 (t, J=7.2 Hz, 6H).

The compounds in Table 1-4 were prepared in an analogous fashion to that described in Scheme 1 and the experimentals described herein. The isomers were separated by preparative HPLC or/and preparative chiral SFC.

TABLE 3

| | | LC/MS | |
|---|---|---|---|
| Example | Structure | (M + 1)⁺ | Name |
| 162 | 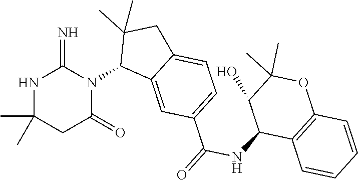 [SMILES: CC1(C)CC(=O)N([C@H]2c3cc(C(=O)N[C@@H]4c5ccccc5OC(C)(C)[C@H]4O)ccc3CC2(C)C)C(=N)N1] | 505.3 | (3R)-N-[(3S,4R)-3-hydroxy-2,2- dimethyl-chroman-4-yl]-3-(2- imino-4,4-dimethyl-6-oxo- hexahydropyrimidin-1-yl)-2,2- dimethyl-indane-5-carboxamide |
| 163 | 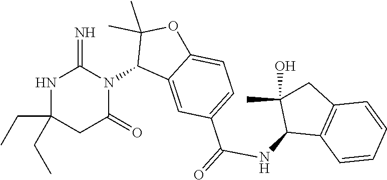 [SMILES: CCC1(CC)CC(=O)N([C@H]2c3cc(C(=O)N[C@@H]4c5ccccc5C[C@@]4(C)O)ccc3OC2(C)C)C(=N)N1] | 505.3 | (3S)-3-(4,4-diethyl-2-imino-6- oxo-hexahydropyrimidin-1-yl)-N- [(1R,2R)-2-hydroxy-2-methyl- indan-1-yl]-2,2-dimethyl-3H- benzofuran-5-carboxamide |
| 164 | 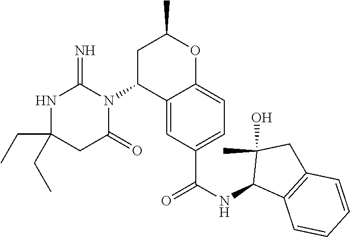 [SMILES: CCC1(CC)CC(=O)N([C@@H]2C[C@@H](C)Oc3ccc(C(=O)N[C@@H]4c5ccccc5C[C@@]4(C)O)cc32)C(=N)N1] | 505.3 | (2R,4R)-4-(4,4-diethyl-2-imino-6- oxo-hexahydropyrimidin-1-yl)-N- [(1R,2R)-2-hydroxy-2-methyl- indan-1-yl]-2-methyl-chromane-6- carboxamide |
| 165 | 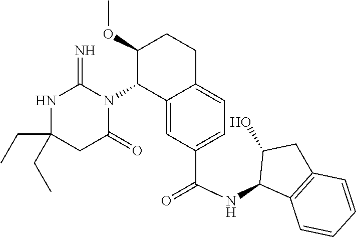 [SMILES: CCC1(CC)CC(=O)N([C@H]2c3cc(C(=O)N[C@@H]4c5ccccc5C[C@H]4O)ccc3CC[C@@H]2OC)C(=N)N1] | 505.1 | (3S,4S)-4-(4,4-diethyl-2-imino-6- oxo-hexahydropyrimidin-1-yl)-N- [(1R,2R)-2-hydroxyindan-1-yl]-3- methoxy-tetralin-6-carboxamide |
| 166 | 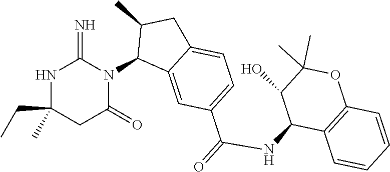 [SMILES: CC[C@]1(C)CC(=O)N([C@@H]2c3cc(C(=O)N[C@@H]4c5ccccc5OC(C)(C)[C@H]4O)ccc3C[C@@H]2C)C(=N)N1] | 505.3 | (2S,3S)-3-[(4R)-4-ethyl-2-imino- 4-methyl-6-oxo- hexahydropyrimidin-1-yl]-N- [(3S,4R)-3-hydroxy-2,2-dimethyl- chroman-4-yl]-2-methyl-indane-5- carboxamide |
| 167 | 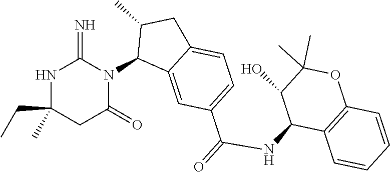 [SMILES: CC[C@]1(C)CC(=O)N([C@@H]2c3cc(C(=O)N[C@@H]4c5ccccc5OC(C)(C)[C@H]4O)ccc3C[C@H]2C)C(=N)N1] | 505.3 | (2R,3S)-3-[(4R)-4-ethyl-2-imino- 4-methyl-6-oxo- hexahydropyrimidin-1-yl]-N- [(3S,4R)-3-hydroxy-2,2-dimethyl- chroman-4-yl]-2-methyl-indane-5- carboxamide |
| 168 | 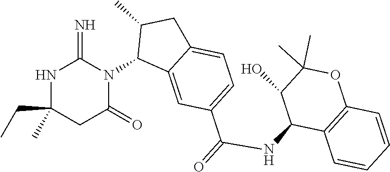 [SMILES: CC[C@]1(C)CC(=O)N([C@H]2c3cc(C(=O)N[C@@H]4c5ccccc5OC(C)(C)[C@H]4O)ccc3C[C@H]2C)C(=N)N1] | 505.3 | (2R,3R)-3-[(4R)-4-ethyl-2-imino- 4-methyl-6-oxo- hexahydropyrimidin-1-yl]-N- [(3S,4R)-3-hydroxy-2,2-dimethyl- chroman-4-yl]-2-methyl-indane-5- carboxamide |
| 169 | 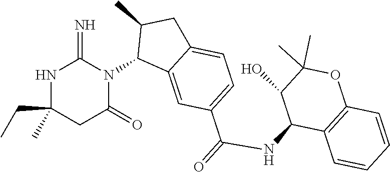 [SMILES: CC[C@]1(C)CC(=O)N([C@H]2c3cc(C(=O)N[C@@H]4c5ccccc5OC(C)(C)[C@H]4O)ccc3C[C@@H]2C)C(=N)N1] | 505.3 | (2S,3R)-3-[(4R)-4-ethyl-2-imino- 4-methyl-6-oxo- hexahydropyrimidin-1-yl]-N- [(3S,4R)-3-hydroxy-2,2-dimethyl- chroman-4-yl]-2-methyl-indane-5- carboxamide |
| 170 | 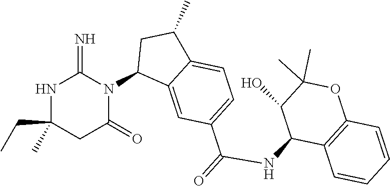 [SMILES: CC[C@]1(C)CC(=O)N([C@H]2C[C@H](C)c3ccc(C(=O)N[C@@H]4c5ccccc5OC(C)(C)[C@H]4O)cc32)C(=N)N1] | 505.3 | (1S,3S)-3-[(4R)-4-ethyl-2-imino- 4-methyl-6-oxo- hexahydropyrimidin-1-yl]-N- [(3S,4R)-3-hydroxy-2,2-dimethyl- chroman-4-yl]-1-methyl-indane-5- carboxamide |
| 171 | 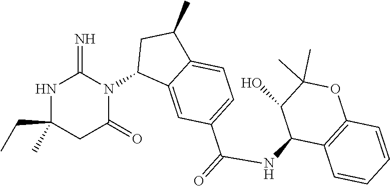 [SMILES: CC[C@]1(C)CC(=O)N([C@@H]2C[C@@H](C)c3ccc(C(=O)N[C@@H]4c5ccccc5OC(C)(C)[C@H]4O)cc32)C(=N)N1] | 505.3 | (1R,3R)-3-[(4R)-4-ethyl-2-imino- 4-methyl-6-oxo- hexahydropyrimidin-1-yl]-N- [(3S,4R)-3-hydroxy-2,2-dimethyl- chroman-4-yl]-1-methyl-indane-5- carboxamide |
| 172 | 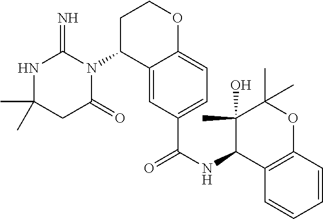 [SMILES: CC1(C)CC(=O)N([C@@H]2CCOc3ccc(C(=O)N[C@@H]4c5ccccc5OC(C)(C)[C@@]4(C)O)cc32)C(=N)N1] | 507.3 | (4R)-N-[(3S,4R)-3-hydroxy-2,2,3- trimethyl-chroman-4-yl]-4-(2- imino-4,4-dimethyl-6-oxo- hexahydropyrimidin-1- yl)chromane-6-carboxamide |
| 173 | 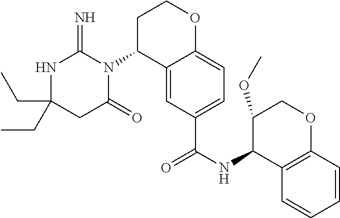 [SMILES: CCC1(CC)CC(=O)N([C@@H]2CCOc3ccc(C(=O)N[C@@H]4c5ccccc5OC[C@H]4OC)cc32)C(=N)N1] | 507.3 | (4R)-4-(4,4-diethyl-2-imino-6- oxo-hexahydropyrimidin-1-yl)-N- [(3S,4R)-3-methoxychroman-4- yl]chromane-6-carboxamide |
| 174 | 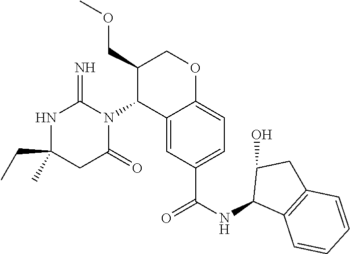 [SMILES: CC[C@]1(C)CC(=O)N([C@H]2c3cc(C(=O)N[C@@H]4c5ccccc5C[C@H]4O)ccc3OC[C@@H]2COC)C(=N)N1] | 507.1 | (3S,4R)-4-[(4R)-4-ethyl-2-imino- 4-methyl-6-oxo- hexahydropyrimidin-1-yl]-N- [(1R,2R)-2-hydroxyindan-1-yl]-3- (methoxymethyl)chromane-6- carboxamide |
| 175 | 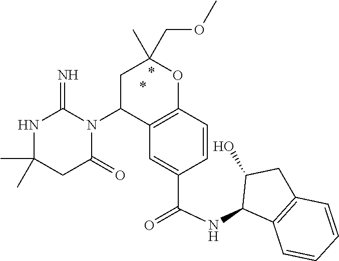 [SMILES: COCC1(C)CC(N2C(=N)NC(C)(C)CC2=O)c2cc(C(=O)N[C@@H]3c4ccccc4C[C@H]3O)ccc2O1] | 507.3 | N-[(1R,2R)-2-hydroxyindan-1-yl]- 4-(2-imino-4,4-dimethyl-6-oxo- hexahydropyrimidin-1-yl)-2- (methoxymethyl)-2-methyl- chromane-6-carboxamide |
| 176A | 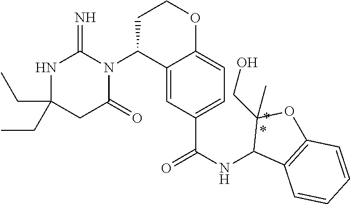 [SMILES: CCC1(CC)CC(=O)N([C@@H]2CCOc3ccc(C(=O)NC4c5ccccc5OC4(C)CO)cc32)C(=N)N1] | 507.2 | (4R)-4-(4,4-diethyl-2-imino-6- oxo-hexahydropyrimidin-1-yl)-N- [2-(hydroxymethyl)-2-methyl-3H- benzofuran-3-yl]chromane-6- carboxamide |
| 176B | 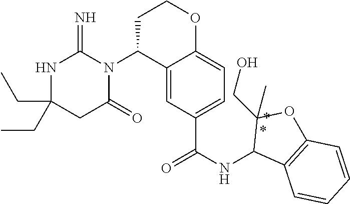 [SMILES: CCC1(CC)CC(=O)N([C@@H]2CCOc3ccc(C(=O)NC4c5ccccc5OC4(C)CO)cc32)C(=N)N1] | 507.3 | (4R)-4-(4,4-diethyl-2-imino-6- oxo-hexahydropyrimidin-1-yl)-N- [2-(hydroxymethyl)-2-methyl-3H- benzofuran-3-yl]chromane-6- carboxamide |
| 176C | 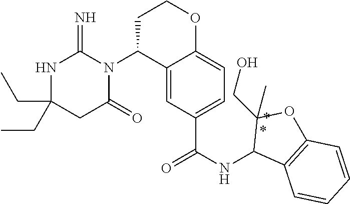 [SMILES: CCC1(CC)CC(=O)N([C@@H]2CCOc3ccc(C(=O)NC4c5ccccc5OC4(C)CO)cc32)C(=N)N1] | 507.3 | (4R)-4-(4,4-diethyl-2-imino-6- oxo-hexahydropyrimidin-1-yl)-N- [2-(hydroxymethyl)-2-methyl-3H- benzofuran-3-yl]chromane-6- carboxamide |
| 176D | 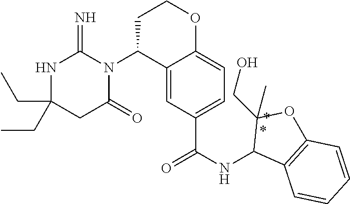 [SMILES: CCC1(CC)CC(=O)N([C@@H]2CCOc3ccc(C(=O)NC4c5ccccc5OC4(C)CO)cc32)C(=N)N1] | 507.3 | (4R)-4-(4,4-diethyl-2-imino-6- oxo-hexahydropyrimidin-1-yl)-N- [2-(hydroxymethyl)-2-methyl-3H- benzofuran-3-yl]chromane-6- carboxamide |
| 177 | 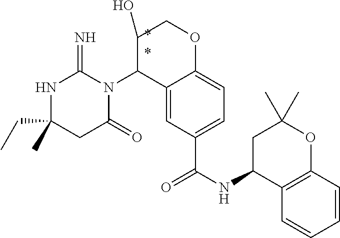 [SMILES: CC[C@@]1(C)CC(=O)N(C2c3cc(C(=O)N[C@H]4CC(C)(C)Oc5ccccc54)ccc3OCC2O)C(=N)N1] | 507.3 | N-[(4S)-2,2-dimethylchroman-4- yl]-4-[(4S)-4-ethyl-2-imino-4- methyl-6-oxo- hexahydropyrimidin-1-yl]-3- hydroxy-chromane-6-carboxamide |
| 178A | 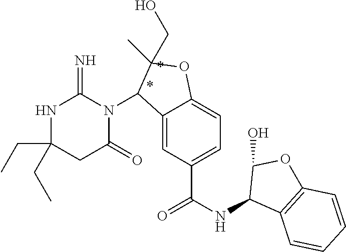 [SMILES: CCC1(CC)CC(=O)N(C2c3cc(C(=O)N[C@@H]4c5ccccc5O[C@H]4O)ccc3OC2(C)CO)C(=N)N1] | 507.3 | 3-(4,4-diethyl-2-imino-6-oxo- hexahydropyrimidin-1-yl)-N- [(1R,2R)-2-hydroxyindan-1-yl]-2- (hydroxymethyl)-2-methyl-3H- benzofuran-5-carboxamide |
| 178B | 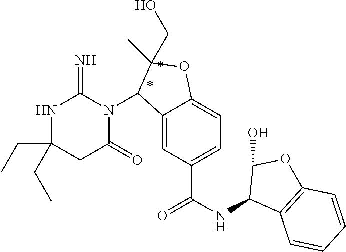 [SMILES: CCC1(CC)CC(=O)N(C2c3cc(C(=O)N[C@@H]4c5ccccc5O[C@H]4O)ccc3OC2(C)CO)C(=N)N1] | 507.3 | 3-(4,4-diethyl-2-imino-6-oxo- hexahydropyrimidin-1-yl)-N- [(1R,2R)-2-hydroxyindan-1-yl]-2- (hydroxymethyl)-2-methyl-3H- benzofuran-5-carboxamide |
| 179 | 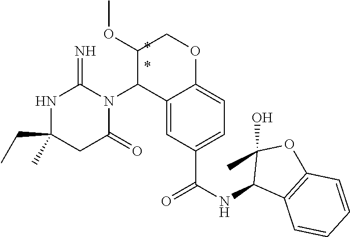 [SMILES: CC[C@]1(C)CC(=O)N(C2c3cc(C(=O)N[C@@H]4c5ccccc5O[C@@]4(C)O)ccc3OCC2OC)C(=N)N1] | 507.3 | 4-[(4R)-4-ethyl-2-imino-4-methyl- 6-oxo-hexahydropyrimidin-1-yl]- N-[(1R,2R)-2-hydroxy-2-methyl- indan-1-yl]-3-methoxy-chromane- 6-carboxamide |
| 180 | 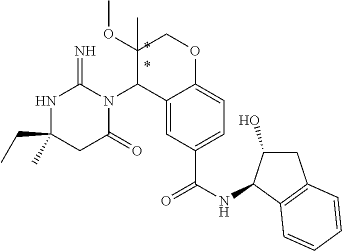 [SMILES: CC[C@]1(C)CC(=O)N(C2c3cc(C(=O)N[C@@H]4c5ccccc5C[C@H]4O)ccc3OCC2(C)OC)C(=N)N1] | 507.3 | 4-[(4R)-4-ethyl-2-imino-4-methyl- 6-oxo-hexahydropyrimidin-1-yl]- N-[(1R,2R)-2-hydroxyindan-1-yl]- 3-methoxy-3-methyl-chromane-6- carboxamide |
| 181A | 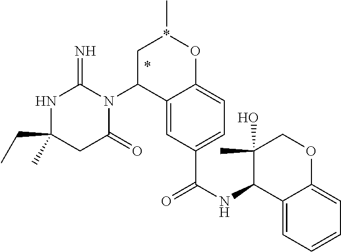 [SMILES: CC[C@]1(C)CC(=O)N(C2C*(C)Oc3ccc(C(=O)N[C@@H]4c5ccccc5OC[C@@]4(C)O)cc32)C(=N)N1] | 507.3 | 4-[(4R)-4-ethyl-2-imino-4-methyl- 6-oxo-hexahydropyrimidin-1-yl]- N-[(3S,4R)-3-hydroxy-3-methyl- chroman-4-yl]-2-methyl- chromane-6-carboxamide |
| 181B | 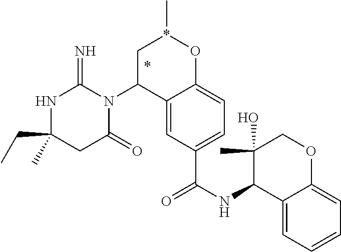 [SMILES: CC[C@]1(C)CC(=O)N(C2C*(C)Oc3ccc(C(=O)N[C@@H]4c5ccccc5OC[C@@]4(C)O)cc32)C(=N)N1] | 507.3 | 4-[(4R)-4-ethyl-2-imino-4-methyl- 6-oxo-hexahydropyrimidin-1-yl]- N-[(3S,4R)-3-hydroxy-3-methyl- chroman-4-yl]-2-methyl- chromane-6-carboxamide |
| 182 | 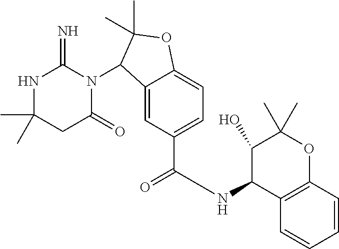 [SMILES: CC1(C)CC(=O)N(C2c3cc(C(=O)N[C@@H]4c5ccccc5OC(C)(C)[C@H]4O)ccc3OC2(C)C)C(=N)N1] | 507.3 | (3S)-N-[(3S,4R)-3-hydroxy-2,2- dimethyl-chroman-4-yl]-3-(2- imino-4,4-dimethyl-6-oxo- hexahydropyrimidin-1-yl)-2,2- dimethyl-3H-benzofuran-5- carboxamide |
| 183 | 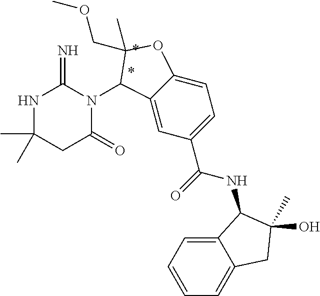 [SMILES: COCC1(C)Oc2ccc(C(=O)N[C@@H]3c4ccccc4C[C@]3(C)O)cc2C1N1C(=N)NC(C)(C)CC1=O] | 507.3 | N-[(1R,2S)-2-hydroxy-2-methyl- indan-1-yl]-3-(2-imino-4,4- dimethyl-6-oxo- hexahydropyrimidin-1-yl)-2- (methoxymethyl)-2-methyl-3H- benzofuran-5-carboxamide |
| 184 | 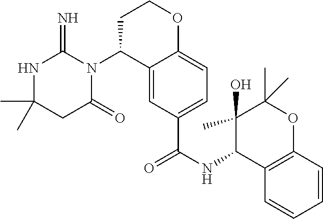 [SMILES: CC1(C)CC(=O)N([C@@H]2CCOc3ccc(C(=O)N[C@H]4c5ccccc5OC(C)(C)[C@]4(C)O)cc32)C(=N)N1] | 507.3 | (4R)-N-[(3R,4S)-3-hydroxy-2,2,3- trimethyl-chroman-4-yl]-4-(2- imino-4,4-dimethyl-6-oxo- hexahydropyrimidin-1- yl)chromane-6-carboxamide |
| 185 | 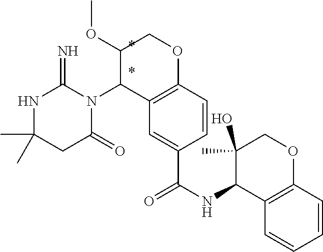 [SMILES: COC1COc2ccc(C(=O)N[C@@H]3c4ccccc4OC[C@]3(C)O)cc2C1N1C(=N)NC(C)(C)CC1=O] | 509.2 | N-[(3R,4R)-3-hydroxy-3-methyl- chroman-4-yl]-4-(2-imino-4,4- dimethyl-6-oxo- hexahydropyrimidin-1-yl)-3- methoxy-chromane-6- carboxamide |
| 186 | 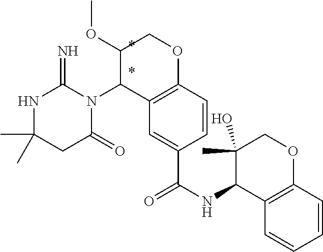 [SMILES: COC1COc2ccc(C(=O)N[C@@H]3c4ccccc4OC[C@@]3(C)O)cc2C1N1C(=N)NC(C)(C)CC1=O] | 509.2 | N-[(3S,4R)-3-hydroxy-3-methyl- chroman-4-yl]-4-(2-imino-4,4- dimethyl-6-oxo- hexahydropyrimidin-1-yl)-3- methoxy-chromane-6- carboxamide |
| 187 | 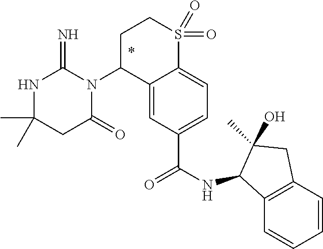 [SMILES: CC1(C)CC(=O)N(C2CCS(=O)(=O)c3ccc(C(=O)N[C@@H]4c5ccccc5C[C@]4(C)O)cc32)C(=N)N1] | 511.3 | N-[(1R,2S)-2-hydroxy-2-methyl- indan-1-yl]-4-(2-imino-4,4- dimethyl-6-oxo- hexahydropyrimidin-1-yl)-1,1- dioxo-3,4-dihydro-2H- thiochromene-6-carboxamide |
| 188 | 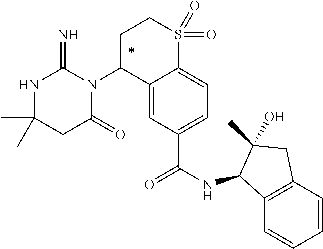 [SMILES: CC1(C)CC(=O)N(C2CCS(=O)(=O)c3ccc(C(=O)N[C@@H]4c5ccccc5C[C@@]4(C)O)cc32)C(=N)N1] | 511.2 | N-[(1R,2R)-2-hydroxy-2-methyl- indan-1-yl]-4-(2-imino-4,4- dimethyl-6-oxo- hexahydropyrimidin-1-yl)-1,1- dioxo-3,4-dihydro-2H- thiochromene-6-carboxamide |
| 189 | 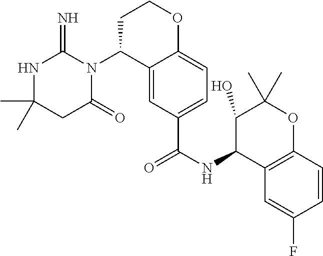 [SMILES: CC1(C)CC(=O)N([C@@H]2CCOc3ccc(C(=O)N[C@@H]4c5cc(F)ccc5OC(C)(C)[C@H]4O)cc32)C(=N)N1] | 511.3 | (4R)-N-[(3S,4R)-6-fluoro-3- hydroxy-2,2-dimethyl-chroman-4- yl]-4-(2-imino-4,4-dimethyl-6- oxo-hexahydropyrimidin-1- yl)chromane-6-carboxamide |
| 190A | 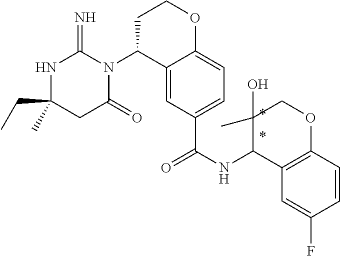 [SMILES: CC[C@]1(C)CC(=O)N([C@@H]2CCOc3ccc(C(=O)NC4c5cc(F)ccc5OCC4(C)O)cc32)C(=N)N1] | 511.2 | (4R)-4-[(4R)-4-ethyl-2-imino-4- methyl-6-oxo- hexahydropyrimidin-1-yl]-N-(6- fluoro-3-hydroxy-3-methyl- chroman-4-yl)chromane-6- carboxamide |
| 190B | 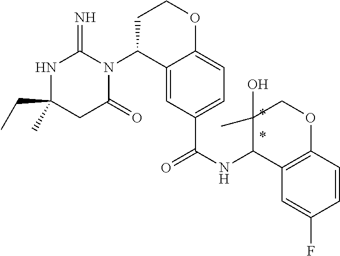 [SMILES: CC[C@]1(C)CC(=O)N([C@@H]2CCOc3ccc(C(=O)NC4c5cc(F)ccc5OCC4(C)O)cc32)C(=N)N1] | 511.2 | (4R)-4-[(4R)-4-ethyl-2-imino-4- methyl-6-oxo- hexahydropyrimidin-1-yl]-N-(6- fluoro-3-hydroxy-3-methyl- chroman-4-yl)chromane-6- carboxamide |
| 190C | 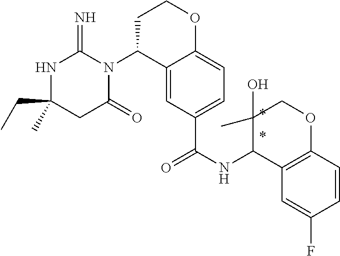 [SMILES: CC[C@]1(C)CC(=O)N([C@@H]2CCOc3ccc(C(=O)NC4c5cc(F)ccc5OCC4(C)O)cc32)C(=N)N1] | 511.2 | (4R)-4-[(4R)-4-ethyl-2-imino-4- methyl-6-oxo- hexahydropyrimidin-1-yl]-N-(6- fluoro-3-hydroxy-3-methyl- chroman-4-yl)chromane-6- carboxamide |
| 191 | 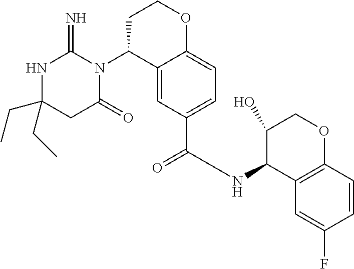 [SMILES: CCC1(CC)CC(=O)N([C@@H]2CCOc3ccc(C(=O)N[C@@H]4c5cc(F)ccc5OC[C@H]4O)cc32)C(=N)N1] | 511.2 | (4R)-4-(4,4-diethyl-2-imino-6- oxo-hexahydropyrimidin-1-yl)-N- [(3S,4R)-6-fluoro-3-hydroxy- chroman-4-yl]chromane-6- carboxamide |
| 192 | 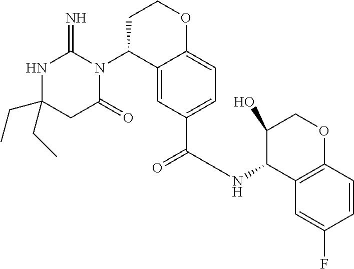 [SMILES: CCC1(CC)CC(=O)N([C@@H]2CCOc3ccc(C(=O)N[C@H]4c5cc(F)ccc5OC[C@@H]4O)cc32)C(=N)N1] | 511.3 | (4R)-4-(4,4-diethyl-2-imino-6- oxo-hexahydropyrimidin-1-yl)-N- [(3R,4S)-6-fluoro-3-hydroxy- chroman-4-yl]chromane-6- carboxamide |
| 193A | 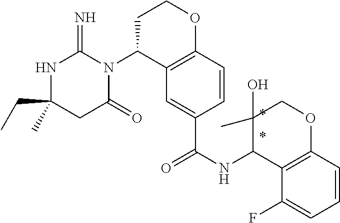 [SMILES: CC[C@]1(C)CC(=O)N([C@@H]2CCOc3ccc(C(=O)NC4c5c(F)cccc5OCC4(C)O)cc32)C(=N)N1] | 511.3 | (4R)-4-[(4R)-4-ethyl-2-imino-4- methyl-6-oxo- hexahydropyrimidin-1-yl]-N-(5- fluoro-3-hydroxy-3-methyl- chroman-4-yl)chromane-6- carboxamide |
| 193B | 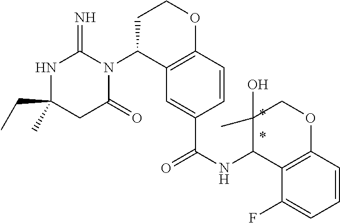 [SMILES: CC[C@]1(C)CC(=O)N([C@@H]2CCOc3ccc(C(=O)NC4c5c(F)cccc5OCC4(C)O)cc32)C(=N)N1] | 511.3 | (4R)-4-[(4R)-4-ethyl-2-imino-4- methyl-6-oxo- hexahydropyrimidin-1-yl]-N-(5- fluoro-3-hydroxy-3-methyl- chroman-4-yl)chromane-6- carboxamide |
| 193C | 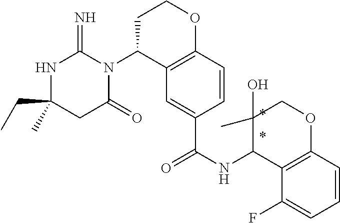 [SMILES: CC[C@]1(C)CC(=O)N([C@@H]2CCOc3ccc(C(=O)NC4c5c(F)cccc5OCC4(C)O)cc32)C(=N)N1] | 511.3 | (4R)-4-[(4R)-4-ethyl-2-imino-4- methyl-6-oxo- hexahydropyrimidin-1-yl]-N-(5- fluoro-3-hydroxy-3-methyl- chroman-4-yl)chromane-6- carboxamide |
| 194 | 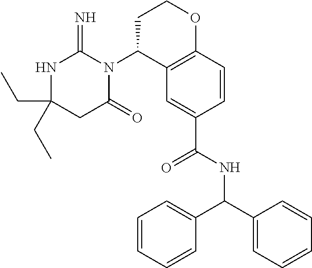 [SMILES: CCC1(CC)CC(=O)N([C@@H]2CCOc3ccc(C(=O)NC(c4ccccc4)c4ccccc4)cc32)C(=N)N1] | 511.3 | (4R)-N-benzhydryl-4-(4,4-diethyl- 2-imino-6-oxo- hexahydropyrimidin-1- yl)chromane-6-carboxamide |
| 195 | 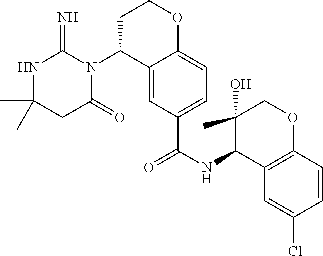 [SMILES: CC1(C)CC(=O)N([C@@H]2CCOc3ccc(C(=O)N[C@@H]4c5cc(Cl)ccc5OC[C@@]4(C)O)cc32)C(=N)N1] | 513.1 | (4R)-N-[(3S,4R)-6-chloro-3- hydroxy-3-methyl-chroman-4-yl]- 4-(2-imino-4,4-dimethyl-6-oxo- hexahydropyrimidin-1- yl)chromane-6-carboxamide |
| 196 | 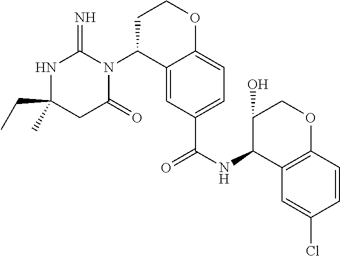 [SMILES: CC[C@]1(C)CC(=O)N([C@@H]2CCOc3ccc(C(=O)N[C@@H]4c5cc(Cl)ccc5OC[C@H]4O)cc32)C(=N)N1] | 513.2 | (4R)-N-[(3S,4R)-6-chloro-3- hydroxy-chroman-4-yl]-4-[(4R)-4- ethyl-2-imino-4-methyl-6-oxo- hexahydropyrimidin-1- yl]chromane-6-carboxamide |
| 197 | 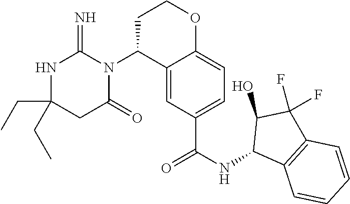 [SMILES: CCC1(CC)CC(=O)N([C@@H]2CCOc3ccc(C(=O)N[C@H]4c5ccccc5C(F)(F)[C@@H]4O)cc32)C(=N)N1] | 513.2 | (4R)-4-(4,4-diethyl-2-imino-6- oxo-hexahydropyrimidin-1-yl)-N- [(1S,2R)-3,3-difluoro-2-hydroxy- indan-1-yl]chromane-6- carboxamide |
| 198 | 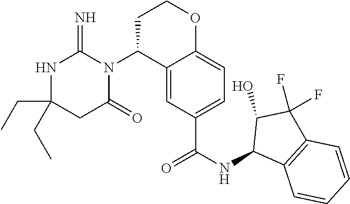 [SMILES: CCC1(CC)CC(=O)N([C@@H]2CCOc3ccc(C(=O)N[C@@H]4c5ccccc5C(F)(F)[C@H]4O)cc32)C(=N)N1] | 513.2 | (4R)-4-(4,4-diethyl-2-imino-6- oxo-hexahydropyrimidin-1-yl)-N- [(1R,2S)-3,3-difluoro-2-hydroxy- indan-1-yl]chromane-6- carboxamide |
| 199 | 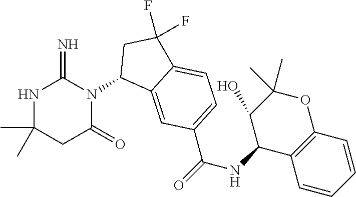 [SMILES: CC1(C)CC(=O)N([C@@H]2CC(F)(F)c3ccc(C(=O)N[C@@H]4c5ccccc5OC(C)(C)[C@H]4O)cc32)C(=N)N1] | 513.3 | (3R)-1,1-difluoro-N-[(3S,4R)-3- hydroxy-2,2-dimethyl-chroman-4- yl]-3-(2-imino-4,4-dimethyl-6- oxo-hexahydropyrimidin-1- yl)indane-5-carboxamide |
| 200 | 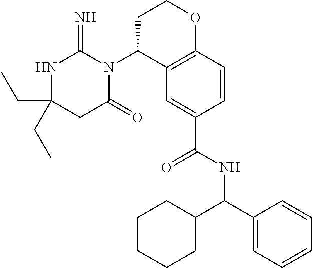 [SMILES: CCC1(CC)CC(=O)N([C@@H]2CCOc3ccc(C(=O)NC(c4ccccc4)C4CCCCC4)cc32)C(=N)N1] | 517.3 | (4R)-N- [cyclohexyl(phenyl)methyl]-4- (4,4-diethyl-2-imino-6-oxo- hexahydropyrimidin-1- yl)chromane-6-carboxamide |
| 201 | 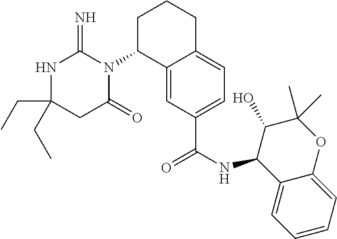 [SMILES: CCC1(CC)CC(=O)N([C@@H]2CCCc3ccc(C(=O)N[C@@H]4c5ccccc5OC(C)(C)[C@H]4O)cc32)C(=N)N1] | 519.3 | (4R)-4-(4,4-diethyl-2-imino-6- oxo-hexahydropyrimidin-1-yl)-N- [(3S,4R)-3-hydroxy-2,2-dimethyl- chroman-4-yl]tetralin-6- carboxamide |
| 202 | 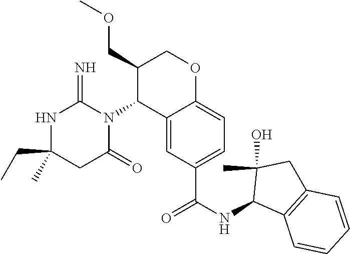 [SMILES: CC[C@]1(C)CC(=O)N([C@H]2c3cc(C(=O)N[C@@H]4c5ccccc5C[C@@]4(C)O)ccc3OC[C@@H]2COC)C(=N)N1] | 521.2 | (3S,4R)-4-[(4R)-4-ethyl-2-imino- 4-methyl-6-oxo- hexahydropyrimidin-1-yl]-N- [(1R,2R)-2-hydroxy-2-methyl- indan-1-yl]-3- (methoxymethyl)chromane-6- carboxamide |
| 203 | 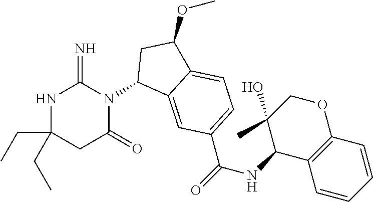 [SMILES: CCC1(CC)CC(=O)N([C@@H]2C[C@@H](OC)c3ccc(C(=O)N[C@@H]4c5ccccc5OC[C@@]4(C)O)cc32)C(=N)N1] | 521.3 | (1R,3R)-3-(4,4-diethyl-2-imino-6- oxo-hexahydropyrimidin-1-yl)-N- [(3S,4R)-3-hydroxy-3-methyl- chroman-4-yl]-1-methoxy-indane- 5-carboxamide |
| 204 | 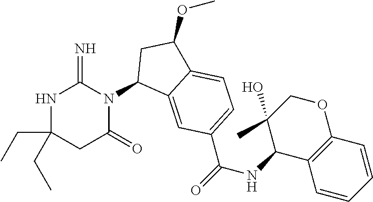 [SMILES: CCC1(CC)CC(=O)N([C@H]2C[C@@H](OC)c3ccc(C(=O)N[C@@H]4c5ccccc5OC[C@@]4(C)O)cc32)C(=N)N1] | 521.3 | (1R,3S)-3-(4,4-diethyl-2-imino-6- oxo-hexahydropyrimidin-1-yl)-N- [(3S,4R)-3-hydroxy-3-methyl- chroman-4-yl]-1-methoxy-indane- 5-carboxamide |
| 205 | 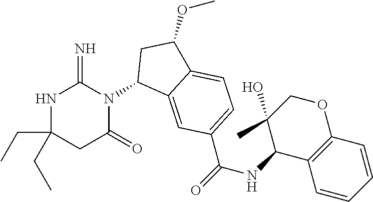 [SMILES: CCC1(CC)CC(=O)N([C@@H]2C[C@H](OC)c3ccc(C(=O)N[C@@H]4c5ccccc5OC[C@@]4(C)O)cc32)C(=N)N1] | 521.3 | (1S,3R)-3-(4,4-diethyl-2-imino-6- oxo-hexahydropyrimidin-1-yl)-N- [(3S,4R)-3-hydroxy-3-methyl- chroman-4-yl]-1-methoxy-indane- 5-carboxamide |
| 206 | 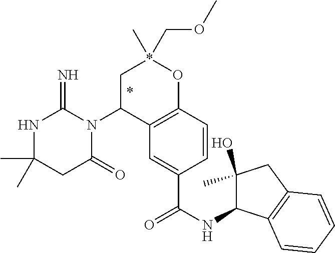 [SMILES: COC*1(C)CC(N2C(=N)NC(C)(C)CC2=O)c2cc(C(=O)N[C@@H]3c4ccccc4C[C@]3(C)O)ccc2O1] | 521.3 | N-[(1R,2S)-2-hydroxy-2-methyl- indan-1-yl]-4-(2-imino-4,4- dimethyl-6-oxo- hexahydropyrimidin-1-yl)-2- (methoxymethyl)-2-methyl- chromane-6-carboxamide |
| 207 | 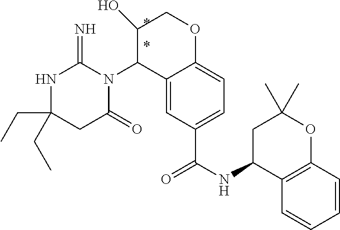 [SMILES: CCC1(CC)CC(=O)N(C2c3cc(C(=O)N[C@H]4CC(C)(C)Oc5ccccc54)ccc3OCC2O)C(=N)N1] | 521.2 | 4-(4,4-diethyl-2-imino-6-oxo- hexahydropyrimidin-1-yl)-N- [(4S)-2,2-dimethylchroman-4-yl]- 3-hydroxy-chromane-6- carboxamide |
| 208 | 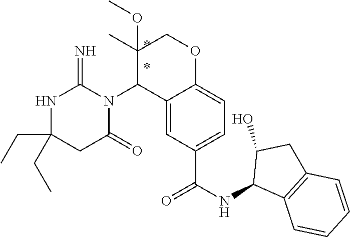 [SMILES: CCC1(CC)CC(=O)N(C2c3cc(C(=O)N[C@@H]4c5ccccc5C[C@H]4O)ccc3OCC2(C)OC)C(=N)N1] | 521.3 | 4-(4,4-diethyl-2-imino-6-oxo- hexahydropyrimidin-1-yl)-N- [(1R,2R)-2-hydroxyindan-1-yl]-3- methoxy-3-methyl-chromane-6- carboxamide |
| 209 | 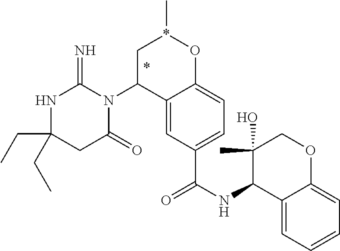 [SMILES: CCC1(CC)CC(=O)N(C2C*(C)Oc3ccc(C(=O)N[C@@H]4c5ccccc5OC[C@@]4(C)O)cc32)C(=N)N1] | 521.3 | 4-(4,4-diethyl-2-imino-6-oxo- hexahydropyrimidin-1-yl)-N- [(3S,4R)-3-hydroxy-3-methyl- chroman-4-yl]-2-methyl- chromane-6-carboxamide |
| 210 | 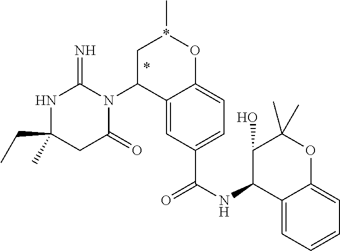 [SMILES: CC[C@]1(C)CC(=O)N(C2C*(C)Oc3ccc(C(=O)N[C@@H]4c5ccccc5OC(C)(C)[C@H]4O)cc32)C(=N)N1] | 521.3 | 4-[(4R)-4-ethyl-2-imino-4-methyl- 6-oxo-hexahydropyrimidin-1-yl]- N-[(3S,4R)-3-hydroxy-2,2- dimethyl-chroman-4-yl]-2-methyl- chromane-6-carboxamide |
| 211 | 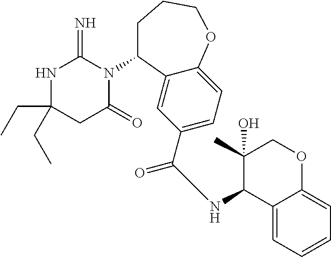 [SMILES: CCC1(CC)CC(=O)N([C@@H]2CCCOc3ccc(C(=O)N[C@@H]4c5ccccc5OC[C@@]4(C)O)cc32)C(=N)N1] | 521.2 | (5R)-5-(4,4-diethyl-2-imino-6- oxo-hexahydropyrimidin-1-yl)-N- [(3S,4R)-3-hydroxy-3-methyl- chroman-4-yl]-2,3,4,5-tetrahydro- 1-benzoxepine-7-carboxamide |
| 212 | 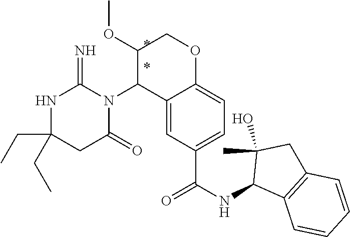 [SMILES: CCC1(CC)CC(=O)N(C2c3cc(C(=O)N[C@@H]4c5ccccc5C[C@@]4(C)O)ccc3OCC2OC)C(=N)N1] | 521.3 | 4-(4,4-diethyl-2-imino-6-oxo- hexahydropyrimidin-1-yl)-N- [(1R,2R)-2-hydroxy-2-methyl- indan-1-yl]-3-methoxy-chromane- 6-carboxamide |
| 213 | 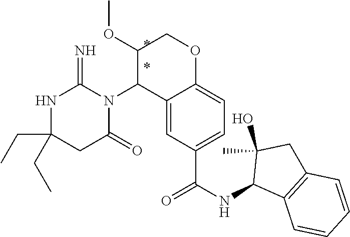 [SMILES: CCC1(CC)CC(=O)N(C2c3cc(C(=O)N[C@@H]4c5ccccc5C[C@]4(C)O)ccc3OCC2OC)C(=N)N1] | 521.3 | 4-(4,4-diethyl-2-imino-6-oxo- hexahydropyrimidin-1-yl)-N- [(1R,2S)-2-hydroxy-2-methyl- indan-1-yl]-3-methoxy-chromane- 6-carboxamide |
| 214A | 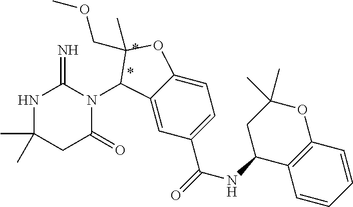 [SMILES: COCC1(C)Oc2ccc(C(=O)N[C@H]3CC(C)(C)Oc4ccccc43)cc2C1N1C(=N)NC(C)(C)CC1=O] | 521.3 | N-[(4S)-2,2-dimethylchroman-4- yl]-3-(2-imino-4,4-dimethyl-6- oxo-hexahydropyrimidin-1-yl)-2- (methoxymethyl)-2-methyl-3H- benzofuran-5-carboxamide |
| 214B | 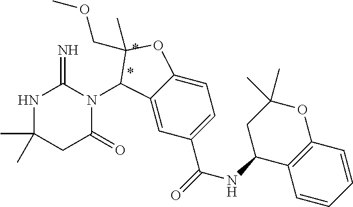 [SMILES: COCC1(C)Oc2ccc(C(=O)N[C@H]3CC(C)(C)Oc4ccccc43)cc2C1N1C(=N)NC(C)(C)CC1=O] | 521.3 | N-[(4S)-2,2-dimethylchroman-4- yl]-3-(2-imino-4,4-dimethyl-6- oxo-hexahydropyrimidin-1-yl)-2- (methoxymethyl)-2-methyl-3H- benzofuran-5-carboxamide |
| 215 | 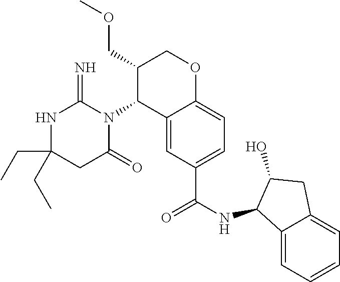 [SMILES: CCC1(CC)CC(=O)N([C@H]2c3cc(C(=O)N[C@@H]4c5ccccc5C[C@H]4O)ccc3OC[C@H]2COC)C(=N)N1] | 521.3 | (3R,4R)-4-(4,4-diethyl-2-imino-6- oxo-hexahydropyrimidin-1-yl)-N- [(1R,2R)-2-hydroxyindan-1-yl]-3- (methoxymethyl)chromane-6- carboxamide |
| 216 | 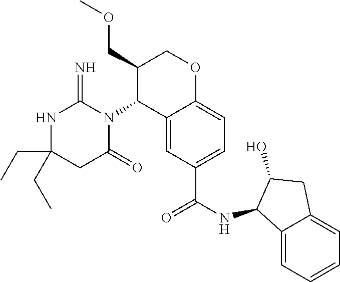 [SMILES: CCC1(CC)CC(=O)N([C@H]2c3cc(C(=O)N[C@@H]4c5ccccc5C[C@H]4O)ccc3OC[C@@H]2COC)C(=N)N1] | 521.3 | (3S,4R)-4-(4,4-diethyl-2-imino-6- oxo-hexahydropyrimidin-1-yl)-N- [(1R,2R)-2-hydroxyindan-1-yl]-3- (methoxymethyl)chromane-6- carboxamide |
| 217 | 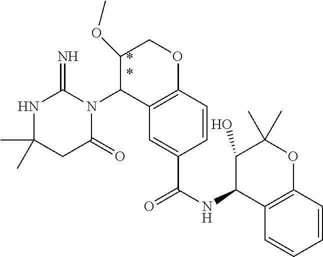 [SMILES: COC1COc2ccc(C(=O)N[C@@H]3c4ccccc4OC(C)(C)[C@H]3O)cc2C1N1C(=N)NC(C)(C)CC1=O] | 523.3 | N-[(3S,4R)-3-hydroxy-2,2- dimethyl-chroman-4-yl]-4-(2- imino-4,4-dimethyl-6-oxo- hexahydropyrimidin-1-yl)-3- methoxy-chromane-6- carboxamide |
| 218 | 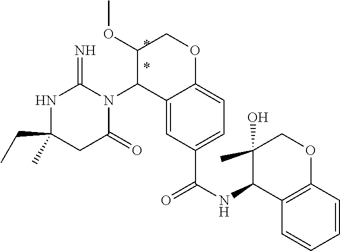 [SMILES: CC[C@]1(C)CC(=O)N(C2c3cc(C(=O)N[C@@H]4c5ccccc5OC[C@@]4(C)O)ccc3OCC2OC)C(=N)N1] | 523.2 | 4-[(4R)-4-ethyl-2-imino-4-methyl- 6-oxo-hexahydropyrimidin-1-yl]- N-[(3S,4R)-3-hydroxy-3-methyl- chroman-4-yl]-3-methoxy- chromane-6-carboxamide |
| 219 | 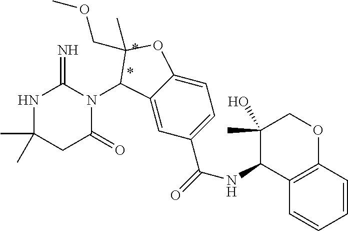 [SMILES: COCC1(C)Oc2ccc(C(=O)N[C@@H]3c4ccccc4OC[C@@]3(C)O)cc2C1N1C(=N)NC(C)(C)CC1=O] | 523.3 | N-[(3S,4R)-3-hydroxy-3-methyl- chroman-4-yl]-3-(2-imino-4,4- dimethyl-6-oxo- hexahydropyrimidin-1-yl)-2- (methoxymethyl)-2-methyl-3H- benzofuran-5-carboxamide |
| 220 | 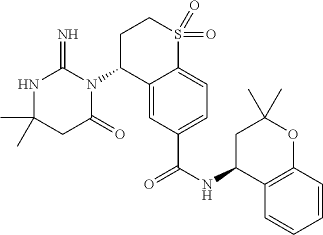 [SMILES: CC1(C)CC(=O)N([C@@H]2CCS(=O)(=O)c3ccc(C(=O)N[C@H]4CC(C)(C)Oc5ccccc54)cc32)C(=N)N1] | 525.2 | (4R)-N-[(4S)-2,2- dimethylchroman-4-yl]-4-(2- imino-4,4-dimethyl-6-oxo- hexahydropyrimidin-1-yl)-1,1- dioxo-3,4-dihydro-2H- thiochromene-6-carboxamide |
| 221 | 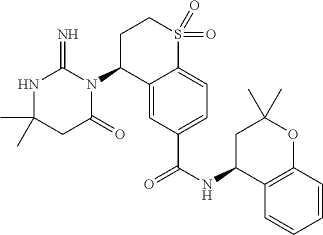 [SMILES: CC1(C)CC(=O)N([C@H]2CCS(=O)(=O)c3ccc(C(=O)N[C@H]4CC(C)(C)Oc5ccccc54)cc32)C(=N)N1] | 525.3 | (4S)-N-[(4S)-2,2- dimethylchroman-4-yl]-4-(2- imino-4,4-dimethyl-6-oxo- hexahydropyrimidin-1-yl)-1,1- dioxo-3,4-dihydro-2H- thiochromene-6-carboxamide |
| 222 | 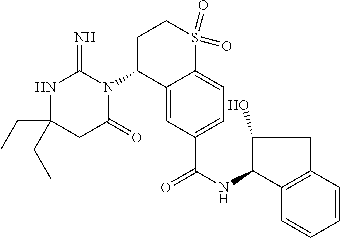 [SMILES: CCC1(CC)CC(=O)N([C@@H]2CCS(=O)(=O)c3ccc(C(=O)N[C@@H]4c5ccccc5C[C@H]4O)cc32)C(=N)N1] | 525.2 | (4R)-4-(4,4-diethyl-2-imino-6- oxo-hexahydropyrimidin-1-yl)-N- [(1R,2R)-2-hydroxyindan-1-yl]- 1,1-dioxo-3,4-dihydro-2H- thiochromene-6-carboxamide |
| 223 | 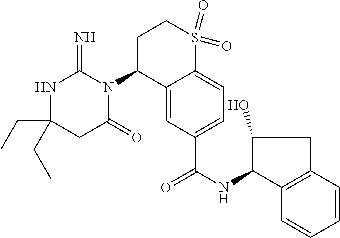 [SMILES: CCC1(CC)CC(=O)N([C@H]2CCS(=O)(=O)c3ccc(C(=O)N[C@@H]4c5ccccc5C[C@H]4O)cc32)C(=N)N1] | 525.3 | (4S)-4-(4,4-diethyl-2-imino-6- oxo-hexahydropyrimidin-1-yl)-N- [(1R,2R)-2-hydroxyindan-1-yl]- 1,1-dioxo-3,4-dihydro-2H- thiochromene-6-carboxamide |
| 224 | 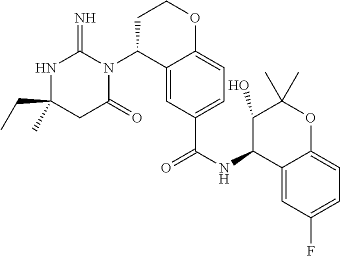 [SMILES: CC[C@]1(C)CC(=O)N([C@@H]2CCOc3ccc(C(=O)N[C@@H]4c5cc(F)ccc5OC(C)(C)[C@H]4O)cc32)C(=N)N1] | 525.3 | (4R)-4-[(4R)-4-ethyl-2-imino-4- methyl-6-oxo- hexahydropyrimidin-1-yl]-N- [(3S,4R)-6-fluoro-3-hydroxy-2,2- dimethyl-chroman-4-yl]chromane- 6-carboxamide |
| 225 | 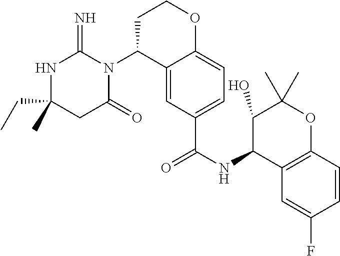 [SMILES: CC[C@@]1(C)CC(=O)N([C@@H]2CCOc3ccc(C(=O)N[C@@H]4c5cc(F)ccc5OC(C)(C)[C@H]4O)cc32)C(=N)N1] | 525.2 | (4R)-4-[(4S)-4-ethyl-2-imino-4- methyl-6-oxo- hexahydropyrimidin-1-yl]-N- [(3S,4R)-6-fluoro-3-hydroxy-2,2- dimethyl-chroman-4-yl]chromane- 6-carboxamide |
| 226A | 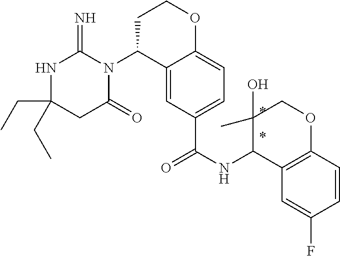 [SMILES: CCC1(CC)CC(=O)N([C@@H]2CCOc3ccc(C(=O)NC4c5cc(F)ccc5OCC4(C)O)cc32)C(=N)N1] | 525.3 | (4R)-4-(4,4-diethyl-2-imino-6- oxo-hexahydropyrimidin-1-yl)-N- (6-fluoro-3-hydroxy-3-methyl- chroman-4-yl)chromane-6- carboxamide |
| 226B | 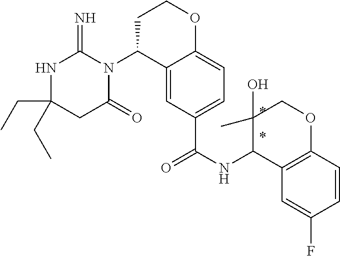 [SMILES: CCC1(CC)CC(=O)N([C@@H]2CCOc3ccc(C(=O)NC4c5cc(F)ccc5OCC4(C)O)cc32)C(=N)N1] | 525.3 | (4R)-4-(4,4-diethyl-2-imino-6- oxo-hexahydropyrimidin-1-yl)-N- (6-fluoro-3-hydroxy-3-methyl- chroman-4-yl)chromane-6- carboxamide |
| 226C | 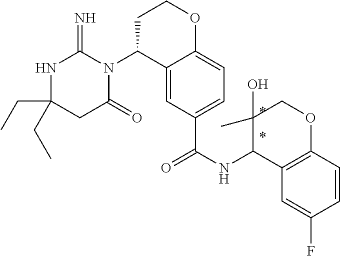 [SMILES: CCC1(CC)CC(=O)N([C@@H]2CCOc3ccc(C(=O)NC4c5cc(F)ccc5OCC4(C)O)cc32)C(=N)N1] | 525.3 | (4R)-4-(4,4-diethyl-2-imino-6- oxo-hexahydropyrimidin-1-yl)-N- (6-fluoro-3-hydroxy-3-methyl- chroman-4-yl)chromane-6- carboxamide |
| 226D | 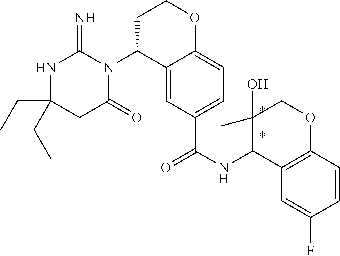 [SMILES: CCC1(CC)CC(=O)N([C@@H]2CCOc3ccc(C(=O)NC4c5cc(F)ccc5OCC4(C)O)cc32)C(=N)N1] | 525.1 | (4R)-4-(4,4-diethyl-2-imino-6- oxo-hexahydropyrimidin-1-yl)-N- (6-fluoro-3-hydroxy-3-methyl- chroman-4-yl)chromane-6- carboxamide |
| 227 | 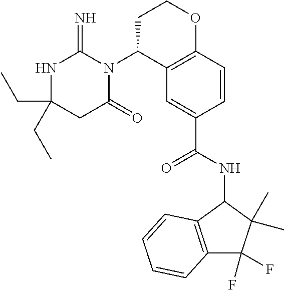 [SMILES: CCC1(CC)CC(=O)N([C@@H]2CCOc3ccc(C(=O)NC4c5ccccc5C(F)(F)C4(C)C)cc32)C(=N)N1] | 525.3 | (4R)-4-(4,4-diethyl-2-imino-6- oxo-hexahydropyrimidin-1-yl)-N- (3,3-difluoro-2,2-dimethyl-indan- 1-yl)chromane-6-carboxamide |
| 228 | 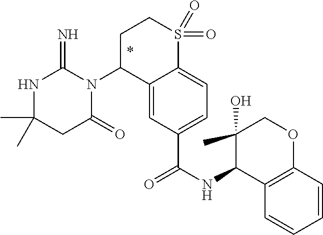 [SMILES: CC1(C)CC(=O)N(C2CCS(=O)(=O)c3ccc(C(=O)N[C@@H]4c5ccccc5OC[C@@]4(C)O)cc32)C(=N)N1] | 527.2 | N-[(3S,4R)-3-hydroxy-3-methyl- chroman-4-yl]-4-(2-imino-4,4- dimethyl-6-oxo- hexahydropyrimidin-1-yl)-1,1- dioxo-3,4-dihydro-2H- thiochromene-6-carboxamide |
| 229 | 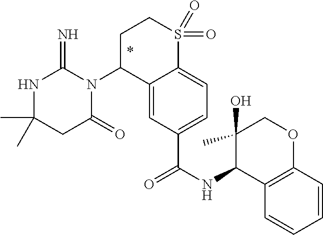 [SMILES: CC1(C)CC(=O)N(C2CCS(=O)(=O)c3ccc(C(=O)N[C@@H]4c5ccccc5OC[C@]4(C)O)cc32)C(=N)N1] | 527.2 | N-[(3R,4R)-3-hydroxy-3-methyl- chroman-4-yl]-4-(2-imino-4,4- dimethyl-6-oxo- hexahydropyrimidin-1-yl)-1,1- dioxo-3,4-dihydro-2H- thiochromene-6-carboxamide |
| 230 | 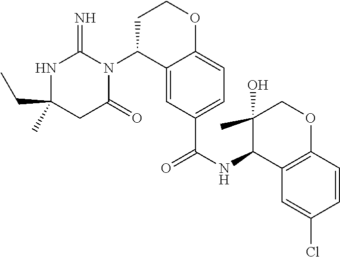 [SMILES: CC[C@]1(C)CC(=O)N([C@@H]2CCOc3ccc(C(=O)N[C@@H]4c5cc(Cl)ccc5OC[C@@]4(C)O)cc32)C(=N)N1] | 527.1 | (4R)-N-[(3S,4R)-6-chloro-3- hydroxy-3-methyl-chroman-4-yl]- 4-[(4R)-4-ethyl-2-imino-4-methyl- 6-oxo-hexahydropyrimidin-1- yl]chromane-6-carboxamide |
| 231 | 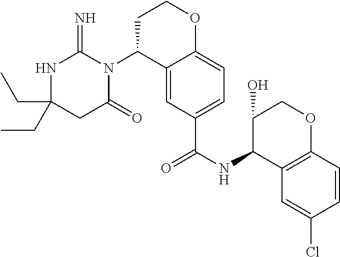 [SMILES: CCC1(CC)CC(=O)N([C@@H]2CCOc3ccc(C(=O)N[C@@H]4c5cc(Cl)ccc5OC[C@H]4O)cc32)C(=N)N1] | 527.3 | (4R)-N-[(3S,4R)-6-chloro-3- hydroxy-chroman-4-yl]-4-(4,4- diethyl-2-imino-6-oxo- hexahydropyrimidin-1- yl)chromane-6-carboxamide |
| 232 | 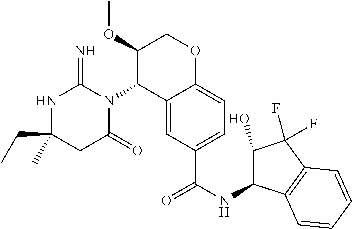 [SMILES: CC[C@]1(C)CC(=O)N([C@H]2c3cc(C(=O)N[C@@H]4c5ccccc5C(F)(F)[C@H]4O)ccc3OC[C@@H]2OC)C(=N)N1] | 529.3 | (3R,4S)-N-[(1R,2S)-3,3-difluoro- 2-hydroxy-indan-1-yl]-4-[(4R)-4- ethyl-2-imino-4-methyl-6-oxo- hexahydropyrimidin-1-yl]-3- methoxy-chromane-6- carboxamide |
| 233 | 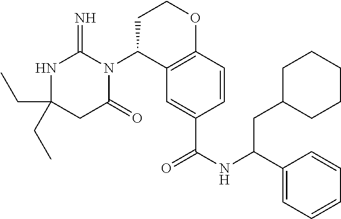 [SMILES: CCC1(CC)CC(=O)N([C@@H]2CCOc3ccc(C(=O)NC(CC4CCCCC4)c4ccccc4)cc32)C(=N)N1] | 531.3 | (4R)-N-(2-cyclohexyl-1-phenyl- ethyl)-4-(4,4-diethyl-2-imino-6- oxo-hexahydropyrimidin-1- yl)chromane-6-carboxamide |
| 234A | 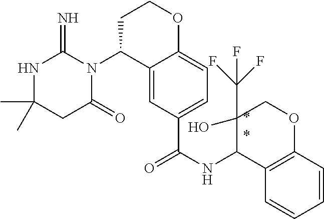 [SMILES: CC1(C)CC(=O)N([C@@H]2CCOc3ccc(C(=O)NC4c5ccccc5OCC4(O)C(F)(F)F)cc32)C(=N)N1] | 533.3 | (4R)-N-[3-hydroxy-3- (trifluoromethyl)chroman-4-yl]-4- (2-imino-4,4-dimethyl-6-oxo- hexahydropyrimidin-1- yl)chromane-6-carboxamide |
| 234B | 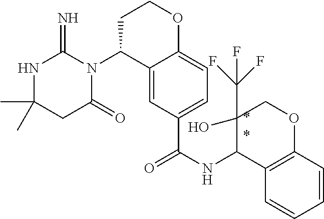 [SMILES: CC1(C)CC(=O)N([C@@H]2CCOc3ccc(C(=O)NC4c5ccccc5OCC4(O)C(F)(F)F)cc32)C(=N)N1] | 533.2 | (4R)-N-[3-hydroxy-3- (trifluoromethyl)chroman-4-yl]-4- (2-imino-4,4-dimethyl-6-oxo- hexahydropyrimidin-1- yl)chromane-6-carboxamide |
| 234C | 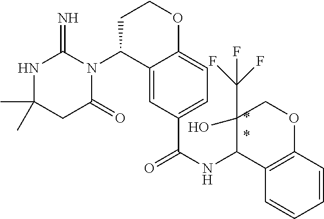 [SMILES: CC1(C)CC(=O)N([C@@H]2CCOc3ccc(C(=O)NC4c5ccccc5OCC4(O)C(F)(F)F)cc32)C(=N)N1] | 533.2 | (4R)-N-[3-hydroxy-3- (trifluoromethyl)chroman-4-yl]-4- (2-imino-4,4-dimethyl-6-oxo- hexahydropyrimidin-1- yl)chromane-6-carboxamide |
| 235 | 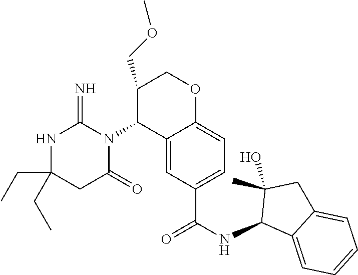 [SMILES: CCC1(CC)CC(=O)N([C@H]2c3cc(C(=O)N[C@@H]4c5ccccc5C[C@@]4(C)O)ccc3OC[C@H]2COC)C(=N)N1] | 535.1 | (3R,4R)-4-(4,4-diethyl-2-imino-6- oxo-hexahydropyrimidin-1-yl)-N- [(1R,2R)-2-hydroxy-2-methyl- indan-1-yl]-3- (methoxymethyl)chromane-6- carboxamide |
| 236 | 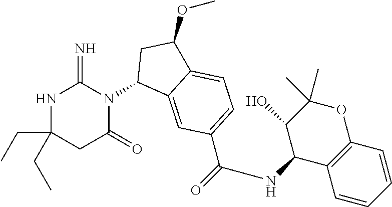 [SMILES: CCC1(CC)CC(=O)N([C@@H]2C[C@@H](OC)c3ccc(C(=O)N[C@@H]4c5ccccc5OC(C)(C)[C@H]4O)cc32)C(=N)N1] | 535.3 | (1R,3R)-3-(4,4-diethyl-2-imino-6- oxo-hexahydropyrimidin-1-yl)-N- [(3S,4R)-3-hydroxy-2,2-dimethyl- chroman-4-yl]-1-methoxy-indane- 5-carboxamide |
| 237 | 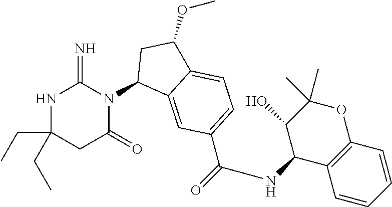 [SMILES: CCC1(CC)CC(=O)N([C@H]2C[C@H](OC)c3ccc(C(=O)N[C@@H]4c5ccccc5OC(C)(C)[C@H]4O)cc32)C(=N)N1] | 535.3 | (1S,3S)-3-(4,4-diethyl-2-imino-6- oxo-hexahydropyrimidin-1-yl)-N- [(3S,4R)-3-hydroxy-2,2-dimethyl- chroman-4-yl]-1-methoxy-indane- 5-carboxamide |
| 238 | 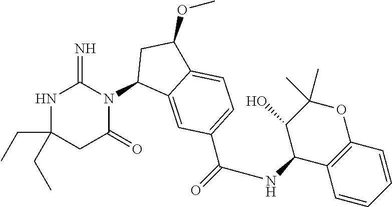 [SMILES: CCC1(CC)CC(=O)N([C@H]2C[C@@H](OC)c3ccc(C(=O)N[C@@H]4c5ccccc5OC(C)(C)[C@H]4O)cc32)C(=N)N1] | 535.4 | (1R,3S)-3-(4,4-diethyl-2-imino-6- oxo-hexahydropyrimidin-1-yl)-N- [(3S,4R)-3-hydroxy-2,2-dimethyl- chroman-4-yl]-1-methoxy-indane- 5-carboxamide |
| 239 | 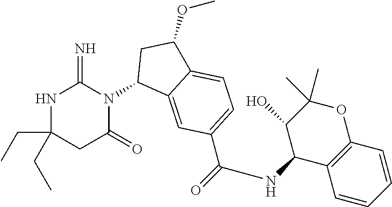 [SMILES: CCC1(CC)CC(=O)N([C@@H]2C[C@H](OC)c3ccc(C(=O)N[C@@H]4c5ccccc5OC(C)(C)[C@H]4O)cc32)C(=N)N1] | 535.3 | (1S,3R)-3-(4,4-diethyl-2-imino-6- oxo-hexahydropyrimidin-1-yl)-N- [(3S,4R)-3-hydroxy-2,2-dimethyl- chroman-4-yl]-1-methoxy-indane- 5-carboxamide |
| 240 | 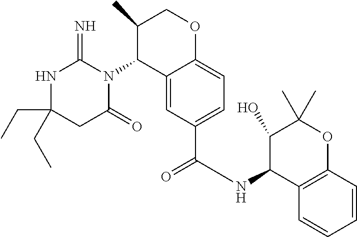 [SMILES: CCC1(CC)CC(=O)N([C@H]2c3cc(C(=O)N[C@@H]4c5ccccc5OC(C)(C)[C@H]4O)ccc3OC[C@@H]2C)C(=N)N1] | 535.4 | (3R,4R)-4-(4,4-diethyl-2-imino-6- oxo-hexahydropyrimidin-1-yl)-N- [(3S,4R)-3-hydroxy-2,2-dimethyl- chroman-4-yl]-3-methyl- chromane-6-carboxamide |
| 241 | 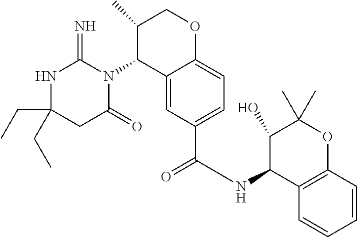 [SMILES: CCC1(CC)CC(=O)N([C@H]2c3cc(C(=O)N[C@@H]4c5ccccc5OC(C)(C)[C@H]4O)ccc3OC[C@H]2C)C(=N)N1] | 535.3 | (3S,4R)-4-(4,4-diethyl-2-imino-6- oxo-hexahydropyrimidin-1-yl)-N- [(3S,4R)-3-hydroxy-2,2-dimethyl- chroman-4-yl]-3-methyl- chromane-6-carboxamide |
| 242A | 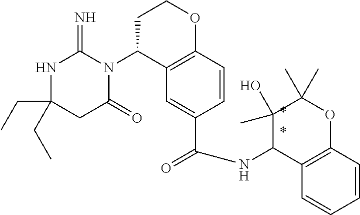 [SMILES: CCC1(CC)CC(=O)N([C@@H]2CCOc3ccc(C(=O)NC4c5ccccc5OC(C)(C)C4(C)O)cc32)C(=N)N1] | 535.3 | (4R)-4-(4,4-diethyl-2-imino-6- oxo-hexahydropyrimidin-1-yl)-N- (3-hydroxy-2,2,3-trimethyl- chroman-4-yl)chromane-6- carboxamide |
| 242B | 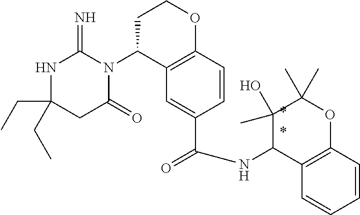 [SMILES: CCC1(CC)CC(=O)N([C@@H]2CCOc3ccc(C(=O)NC4c5ccccc5OC(C)(C)C4(C)O)cc32)C(=N)N1] | 535.3 | (4R)-4-(4,4-diethyl-2-imino-6- oxo-hexahydropyrimidin-1-yl)-N- (3-hydroxy-2,2,3-trimethyl- chroman-4-yl)chromane-6- carboxamide |
| 243 | 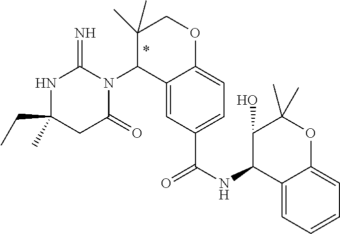 [SMILES: CC[C@]1(C)CC(=O)N(C2c3cc(C(=O)N[C@@H]4c5ccccc5OC(C)(C)[C@H]4O)ccc3OCC2(C)C)C(=N)N1] | 535.3 | 4-[(4R)-4-ethyl-2-imino-4-methyl- 6-oxo-hexahydropyrimidin-1-yl]- N-[(3S,4R)-3-hydroxy-2,2- dimethyl-chroman-4-yl]-3,3- dimethyl-chromane-6- carboxamide |
| 244 | 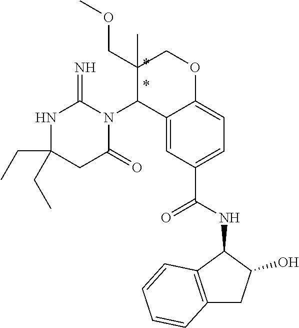 [SMILES: CCC1(CC)CC(=O)N(C2c3cc(C(=O)N[C@@H]4c5ccccc5C[C@H]4O)ccc3OCC2(C)COC)C(=N)N1] | 535.3 | 4-(4,4-diethyl-2-imino-6-oxo- hexahydropyrimidin-1-yl)-N- [(1R,2R)-2-hydroxyindan-1-yl]-3- (methoxymethyl)-3-methyl- chromane-6-carboxamide |
| 245 | 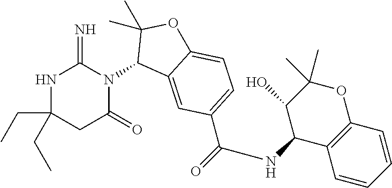 [SMILES: CCC1(CC)CC(=O)N([C@H]2c3cc(C(=O)N[C@@H]4c5ccccc5OC(C)(C)[C@H]4O)ccc3OC2(C)C)C(=N)N1] | 535.4 | (3S)-3-(4,4-diethyl-2-imino-6- oxo-hexahydropyrimidin-1-yl)-N- [(3S,4R)-3-hydroxy-2,2-dimethyl- chroman-4-yl]-2,2-dimethyl-3H- benzofuran-5-carboxamide |
| 246 | 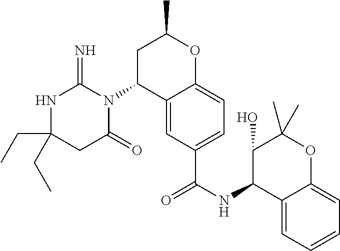 [SMILES: CCC1(CC)CC(=O)N([C@@H]2C[C@@H](C)Oc3ccc(C(=O)N[C@@H]4c5ccccc5OC(C)(C)[C@H]4O)cc32)C(=N)N1] | 535.3 | (2R,4R)-4-(4,4-diethyl-2-imino-6- oxo-hexahydropyrimidin-1-yl)-N- [(3S,4R)-3-hydroxy-2,2-dimethyl- chroman-4-yl]-2-methyl- chromane-6-carboxamide |
| 247 | 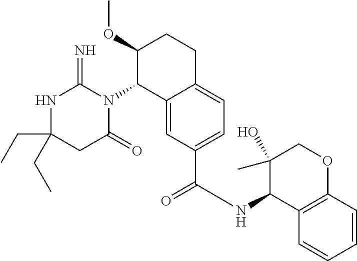 [SMILES: CCC1(CC)CC(=O)N([C@H]2c3cc(C(=O)N[C@@H]4c5ccccc5OC[C@@]4(C)O)ccc3CC[C@@H]2OC)C(=N)N1] | 535.1 | (3S,4S)-4-(4,4-diethyl-2-imino-6- oxo-hexahydropyrimidin-1-yl)-N- [(3S,4R)-3-hydroxy-3-methyl- chroman-4-yl]-3-methoxy-tetralin- 6-carboxamide |
| 248 | 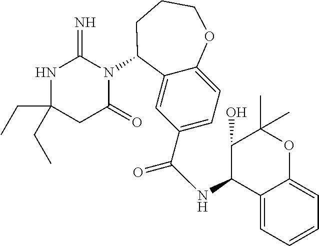 [SMILES: CCC1(CC)CC(=O)N([C@@H]2CCCOc3ccc(C(=O)N[C@@H]4c5ccccc5OC(C)(C)[C@H]4O)cc32)C(=N)N1] | 535.2 | (5R)-5-(4,4-diethyl-2-imino-6- oxo-hexahydropyrimidin-1-yl)-N- [(3S,4R)-3-hydroxy-2,2-dimethyl- chroman-4-yl]-2,3,4,5-tetrahydro- 1-benzoxepine-7-carboxamide |
| 249 | 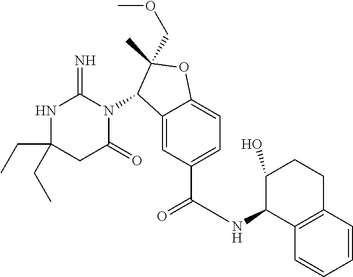 [SMILES: CCC1(CC)CC(=O)N([C@H]2c3cc(C(=O)N[C@@H]4c5ccccc5CC[C@H]4O)ccc3O[C@@]2(C)COC)C(=N)N1] | 535.3 | (2R,3S)-3-(4,4-diethyl-2-imino-6- oxo-hexahydropyrimidin-1-yl)-N- [(1R,2R)-2-hydroxytetralin-1-yl]- 2-(methoxymethyl)-2-methyl-3H- benzofuran-5-carboxamide |
| 250 | 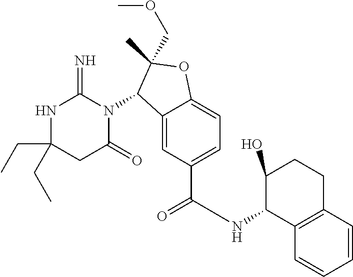 [SMILES: CCC1(CC)CC(=O)N([C@H]2c3cc(C(=O)N[C@H]4c5ccccc5CC[C@@H]4O)ccc3O[C@@]2(C)COC)C(=N)N1] | 535.3 | (2R,3S)-3-(4,4-diethyl-2-imino-6- oxo-hexahydropyrimidin-1-yl)-N- [(1S,2S)-2-hydroxytetralin-1-yl]- 2-(methoxymethyl)-2-methyl-3H- benzofuran-5-carboxamide |
| 251A | 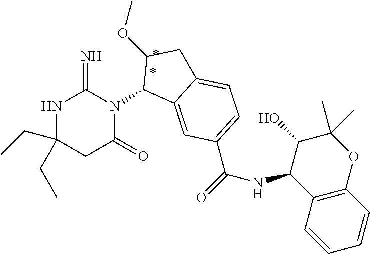 [SMILES: CCC1(CC)CC(=O)N([C@H]2c3cc(C(=O)N[C@@H]4c5ccccc5OC(C)(C)[C@H]4O)ccc3CC2OC)C(=N)N1] | 535.3 | 3-(4,4-diethyl-2-imino-6-oxo- hexahydropyrimidin-1-yl)-N- [(3S,4R)-3-hydroxy-2,2-dimethyl- chroman-4-yl]-2-methoxy-indane- 5-carboxamide |
| 251B | 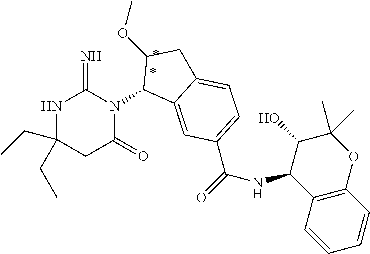 [SMILES: CCC1(CC)CC(=O)N([C@H]2c3cc(C(=O)N[C@@H]4c5ccccc5OC(C)(C)[C@H]4O)ccc3CC2OC)C(=N)N1] | 535.3 | 3-(4,4-diethyl-2-imino-6-oxo- hexahydropyrimidin-1-yl)-N- [(3S,4R)-3-hydroxy-2,2-dimethyl- chroman-4-yl]-2-methoxy-indane- 5-carboxamide |
| 251C | 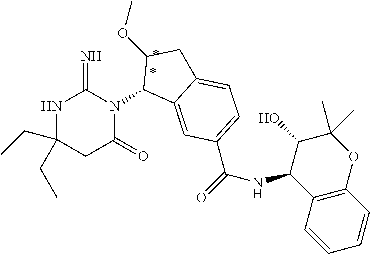 [SMILES: CCC1(CC)CC(=O)N([C@H]2c3cc(C(=O)N[C@@H]4c5ccccc5OC(C)(C)[C@H]4O)ccc3CC2OC)C(=N)N1] | 535.4 | 3-(4,4-diethyl-2-imino-6-oxo- hexahydropyrimidin-1-yl)-N- [(3S,4R)-3-hydroxy-2,2-dimethyl- chroman-4-yl]-2-methoxy-indane- 5-carboxamide |
| 252 | 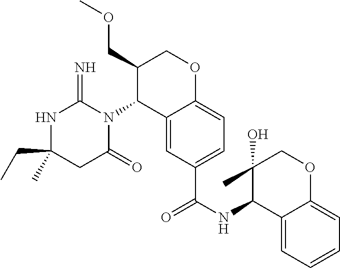 [SMILES: CC[C@]1(C)CC(=O)N([C@H]2c3cc(C(=O)N[C@@H]4c5ccccc5OC[C@@]4(C)O)ccc3OC[C@@H]2COC)C(=N)N1] | 537.2 | (3S,4R)-4-[(4R)-4-ethyl-2-imino- 4-methyl-6-oxo- hexahydropyrimidin-1-yl]-N- [(3S,4R)-3-hydroxy-3-methyl- chroman-4-yl]-3- (methoxymethyl)chromane-6- carboxamide |
| 253 | 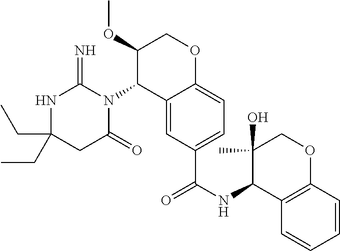 [SMILES: CCC1(CC)CC(=O)N([C@H]2c3cc(C(=O)N[C@@H]4c5ccccc5OC[C@]4(C)O)ccc3OC[C@@H]2OC)C(=N)N1] | 537.3 | (3R,4S)-4-(4,4-diethyl-2-imino-6- oxo-hexahydropyrimidin-1-yl)-N- [(3R,4R)-3-hydroxy-3-methyl- chroman-4-yl]-3-methoxy- chromane-6-carboxamide |
| 254 | 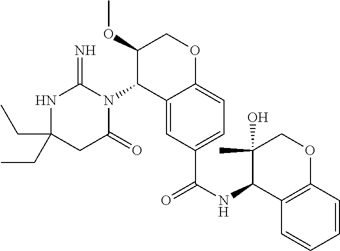 [SMILES: CCC1(CC)CC(=O)N([C@H]2c3cc(C(=O)N[C@@H]4c5ccccc5OC[C@@]4(C)O)ccc3OC[C@@H]2OC)C(=N)N1] | 537.3 | (3R,4S)-4-(4,4-diethyl-2-imino-6- oxo-hexahydropyrimidin-1-yl)-N- [(3S,4R)-3-hydroxy-3-methyl- chroman-4-yl]-3-methoxy- chromane-6-carboxamide |
| 255 | 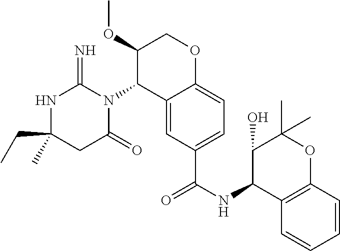 [SMILES: CC[C@]1(C)CC(=O)N([C@H]2c3cc(C(=O)N[C@@H]4c5ccccc5OC(C)(C)[C@H]4O)ccc3OC[C@@H]2OC)C(=N)N1] | 537.2 | (3R,4S)-4-[(4R)-4-ethyl-2-imino- 4-methyl-6-oxo- hexahydropyrimidin-1-yl]-N- [(3S,4R)-3-hydroxy-2,2-dimethyl- chroman-4-yl]-3-methoxy- chromane-6-carboxamide |
| 256 | 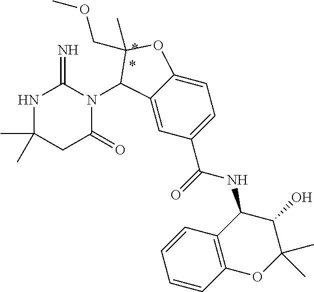 | 537.2 | N-[(3S,4R)-3-hydroxy-2,2- dimethyl-chroman-4-yl]-3-(2- imino-4,4-dimethyl-6-oxo- hexahydropyrimidin-1-yl)-2- (methoxymethyl)-2-methyl-3H- benzofuran-5-carboxamide |
| 257 | 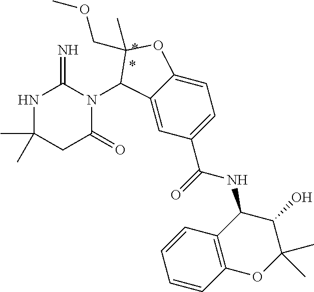 | 537.3 | N-[(3S,4R)-3-hydroxy-2,2- dimethyl-chroman-4-yl]-3-(2- imino-4,4-dimethyl-6-oxo- hexahydropyrimidin-1-yl)-2- (methoxymethyl)-2-methyl-3H- benzofuran-5-carboxamide |
| 258A | 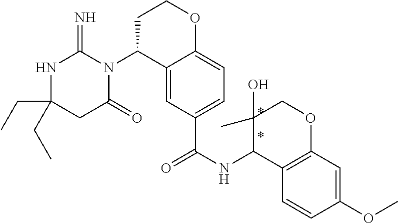 [SMILES: CCC1(CC)CC(=O)N([C@@H]2CCOc3ccc(C(=O)NC4c5ccc(OC)cc5OCC4(C)O)cc32)C(=N)N1] | 537.3 | (4R)-4-(4,4-diethyl-2-imino-6- oxo-hexahydropyrimidin-1-yl)-N- (3-hydroxy-7-methoxy-3-methyl- chroman-4-yl)chromane-6- carboxamide |
| 258B | 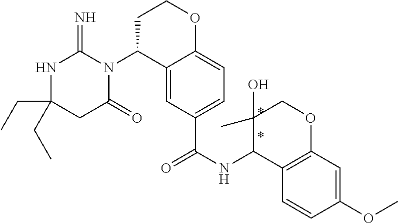 [SMILES: CCC1(CC)CC(=O)N([C@@H]2CCOc3ccc(C(=O)NC4c5ccc(OC)cc5OCC4(C)O)cc32)C(=N)N1] | 537.3 | (4R)-4-(4,4-diethyl-2-imino-6- oxo-hexahydropyrimidin-1-yl)-N- (3-hydroxy-7-methoxy-3-methyl- chroman-4-yl)chromane-6- carboxamide |
| 258C | 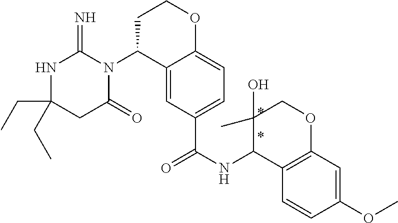 [SMILES: CCC1(CC)CC(=O)N([C@@H]2CCOc3ccc(C(=O)NC4c5ccc(OC)cc5OCC4(C)O)cc32)C(=N)N1] | 537.3 | (4R)-4-(4,4-diethyl-2-imino-6- oxo-hexahydropyrimidin-1-yl)-N- (3-hydroxy-7-methoxy-3-methyl- chroman-4-yl)chromane-6- carboxamide |
| 258D | 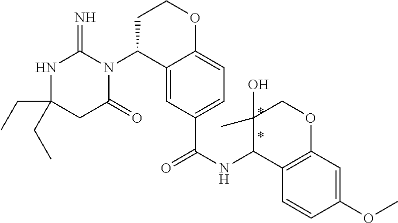 [SMILES: CCC1(CC)CC(=O)N([C@@H]2CCOc3ccc(C(=O)NC4c5ccc(OC)cc5OCC4(C)O)cc32)C(=N)N1] | 537.3 | (4R)-4-(4,4-diethyl-2-imino-6- oxo-hexahydropyrimidin-1-yl)-N- (3-hydroxy-7-methoxy-3-methyl- chroman-4-yl)chromane-6- carboxamide |
| 259A | 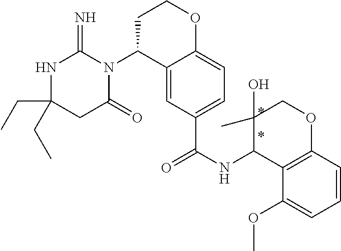 [SMILES: CCC1(CC)CC(=O)N([C@@H]2CCOc3ccc(C(=O)NC4c5c(OC)cccc5OCC4(C)O)cc32)C(=N)N1] | 537.3 | (4R)-4-(4,4-diethyl-2-imino-6- oxo-hexahydropyrimidin-1-yl)-N- (3-hydroxy-5-methoxy-3-methyl- chroman-4-yl)chromane-6- carboxamide |
| 259B | 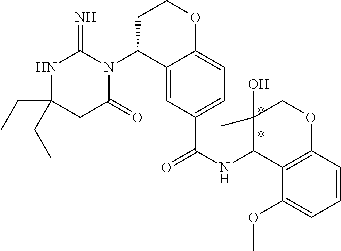 [SMILES: CCC1(CC)CC(=O)N([C@@H]2CCOc3ccc(C(=O)NC4c5c(OC)cccc5OCC4(C)O)cc32)C(=N)N1] | 537.3 | (4R)-4-(4,4-diethyl-2-imino-6- oxo-hexahydropyrimidin-1-yl)-N- (3-hydroxy-5-methoxy-3-methyl- chroman-4-yl)chromane-6- carboxamide |
| 259C | 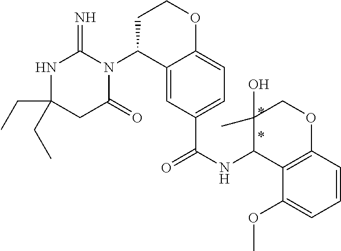 [SMILES: CCC1(CC)CC(=O)N([C@@H]2CCOc3ccc(C(=O)NC4c5c(OC)cccc5OCC4(C)O)cc32)C(=N)N1] | 537.3 | (4R)-4-(4,4-diethyl-2-imino-6- oxo-hexahydropyrimidin-1-yl)-N- (3-hydroxy-5-methoxy-3-methyl- chroman-4-yl)chromane-6- carboxamide |
| 259D | 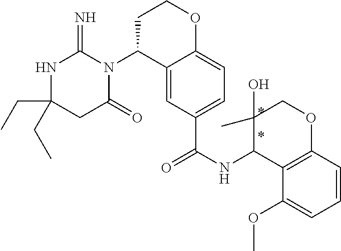 [SMILES: CCC1(CC)CC(=O)N([C@@H]2CCOc3ccc(C(=O)NC4c5c(OC)cccc5OCC4(C)O)cc32)C(=N)N1] | 537.3 | (4R)-4-(4,4-diethyl-2-imino-6- oxo-hexahydropyrimidin-1-yl)-N- (3-hydroxy-5-methoxy-3-methyl- chroman-4-yl)chromane-6- carboxamide |
| 260 | 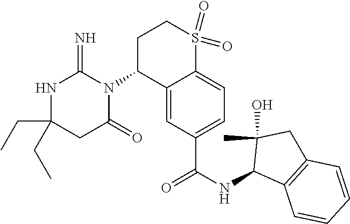 [SMILES: CCC1(CC)CC(=O)N([C@@H]2CCS(=O)(=O)c3ccc(C(=O)N[C@@H]4c5ccccc5C[C@@]4(C)O)cc32)C(=N)N1] | 539.3 | (4R)-4-(4,4-diethyl-2-imino-6- oxo-hexahydropyrimidin-1-yl)-N- [(1R,2R)-2-hydroxy-2-methyl- indan-1-yl]-1,1-dioxo-3,4- dihydro-2H-thiochromene-6- carboxamide |
| 261 | 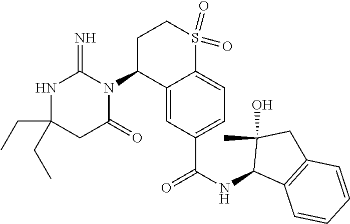 [SMILES: CCC1(CC)CC(=O)N([C@H]2CCS(=O)(=O)c3ccc(C(=O)N[C@@H]4c5ccccc5C[C@@]4(C)O)cc32)C(=N)N1] | 539.3 | (4S)-4-(4,4-diethyl-2-imino-6- oxo-hexahydropyrimidin-1-yl)-N- [(1R,2R)-2-hydroxy-2-methyl- indan-1-yl]-1,1-dioxo-3,4- dihydro-2H-thiochromene-6- carboxamide |
| 262 | 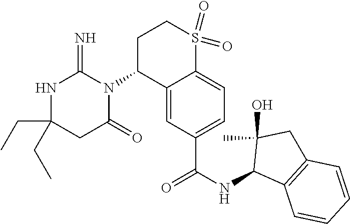 [SMILES: CCC1(CC)CC(=O)N([C@@H]2CCS(=O)(=O)c3ccc(C(=O)N[C@@H]4c5ccccc5C[C@]4(C)O)cc32)C(=N)N1] | 539.3 | (4R)-4-(4,4-diethyl-2-imino-6- oxo-hexahydropyrimidin-1-yl)-N- [(1R,2S)-2-hydroxy-2-methyl- indan-1-yl]-1,1-dioxo-3,4- dihydro-2H-thiochromene-6- carboxamide |
| 263 | 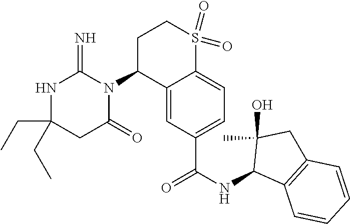 [SMILES: CCC1(CC)CC(=O)N([C@H]2CCS(=O)(=O)c3ccc(C(=O)N[C@@H]4c5ccccc5C[C@]4(C)O)cc32)C(=N)N1] | 539.3 | (4S)-4-(4,4-diethyl-2-imino-6- oxo-hexahydropyrimidin-1-yl)-N- [(1R,2S)-2-hydroxy-2-methyl- indan-1-yl]-1,1-dioxo-3,4- dihydro-2H-thiochromene-6- carboxamide |
| 264 | 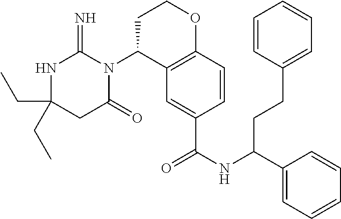 [SMILES: CCC1(CC)CC(=O)N([C@@H]2CCOc3ccc(C(=O)NC(CCc4ccccc4)c4ccccc4)cc32)C(=N)N1] | 539.3 | (4R)-4-(4,4-diethyl-2-imino-6- oxo-hexahydropyrimidin-1-yl)-N- (1,3-diphenylpropyl)chromane-6- carboxamide |
| 265 | 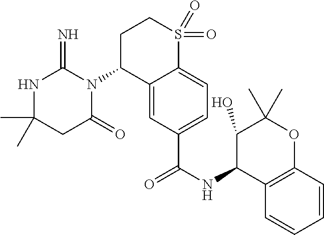 [SMILES: CC1(C)CC(=O)N([C@@H]2CCS(=O)(=O)c3ccc(C(=O)N[C@@H]4c5ccccc5OC(C)(C)[C@H]4O)cc32)C(=N)N1] | 541.3 | (4R)-N-[(3S,4R)-3-hydroxy-2,2- dimethyl-chroman-4-yl]-4-(2- imino-4,4-dimethyl-6-oxo- hexahydropyrimidin-1-yl)-1,1- dioxo-3,4-dihydro-2H- thiochromene-6-carboxamide |
| 266 | 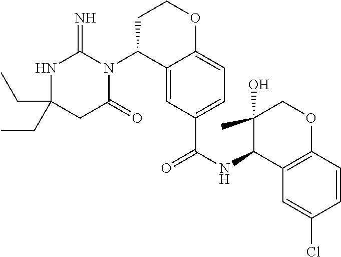 [SMILES: CCC1(CC)CC(=O)N([C@@H]2CCOc3ccc(C(=O)N[C@@H]4c5cc(Cl)ccc5OC[C@@]4(C)O)cc32)C(=N)N1] | 541.2 | (4R)-N-[(3S,4R)-6-chloro-3- hydroxy-3-methyl-chroman-4-yl]- 4-(4,4-diethyl-2-imino-6-oxo- hexahydropyrimidin-1- yl)chromane-6-carboxamide |
| 267 | 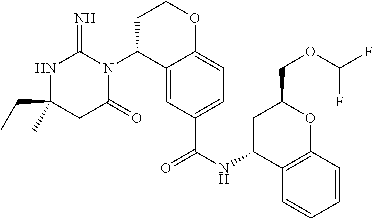 [SMILES: CC[C@]1(C)CC(=O)N([C@@H]2CCOc3ccc(C(=O)N[C@@H]4C[C@@H](COC(F)F)Oc5ccccc54)cc32)C(=N)N1] | 543.3 | (4R)-N-[(2S,4R)-2- (difluoromethoxymethyl)chroman- 4-yl]-4-[(4R)-4-ethyl-2-imino-4- methyl-6-oxo- hexahydropyrimidin-1- yl]chromane-6-carboxamide |
| 268 | 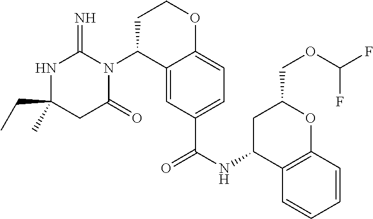 [SMILES: CC[C@]1(C)CC(=O)N([C@@H]2CCOc3ccc(C(=O)N[C@@H]4C[C@H](COC(F)F)Oc5ccccc54)cc32)C(=N)N1] | 543.3 | (4R)-N-[(2R,4R)-2- (difluoromethoxymethyl)chroman- 4-yl]-4-[(4R)-4-ethyl-2-imino-4- methyl-6-oxo- hexahydropyrimidin-1- yl]chromane-6-carboxamide |
| 269 | 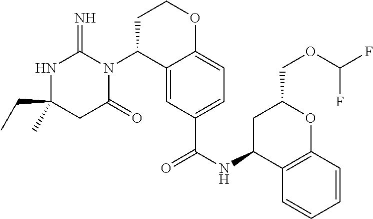 [SMILES: CC[C@]1(C)CC(=O)N([C@@H]2CCOc3ccc(C(=O)N[C@H]4C[C@H](COC(F)F)Oc5ccccc54)cc32)C(=N)N1] | 543.3 | (4R)-N-[(2R,4S)-2- (difluoromethoxymethyl)chroman- 4-yl]-4-[(4R)-4-ethyl-2-imino-4- methyl-6-oxo- hexahydropyrimidin-1- yl]chromane-6-carboxamide |
| 270 | 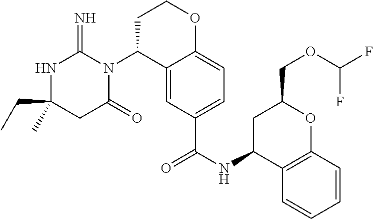 [SMILES: CC[C@]1(C)CC(=O)N([C@@H]2CCOc3ccc(C(=O)N[C@H]4C[C@@H](COC(F)F)Oc5ccccc54)cc32)C(=N)N1] | 543.3 | (4R)-N-[(2S,4S)-2- (difluoromethoxymethyl)chroman- 4-yl]-4-[(4R)-4-ethyl-2-imino-4- methyl-6-oxo- hexahydropyrimidin-1- yl]chromane-6-carboxamide |
| 271 | 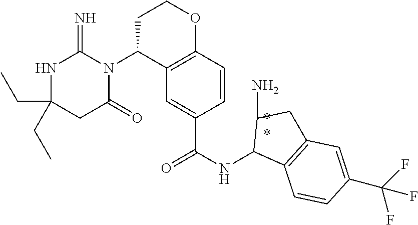 [SMILES: CCC1(CC)CC(=O)N([C@@H]2CCOc3ccc(C(=O)NC4c5ccc(C(F)(F)F)cc5CC4N)cc32)C(=N)N1] | 544.3 | (4R)-N-[2-amino-5- (trifluoromethyl)indan-1-yl]-4- (4,4-diethyl-2-imino-6-oxo- hexahydropyrimidin-1- yl)chromane-6-carboxamide |
| 272A | 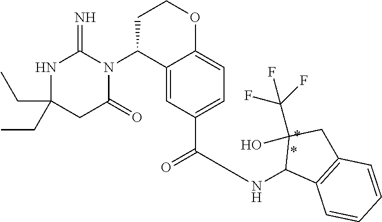 [SMILES: CCC1(CC)CC(=O)N([C@@H]2CCOc3ccc(C(=O)NC4c5ccccc5CC4(O)C(F)(F)F)cc32)C(=N)N1] | 545.3 | (4R)-4-(4,4-diethyl-2-imino-6- oxo-hexahydropyrimidin-1-yl)-N- [2-hydroxy-2- (trifluoromethyl)indan-1- yl]chromane-6-carboxamide |
| 272B | 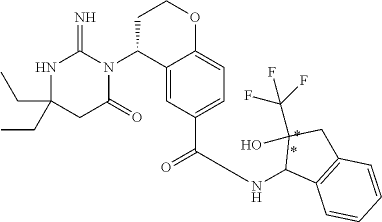 [SMILES: CCC1(CC)CC(=O)N([C@@H]2CCOc3ccc(C(=O)NC4c5ccccc5CC4(O)C(F)(F)F)cc32)C(=N)N1] | 545.3 | (4R)-4-(4,4-diethyl-2-imino-6- oxo-hexahydropyrimidin-1-yl)-N- [2-hydroxy-2- (trifluoromethyl)indan-1- yl]chromane-6-carboxamide |
| 272C | 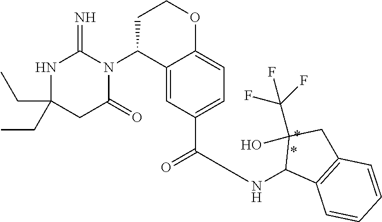 [SMILES: CCC1(CC)CC(=O)N([C@@H]2CCOc3ccc(C(=O)NC4c5ccccc5CC4(O)C(F)(F)F)cc32)C(=N)N1] | 545.3 | (4R)-4-(4,4-diethyl-2-imino-6- oxo-hexahydropyrimidin-1-yl)-N- [2-hydroxy-2- (trifluoromethyl)indan-1- yl]chromane-6-carboxamide |
| 272D | 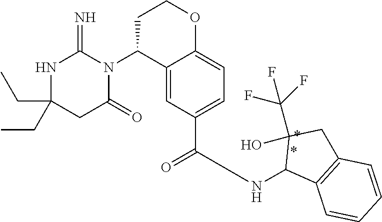 [SMILES: CCC1(CC)CC(=O)N([C@@H]2CCOc3ccc(C(=O)NC4c5ccccc5CC4(O)C(F)(F)F)cc32)C(=N)N1] | 545.3 | (4R)-4-(4,4-diethyl-2-imino-6- oxo-hexahydropyrimidin-1-yl)-N- [2-hydroxy-2- (trifluoromethyl)indan-1- yl]chromane-6-carboxamide |
| 273 | 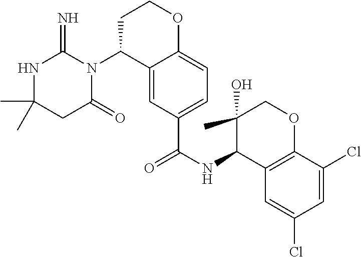 [SMILES: CC1(C)CC(=O)N([C@@H]2CCOc3ccc(C(=O)N[C@@H]4c5cc(Cl)cc(Cl)c5OC[C@@]4(C)O)cc32)C(=N)N1] | 547.1 | (4R)-N-[(3S,4R)-6,8-dichloro-3- hydroxy-3-methyl-chroman-4-yl]- 4-(2-imino-4,4-dimethyl-6-oxo- hexahydropyrimidin-1- yl)chromane-6-carboxamide |
| 274 | 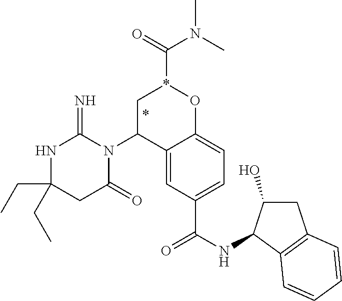 [SMILES: CCC1(CC)CC(=O)N(C2C*(C(=O)N(C)C)Oc3ccc(C(=O)N[C@@H]4c5ccccc5C[C@H]4O)cc32)C(=N)N1] | 548.3 | 4-(4,4-diethyl-2-imino-6-oxo- hexahydropyrimidin-1-yl)-N6- [(1R,2R)-2-hydroxyindan-1-yl]- N2,N2-dimethyl-chromane-2,6- dicarboxamide |
| 275 | 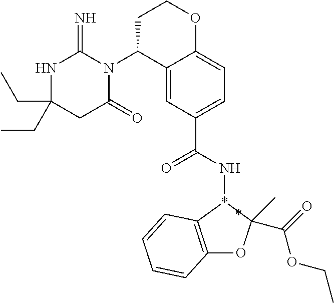 [SMILES: CCOC(=O)C1(C)Oc2ccccc2*1NC(=O)c1ccc2c(c1)[C@H](N1C(=N)NC(CC)(CC)CC1=O)CCO2] | 549.4 | ethyl 3-[[(4R)-4-(4,4-diethyl-2- imino-6-oxo-hexahydropyrimidin- 1-yl)chromane-6- carbonyl]amino]-2-methyl-3H- benzofuran-2-carboxylate |
| 276 | 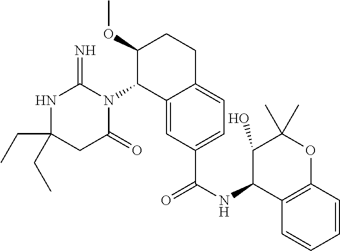 [SMILES: CCC1(CC)CC(=O)N([C@H]2c3cc(C(=O)N[C@@H]4c5ccccc5OC(C)(C)[C@H]4O)ccc3CC[C@@H]2OC)C(=N)N1] | 549.2 | (3S,4S)-4-(4,4-diethyl-2-imino-6- oxo-hexahydropyrimidin-1-yl)-N- [(3S,4R)-3-hydroxy-2,2-dimethyl- chroman-4-yl]-3-methoxy-tetralin- 6-carboxamide |
| 277 | 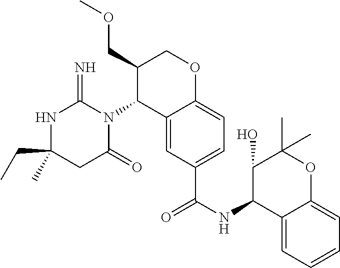 [SMILES: CC[C@]1(C)CC(=O)N([C@H]2c3cc(C(=O)N[C@@H]4c5ccccc5OC(C)(C)[C@H]4O)ccc3OC[C@@H]2COC)C(=N)N1] | 551.1 | (3S,4R)-4-[(4R)-4-ethyl-2-imino- 4-methyl-6-oxo- hexahydropyrimidin-1-yl]-N- [(3S,4R)-3-hydroxy-2,2-dimethyl- chroman-4-yl]-3- (methoxymethyl)chromane-6- carboxamide |
| 278 | 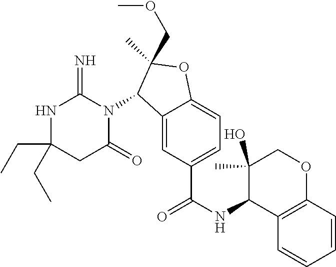 [SMILES: CCC1(CC)CC(=O)N([C@H]2c3cc(C(=O)N[C@@H]4c5ccccc5OC[C@]4(C)O)ccc3O[C@]2(C)COC)C(=N)N1] | 551.3 | (2S,3S)-3-(4,4-diethyl-2-imino-6- oxo-hexahydropyrimidin-1-yl)-N- [(3R,4R)-3-hydroxy-3-methyl- chroman-4-yl]-2- (methoxymethyl)-2-methyl-3H- benzofuran-5-carboxamide |
| 279 | 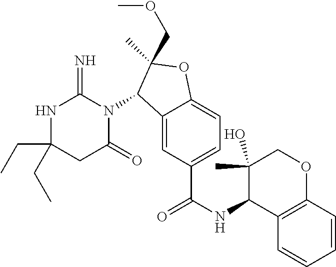 [SMILES: CCC1(CC)CC(=O)N([C@H]2c3cc(C(=O)N[C@@H]4c5ccccc5OC[C@@]4(C)O)ccc3O[C@]2(C)COC)C(=N)N1] | 551.3 | (2S,3S)-3-(4,4-diethyl-2-imino-6- oxo-hexahydropyrimidin-1-yl)-N- [(3S,4R)-3-hydroxy-3-methyl- chroman-4-yl]-2- (methoxymethyl)-2-methyl-3H- benzofuran-5-carboxamide |
| 280 | 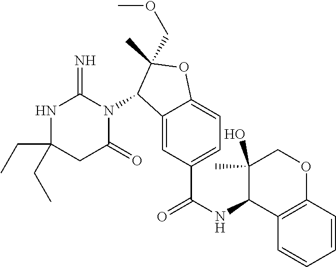 [SMILES: CCC1(CC)CC(=O)N([C@H]2c3cc(C(=O)N[C@@H]4c5ccccc5OC[C@]4(C)O)ccc3O[C@@]2(C)COC)C(=N)N1] | 551.3 | (2R,3S)-3-(4,4-diethyl-2-imino-6- oxo-hexahydropyrimidin-1-yl)-N- [(3R,4R)-3-hydroxy-3-methyl- chroman-4-yl]-2- (methoxymethyl)-2-methyl-3H- benzofuran-5-carboxamide |
| 281 | 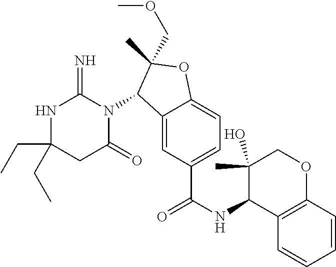 [SMILES: CCC1(CC)CC(=O)N([C@H]2c3cc(C(=O)N[C@@H]4c5ccccc5OC[C@@]4(C)O)ccc3O[C@@]2(C)COC)C(=N)N1] | 551.3 | (2R,3S)-3-(4,4-diethyl-2-imino-6- oxo-hexahydropyrimidin-1-yl)-N- [(3S,4R)-3-hydroxy-3-methyl- chroman-4-yl]-2- (methoxymethyl)-2-methyl-3H- benzofuran-5-carboxamide |
| 282A | 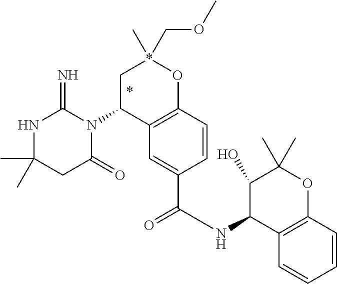 [SMILES: COC*1(C)C[C@@H](N2C(=N)NC(C)(C)CC2=O)c2cc(C(=O)N[C@@H]3c4ccccc4OC(C)(C)[C@H]3O)ccc2O1] | 551.3 | N-[(3S,4R)-3-hydroxy-2,2- dimethyl-chroman-4-yl]-4-(2- imino-4,4-dimethyl-6-oxo- hexahydropyrimidin-1-yl)-2- (methoxymethyl)-2-methyl- chromane-6-carboxamide |
| 282B | 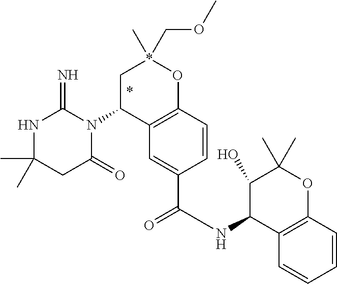 [SMILES: COC*1(C)C[C@@H](N2C(=N)NC(C)(C)CC2=O)c2cc(C(=O)N[C@@H]3c4ccccc4OC(C)(C)[C@H]3O)ccc2O1] | 551.1 | N-[(3S,4R)-3-hydroxy-2,2- dimethyl-chroman-4-yl]-4-(2- imino-4,4-dimethyl-6-oxo- hexahydropyrimidin-1-yl)-2- (methoxymethyl)-2-methyl- chromane-6-carboxamide |
| 283 | 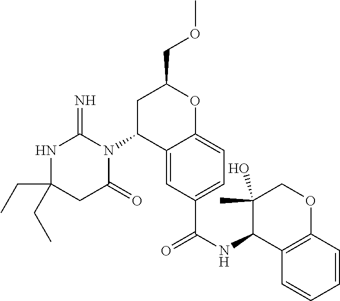 [SMILES: CCC1(CC)CC(=O)N([C@@H]2C[C@@H](COC)Oc3ccc(C(=O)N[C@@H]4c5ccccc5OC[C@@]4(C)O)cc32)C(=N)N1] | 551.2 | (2S,4R)-4-(4,4-diethyl-2-imino-6- oxo-hexahydropyrimidin-1-yl)-N- [(3S,4R)-3-hydroxy-3-methyl- chroman-4-yl]-2- (methoxymethyl)chromane-6- carboxamide |
| 284 | 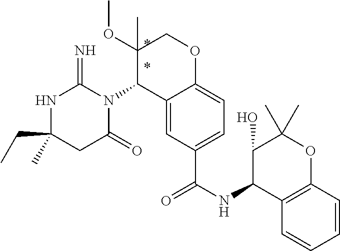 [SMILES: CC[C@]1(C)CC(=O)N([C@H]2c3cc(C(=O)N[C@@H]4c5ccccc5OC(C)(C)[C@H]4O)ccc3OCC2(C)OC)C(=N)N1] | 551.1 | 4-[(4R)-4-ethyl-2-imino-4-methyl- 6-oxo-hexahydropyrimidin-1-yl]- N-[(3S,4R)-3-hydroxy-2,2- dimethyl-chroman-4-yl]-3- methoxy-3-methyl-chromane-6- carboxamide |
| 285 | 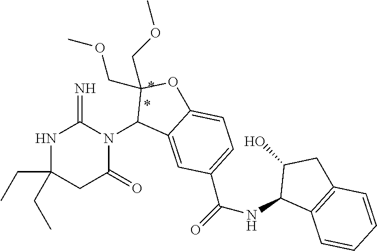 [SMILES: CCC1(CC)CC(=O)N(C2c3cc(C(=O)N[C@@H]4c5ccccc5C[C@H]4O)ccc3OC2(COC)COC)C(=N)N1] | 551.3 | 3-(4,4-diethyl-2-imino-6-oxo- hexahydropyrimidin-1-yl)-N- [(1R,2R)-2-hydroxyindan-1-yl]- 2,2-bis(methoxymethyl)-3H- benzofuran-5-carboxamide |
| 286 | 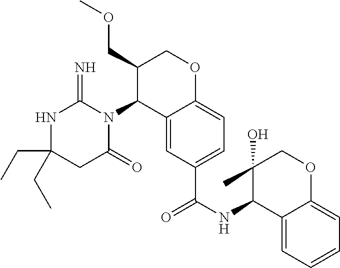 [SMILES: CCC1(CC)CC(=O)N([C@@H]2c3cc(C(=O)N[C@@H]4c5ccccc5OC[C@@]4(C)O)ccc3OC[C@@H]2COC)C(=N)N1] | 551.3 | (3S,4S)-4-(4,4-diethyl-2-imino-6- oxo-hexahydropyrimidin-1-yl)-N- [(3S,4R)-3-hydroxy-3-methyl- chroman-4-yl]-3- (methoxymethyl)chromane-6- carboxamide |
| 287 | 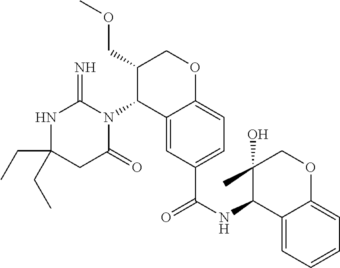 [SMILES: CCC1(CC)CC(=O)N([C@H]2c3cc(C(=O)N[C@@H]4c5ccccc5OC[C@@]4(C)O)ccc3OC[C@H]2COC)C(=N)N1] | 551.3 | (3R,4R)-4-(4,4-diethyl-2-imino-6- oxo-hexahydropyrimidin-1-yl)-N- [(3S,4R)-3-hydroxy-3-methyl- chroman-4-yl]-3- (methoxymethyl)chromane-6- carboxamide |
| 288 | 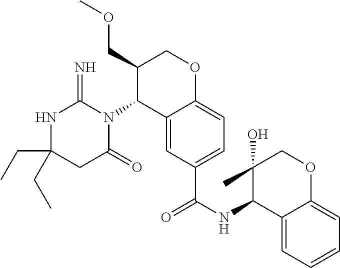 [SMILES: CCC1(CC)CC(=O)N([C@H]2c3cc(C(=O)N[C@@H]4c5ccccc5OC[C@@]4(C)O)ccc3OC[C@@H]2COC)C(=N)N1] | 551.4 | (3S,4R)-4-(4,4-diethyl-2-imino-6- oxo-hexahydropyrimidin-1-yl)-N- [(3S,4R)-3-hydroxy-3-methyl- chroman-4-yl]-3- (methoxymethyl)chromane-6- carboxamide |
| 289 | 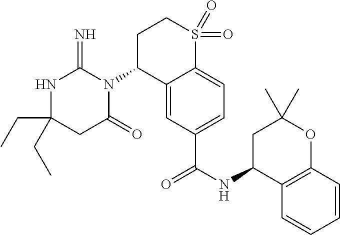 [SMILES: CCC1(CC)CC(=O)N([C@@H]2CCS(=O)(=O)c3ccc(C(=O)N[C@H]4CC(C)(C)Oc5ccccc54)cc32)C(=N)N1] | 553.3 | (4R)-4-(4,4-diethyl-2-imino-6- oxo-hexahydropyrimidin-1-yl)-N- [(4S)-2,2-dimethylchroman-4-yl]- 1,1-dioxo-3,4-dihydro-2H- thiochromene-6-carboxamide |
| 290 | 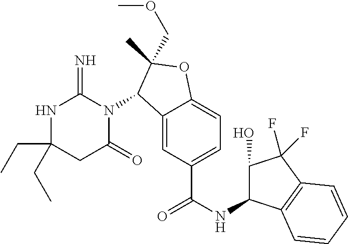 [SMILES: CCC1(CC)CC(=O)N([C@H]2c3cc(C(=O)N[C@@H]4c5ccccc5C(F)(F)[C@H]4O)ccc3O[C@@]2(C)COC)C(=N)N1] | 557.3 | (2R,3S)-3-(4,4-diethyl-2-imino-6- oxo-hexahydropyrimidin-1-yl)-N- [(1R,2S)-3,3-difluoro-2-hydroxy- indan-1-yl]-2-(methoxymethyl)-2- methyl-3H-benzofuran-5- carboxamide |
| 291 | 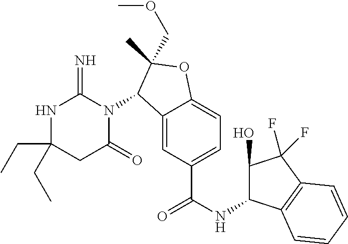 [SMILES: CCC1(CC)CC(=O)N([C@H]2c3cc(C(=O)N[C@H]4c5ccccc5C(F)(F)[C@@H]4O)ccc3O[C@@]2(C)COC)C(=N)N1] | 557.2 | (2R,3S)-3-(4,4-diethyl-2-imino-6- oxo-hexahydropyrimidin-1-yl)-N- [(1S,2R)-3,3-difluoro-2-hydroxy- indan-1-yl]-2-(methoxymethyl)-2- methyl-3H-benzofuran-5- carboxamide |
| 292 | 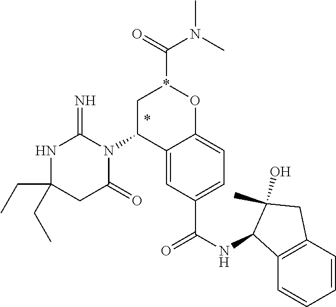 [SMILES: CCC1(CC)CC(=O)N([C@@H]2C*(C(=O)N(C)C)Oc3ccc(C(=O)N[C@@H]4c5ccccc5C[C@@]4(C)O)cc32)C(=N)N1] | 562.4 | 4-(4,4-diethyl-2-imino-6-oxo- hexahydropyrimidin-1-yl)-N6- [(1R,2R)-2-hydroxy-2-methyl- indan-1-yl]-N2,N2-dimethyl- chromane-2,6-dicarboxamide |
| 293 | 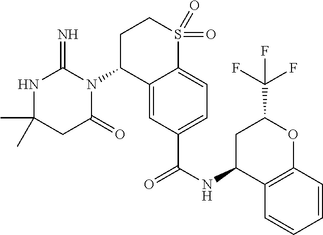 [SMILES: CC1(C)CC(=O)N([C@@H]2CCS(=O)(=O)c3ccc(C(=O)N[C@H]4C[C@H](C(F)(F)F)Oc5ccccc54)cc32)C(=N)N1] | 565.2 | (4R)-4-(2-imino-4,4-dimethyl-6- oxo-hexahydropyrimidin-1-yl)- 1,1-dioxo-N-[(2R,4S)-2- (trifluoromethyl)chroman-4-yl]- 3,4-dihydro-2H-thiochromene-6- carboxamide |
| 294 | 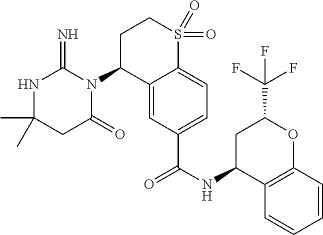 [SMILES: CC1(C)CC(=O)N([C@H]2CCS(=O)(=O)c3ccc(C(=O)N[C@H]4C[C@H](C(F)(F)F)Oc5ccccc54)cc32)C(=N)N1] | 565.2 | (4S)-4-(2-imino-4,4-dimethyl-6- oxo-hexahydropyrimidin-1-yl)- 1,1-dioxo-N-[(2R,4S)-2- (trifluoromethyl)chroman-4-yl]- 3,4-dihydro-2H-thiochromene-6- carboxamide |
| 295 | 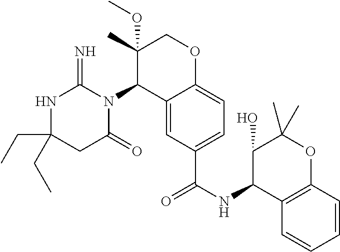 [SMILES: CCC1(CC)CC(=O)N([C@@H]2c3cc(C(=O)N[C@@H]4c5ccccc5OC(C)(C)[C@H]4O)ccc3OC[C@@]2(C)OC)C(=N)N1] | 565.3 | (3S,4R)-4-(4,4-diethyl-2-imino-6- oxo-hexahydropyrimidin-1-yl)-N- [(3S,4R)-3-hydroxy-2,2-dimethyl- chroman-4-yl]-3-methoxy-3- methyl-chromane-6-carboxamide |
| 296 | 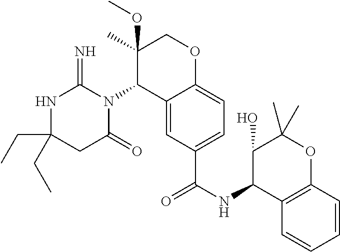 [SMILES: CCC1(CC)CC(=O)N([C@H]2c3cc(C(=O)N[C@@H]4c5ccccc5OC(C)(C)[C@H]4O)ccc3OC[C@]2(C)OC)C(=N)N1] | 565.3 | (3R,4S)-4-(4,4-diethyl-2-imino-6- oxo-hexahydropyrimidin-1-yl)-N- [(3S,4R)-3-hydroxy-2,2-dimethyl- chroman-4-yl]-3-methoxy-3- methyl-chromane-6-carboxamide |
| 297 | 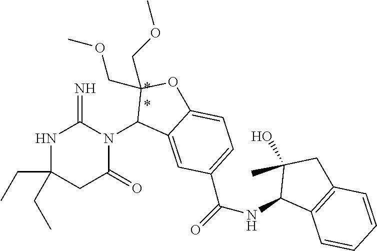 [SMILES: CCC1(CC)CC(=O)N(C2c3cc(C(=O)N[C@@H]4c5ccccc5C[C@@]4(C)O)ccc3OC2(COC)COC)C(=N)N1] | 565.3 | 3-(4,4-diethyl-2-imino-6-oxo- hexahydropyrimidin-1-yl)-N- [(1R,2R)-2-hydroxy-2-methyl- indan-1-yl]-2,2- bis(methoxymethyl)-3H- benzofuran-5-carboxamide |

### Example 298

### (3R,4R)-4-(4,4-diethyl-2-imino-6-oxotetrahydropyrimidin-1(2H)-yl)-N-((3 S,4R)-3-hydroxy-2,2-dimethyl chromian-4-yl)-3-(methoxymethyl)chromane-6-carboxamiide

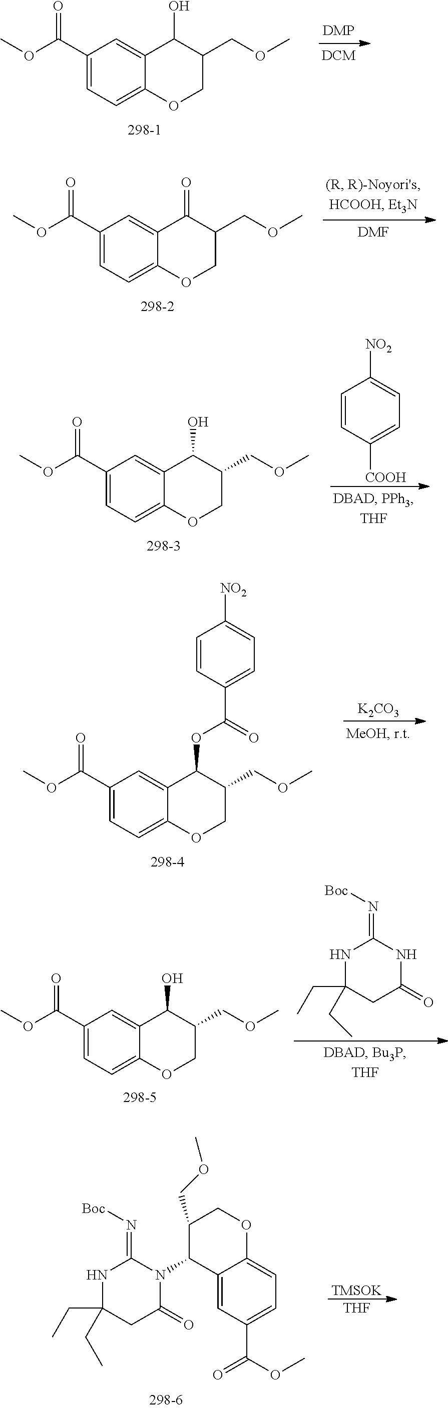

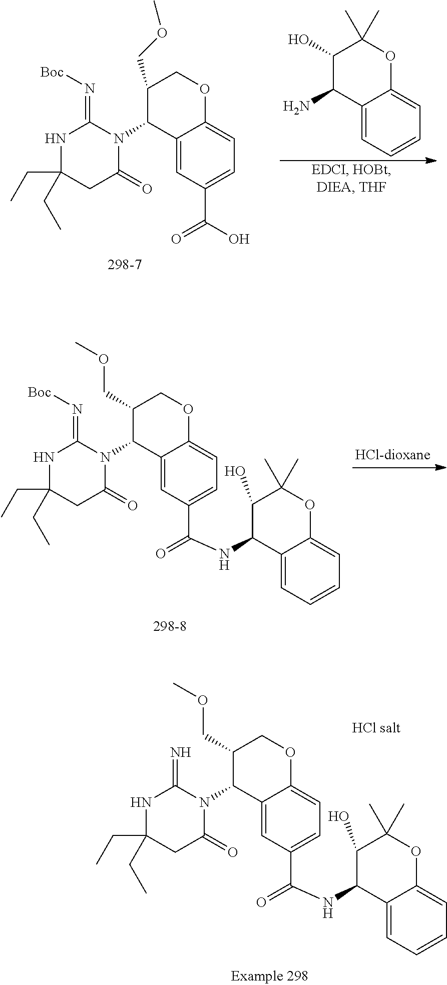

### Step A: methyl 3-(methoxymethyl)-4-oxochromane-6-carboxylate 298-2

DMP (121 g, 285 mmol) was added to a solution of methyl 4-hydroxy-3-(methoxymethyl)chromane-6-carboxylate 298-1 (60 g, 238 mmol) in CH₂Cl₂ (1500 mL) under nitrogen. The mixture was stirred at 25° C. for 2 h. The mixture was filtered, then concentrated and purified by column chromatography (petroleum ether/EtOAc=100:0 to 10:1) to afford product methyl 3-(methoxymethyl)-4-oxochromane-6-carboxylate 298-2.

MS (ESI) m/z 251.3 (M+H⁺)

¹H NMR (400 MHz, chloroform-d) δ 8.57 (d, J=2.0 Hz, 1H), 8.13 (d, J=2.4, 8.4 Hz, 1H), 7.02 (d, J=8.4 Hz, 1H), 4.70 (dd, J=5.2, 11.2 Hz, 1H), 4.47 (dd, J=5.6, 11.6 Hz, 1H), 3.90 (s, 3H), 3.76-3.84 (m, 1H), 3.65-3.75 (m, 1H), 3.37 (s, 3H), 3.06-3.07 (m, 1H)

### Step B: methyl (3R,4R)-4-hydroxy-3-(methoxymethyl)chromane-6-carboxylate 298-3

A solution of formic acid (26.7 mL, 707 mmol) and triethylamine (197 mL, 1415 mmol) in DMF (200 mL) was stirred for 15 min, then methyl 3-(methoxymethyl)-4-oxochromane-6-carboxylate 298-2 (59 g, 236 mmol) in DMF (200 mL) and RuCl(p-cymene)[(R,R)-Ts-DPEN](C₃₁H₃₅ClN₂O₂RuS) (4.50 g, 7.07 mmol) was added and stirred for 12 h at 25° C. under N₂ atmosphere. The mixture was diluted with water (600 mL), extracted with EtOAc (600 mL*3). The organic layers were washed with water (1200 mL) and brine (800 mL), dried over anhydrous Na₂SO₄, filtered and concentrated. The residue which was purified by flash silica gel chromatography (ISCO®; Agela® Flash Column Silica-CS (330 g), Eluent of 20% Ethyl acetate/petroleum ether gradient @100 mL/min) to afford methyl (3R,4R)-4-hydroxy-3-(methoxymethyl)chromane-6-carboxylate 298-3.

MS (ESI) m/z 235.2 (M−18+H⁺)

¹H NMR (500 MHz, chloroform-d) δ 8.05 (d, J=2.0 Hz, 1H), 7.89 (dd, J=2.0, 8.5 Hz, 1H), 6.87 (d, J=8.5 Hz, 1H), 4.86 (t, J=3.5 Hz, 1H), 4.20-4.29 (m, 2H), 3.88 (s, 3H), 3.66-3.73 (m, 1H), 3.56-3.65 (m, 1H), 3.39 (s, 3H), 2.80 (d, J=4.0 Hz, 1H), 2.32-2.47 (m, 1H)

### Step C: (3R,4S)-3-(methoxymethyl)-4-((4-nitrobenzoyl)oxy)chromane-6-carboxylate 298-4

4-nitrobenzoic acid (37.1 g, 222 mmol), triphenylphosphane (116 g, 444 mmol) and di-tert-butyl azodicarboxylate (102 g, 444 mmol) were added to a solution of methyl (3R,4R)-4-hydroxy-3-(methoxymethyl)chromane-6-carbxylate 298-3 (56 g, 222 mmol) in THF (600 mL) at 0° C. under N₂ atmosphere. The mixture was stirred at 25° C. for 1.5 h. The solvent was evaporated under reduced pressure to give the crude product. The crude product was purified by flash silica gel chromatography (ISCO®; 330 g SepaFlash® Silica Flash Column, Eluent of 24% EtOAc/petroleum ether gradient @ 100 mL/min) to give methyl (3R,4S)-3-(methoxymethyl)-4-((4-nitrobenzoyl)oxy)chromane-6-carboxylate 298-4.

### Step D: methyl (3R,4S)-4-hydroxy-3-(methoxymethyl)chromane-6-carboxylate 298-5

K₂CO₃ (59.6 g, 431 mmol) was added to a solution of methyl (3R,4S)-3-(methoxymethyl)-4-((4-nitrobenzoyl)oxy)chromane-6-carboxylate 298-4 (86.5 g, 216 mmol) in MeOH (500 mL) and CH₂Cl₂ (300 mL) at 25° C. under N₂ atmosphere. The mixture was stirred at 25° C. for 12 h. The mixture was diluted with water (600 mL), extracted with DCM (800 mL×3), dried over Na₂SO₄, and filtered. The solvent was evaporated under reduced pressure. The residue was purified by flash silica gel chromatography (ISCO®; 330 g Sepa Flash® Silica Flash Column, Eluent of 30% EtOAc/petroleum ether gradient @ 100 mL/min) to give methyl (3R,4S)-4-hydroxy-3-(methoxymethyl)chromane-6-carboxylate 298-5.

MS (ESI) m/z 253.1 (M+H⁺)

¹H NMR (500 MHz, chloroform-d) δ 8.12 (d, J=2.0 Hz, 1H), 7.87 (dd, J=2.5, 8.5 Hz, 1H), 6.83-6.89 (m, 1H), 4.69-4.75 (m, 1H), 4.33 (dd, J=3.0, 11.0 Hz, 1H), 4.09-4.13 (m, 1H), 3.88 (s, 3H), 3.44-3.50 (m, 1H), 3.39-3.43 (m, 1H), 3.37 (s, 3H), 2.68 (d, J=5.0 Hz, 1H), 2.31-2.33 (m, 1H).

### Step E: methyl (3R,4R)-4-((E)-2-((tert-butoxycarbonyl)imino)-4,4-diethyl-6-oxotetrahydropyrimidin-1(2H)-yl)-3-(methoxymethyl)chromane-6-carboxylate 298-6

Di-tert-butyl azodicarboxylate (63.9 g, 277 mmol) was added to a solution of methyl (3R,4S)-4-hydroxy-3-(methoxymethyl)chromane-6-carboxylate 298-5 (35 g, 139 mmol), tert-butyl (E)-(4,4-diethyl-6-oxotetrahydropyrimidin-2(1H)-ylidene)carbamate (37.4 g, 139 mmol) and Bu₃P (56.1 g, 277 mmol) in THF (500 mL) at 0° C. under N₂ atmosphere. Then the mixture was stirred at 25° C. for 30 min. The mixture was concentrated in vacuo and purified by column chromatography (petroleum ether/EtOAc=50:1-5:1) to give a crude product, which was dissolved in pet. ether (800 mL), stirred and filtered. The filtrate was concentrated to afford product methyl (3R,4R)-4-((E)-2-((tert-butoxycarbonyl)imino)-4,4-diethyl-6-oxotetrahydropyrimidin-1(2H)-yl)-3-(methoxymethyl) chromane-6-carboxylate 298-6.

MS (ESI) m/z 504.5 (M+H⁺)

1H NMR (500 MHz, chloroform-d) δ 7.76-7.85 (m, 1H), 7.68 (d, J=2.0 Hz, 1H), 6.87 (d, J=8.5 Hz, 1H), 6.48 (d, J=7.5 Hz, 1H), 4.44-4.57 (m, 1H), 4.31-4.32 (m, 1H), 3.83 (s, 3H), 3.57-3.58 (m, 1H), 3.47-3.48 (m, 1H), 3.27 (s, 3H), 2.66-2.78 (m, 1H), 2.41-2.53 (m, 2H), 1.63-1.71 (m, 4H), 1.47 (s, 9H), 0.93-0.96 (m, 6H)

### Step F: (3R,4R)-4-((E)-2-((tert-butoxycarbonyl)imino)-4,4-diethyl-6-oxotetrahydropyrimidin-1(2H)-yl)-3-(methoxymethyl)chromane-6-carboxylic acid 298-7

Potassium trimethylsilanolate (28.1 g, 219 mmol) was added to a solution of methyl (3R,4R)-4-((E)-2-((tert-butoxycarbonyl)imino)-4,4-diethyl-6-oxotetrahydropyrimidin-1(2H)-yl)-3-(methoxymethyl)chromane-6-carboxylate 298-6 (23 g, 36.5 mmol) in THF (400 mL) at 0° C. under N₂ atmosphere. The mixture was stirred at 25° C. for 1 h. (3R,4R)-4-((E)-2-((tert-butoxycarbonyl)imino)-4,4-diethyl-6-oxotetrahydropyrimidin-1(2H)-yl)-3-(methoxymethyl)chromane-6-carboxylic acid 298-7 in THF was used for the next step directly without further purification.

MS (ESI) m/z: 490.3 (M+H⁺)

### Step G: tert-butyl ((E)-4,4-diethyl-1-((3R,4R)-6-(((3S,4R)-3-hydroxy-2,2-dimethylchroman-4-yl)carbamoyl)-3-(methoxymethyl)chroman-4-yl)-6-oxotetrahydropyrimidin-2(1H)-ylidene)carbamate 298-8

DIEA (31.2 mL, 179 mmol) was added to a solution of (3R,4R)-4-((E)-2-((tert-butoxycarbonyl)imino)-4,4-diethyl-6-oxotetrahydropyrimidin-1(2H)-yl)-3-(methoxymethyl)chromane-6-carboxylic acid 298-7 (17.5 g, 35.7 mmol), EDCI (20.56 g, 107 mmol), HOBt (7.25 g, 53.6 mmol) and (3S,4R)-4-amino-2,2-dimethylchroman-3-ol (7 g, 36.2 mmol) in THF (600 mL). The reaction was stirred at 25° C. for 2 h. The mixture was quenched with water (400 mL) and extracted with EtOAc (500 mL*3). The organic layers were washed with brine (600 mL), dried over Na₂SO₄, filtered, and concentrated in vacuo. The crude was purified by flash silica gel chromatography (ISCO®; 330 g Sepa Flash® Silica Flash Column, Eluent of 40% EtOAc/petroleum ether gradient @ 100 mL/min) to give tert-butyl ((E)-4,4-diethyl-1-((3R,4R)-6-(((3S,4R)-3-hydroxy-2,2-dimethylchroman-4-yl)carbamoyl)-3-(methoxymethyl)chroman-4-yl)-6-oxotetrahydropyrimidin-2(1H)-ylidene)carbamate 298-8.

MS (ESI) m/z 665.4 (M+H⁺)

¹H NMR (400 MHz, chloroform-d) δ 10.17 (s, 1H), 7.61 (d, J=1.2 Hz, 1H), 7.45-7.47 (m, 1H), 7.27-7.30 (m, 1H), 7.15-7.25 (m, 1H), 6.96 (t, J=7.34 Hz, 1H), 6.52 (br d, J=6.8 Hz, 1H), 6.34 (br d, J=7.2 Hz, 1H), 5.17 (br t, J=8.0 Hz, 1H), 4.76 (br d, J=1.6 Hz, 1H), 4.50 (t, J=10.4 Hz, 1H), 4.32 (br dd, J=4.0, 10.6 Hz, 1H), 3.69-3.78 (m, 1H), 3.58 (dd, J=4.8, 9.6 Hz, 1H), 3.44-3.53 (m, 1H), 3.27 (s, 3H), 2.69-2.78 (m, 1H), 2.43-2.55 (m, 2H), 1.63-1.69 (m, 4H), 1.53 (s, 9H), 1.29 (d, J=5.2 Hz, 6H), 0.89-1.00 (m, 6H)

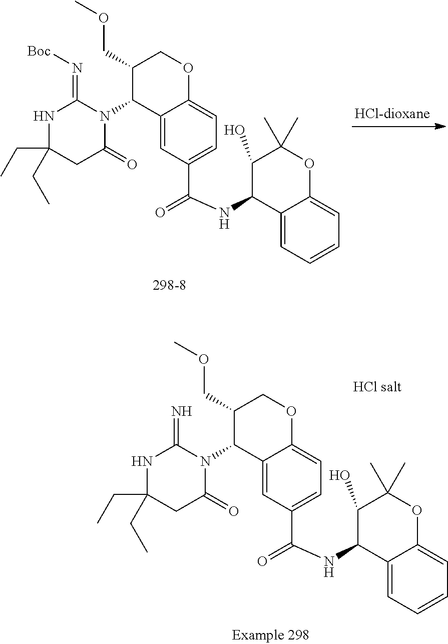 [SMILES: CCC1(CC)CC(=O)N([C@H]2c3cc(C(=O)N[C@@H]4c5ccccc5OC(C)(C)[C@H]4O)ccc3OC[C@H]2COC)/C(=N/C(=O)OC(C)(C)C)N1 ; CCC1(CC)CC(=O)N([C@H]2c3cc(C(=O)N[C@@H]4c5ccccc5OC(C)(C)[C@H]4O)ccc3OC[C@H]2COC)C(=N)N1]

### Step H: product (3R,4R)-4-(4,4-diethyl-2-imino-6-oxotetrahydropyrimidin-1(2H)-yl)-N-((3S,4R)-3-hydroxy-2,2-dimethylchroman-4-yl)-3-(methoxymethyl)chromane-6-carboxamide Example 298

A solution of tert-butyl ((E)-4,4-diethyl-1-((3R,4R)-6-(((3S,4R)-3-hydroxy-2,2-dimethylchroman-4-yl)carbamoyl)-3-(methoxymethyl)chroman-4-yl)-6-oxotetrahydropyrimidin-2(1H)-ylidene)carbamate 298-8 (16.8 g, 25.3 mmol) in HCl-dioxane (4 N) (500 mL) was stirred at 25° C. for 12 hrs. The mixture was concentrated under reduced pressure, and the residue was purified by Prep-HPLC (Instrument L-Y Method Column YMC Exphere C18 250*50 mm*10 um Condition water (0.05% HCl)-ACN Begin B 10 End B 50 Gradient Time (min) 20 100% B Hold Time (min) 3 Flow Rate (mL/min) 120 Injections 9) to afford product (3R,4R)-4-(4,4-diethyl-2-imino-6-oxotetrahydropyrimidin-1(2H)-yl)-N-((3S,4R)-3-hydroxy-2,2-dimethylchroman-4-yl)-3-(methoxymethyl)chromane-6-carboxamide Example 298.

MS (ESI) m/z 565.2 (M+H⁺)

¹H NMR (500 MHz, methanol-d₄) δ 8.55 (d, J=8.5 Hz, 1H), 7.77 (dd, J=2.0, 9.0 Hz, 1H), 7.74 (d, J=2.0 Hz, 1H), 7.10-7.16 (m, 2H), 6.91 (d, J=8.5 Hz, 1H), 6.83-6.88 (m, 1H), 6.76 (d, J=8.5 Hz, 1H), 5.39 (d, J=6.0 Hz, 1H), 5.20-5.28 (m, 1H), 4.29-4.38 (m, 1H), 4.24-4.25 (m, 1H), 3.78 (d, J=10.0 Hz, 1H), 3.58-3.60 (m, 1H), 3.47-3.53 (m, 1H), 3.40 (s, 3H), 2.88-2.96 (m, 1H), 2.83 (d, J=16.0 Hz, 1H), 2.63 (d, J=16.0 Hz, 1H), 1.56-1.77 (m, 4H), 1.48 (s, 3H), 1.27 (s, 3H), 0.97 (t, J=8.0 Hz, 3H), 0.92 (t, J=7.5 Hz, 3H)

TABLE 4

| Ex- | | LC/MS | |
|---|---|---|---|
| ample | Structure | (M + 1)⁺ | Name |
| 299 | 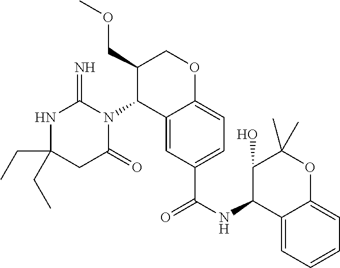 [SMILES: CCC1(CC)CC(=O)N([C@H]2c3cc(C(=O)N[C@@H]4c5ccccc5OC(C)(C)[C@H]4O)ccc3OC[C@@H]2COC)C(=N)N1] | 565.3 | (3S,4R)-4-(4,4-diethyl-2- imino-6-oxo- hexahydropyrimidin-1-yl)-N- [(3S,4R)-3-hydroxy-2,2- dimethyl-chroman-4-yl]-3- (methoxymethyl)chromane-6- carboxamide |
| 300 | 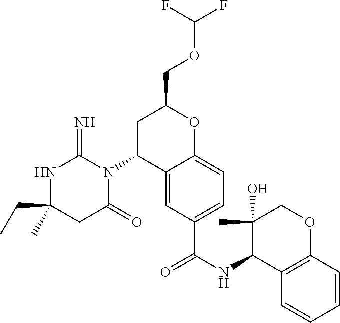 [SMILES: CC[C@]1(C)CC(=O)N([C@@H]2C[C@@H](COC(F)F)Oc3ccc(C(=O)N[C@@H]4c5ccccc5OC[C@@]4(C)O)cc32)C(=N)N1] | 573.3 | (2S,4R)-2- (difluoromethoxymethyl)-4- [(4R)-4-ethyl-2-imino-4- methyl-6-oxo- hexahydropyrimidin-1-yl]-N- [(3S,4R)-3-hydroxy-3-methyl- chroman-4-yl]chromane-6- carboxamide |
| 301 | 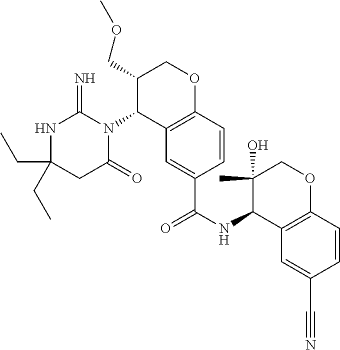 [SMILES: CCC1(CC)CC(=O)N([C@H]2c3cc(C(=O)N[C@@H]4c5cc(C#N)ccc5OC[C@@]4(C)O)ccc3OC[C@H]2COC)C(=N)N1] | 576.2 | (3R,4R)-N-[(3S,4R)-6-cyano- 3-hydroxy-3-methyl-chroman- 4-yl]-4-(4,4-diethyl-2-imino-6- oxo-hexahydropyrimidin-1- yl)-3- (methoxymethyl)chromane-6- carboxamide |
| 302 | 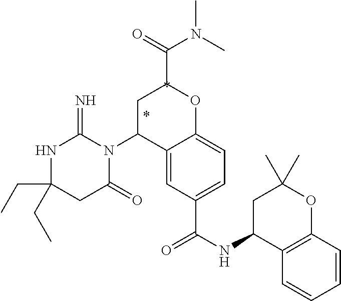 [SMILES: CCC1(CC)CC(=O)N(C2CC(C(=O)N(C)C)Oc3ccc(C(=O)N[C@H]4CC(C)(C)Oc5ccccc54)cc32)C(=N)N1] | 576.4 | 4-(4,4-diethyl-2-imino-6-oxo- hexahydropyrimidin-1-yl)-N6- [(4S)-2,2-dimethylchroman-4- yl]-N2,N2-dimethyl- chromane-2,6-dicarboxamide |
| 303 | 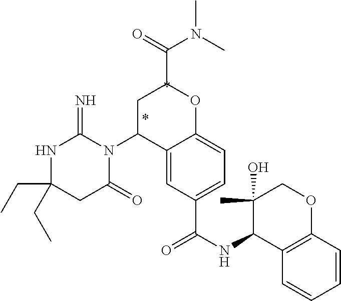 [SMILES: CCC1(CC)CC(=O)N(C2CC(C(=O)N(C)C)Oc3ccc(C(=O)N[C@@H]4c5ccccc5OC[C@@]4(C)O)cc32)C(=N)N1] | 578.3 | 4-(4,4-diethyl-2-imino-6-oxo- hexahydropyrimidin-1-yl)-N6- [(3S,4R)-3-hydroxy-3-methyl- chroman-4-yl]-N2,N2- dimethyl-chromane-2,6- dicarboxamide |
| 304 | 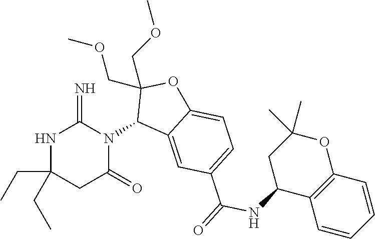 [SMILES: CCC1(CC)CC(=O)N([C@H]2c3cc(C(=O)N[C@H]4CC(C)(C)Oc5ccccc54)ccc3OC2(COC)COC)C(=N)N1] | 579.3 | (3S)-3-(4,4-diethyl-2-imino-6- oxo-hexahydropyrimidin-1- yl)-N-[(4S)-2,2- dimethylchroman-4-yl]-2,2- bis(methoxymethyl)-3H- benzofuran-5-carboxamide |
| 305 | 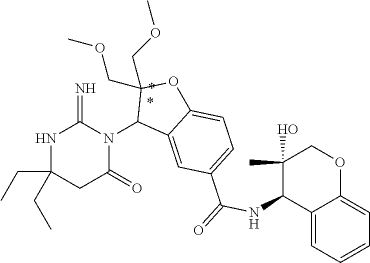 [SMILES: CCC1(CC)CC(=O)N(C2c3cc(C(=O)N[C@@H]4c5ccccc5OC[C@@]4(C)O)ccc3OC2(COC)COC)C(=N)N1] | 581.3 | 3-(4,4-diethyl-2-imino-6-oxo- hexahydropyrimidin-1-yl)-N- [(3S,4R)-3-hydroxy-3-methyl- chroman-4-yl]-2,2- bis(methoxymethyl)-3H- benzofuran-5-carboxamide |
| 306 | 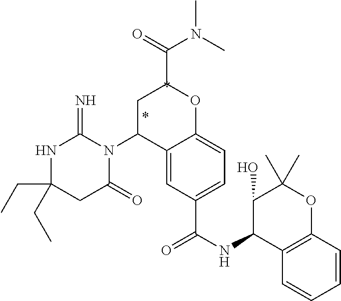 [SMILES: CCC1(CC)CC(=O)N(C2CC(C(=O)N(C)C)Oc3ccc(C(=O)N[C@@H]4c5ccccc5OC(C)(C)[C@H]4O)cc32)C(=N)N1] | 592.4 | 4-(4,4-diethyl-2-imino-6-oxo- hexahydropyrimidn-1-yl)-N6- [(3S,4R)-3-hydroxy-2,2- dimethyl-chroman-4-yl]- N2,N2-dimethyl-chromane- 2,6-dicarboxamide |
| 307 | 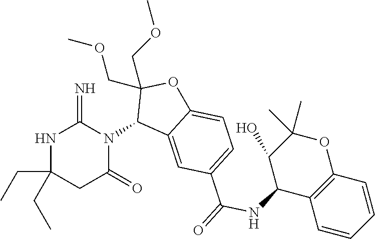 [SMILES: CCC1(CC)CC(=O)N([C@H]2c3cc(C(=O)N[C@@H]4c5ccccc5OC(C)(C)[C@H]4O)ccc3OC2(COC)COC)C(=N)N1] | 595.3 | (3S)-3-(4,4-diethyl-2-imino-6- oxo-hexahydropyrimidin-1- yl)-N-[(3S,4R)-3-hydroxy-2,2- dimethyl-chroman-4-yl]-2,2- bis(methoxymethyl)-3H- benzofuran-5-carboxamide |
| 308 | 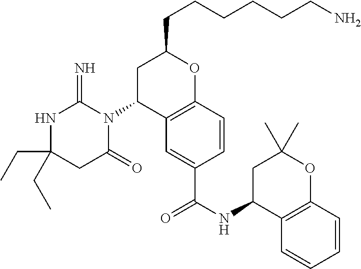 [SMILES: CCC1(CC)CC(=O)N([C@@H]2C[C@@H](CCCCCCN)Oc3ccc(C(=O)N[C@H]4CC(C)(C)Oc5ccccc54)cc32)C(=N)N1] | 604.4 | (2R,4R)-2-(6-aminohexyl)-4- (4,4-diethyl-2-imino-6-oxo- hexahydropyrimidin-1-yl)-N- [(4S)-2,2-dimethylchroman-4- yl]chromane-6-carboxamide |
| 309 | 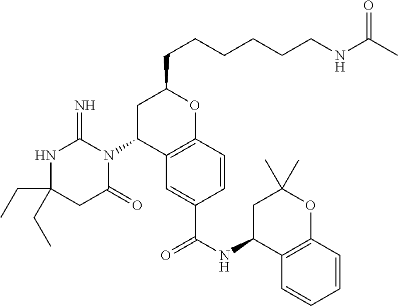 [SMILES: CCC1(CC)CC(=O)N([C@@H]2C[C@@H](CCCCCCNC(C)=O)Oc3ccc(C(=O)N[C@H]4CC(C)(C)Oc5ccccc54)cc32)C(=N)N1] | 646.3 | (2R,4R)-2-(6- acetamidohexyl)-4-(4,4- diethyl-2-imino-6-oxo- hexahydropyrimidin-1-yl)-N- [(4S)-2,2-dimethylchroman-4- yl]chromane-6-carboxamide |
| 310 | 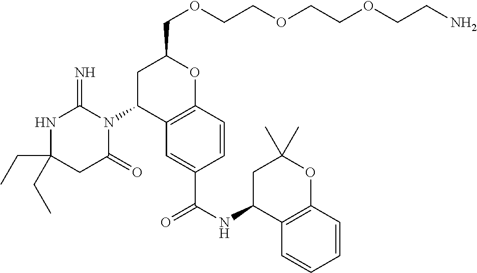 [SMILES: CCC1(CC)CC(=O)N([C@@H]2C[C@@H](COCCOCCOCCN)Oc3ccc(C(=O)N[C@H]4CC(C)(C)Oc5ccccc54)cc32)C(=N)N1] | 666.5 | (2S,4R)-2-[2-[2-(2- aminoethoxy)ethoxy]ethoxy- methyl]-4-(4,4-diethyl-2- imino-6-oxo-hexahydro- pyrimidin-1-yl)-N-[(4S)- 2,2-dimethylchroman-4- yl]chromane-6-carboxamide |
| 311 | 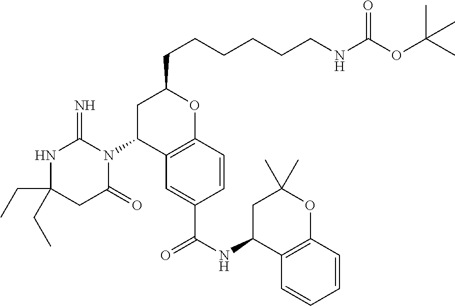 [SMILES: CCC1(CC)CC(=O)N([C@@H]2C[C@@H](CCCCCCNC(=O)OC(C)(C)C)Oc3ccc(C(=O)N[C@H]4CC(C)(C)Oc5ccccc54)cc32)C(=N)N1] | 704.5 | tert-butyl N-[6-[(2R,4R)-4- (4,4-diethyl-2-imino-6-oxo- hexahydropyrimidin-1-yl)-6- [[(4S)-2,2-dimethylchroman- 4-yl]carbamoyl]chroman-2- yl]hexyl]carbamate |
| 312 | 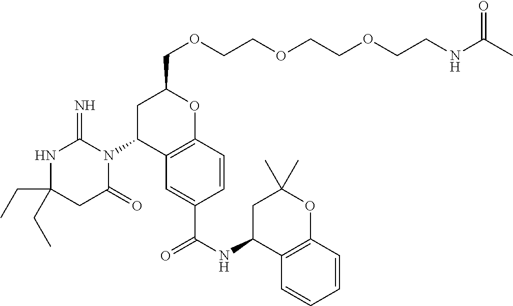 [SMILES: CCC1(CC)CC(=O)N([C@@H]2C[C@@H](COCCOCCOCCNC(C)=O)Oc3ccc(C(=O)N[C@H]4CC(C)(C)Oc5ccccc54)cc32)C(=N)N1] | 708.5 | (2S,4R)-2-[2-[2-(2- acetamidoethoxy)ethoxy] ethoxymethyl]-4-(4,4- diethyl-2-imino-2-oxo- hexahydropyrimidin-1-yl)-N- [(4S)-2,2-dimethylchroman-4- yl]chromane-6-carboxamide |
| 313 | 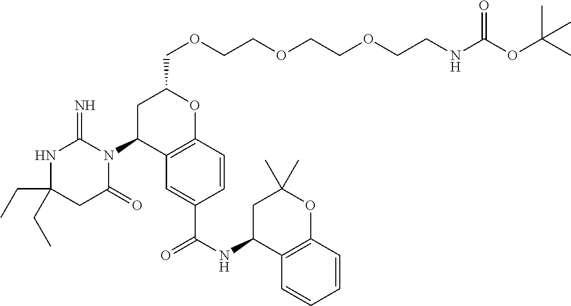 [SMILES: CCC1(CC)CC(=O)N([C@H]2C[C@H](COCCOCCOCCNC(=O)OC(C)(C)C)Oc3ccc(C(=O)N[C@H]4CC(C)(C)Oc5ccccc54)cc32)C(=N)N1] | 766.5 | tert-butyl N-[2-[2-[2- [[(2R,4S)-4-(4,4-diethyl-2- imino-6-oxo- hexahydropyrimidin-1-yl)-6- [[(4S)-2,2-dimethylchroman-4- yl]carbamoyl]chroman-2- yl]methoxy]ethoxy]ethoxy] ethyl]carbamate |
| 314 | 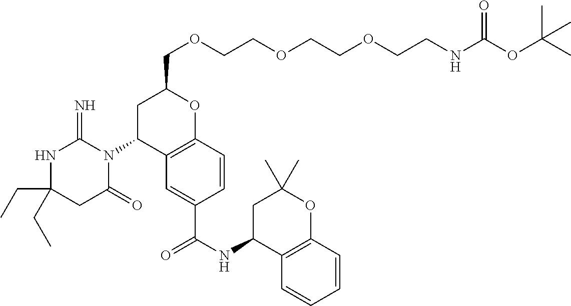 [SMILES: CCC1(CC)CC(=O)N([C@@H]2C[C@@H](COCCOCCOCCNC(=O)OC(C)(C)C)Oc3ccc(C(=O)N[C@H]4CC(C)(C)Oc5ccccc54)cc32)C(=N)N1] | 766.5 | tert-butyl N-[2-[2-[2- [[(2S,4R)-4-(4,4-diethyl-2- imino-6-oxo- hexahydropyrimidin-1-yl)-6- [[(4S)-2,2-dimethylchroman-4- yl]carbamoyl]chroman-2- yl]methoxy]ethoxy]ethoxy] ethyl]carbamate |

### EXAMPLES

### Assessing Antiparasite Potency in a Parasite LDH Growth Assay (Parasite Assay)

The parasite stock was maintained at 4% haematocrit in RPMI-Hepes media buffered with sodium bicarbonate and supplemented with 5% heat inactivated human serum and 0.5% albumax.

Approximately 42 hours prior to the potency assay being set up, parasites were synchronized with 5% sorbitol to select for ring stage parasites. On the day of assay set up, a blood smear of the parasite culture was Giemsa stained and counted. The parasitemia was adjusted to 0.7% rings and the haematocrit was diluted to 2% in RPMI-Hepes media buffered with sodium bicarbonate and supplemented with 5% heat inactivated human serum and 0.5% albumax. 30 ul of diluted parasites are then added into 10 ul of media+compound in pre-prepared Greiner TC assay plates. Parasite assay plates were placed in gassed humidified boxes in single layer and allowed to incubate at 37° C. for 72 hours. After 72 hours growth, assay plates are sealed with parafilm and frozen flat, in single file at −80° C. overnight. On the following day, assay plates are allowed to thaw at room temperature for 4 hours to which an LDH assay is performed to measure parasite growth.

Assay EC₅₀ results are shown in Table 5.

TABLE 5

| Example | EC₅₀ (nM) |
|---|---|
| 1A | 0.5 |
| 1B | 163 |
| 2A | 0.5 |
| 2B | 73 |
| 3A | 2.2 |
| 4A | 87 |
| 5A | 5.9 |
| 6A | 1.1 |
| 6B | 8.8 |
| 7A | 0.4 |
| 7B | 19 |
| 8A | 10 |
| 8B | 22 |
| 8C | 120 |
| 8D | 86 |
| 9A | 0.9 |
| 9B | 170 |
| 10A | 0.5 |
| 11A | 0.9 |
| 11B | 84 |
| 12A | 1.1 |
| 13A | 0.8 |
| 13B | 54 |
| 14A | 1 |
| 15A | 0.3 |
| 16A | 0.6 |
| 16B | 95 |
| 17A | 2.7 |
| 18A | 0.8 |
| 19A | 0.3 |
| 20A | 0.8 |
| 21A | 0.8 |
| 22A | 0.7 |
| 23A | 5 |
| 24A | 9.3 |
| 25A | 0.5 |
| 25B | 59.8 |
| 26A | 1.3 |
| 26B | 180 |
| 27A | 1.3 |
| 28A | 1.8 |
| 29A | 1 |
| 29B | 39.7 |
| 30A | 0.8 |
| 31A | 22 |
| 31B | 1 |
| 32A | 7.9 |
| 32B | 290 |
| 33A | 28.3 |
| 34A | 1.2 |
| 35A | 2.4 |
| 36A | 8.9 |
| 36B | 2.2 |
| 37A | 74.4 |
| 38A | 2.8 |
| 39A | 29.9 |
| 40A | 69.5 |
| 41A | 2.8 |
| 42A | 1.0 |
| 42B | 3.2 |
| 43A | 8.1 |
| 43B | 21 |
| 44A | 1.5 |
| 44B | 2.8 |
| 45A | 2.6 |
| 46A | 130 |
| 47A | 4.7 |
| 48A | 37.5 |
| 49A | 49 |
| 50A | 3.7 |
| 51A | 22.5 |
| 52A | 14.4 |
| 53A | 0.3 |
| 54A | 190 |
| 54B | 160 |
| 55A | 24.5 |
| 55B | 83.1 |
| 56A | 26.9 |
| 57A | 18.3 |
| 58A | 250 |
| 58B | 3.8 |
| 59A | 210 |
| 59B | 1.6 |
| 60A | 1.3 |
| 60B | 1.7 |
| 61A | 8.4 |
| 62A | 1.2 |
| 63A | 220 |
| 64A | 12 |
| 64B | 0.9 |
| 64C | 64 |
| 64D | 42 |
| 65A | 68 |
| 65B | 2.6 |
| 65C | 26 |
| 66A | 76 |
| 67A | 170 |
| 67B | 0.8 |
| 68A | 1.0 |
| 69A | 2.6 |
| 70A | 9.3 |
| 71A | 3.5 |
| 72A | 0.5 |
| 73A | 0.3 |
| 73B | 1.7 |
| 74A | 0.3 |
| 74B | 6.5 |
| 75A | 0.4 |
| 75B | 9.5 |
| 76A | 8.5 |
| 76B | 1.1 |
| 77A | 57.1 |
| 77B | 21.4 |
| 78 | 8.9 |
| 79 | 9.6 |
| 80 | 18.0 |
| 81 | 6.6 |
| 82 | 15.5 |
| 83 | 20.0 |
| 84 | 0.9 |
| 85 | 10.7 |
| 86 | 87.8 |
| 87 | 105 |
| 88 | 3.4 |
| 89 | 43.5 |
| 90 | 26.8 |
| 91 | 45.2 |
| 92 | 97.2 |
| 93 | 190 |
| 94 | 1.9 |
| 95 | 8.8 |
| 96 | 6.5 |
| 97 | 19.0 |
| 98 | 1.5 |
| 99 | 4.2 |
| 100 | 5.3 |
| 101 | 46.7 |
| 102 | 14.0 |
| 103 | 6.1 |
| 104 | 5.0 |
| 105 | 8.8 |
| 106 | 6.6 |
| 107 | 222 |
| 108 | 74.3 |
| 109 | 14.0 |
| 110 | 60.0 |
| 111 | 90.0 |
| 112 | 11.4 |
| 113 | 16.4 |
| 114 | 1.8 |
| 115 | 3.6 |
| 116 | 210 |
| 117 | 2.5 |
| 118 | 0.9 |
| 119 | 2.8 |
| 120 | 0.8 |
| 121 | 0.6 |
| 122 | 3.9 |
| 123 | 2.8 |
| 124 | 57.5 |
| 125 | 2.1 |
| 126 | 14.3 |
| 127A | 202 |
| 127B | 222 |
| 128 | 2.8 |
| 129 | 3.5 |
| 130 | 2.1 |
| 131 | 2.0 |
| 132 | 1.6 |
| 133A | 1.4 |
| 133B | 17.0 |
| 134 | 8.8 |
| 135 | 0.9 |
| 136 | 1.3 |
| 137 | 239 |
| 138 | 0.9 |
| 139 | 180 |
| 140A | 1.4 |
| 140B | 1.7 |
| 141 | 8.4 |
| 142 | 98.5 |
| 143 | 178 |
| 144 | 194 |
| 145 | 40.8 |
| 146 | 35.5 |
| 147 | 160 |
| 148 | 2.7 |
| 149 | 1.1 |
| 150 | 49.0 |
| 151 | 5.4 |
| 152 | 7.7 |
| 153 | 11.5 |
| 154 | 0.7 |
| 155 | 82.5 |
| 156 | 1.1 |
| 157 | 19.9 |
| 158 | 0.9 |
| 159 | 0.5 |
| 160 | 8.3 |
| 161 | 0.8 |
| 162 | 7.5 |
| 163 | 8.4 |
| 164 | 2.4 |
| 165 | 3.4 |
| 166 | 154 |
| 167 | 250 |
| 168 | 0.4 |
| 169 | 0.3 |
| 170 | 184 |
| 171 | 0.7 |
| 172 | 0.9 |
| 173 | 2.8 |
| 174 | 1.9 |
| 175 | 31.0 |
| 176A | 29.2 |
| 176B | 7.8 |
| 176C | 2.4 |
| 176D | 114 |
| 177 | 66.9 |
| 178A | 108 |
| 178B | 2.2 |
| 179 | 65.4 |
| 180 | 65.4 |
| 181A | 189 |
| 181B | 7.9 |
| 182 | 6.1 |
| 183 | 71.4 |
| 184 | 105 |
| 185 | 50.3 |
| 186 | 70.0 |
| 187 | 66.7 |
| 188 | 203 |
| 189 | 1.0 |
| 190A | 261 |
| 190B | 2.6 |
| 190C | 4.9 |
| 191 | 2.6 |
| 192 | 232 |
| 193A | 96.4 |
| 193B | 0.8 |
| 193C | 19.5 |
| 194 | 0.9 |
| 195 | 3.5 |
| 196 | 8.6 |
| 197 | 125 |
| 198 | 11.3 |
| 199 | 1.1 |
| 200 | 2.9 |
| 201 | 1.4 |
| 202 | 0.8 |
| 203 | 5.9 |
| 204 | 247 |
| 205 | 2.1 |
| 206 | 66.0 |
| 207 | 22.0 |
| 208 | 3.7 |
| 209 | 7.9 |
| 210 | 195 |
| 211 | 8.9 |
| 212 | 6.5 |
| 213 | 9.0 |
| 214A | 8.2 |
| 214B | 260 |
| 215 | 4.2 |
| 216 | 2.7 |
| 217 | 4.9 |
| 218 | 64 |
| 219 | 208 |
| 220 | 2.9 |
| 221 | 190 |
| 222 | 15.6 |
| 223 | 300 |
| 224 | 0.3 |
| 225 | 0.8 |
| 226A | 2.6 |
| 226B | 69.0 |
| 226C | 273 |
| 226D | 1.0 |
| 227 | 1.3 |
| 228 | 282 |
| 229 | 59.3 |
| 230 | 2.7 |
| 231 | 7.4 |
| 232 | 21.8 |
| 233 | 7.9 |
| 234A | 22.5 |
| 234B | 3.9 |
| 234C | 43.2 |
| 235 | 0.7 |
| 236 | 0.9 |
| 237 | 240 |
| 238 | 47.8 |
| 239 | 0.9 |
| 240 | 0.6 |
| 241 | 0.4 |
| 242A | 0.6 |
| 242B | 10.5 |
| 243 | 6.1 |
| 244 | 7.8 |
| 245 | 0.8 |
| 246 | 2.0 |
| 247 | 4.5 |
| 248 | 0.8 |
| 249 | 1.1 |
| 250 | 219 |
| 251A | 0.4 |
| 251B | 1.5 |
| 251C | 84.4 |
| 252 | 2.1 |
| 253 | 4.3 |
| 254 | 3.4 |
| 255 | 3.3 |
| 256 | 8.9 |
| 257 | 189 |
| 258A | 85.9 |
| 258B | 2.4 |
| 258C | 226 |
| 258D | 6.7 |
| 259A | 5.5 |
| 259B | 24.2 |
| 259C | 3.6 |
| 259D | 10.6 |
| 260 | 8.2 |
| 261 | 133 |
| 262 | 7.4 |
| 263 | 74.8 |
| 264 | 3.3 |
| 265 | 31.1 |
| 266 | 2.6 |
| 267 | 24.0 |
| 268 | 25.0 |
| 269 | 2.9 |
| 270 | 1.8 |
| 271 | 182 |
| 272A | 62.5 |
| 272B | 14.3 |
| 272C | 0.9 |
| 272D | 2.3 |
| 273 | 7.8 |
| 274 | 98.5 |
| 275 | 25.7 |
| 276 | 0.6 |
| 277 | 0.4 |
| 278 | 12.2 |
| 279 | 31.4 |
| 28 | 0.4 |
| 281 | 1.1 |
| 282A | 7.8 |
| 282B | 40.5 |
| 283 | 2.6 |
| 284 | 8.5 |
| 285 | 35.5 |
| 286 | 240 |
| 287 | 1.9 |
| 288 | 1.8 |
| 289 | 0.6 |
| 290 | 0.3 |
| 291 | 240 |
| 292 | 49.7 |
| 293 | 2.8 |
| 294 | 259 |
| 295 | 100 |
| 296 | 0.3 |
| 297 | 241 |
| 298 | 0.5 |
| 299 | 0.8 |
| 300 | 3.8 |
| 301 | 1.8 |
| 302 | 1.7 |
| 303 | 50.8 |
| 304 | 5.6 |
| 305 | 75.8 |
| 306 | 27.4 |
| 307 | 3.2 |
| 308 | 40.5 |
| 309 | 3.2 |
| 310 | 211 |
| 311 | 2.4 |
| 312 | 53.9 |
| 313 | 260 |
| 314 | 7.3 |

### Example 1A as an Inhibitor of PMIX and PMX

To confirm that Example 1A blocks PMX function in the P. falciparum parasite, the ability to inhibit cleavage of a known substrate for this protease was tested. SERA5 is a 120 kDa protein required for merozoite egress and is processed by subtilisin-like protease subtilisin 1 (SUB1) to release a soluble polypeptide of approximately 50 kDa (Pino, P., Caldelari, R., Mukherjee, B., Vahokoski, J., Klages, N., Maco, B., et al. (2017). A multistage antimalarial targets the plasmepsins IX and X essential for invasion and egress. Science 358, 522-528). The protease inhibitor E64, which prevents schizont rupture, but does not affect SERA5 processing was used as a negative control (Salmon, B. L., Oksman, A. and Goldberg, D. E. (2001). Malaria parasite exit from the host erythrocyte: A two-step process requiring extraerythrocytic proteolysis. Proc Natl Acad Sci USA 98, 271-276). However, following incubation with Example 1A, there was an accumulation of unprocessed SERA5 at 120 kDa confirming that SUB1 activation requires prior processing. SERA5 was included as a control in all subsequent experiments as a proxy for PMX-mediated activation of SUB1.

To confirm that Example 1A was a dual inhibitor of PMIX and PMX function, the ability of this compound to inhibit cleavage of the known PMIX substrate Rhoptry Associated Protein 1 (RAPI) in parasite extracts was tested (Pino et al., 2017). RAP1 is a merozoite rhoptry protein that is localized to the parasitophorous vacuole after invasion and processed by both PMIX and SUB1 (Pino et al., 2017). The processed forms of RAP1 (p82 and p69) are detected in untreated and E64-treated merozoites, showing that this protein was processed normally by PMIX and SUB1 under these conditions. RAP1 was not released into the parasite supernatant because it was deposited in the parasitophorous vacuole during the invasion process (Baldi, D., Andrews, K., Waller, R., Roos, D., Howard, R., Crabb, B. and Cowman, A. (2000)). RAP1 controls rhoptry targeting of RAP2 in the malaria parasite Plasmodium falciparum. Embo Journal 19, 2435-2443. In parasites treated with Example 1A only the unprocessed p87 form of RAP1 was present, indicating that both SUB1 and PMIX cleavage have been blocked. Similar results were obtained for Apical Sushi Protein (ASP) which has also been shown to be cleaved by PMIX (Pino et al., 2017). ASP was processed into a 47 kDa polypeptide (p47) in untreated and E64-treated parasites. However, this cleavage event was inhibited by Example 1A and the full-length protein of 87 kDa (p87) was observed, indicating that PMIX processing of this protein was blocked by Example 1A. These findings confirm that Example 1A acts as a dual inhibitor and blocks both PMX and PMIX protease activity in the P. falciparum parasite.

The development of Example 1A as a PMIX/PMX dual inhibitor of the P. falciparum parasite has allowed for the analysis of the function of these aspartic acid proteases. To do this, PMIX was processed to a 55 kDa (p55) protein and this was not inhibited by E64. Example 1 was used to inhibit this processing event, indicating that autocatalytic cleavage of PMIX was important for activation of this protease. The development of a PMX-specific inhibitor and a PMX/PIX dual inhibitor confirms the PMIX specific processing of ASP and RAP1, and also shows that PMIX was autocatalytically processed and activated.

### Chemovaccination of Mice

### General Experimental Procedure

40,000 PbmCherryLuci sporozoites (or the indicated number of sporozoites) were inoculated i.v. and compounds were administered by oral gavage at indicated time points during liver infection. Liver infection levels and egress of parasites from the liver were measured by bioluminescence signal from the parasites using an In Vivo Imaging System (IVIS, Perkin Elmer) at the peak of liver infection (52 hours post infection, hpi), during liver egress (55 hpi) as well as during the first round of blood infection (65 hpi). Initiation of blood infection was also measured by flow cytometry at 65 hpi using the mCherry reporter to determine the parasitemia of this first round of blood infection. These analyses allowed quantification of the efficacy of drug killing of parasites in the liver (52 hpi), preventing their egress from the liver (55 hpi) or preventing their successful infection of the blood (65 hpi). Mice were monitored by giemsa stained thin blood smears for the presence of parasites in the blood for 30 days post infection. If no blood infection was seen during the subsequent 30 days, mice were declared cured of malaria infection.

Chemovaccination of Mice with Example 1A

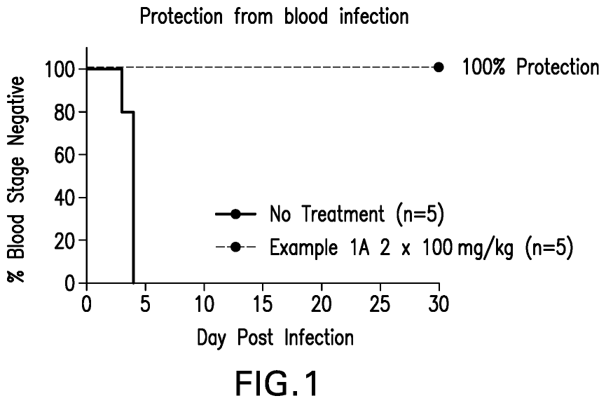

Mice were infected with 40,000 P. berghei sporozoites expressing mCherry and Luciferase (PbmCherryLuci) and treated with Example 1A as described above. Example IA was dissolved in vehicle and mice were treated twice at the indicated doses 12 hours apart (36 and 48 hours) during liver infection. As shown in FIG. 1, the treatment cured mice of infection, demonstrating chemoprotection and the prevention of the subsequent blood infection. This chemoprotection could also be considered an immunization, since the parasites are arrested in the latest stages of liver development and egress thereby allowing an immune response to develop.

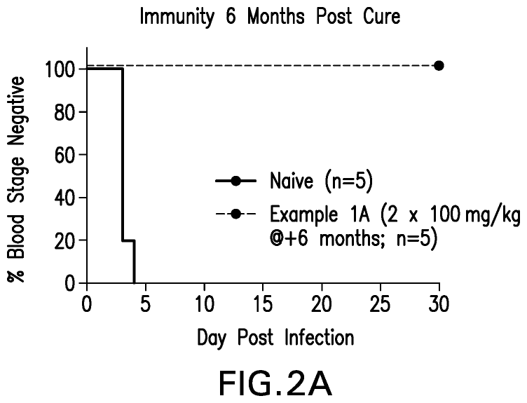

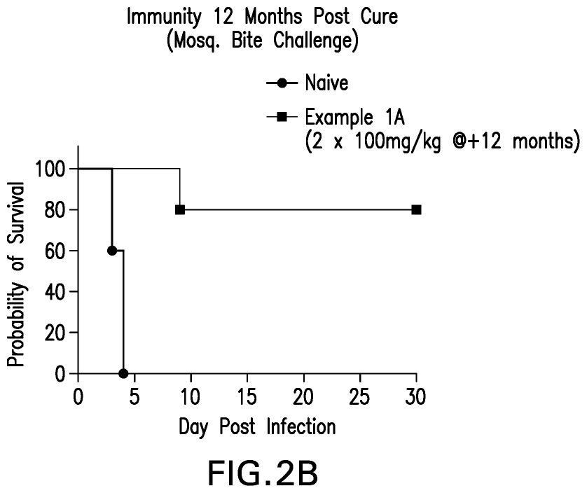

Previously chemo-protected mice were rechallenged with PbmCherryLuci 6 months later by mosquito bite infection. As shown in FIG. 2A, all mice were subsequently immune to this reinfection challenge and were completely protected from developing malaria symptoms. As shown in FIG. 2B, these mice were again challenged by mosquito bite infection 12 months after chemoprotected. Four of five mice were again fully protected one year later, while the fifth mouse showed immunity as evidenced by a multi-day delay to the appearance of parasites in the blood. In both experiments naïve, sex matched control mice all became infected as expected. Thus in addition to chemoprotection, Example IA could possibly act as a chemovaccination to protect against future infections for one year or more.

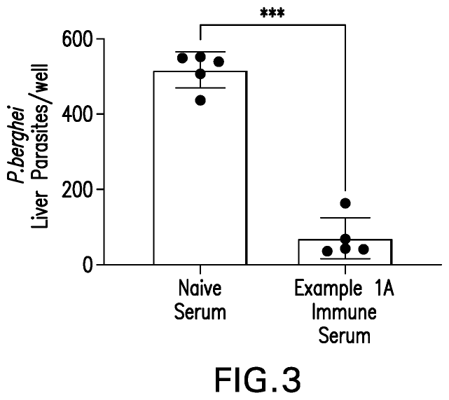

These data show that inhibition of both PMIX/X can induce long lasting, sterile immunity to a homologous parasite challenge. That challenge of chemovaccinated mice by mosquito bites did not result in liver infection and implicates antibody and T-cell responses that protect the host against sporozoite infection of the liver. Indeed, serum from protected mice contained antibodies that cross-reacted with the surface of Plasmodium sporozoites and also reacted with parasites residing within infected liver cells, suggesting that the mechanism of protection involves anti-parasitic antibodies. To demonstrate this, immune serum from five female BALB/c mice immunised with Example 1A as above ten months prior were analysed using an in vitro infection assay that measures the ability of antibodies to inhibit infection. To initiate the in vitro infection assay, 30,000 HepG2 human hepatoma cells were seeded into a 96-well plate format the day before infection. The day of infection, freshly dissected PbmCherryLuci salivary gland sporozoites were incubated with a 1 in 20 dilution of either naïve BALB/c serum (from n=5 mice) or immune serum (from n=5 mice) in culture media and added to HepG2 wells in technical triplicate. Serum samples were not pooled, so the response from five individual mice was analysed independently. Forty-eight hours after infection the infected cells were fixed with 4% formaldehyde and imaged using an Opera Phenix High Content Imager (PerkinElmer). The number of parasites in each well was counted using the mCherry reporter expressed by these parasites, the average number of parasites between the technical triplicates averaged for each serum sample, and the average for each mouse presented. As shown in FIG. 3, there was inhibition of infection using the serum from each of the five immunised mice compared to naïve mice, demonstrating that chemovaccination induces a functional antibody response capable of significantly inhibiting sporozoite infection of liver cells.

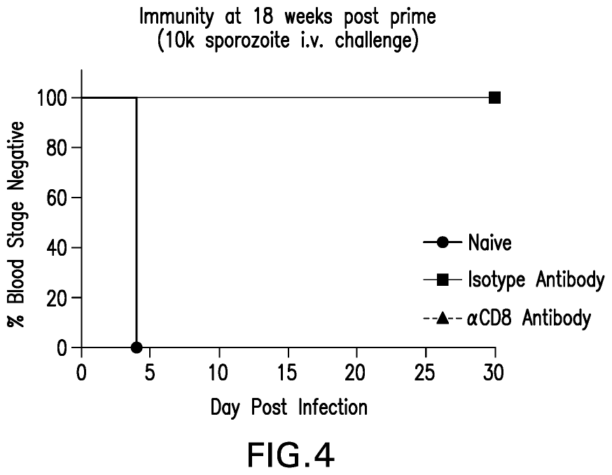

In addition to these protective antibody responses, the contribution of CD8 T-cells to the mechanism of immune protection was tested. Female C57BL/6 mice were immunized using a prime/boost immunisation regimen. The first immunisation was with 40,000 PbmCherryLuci salivary gland sporozoites i.v. followed by two doses of Example 1A at 100 mg/kg P.O. (given at 36 and 48 hours post infection). The second immunisation (boost) was eight weeks later, this time with 20,000 PbmCherryLuci sporozoites but with Example IA treatment as for the prime immunisation. Ten weeks after the boost immunisation (eighteen weeks after the prime immunisation) the mice were injected with 150 μg of a CD8 depleting antibody, or an isotype control non-depleting antibody the day before the mice were challenged with the bites of 10 PbmCherryLuci infected mosquitoes for 15 minutes. Giemsa stained thin blood smears were analysed until 30 days after challenge for the presence of parasites in the blood and mice were considered to be protected from infection if no blood parasites were observed within the 30 days post intravenous sporozoite challenge. As shown in FIG. 4, all naïve mice developed blood infection 4 days after challenge. The mice that received the CD8 depleting antibody likewise all had blood parasites 4 days after challenge, whereas the mice that received the isotype control non-depleting antibody were negative for blood parasites throughout the 30-day observation period. These data show that CD8 T-cells are required for sterile immunity against reinfection 4 months later, and furthermore demonstrate the chemovaccination effect in a second mouse strain. Combined, these data show that chemovaccination with Example 1A acting at this late stage of liver development (liver to blood transition) induces strong antibody and T-cell responses that protect mice from reinfection.

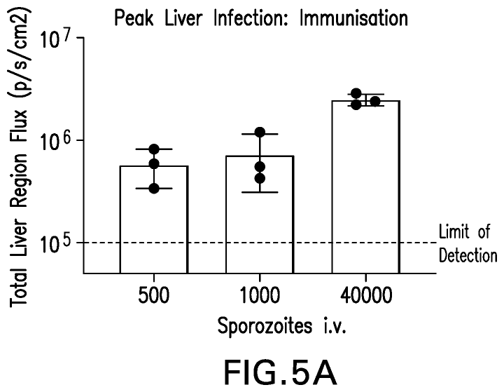

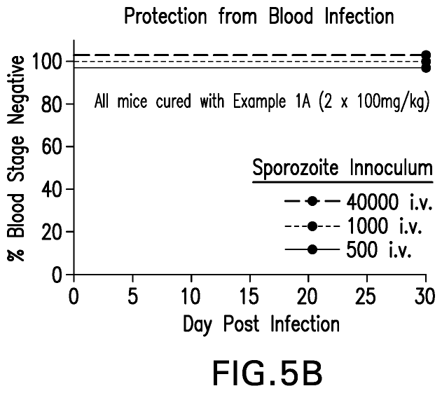

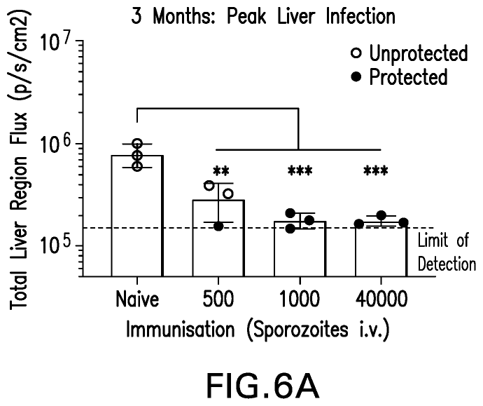

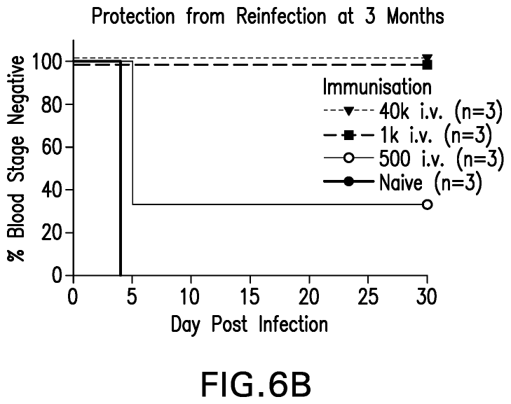

To investigate how many sporozoites are required to develop immunity, female BALB/c mice (n=3/group) were infected intravenously (i.v.) with increasing numbers of PbmCherryLuci sporozoites and cured with 2×100 mg/kg doses of Example 1A. Results are shown in FIGS. 5A and 5B. Three months later, the chemovaccinated mice were challenged with 10 PbmCherryLuci infected mosquito bites. Some level of immunity was seen in all treatment groups. As shown in FIGS. 6A and 6B, 1/3 of the mice immunized with 500 sporozoites i.v. showed sterile protection from blood stage malaria infection; the other 2/3 of the mice immunized with 500 sporozoites i.v. developed malaria symptoms but showed partial immunity with a lower level of liver infection as well as a delay to detection of blood parasitemia as measured by Giemsa stained thin blood smears. All mice immunized with 1,000 and 40,000 sporozoites i.v. showed sterile protection from blood stage malaria infection at this 3-month challenge. Twelve months later, the chemovaccinated mice were challenged with 10 PbmCherryLuci infected mosquito bites. Some level of immunity was seen in all treatment groups.

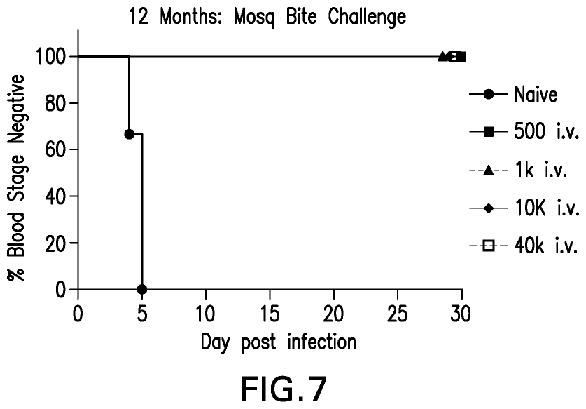

All mice that were immune at 3 months were re-challenged with PbmCherryLuci at 6 and 12 months post initial immunization and were likewise found to be completely immune. As shown in FIG. 7, at 12 months, all mice showed sterile protection from blood stage malaria infection. These data show that long lasting sterile immunity could be induced with as few as 500 sporozoites i.v., but that higher sporozoite inoculum were required for 100% sterile immunity.

Chemovaccination of C57BL/6 mice with Examples 1A and Example 75A

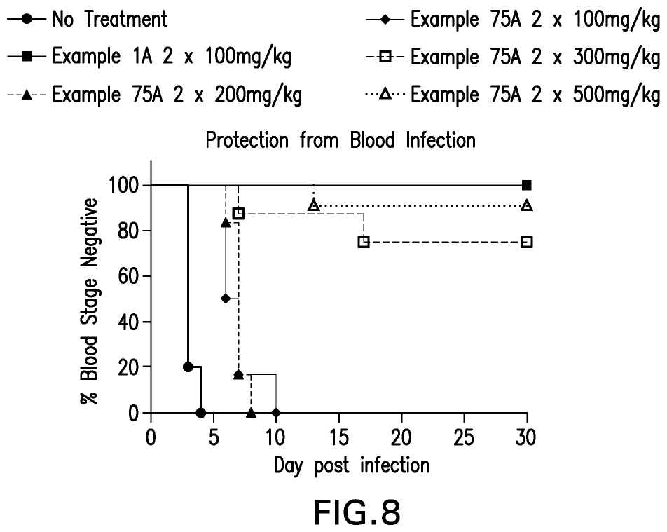

C57BL/6 mice were administered Example 1A or Example 75A as described in the general experimental procedure. As shown in FIG. 8, Example 1A and Example 75A inhibited transition from liver to blood infection in C57BL/6 mice. Example 1A completely prevented infection of the blood in all mice. Protection with Example 75A was seen with doses of 300 mg/kg (8 mice protected at this dose) and 500 mg/kg (10/11 mice protected at this dose). Lower doses of Example 75A gave large delays in the detection of parasites in the blood, demonstrating efficacy, but not sterile protection.

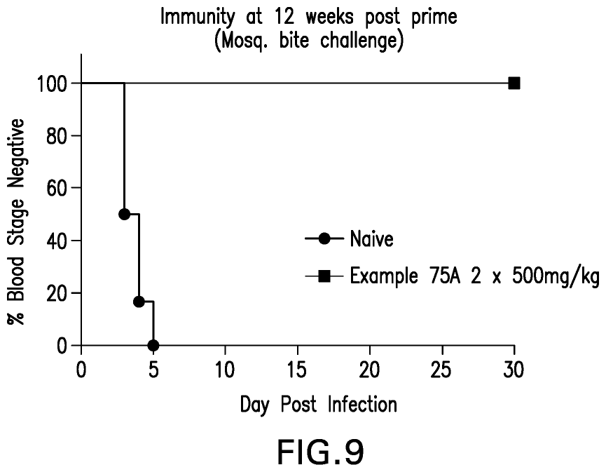

To investigate if chemoprophylaxis with Example 75A would serve as a chemovaccination as does Example IA, female C57BL/6 mice were immunized using a prime/boost regimen. The first immunisation was with 40,000 PbmCherryLuci salivary gland sporozoites i.v. followed by two doses of Example 75A at 500 mg/kg P.O. (given at 36 and 48 hours post infection). The second immunisation (boost) was six weeks later, this time with 20,000 PbmCherryLuci sporozoites but with Example 75A treatment as for the prime immunisation. Six weeks after the boost immunisation (twelve weeks after the prime immunisation) the immunised mice were challenged with the bites of 10 PbmCherryLuci infected mosquitoes for 15 minutes. Giemsa stained thin blood smears were analysed until day 30 for the presence of parasites in the blood and mice were considered to be protected from infection if no blood parasites were observed within the 30 days post mosquito bite challenge. As shown in FIG. 9, all naïve mice developed blood infection by 5 days after challenge, whereas all mice immunised using Example 75A were completely protected from reinfection at this 12-week time point, demonstrating 100% sterile protection. These data show that immunity can be induced by multiple PMIX/PMX inhibitors in two unique mouse strains.

Chemoprophylaxis of Humanised (FRG) Mice with Example 1A

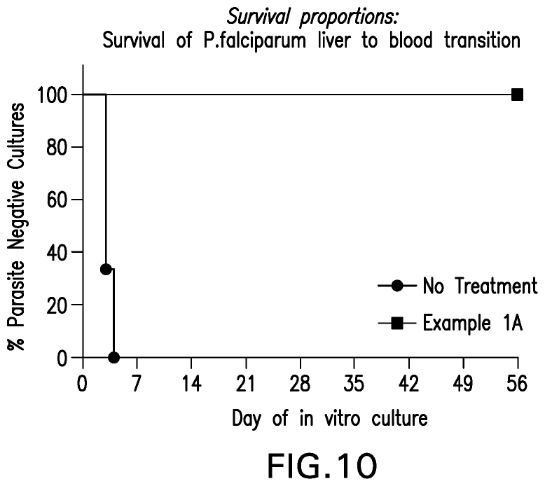

Four immunocompromised mice engrafted with human hepatocytes (FRG mice, Yecuris, OR, USA) were each infected intravenously with 300,000 freshly dissected P. falciparum salivary gland sporozoites. Three mice received no treatment, while one mouse received four oral treatments with 100 mg/kg Example 1A during P. falciparum liver stage development (at day 5, day 5.5, day 6 and day 6.5 post infection). To capture viable parasites leaving the liver after completing liver stage development (between 6-7 days post infection), all mice were reconstituted with human red blood cells so that the egressing parasites had a host cell pool they could infect. On day 7 post infection all mice were bled, and the blood cultured under standard culture conditions at 4% hematocrit with 1% oxygen. Fresh hematocrit was added to the cultures each week to provide fresh host cells for infection, and cultures were analysed for the presence of blood parasites 2-3 times per week by inspection of Giemsa-stained thin blood smears. As shown in FIG. 10, all mice in the no treatment group were positive for blood parasites after 4 days in culture, whereas the mouse treated with Example 1A remained negative for blood parasites over an eight-week observation period. These data demonstrate that Example IA is effective as a chemoprophylactic against the human infectious parasite P. falciparum, fully attenuating the liver parasites and preventing their liver to blood transition. This demonstration is important since effective and full attenuation of the liver to blood transition is a critical feature of the chemovaccination we describe for the rodent parasite P. berghei. As such, these data provide strong preclinical data supporting the use of chemovaccination with human infectious Plasmodium parasites with PMIX/PMX inhibition as a powerful malaria vaccine in humans.

1. A method of chemovaccination against Plasmodium infections comprising administering to a patient, an effective amount of a dual inhibitor of plasmepsin IX and X.

2. The method of claim 1, wherein the dual inhibitor of plasmepsin IX and X is a compound of structural Formula (I′): 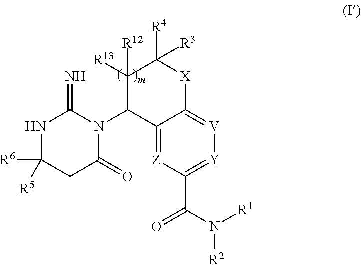
or a pharmaceutically acceptable salt thereof, wherein:
X is a bond, C(R¹⁴)₂, O, S, SO, SO₂ or NH;
Y is CR⁹ or N, wherein when Y is N, Z is CR¹¹ and V is CR¹⁰;
V is CR¹⁰ or N, wherein when V is N, Z is CR¹¹ and Y is CR⁹;
Z is CR¹¹ or N, wherein when Z is N, V is CR¹⁰ and Y is CR⁹;
R¹ is a heterocycloalkyl, C₃-C₁₂cycloalkyl, aryl, C₁-C₆alkylaryl or when taken with R², and the nitrogen which they are bonded, forms nitrogen-containing ring, wherein the heterocycloalkyl, C₃-C₁₂cycloalkyl, aryl, C₁-C₆alkylaryl or nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, aryl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R² is hydrogen, C₁-C₆alkylCOOH, COOH, C₃-C₆cycloalkyl, C₁-C₆alkyl, haloC₁-C₆alkyl or C₁-C₆alkylOH or when taken with R¹, and the nitrogen which they are bonded, forms a nitrogen-containing ring, wherein the nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl;
R⁴ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl;
R⁵ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁶ forms a C₃-C₆cycloalkyl or C₃-C₆heterocycloalkyl;
R⁶ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁵ forms a C₃-C₆cycloalkyl or C₃-C₆heterocycloalkyl;
R⁷ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁸ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹⁰ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹¹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹² is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
each occurrence of R¹⁴ is independently selected from the group consisting of hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
n is 1, 2, 3 or 4; and
m is 0, 1 or 2.

3. The method of claim 2, wherein, in the compound of structural Formula (I′), or a pharmaceutically acceptable salt thereof, m is 1 and X is O.

4. The method of claim 2, wherein, in the compound of structural Formula (I′), or a pharmaceutically acceptable salt thereof, m is 1 and X is CH₂.

5. The method of claim 2, wherein, in the compound of structural Formula (I′), or a pharmaceutically acceptable salt thereof, m is 0 and X is O.

6. The method of claim 2, wherein, in the compound of structural Formula (I′), or a pharmaceutically acceptable salt thereof, m is 1 and X is SO₂.

7. The method of claim 2, wherein, in the compound of structural Formula (I′), or a pharmaceutically acceptable salt thereof, m is 0 and X is C(R¹⁴)₂, wherein each occurrence of R¹⁴ is independently selected from the group consisting of hydrogen, halogen, OH, C₁-C₆alkoxy and C₁-C₆alkyl.

8. The method of claim 2, wherein, in the compound of structural Formula (I′), or a pharmaceutically acceptable salt thereof, m is 1 and X is C(R¹⁴)₂, wherein each occurrence of R¹⁴ is independently selected from the group consisting of hydrogen, halogen, OH, C₁-C₆alkoxy and C₁-C₆alkyl.

9. The method of claim 2, wherein, in the compound of structural Formula (I′), or a pharmaceutically acceptable salt thereof, m is 0 and X is a bond.

10. The method of claim 2, wherein, in the compound of structural Formula (I′), or a pharmaceutically acceptable salt thereof, R¹ is selected from the group consisting of: 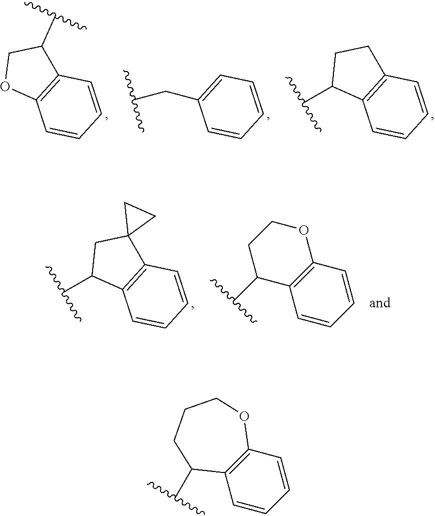
wherein R¹ is unsubstituted or substituted with 1 to 5 substituents selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₃-C₆cycloalkyl, C₁-C₆alkylC₃-C₆cycloalkyl, aryl, C₁-C₆alkyl, haloC₁-C₆alkyl, C₁-C₆alkylOhaloC₁-C₆alkyl, C₁-C₆alkylOH, CON(R⁷)(R⁸), N(R⁷)(R⁸) and C₁-C₆alkylN(R⁷)(R⁸);
R⁷ is hydrogen, C₁-C₆alkylCOOH, COOH, C₃-C₆cycloalkyl, C₁-C₆alkyl, haloC₁-C₆alkyl or C₁-C₆alkylOH; and
R⁸ is hydrogen, C₁-C₆alkylCOOH, COOH, C₃-C₆cycloalkyl, C₁-C₆alkyl, haloC₁-C₆alkyl or C₁-C₆alkylOH.

11. The method of claim 2, wherein, in the compound of structural Formula (I′), or a pharmaceutically acceptable salt thereof, R¹ is: 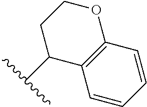
wherein R¹ is unsubstituted or substituted with 1 to 5 substituents selected from the group consisting of halogen, CN, OH, C₁-C₆alkoxy, C₁-C₆alkylOC₁-C₆alkyl, C₁-C₆alkylOhaloC₁-C₆alkyl, oxo, COOC₁-C₆alkyl, C₁-C₆alkyl, haloC₁-C₆alkyl and C₁-C₆alkylOH.

12. The method of claim 1, wherein, in the compound of structural Formula (I′), or a pharmaceutically acceptable salt thereof, R¹ is: 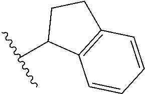
wherein R¹ is unsubstituted or substituted with 1 to 5 substituents selected from the group consisting of halogen, CN, OH, C₁-C₆alkoxy, C₁-C₆alkylOC₁-C₆alkyl, C₁-C₆alkylOhaloC₁-C₆alkyl, oxo, COOC₁-C₆alkyl, C₁-C₆alkyl, haloC₁-C₆alkyl, NH₂, and C₁-C₆alkylOH.

13. The method of claim 2, wherein, in the compound of structural Formula (I′), or a pharmaceutically acceptable salt thereof, R² is hydrogen.

14. The method of claim 2, wherein, in the compound of structural Formula (I′), or a pharmaceutically acceptable salt thereof, R¹ is taken with R² and the nitrogen to which they are bonded, to form a nitrogen-containing ring.

15. The method of claim 14, wherein, in the compound of structural Formula (I′), or a pharmaceutically acceptable salt thereof, the nitrogen-containing ring is:

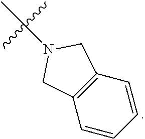 [SMILES: *N1Cc2ccccc2C1]

16. The method of claim 2, wherein, in the compound of structural Formula (I′), or a pharmaceutically acceptable salt thereof, R³ is hydrogen, C₁-C₆alkylOC₁-C₆alkyl or C₁-C₆alkyl or when taken with R⁴ forms a C₃-C₆heterocycloalkyl.

17. (canceled)

18. The method of claim 2, wherein, in the compound of structural Formula (I′), or a pharmaceutically acceptable salt thereof, R⁵ is hydrogen or C₁-C₆alkyl and R⁶ is hydrogen or C₁-C₆alkyl.

19. (canceled)

20. The method of claim 2, wherein, in the compound of structural Formula (I′), or a pharmaceutically acceptable salt thereof, R³ and R⁴ are independently selected from the group consisting of hydrogen, halogen, OH, C₁-C₆alkylOH, C₁-C₆alkylalkoxy, C₁-C₆alkylOC₁-C₆alkyl, C₁-C₆alkyl, C₁-C₆alkylOhaloC₁-C₆alkyl, CON(C₁-C₆alkyl)₂, C₁-C₆alkylN(R⁷)(R⁸) and C₁-C₆alkyl(OCH₂CH₂)ₙN(R⁷)(R⁸);
R⁷ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁸ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl; and
n is 3.

21. The method of claim 2, wherein, in the compound of structural Formula (I′), or a pharmaceutically acceptable salt thereof, m is 1 or 2, R¹² and R¹³ are independently selected from the group consisting of hydrogen, halogen, OH, C₁-C₆alkylOH, C₁-C₆alkylalkoxy and C₁-C₆alkylOC₁-C₆alkyl, C₁-C₆alkyl, and X is C(R¹⁴)₂, R¹⁴ is independently selected from the group consisting of hydrogen, halogen, OH, C₁-C₆alkylOH, C₁-C₆alkylalkoxy, C₁-C₆alkylOC₁-C₆alkyl and C₁-C₆alkyl.

22. (canceled)

23. The method of claim 2, wherein, in the compound of structural Formula (I′), or a pharmaceutically acceptable salt thereof, Y is CH.

24. The method of claim 2, wherein, in the compound of structural Formula (I′), or a pharmaceutically acceptable salt thereof, Z is CH.

25. The method of claim 2, wherein, in the compound of structural Formula (I′), or a pharmaceutically acceptable salt thereof, V is CH.

26. The method of claim 2, wherein, in the compound of structural Formula (I′), or a pharmaceutically acceptable salt thereof, Z is N and Y and V are both CH, or V is N and Y and Z are both CH.

27. (canceled)

28. The method of chemovaccination against Plasmodium infections according to claim 2, wherein compound is selected from the group consisting of: 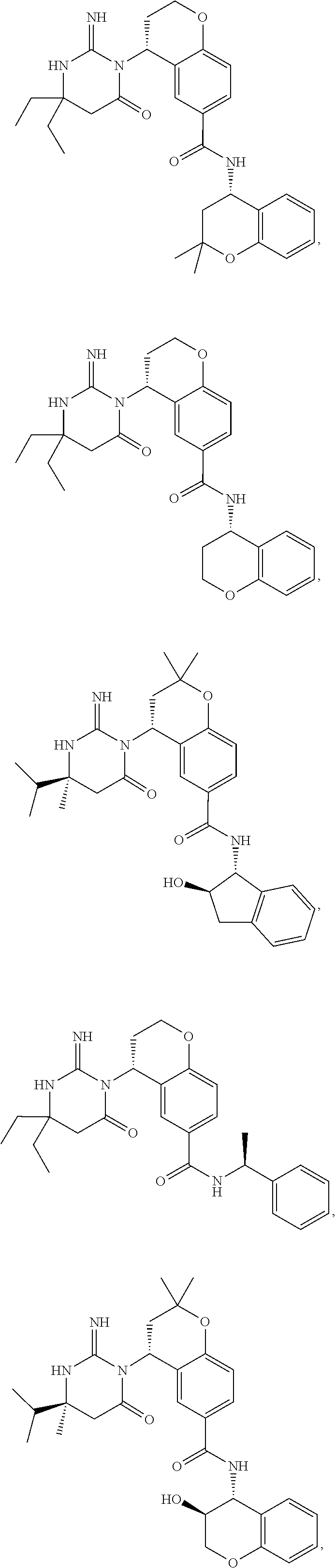 [SMILES: CCC1(CC)CC(=O)N([C@@H]2CCOc3ccc(C(=O)N[C@H]4CC(C)(C)Oc5ccccc54)cc32)C(=N)N1 ; CCC1(CC)CC(=O)N([C@@H]2CCOc3ccc(C(=O)N[C@H]4CCOc5ccccc54)cc32)C(=N)N1 ; CC(C)[C@]1(C)CC(=O)N([C@@H]2CC(C)(C)Oc3ccc(C(=O)N[C@@H]4c5ccccc5C[C@H]4O)cc32)C(=N)N1 ; CCC1(CC)CC(=O)N([C@@H]2CCOc3ccc(C(=O)N[C@@H](C)c4ccccc4)cc32)C(=N)N1 ; CC(C)[C@]1(C)CC(=O)N([C@@H]2CC(C)(C)Oc3ccc(C(=O)N[C@@H]4c5ccccc5OC[C@H]4O)cc32)C(=N)N1] 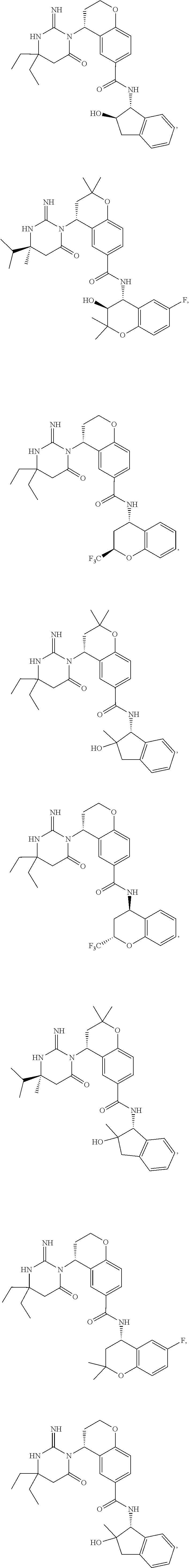 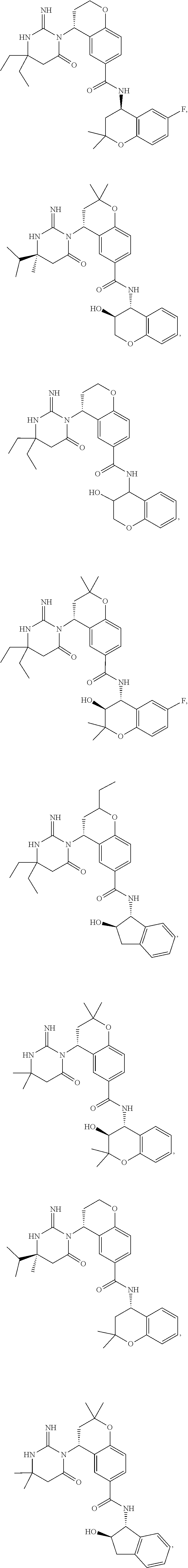 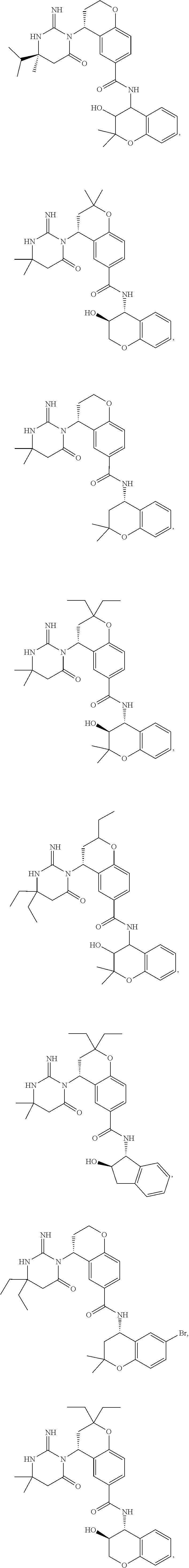 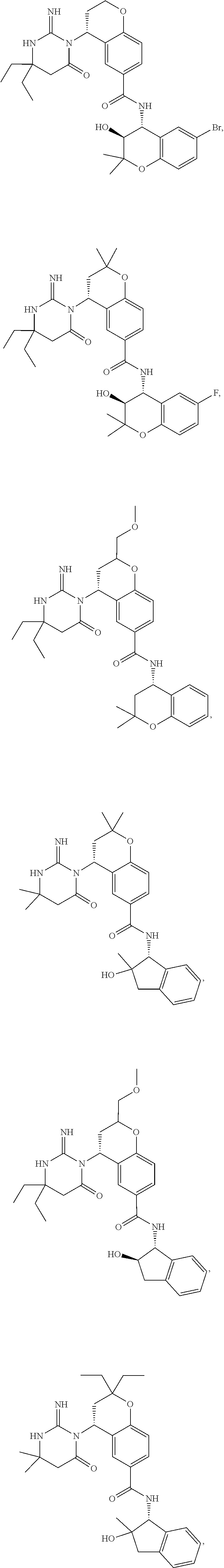 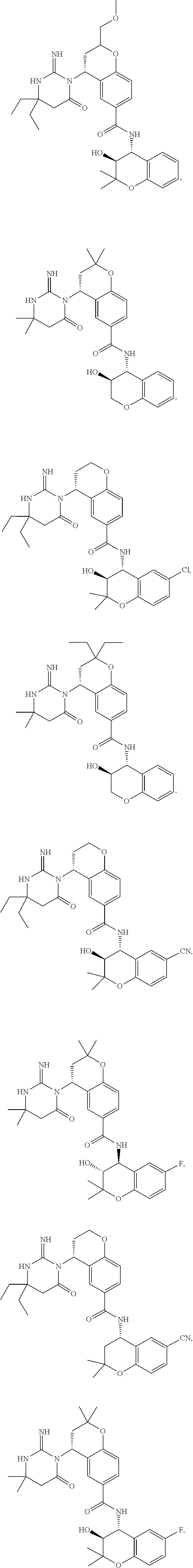 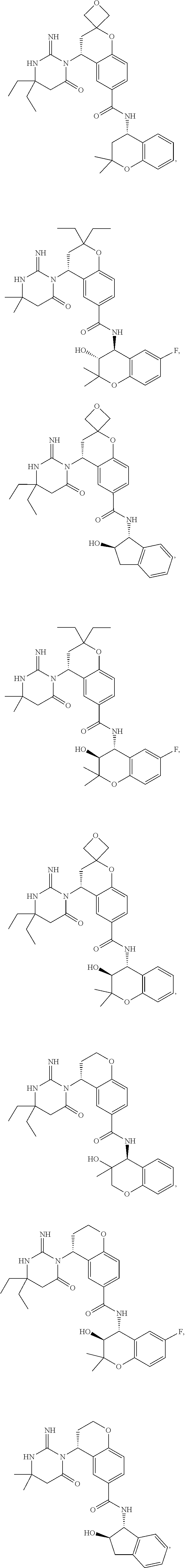 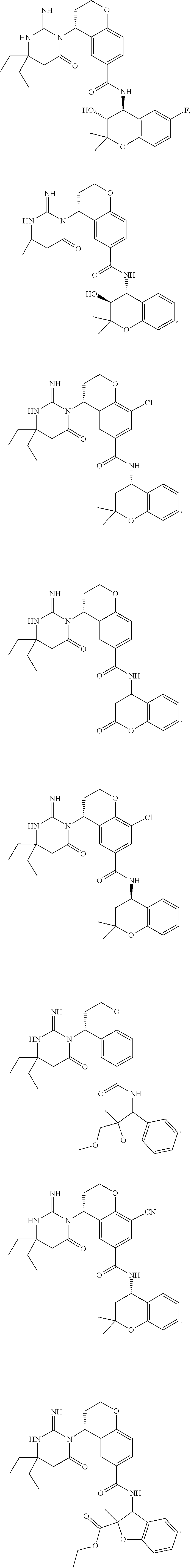 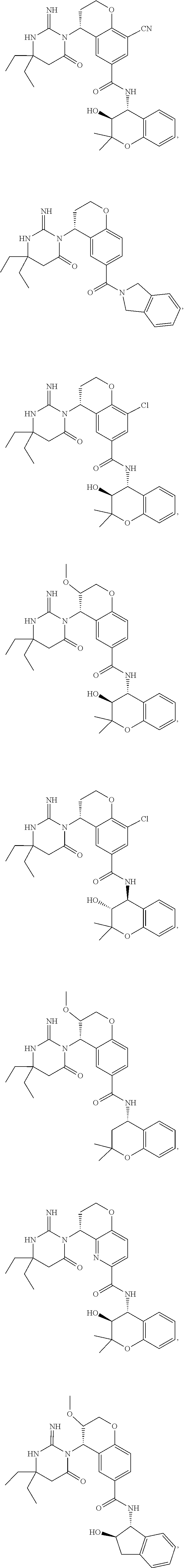 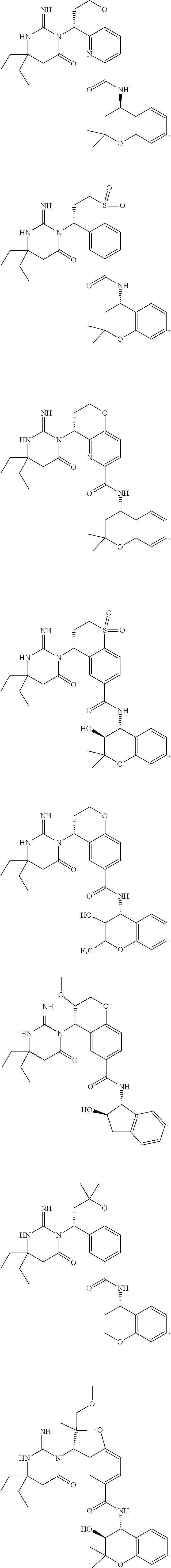 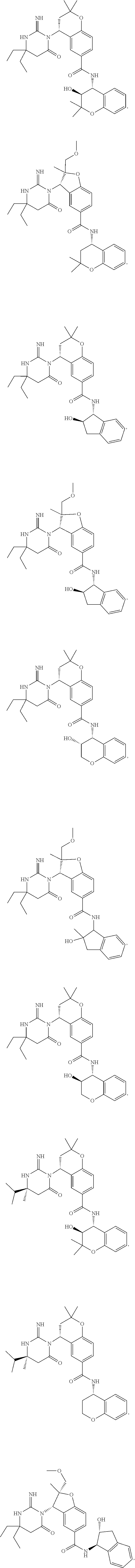 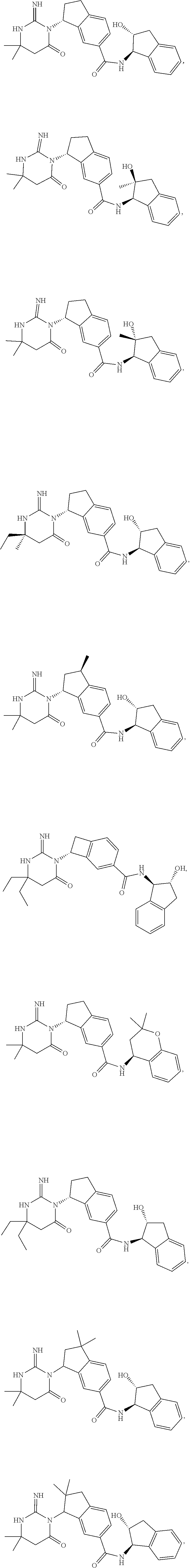 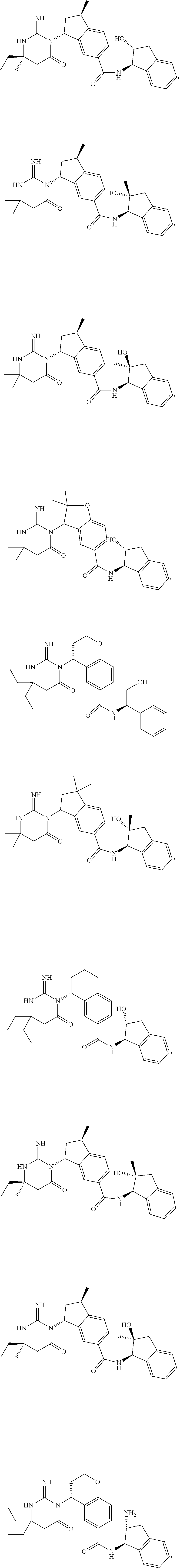 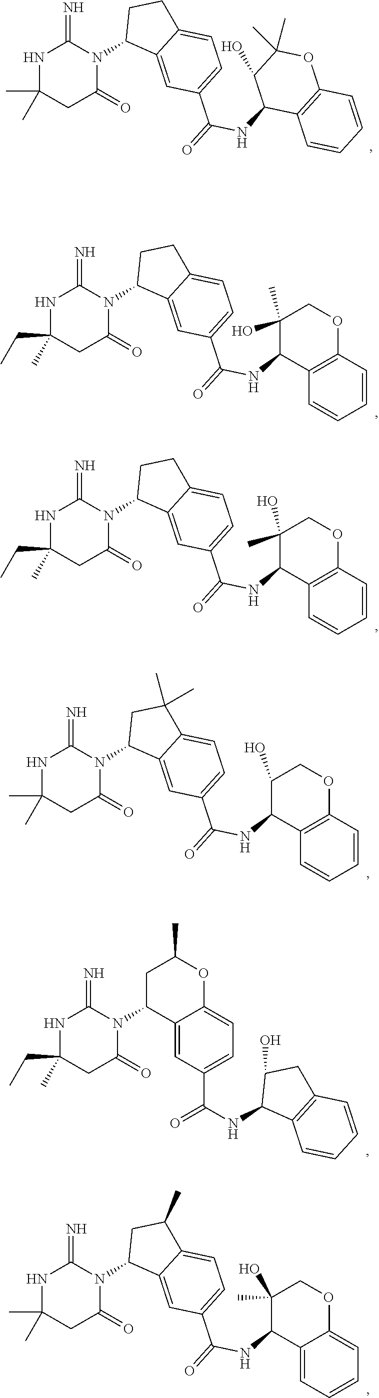 [SMILES: CC1(C)CC(=O)N([C@@H]2CCc3ccc(C(=O)N[C@@H]4c5ccccc5OC(C)(C)[C@H]4O)cc32)C(=N)N1 ; CC[C@]1(C)CC(=O)N([C@@H]2CCc3ccc(C(=O)N[C@@H]4c5ccccc5OC[C@]4(C)O)cc32)C(=N)N1 ; CC[C@]1(C)CC(=O)N([C@@H]2CCc3ccc(C(=O)N[C@@H]4c5ccccc5OC[C@@]4(C)O)cc32)C(=N)N1 ; CC1(C)CC(=O)N([C@@H]2CC(C)(C)c3ccc(C(=O)N[C@@H]4c5ccccc5OC[C@H]4O)cc32)C(=N)N1 ; CC[C@]1(C)CC(=O)N([C@@H]2C[C@@H](C)Oc3ccc(C(=O)N[C@@H]4c5ccccc5C[C@H]4O)cc32)C(=N)N1 ; C[C@@H]1C[C@@H](N2C(=N)NC(C)(C)CC2=O)c2cc(C(=O)N[C@@H]3c4ccccc4OC[C@]3(C)O)ccc21] 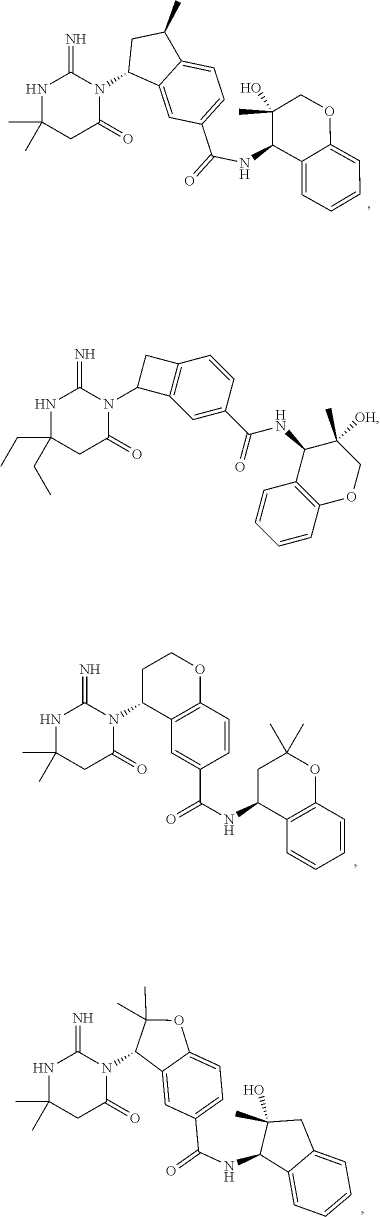 [SMILES: C[C@@H]1C[C@@H](N2C(=N)NC(C)(C)CC2=O)c2cc(C(=O)N[C@@H]3c4ccccc4OC[C@@]3(C)O)ccc21 ; CCC1(CC)CC(=O)N(C2Cc3ccc(C(=O)N[C@@H]4c5ccccc5OC[C@@]4(C)O)cc32)C(=N)N1 ; CC1(C)CC(=O)N([C@@H]2CCOc3ccc(C(=O)N[C@H]4CC(C)(C)Oc5ccccc54)cc32)C(=N)N1 ; CC1(C)CC(=O)N([C@H]2c3cc(C(=O)N[C@@H]4c5ccccc5C[C@@]4(C)O)ccc3OC2(C)C)C(=N)N1] 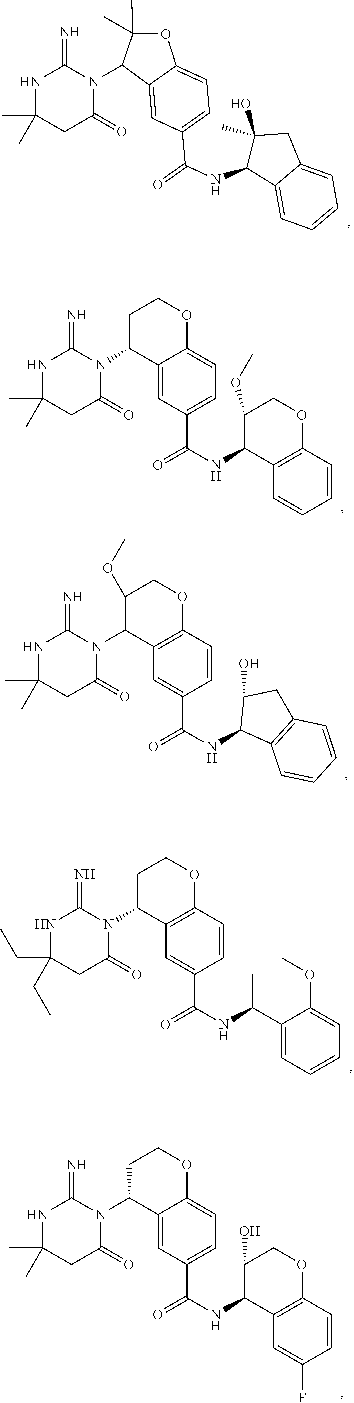 [SMILES: CC1(C)CC(=O)N(C2c3cc(C(=O)N[C@@H]4c5ccccc5C[C@]4(C)O)ccc3OC2(C)C)C(=N)N1 ; CO[C@@H]1COc2ccccc2[C@H]1NC(=O)c1ccc2c(c1)[C@H](N1C(=N)NC(C)(C)CC1=O)CCO2 ; COC1COc2ccc(C(=O)N[C@@H]3c4ccccc4C[C@H]3O)cc2C1N1C(=N)NC(C)(C)CC1=O ; CCC1(CC)CC(=O)N([C@@H]2CCOc3ccc(C(=O)N[C@@H](C)c4ccccc4OC)cc32)C(=N)N1 ; CC1(C)CC(=O)N([C@@H]2CCOc3ccc(C(=O)N[C@@H]4c5cc(F)ccc5OC[C@H]4O)cc32)C(=N)N1] 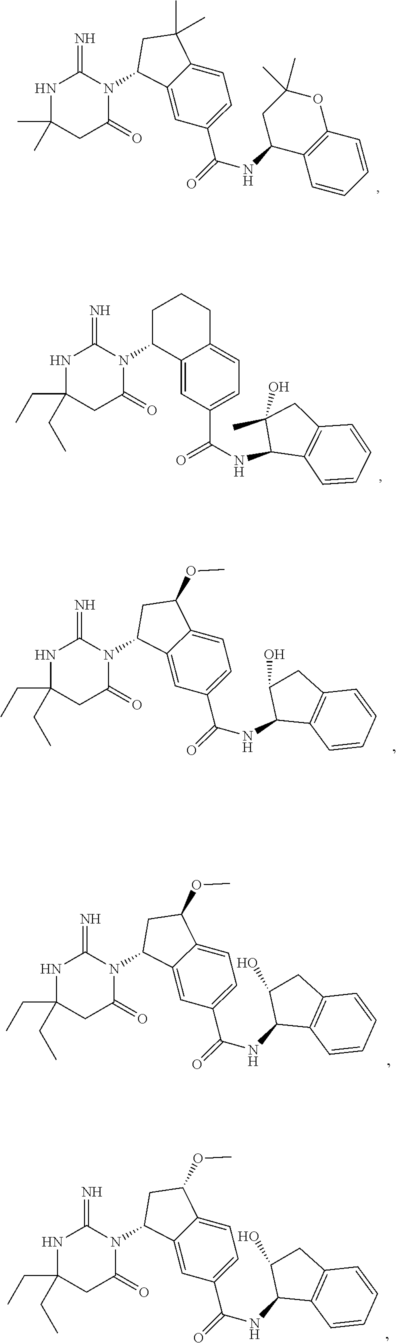 [SMILES: CC1(C)CC(=O)N([C@@H]2CC(C)(C)c3ccc(C(=O)N[C@H]4CC(C)(C)Oc5ccccc54)cc32)C(=N)N1 ; CCC1(CC)CC(=O)N([C@@H]2CCCc3ccc(C(=O)N[C@@H]4c5ccccc5C[C@@]4(C)O)cc32)C(=N)N1 ; CCC1(CC)CC(=O)N([C@@H]2C[C@@H](OC)c3ccc(C(=O)N[C@@H]4c5ccccc5C[C@H]4O)cc32)C(=N)N1 ; CCC1(CC)CC(=O)N([C@@H]2C[C@@H](OC)c3ccc(C(=O)N[C@@H]4c5ccccc5C[C@H]4O)cc32)C(=N)N1 ; CCC1(CC)CC(=O)N([C@@H]2C[C@H](OC)c3ccc(C(=O)N[C@@H]4c5ccccc5C[C@H]4O)cc32)C(=N)N1] 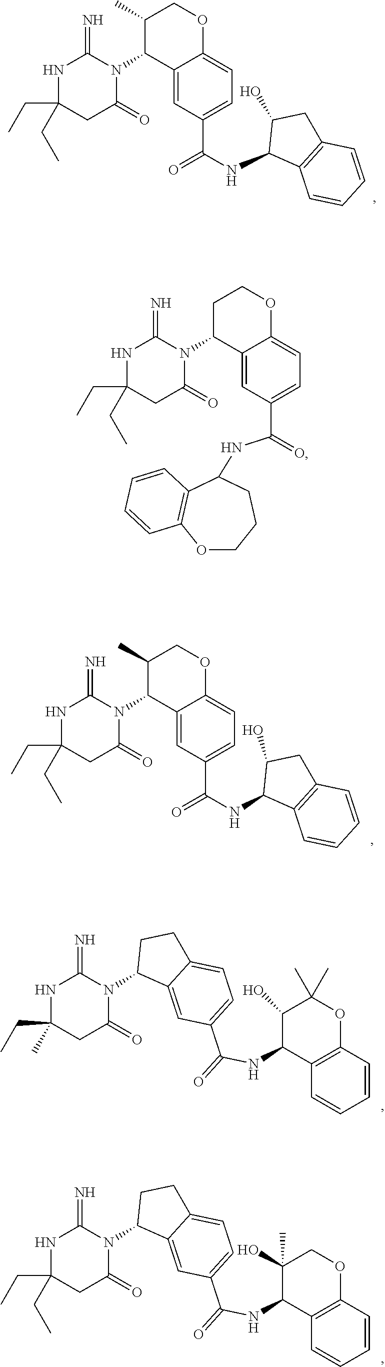 [SMILES: CCC1(CC)CC(=O)N([C@H]2c3cc(C(=O)N[C@@H]4c5ccccc5C[C@H]4O)ccc3OC[C@H]2C)C(=N)N1 ; CCC1(CC)CC(=O)N([C@@H]2CCOc3ccc(C(=O)NC4CCCOc5ccccc54)cc32)C(=N)N1 ; CCC1(CC)CC(=O)N([C@H]2c3cc(C(=O)N[C@@H]4c5ccccc5C[C@H]4O)ccc3OC[C@@H]2C)C(=N)N1 ; CC[C@]1(C)CC(=O)N([C@@H]2CCc3ccc(C(=O)N[C@@H]4c5ccccc5OC(C)(C)[C@H]4O)cc32)C(=N)N1 ; CCC1(CC)CC(=O)N([C@@H]2CCc3ccc(C(=O)N[C@@H]4c5ccccc5OC[C@]4(C)O)cc32)C(=N)N1] 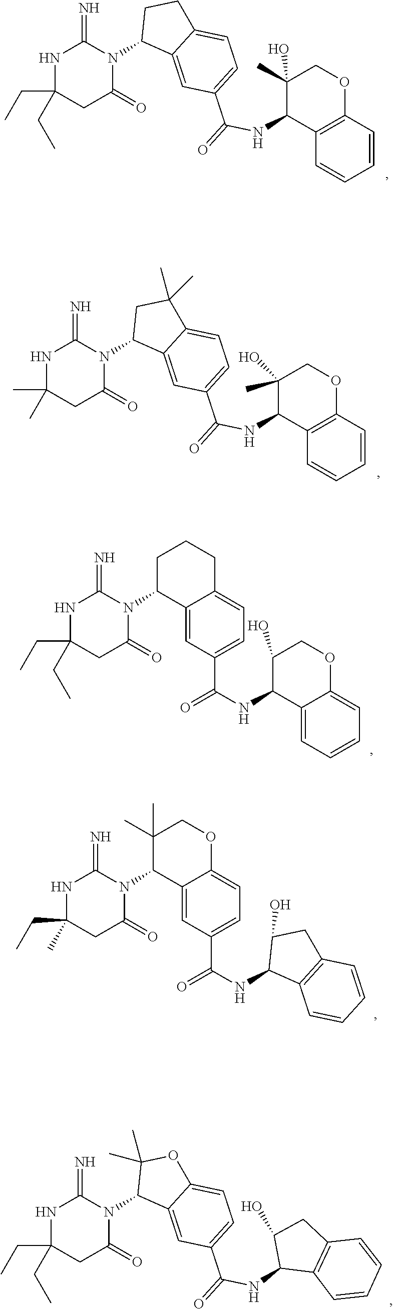 [SMILES: CCC1(CC)CC(=O)N([C@@H]2CCc3ccc(C(=O)N[C@@H]4c5ccccc5OC[C@@]4(C)O)cc32)C(=N)N1 ; CC1(C)CC(=O)N([C@@H]2CC(C)(C)c3ccc(C(=O)N[C@@H]4c5ccccc5OC[C@@]4(C)O)cc32)C(=N)N1 ; CCC1(CC)CC(=O)N([C@@H]2CCCc3ccc(C(=O)N[C@@H]4c5ccccc5OC[C@H]4O)cc32)C(=N)N1 ; CC[C@]1(C)CC(=O)N([C@H]2c3cc(C(=O)N[C@@H]4c5ccccc5C[C@H]4O)ccc3OCC2(C)C)C(=N)N1 ; CCC1(CC)CC(=O)N([C@H]2c3cc(C(=O)N[C@@H]4c5ccccc5C[C@H]4O)ccc3OC2(C)C)C(=N)N1] 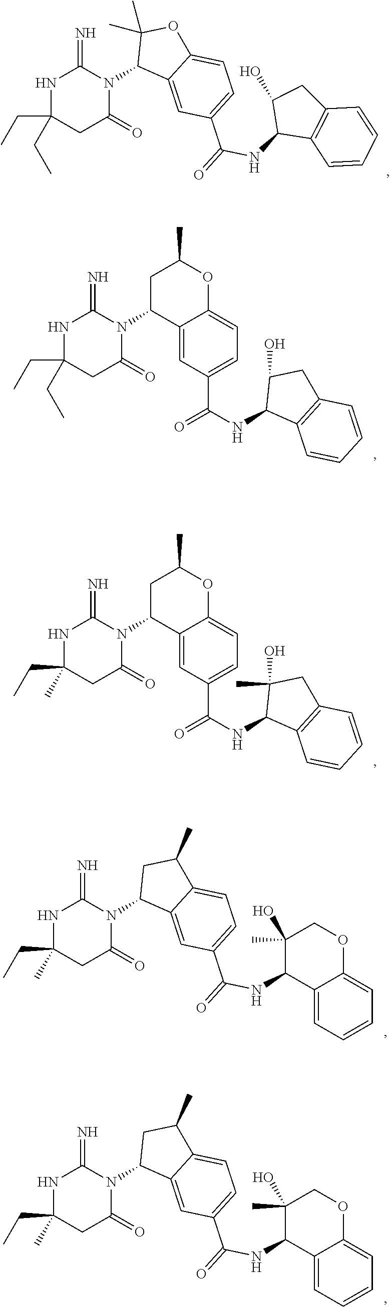 [SMILES: CCC1(CC)CC(=O)N([C@H]2c3cc(C(=O)N[C@@H]4c5ccccc5C[C@H]4O)ccc3OC2(C)C)C(=N)N1 ; CCC1(CC)CC(=O)N([C@@H]2C[C@@H](C)Oc3ccc(C(=O)N[C@@H]4c5ccccc5C[C@H]4O)cc32)C(=N)N1 ; CC[C@]1(C)CC(=O)N([C@@H]2C[C@@H](C)Oc3ccc(C(=O)N[C@@H]4c5ccccc5C[C@@]4(C)O)cc32)C(=N)N1 ; CC[C@]1(C)CC(=O)N([C@@H]2C[C@@H](C)c3ccc(C(=O)N[C@@H]4c5ccccc5OC[C@]4(C)O)cc32)C(=N)N1 ; CC[C@]1(C)CC(=O)N([C@@H]2C[C@@H](C)c3ccc(C(=O)N[C@@H]4c5ccccc5OC[C@@]4(C)O)cc32)C(=N)N1] 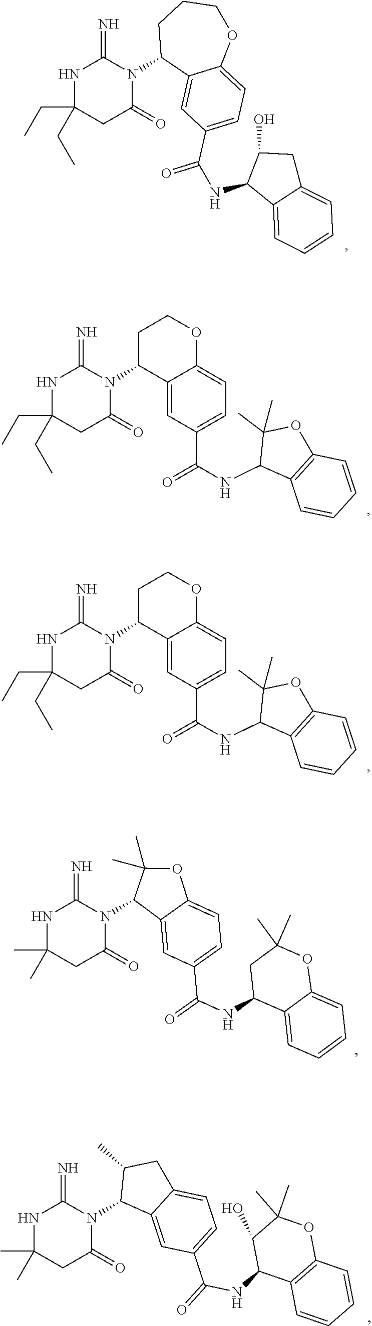 [SMILES: CCC1(CC)CC(=O)N([C@@H]2CCCOc3ccc(C(=O)N[C@@H]4c5ccccc5C[C@H]4O)cc32)C(=N)N1 ; CCC1(CC)CC(=O)N([C@@H]2CCOc3ccc(C(=O)NC4c5ccccc5OC4(C)C)cc32)C(=N)N1 ; CCC1(CC)CC(=O)N([C@@H]2CCOc3ccc(C(=O)NC4c5ccccc5OC4(C)C)cc32)C(=N)N1 ; CC1(C)CC(=O)N([C@H]2c3cc(C(=O)N[C@H]4CC(C)(C)Oc5ccccc54)ccc3OC2(C)C)C(=N)N1 ; C[C@@H]1Cc2ccc(C(=O)N[C@@H]3c4ccccc4OC(C)(C)[C@H]3O)cc2[C@@H]1N1C(=N)NC(C)(C)CC1=O] 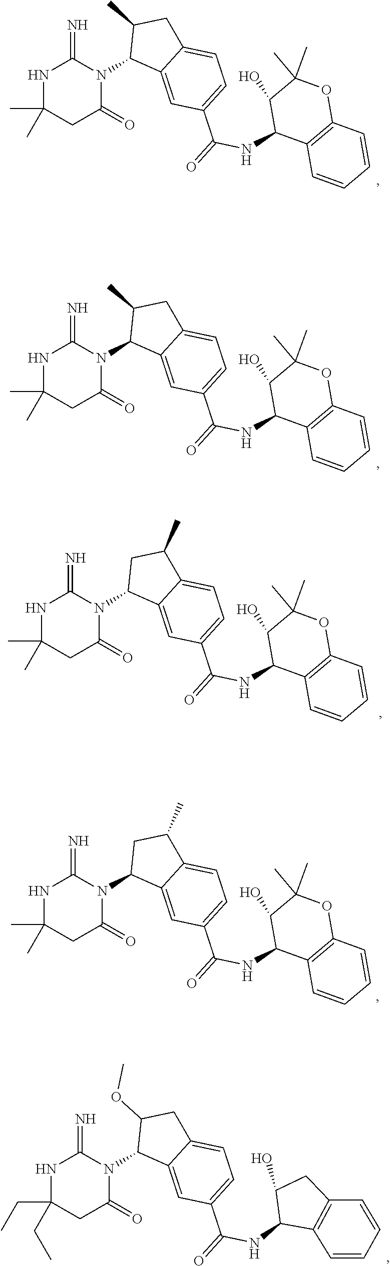 [SMILES: C[C@H]1Cc2ccc(C(=O)N[C@@H]3c4ccccc4OC(C)(C)[C@H]3O)cc2[C@@H]1N1C(=N)NC(C)(C)CC1=O ; C[C@H]1Cc2ccc(C(=O)N[C@@H]3c4ccccc4OC(C)(C)[C@H]3O)cc2[C@H]1N1C(=N)NC(C)(C)CC1=O ; C[C@@H]1C[C@@H](N2C(=N)NC(C)(C)CC2=O)c2cc(C(=O)N[C@@H]3c4ccccc4OC(C)(C)[C@H]3O)ccc21 ; C[C@H]1C[C@H](N2C(=N)NC(C)(C)CC2=O)c2cc(C(=O)N[C@@H]3c4ccccc4OC(C)(C)[C@H]3O)ccc21 ; CCC1(CC)CC(=O)N([C@H]2c3cc(C(=O)N[C@@H]4c5ccccc5C[C@H]4O)ccc3CC2OC)C(=N)N1] 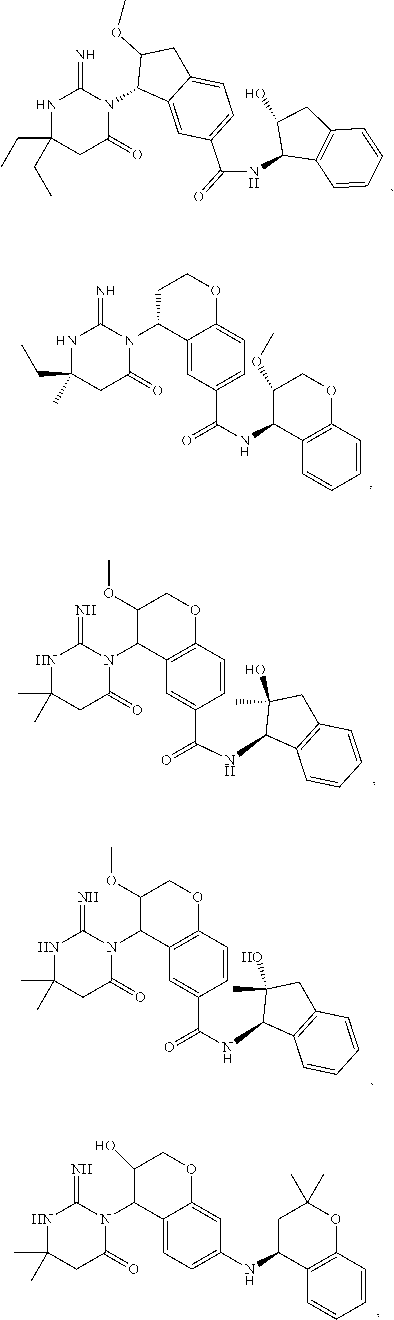 [SMILES: CCC1(CC)CC(=O)N([C@H]2c3cc(C(=O)N[C@@H]4c5ccccc5C[C@H]4O)ccc3CC2OC)C(=N)N1 ; CC[C@]1(C)CC(=O)N([C@@H]2CCOc3ccc(C(=O)N[C@@H]4c5ccccc5OC[C@H]4OC)cc32)C(=N)N1 ; COC1COc2ccc(C(=O)N[C@@H]3c4ccccc4C[C@]3(C)O)cc2C1N1C(=N)NC(C)(C)CC1=O ; COC1COc2ccc(C(=O)N[C@@H]3c4ccccc4C[C@@]3(C)O)cc2C1N1C(=N)NC(C)(C)CC1=O ; CC1(C)CC(=O)N(C2c3ccc(N[C@H]4CC(C)(C)Oc5ccccc54)cc3OCC2O)C(=N)N1] 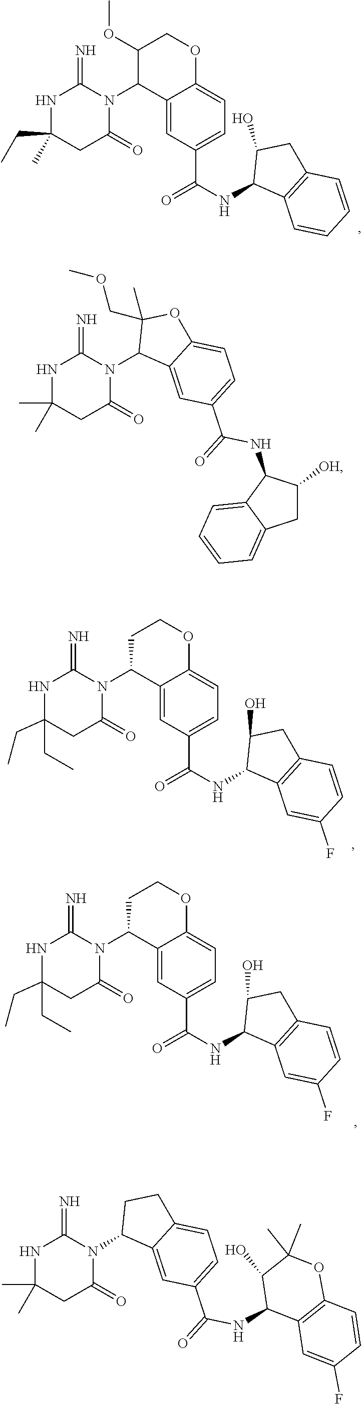 [SMILES: CC[C@]1(C)CC(=O)N(C2c3cc(C(=O)N[C@@H]4c5ccccc5C[C@H]4O)ccc3OCC2OC)C(=N)N1 ; COCC1(C)Oc2ccc(C(=O)N[C@@H]3c4ccccc4C[C@H]3O)cc2C1N1C(=N)NC(C)(C)CC1=O ; CCC1(CC)CC(=O)N([C@@H]2CCOc3ccc(C(=O)N[C@H]4c5cc(F)ccc5C[C@@H]4O)cc32)C(=N)N1 ; CCC1(CC)CC(=O)N([C@@H]2CCOc3ccc(C(=O)N[C@@H]4c5cc(F)ccc5C[C@H]4O)cc32)C(=N)N1 ; CC1(C)CC(=O)N([C@@H]2CCc3ccc(C(=O)N[C@@H]4c5cc(F)ccc5OC(C)(C)[C@H]4O)cc32)C(=N)N1] 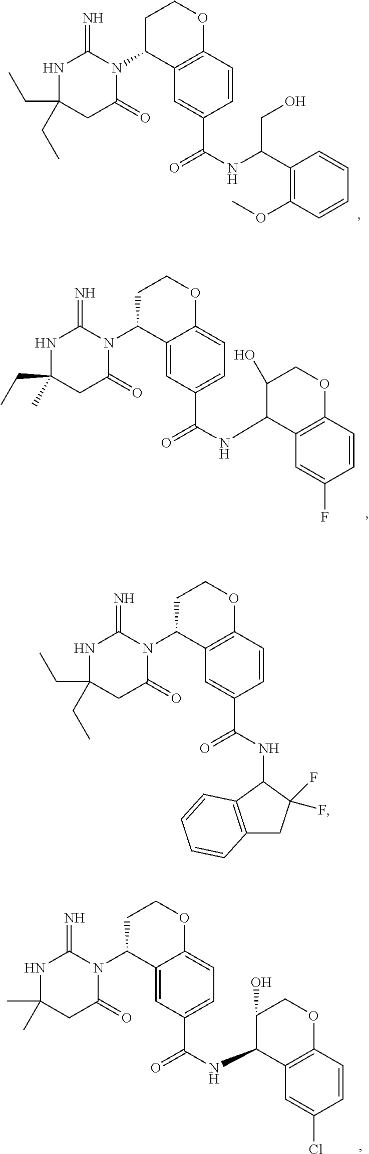 [SMILES: CCC1(CC)CC(=O)N([C@@H]2CCOc3ccc(C(=O)NC(CO)c4ccccc4OC)cc32)C(=N)N1 ; CC[C@]1(C)CC(=O)N([C@@H]2CCOc3ccc(C(=O)NC4c5cc(F)ccc5OCC4O)cc32)C(=N)N1 ; CCC1(CC)CC(=O)N([C@@H]2CCOc3ccc(C(=O)NC4c5ccccc5CC4(F)F)cc32)C(=N)N1 ; CC1(C)CC(=O)N([C@@H]2CCOc3ccc(C(=O)N[C@@H]4c5cc(Cl)ccc5OC[C@H]4O)cc32)C(=N)N1] 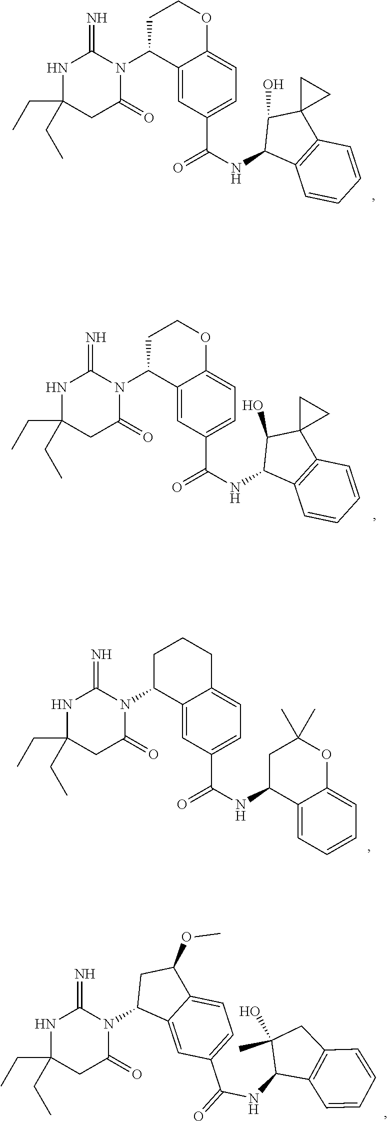 [SMILES: CCC1(CC)CC(=O)N([C@@H]2CCOc3ccc(C(=O)N[C@@H]4c5ccccc5C5(CC5)[C@H]4O)cc32)C(=N)N1 ; CCC1(CC)CC(=O)N([C@@H]2CCOc3ccc(C(=O)N[C@H]4c5ccccc5C5(CC5)[C@@H]4O)cc32)C(=N)N1 ; CCC1(CC)CC(=O)N([C@@H]2CCCc3ccc(C(=O)N[C@H]4CC(C)(C)Oc5ccccc54)cc32)C(=N)N1 ; CCC1(CC)CC(=O)N([C@@H]2C[C@@H](OC)c3ccc(C(=O)N[C@@H]4c5ccccc5C[C@@]4(C)O)cc32)C(=N)N1] 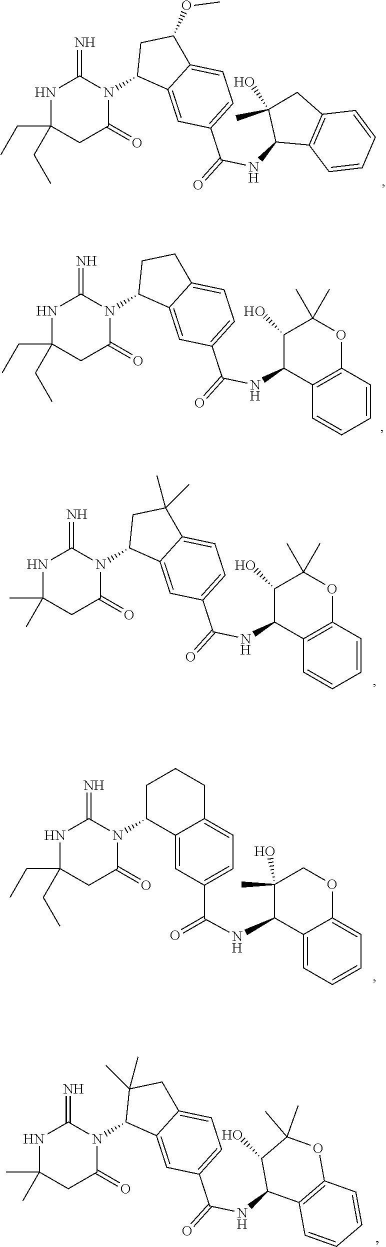 [SMILES: CCC1(CC)CC(=O)N([C@@H]2C[C@H](OC)c3ccc(C(=O)N[C@@H]4c5ccccc5C[C@@]4(C)O)cc32)C(=N)N1 ; CCC1(CC)CC(=O)N([C@@H]2CCc3ccc(C(=O)N[C@@H]4c5ccccc5OC(C)(C)[C@H]4O)cc32)C(=N)N1 ; CC1(C)CC(=O)N([C@@H]2CC(C)(C)c3ccc(C(=O)N[C@@H]4c5ccccc5OC(C)(C)[C@H]4O)cc32)C(=N)N1 ; CCC1(CC)CC(=O)N([C@@H]2CCCc3ccc(C(=O)N[C@@H]4c5ccccc5OC[C@@]4(C)O)cc32)C(=N)N1 ; CC1(C)CC(=O)N([C@H]2c3cc(C(=O)N[C@@H]4c5ccccc5OC(C)(C)[C@H]4O)ccc3CC2(C)C)C(=N)N1] 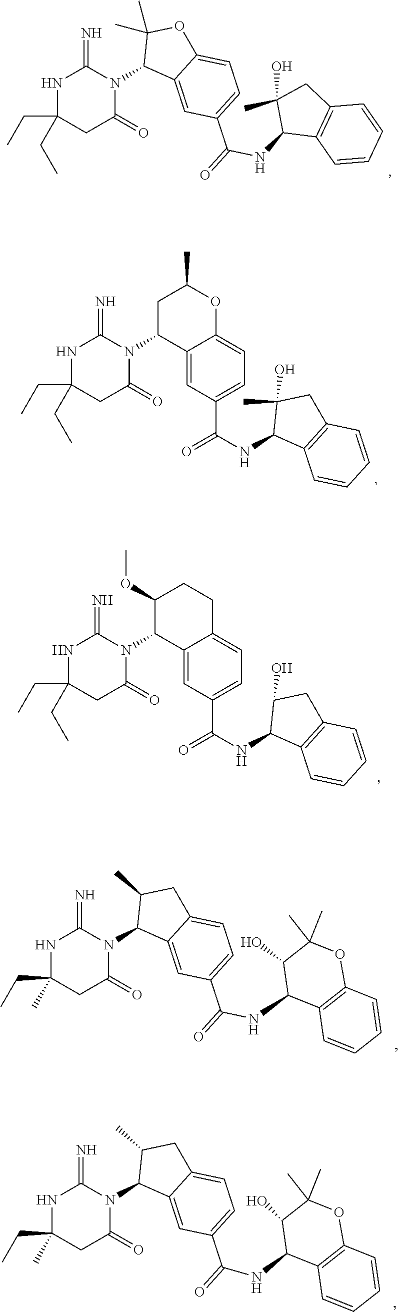 [SMILES: CCC1(CC)CC(=O)N([C@H]2c3cc(C(=O)N[C@@H]4c5ccccc5C[C@@]4(C)O)ccc3OC2(C)C)C(=N)N1 ; CCC1(CC)CC(=O)N([C@@H]2C[C@@H](C)Oc3ccc(C(=O)N[C@@H]4c5ccccc5C[C@@]4(C)O)cc32)C(=N)N1 ; CCC1(CC)CC(=O)N([C@H]2c3cc(C(=O)N[C@@H]4c5ccccc5C[C@H]4O)ccc3CC[C@@H]2OC)C(=N)N1 ; CC[C@]1(C)CC(=O)N([C@@H]2c3cc(C(=O)N[C@@H]4c5ccccc5OC(C)(C)[C@H]4O)ccc3C[C@@H]2C)C(=N)N1 ; CC[C@]1(C)CC(=O)N([C@@H]2c3cc(C(=O)N[C@@H]4c5ccccc5OC(C)(C)[C@H]4O)ccc3C[C@H]2C)C(=N)N1] 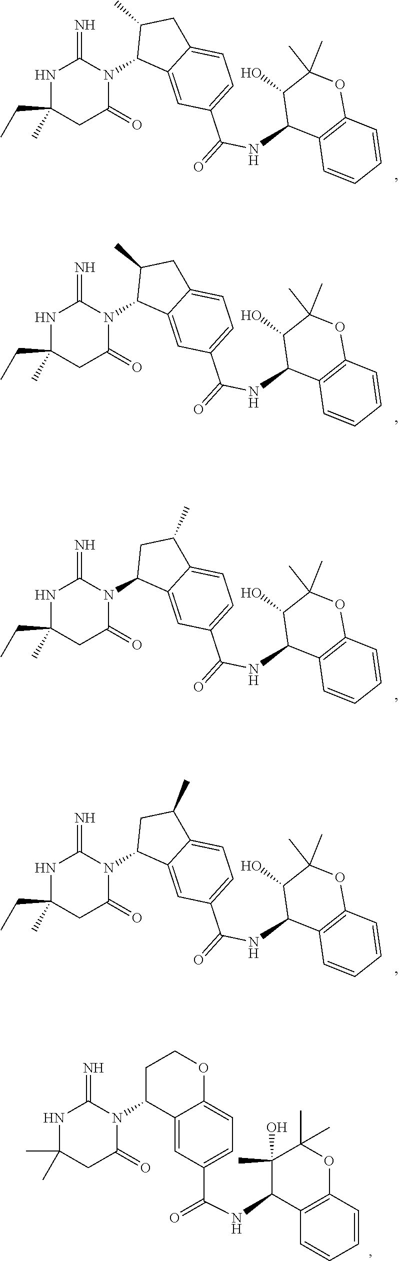 [SMILES: CC[C@]1(C)CC(=O)N([C@H]2c3cc(C(=O)N[C@@H]4c5ccccc5OC(C)(C)[C@H]4O)ccc3C[C@H]2C)C(=N)N1 ; CC[C@]1(C)CC(=O)N([C@H]2c3cc(C(=O)N[C@@H]4c5ccccc5OC(C)(C)[C@H]4O)ccc3C[C@@H]2C)C(=N)N1 ; CC[C@]1(C)CC(=O)N([C@H]2C[C@H](C)c3ccc(C(=O)N[C@@H]4c5ccccc5OC(C)(C)[C@H]4O)cc32)C(=N)N1 ; CC[C@]1(C)CC(=O)N([C@@H]2C[C@@H](C)c3ccc(C(=O)N[C@@H]4c5ccccc5OC(C)(C)[C@H]4O)cc32)C(=N)N1 ; CC1(C)CC(=O)N([C@@H]2CCOc3ccc(C(=O)N[C@@H]4c5ccccc5OC(C)(C)[C@@]4(C)O)cc32)C(=N)N1] 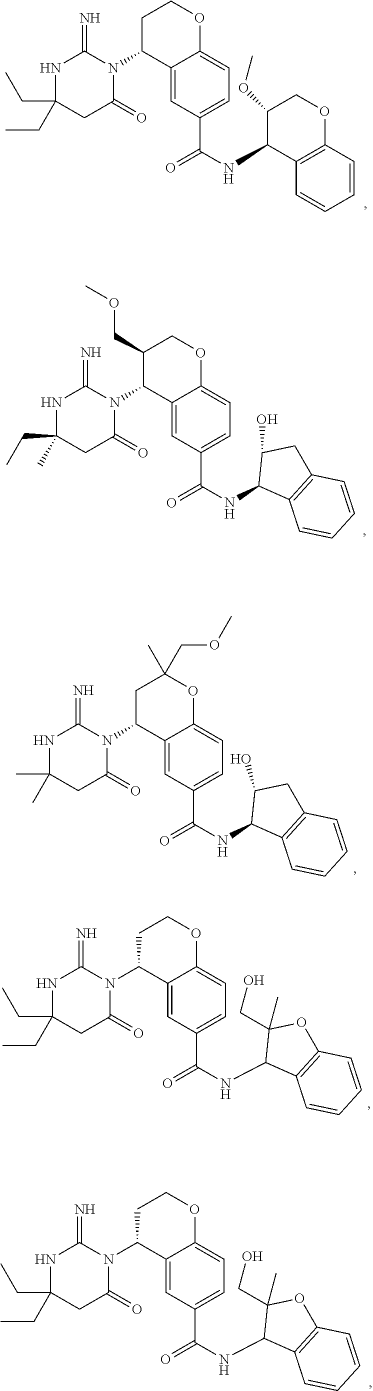 [SMILES: CCC1(CC)CC(=O)N([C@@H]2CCOc3ccc(C(=O)N[C@@H]4c5ccccc5OC[C@H]4OC)cc32)C(=N)N1 ; CC[C@]1(C)CC(=O)N([C@H]2c3cc(C(=O)N[C@@H]4c5ccccc5C[C@H]4O)ccc3OC[C@@H]2COC)C(=N)N1 ; COCC1(C)C[C@@H](N2C(=N)NC(C)(C)CC2=O)c2cc(C(=O)N[C@@H]3c4ccccc4C[C@H]3O)ccc2O1 ; CCC1(CC)CC(=O)N([C@@H]2CCOc3ccc(C(=O)NC4c5ccccc5OC4(C)CO)cc32)C(=N)N1 ; CCC1(CC)CC(=O)N([C@@H]2CCOc3ccc(C(=O)NC4c5ccccc5OC4(C)CO)cc32)C(=N)N1] 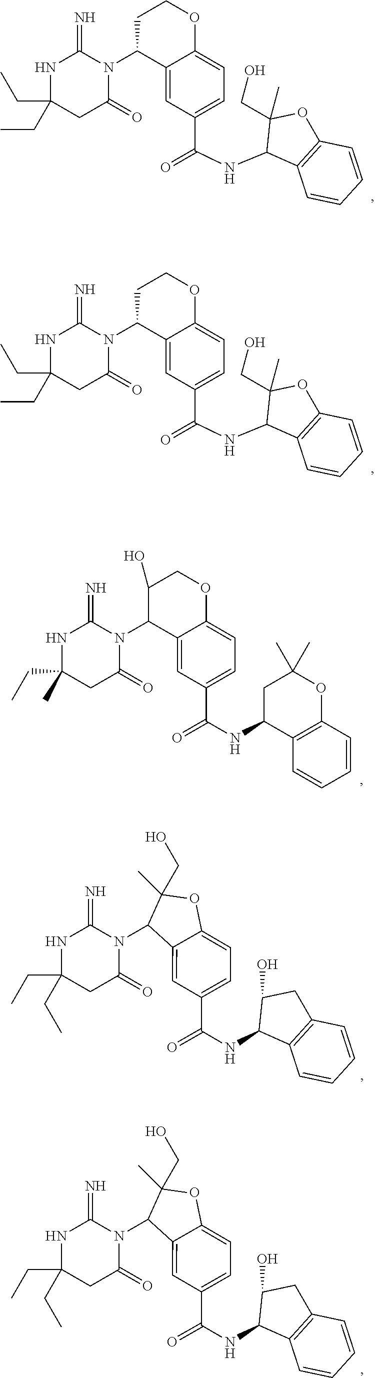 [SMILES: CCC1(CC)CC(=O)N([C@@H]2CCOc3ccc(C(=O)NC4c5ccccc5OC4(C)CO)cc32)C(=N)N1 ; CCC1(CC)CC(=O)N([C@@H]2CCOc3ccc(C(=O)NC4c5ccccc5OC4(C)CO)cc32)C(=N)N1 ; CC[C@@]1(C)CC(=O)N(C2c3cc(C(=O)N[C@H]4CC(C)(C)Oc5ccccc54)ccc3OCC2O)C(=N)N1 ; CCC1(CC)CC(=O)N(C2c3cc(C(=O)N[C@@H]4c5ccccc5C[C@H]4O)ccc3OC2(C)CO)C(=N)N1 ; CCC1(CC)CC(=O)N(C2c3cc(C(=O)N[C@@H]4c5ccccc5C[C@H]4O)ccc3OC2(C)CO)C(=N)N1] 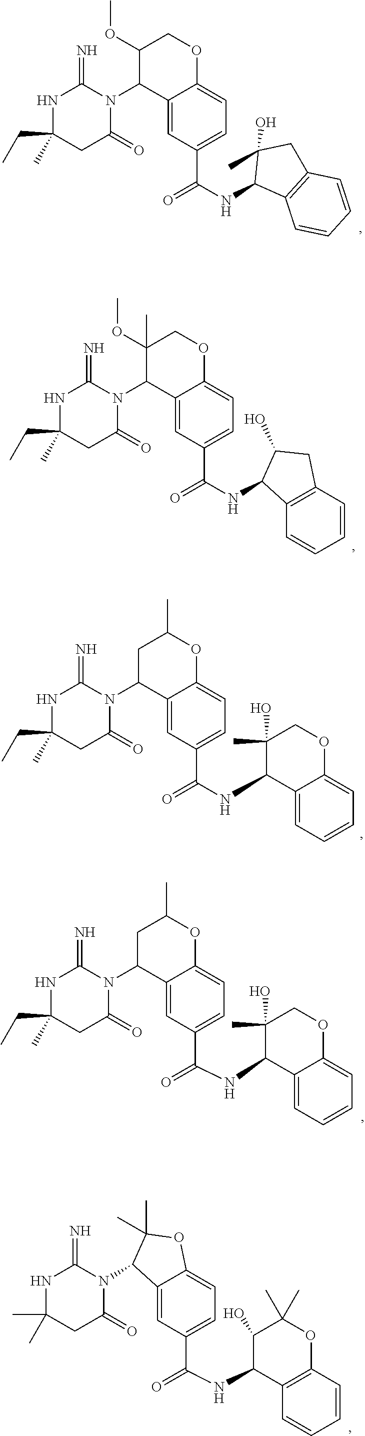 [SMILES: CC[C@]1(C)CC(=O)N(C2c3cc(C(=O)N[C@@H]4c5ccccc5C[C@@]4(C)O)ccc3OCC2OC)C(=N)N1 ; CC[C@]1(C)CC(=O)N(C2c3cc(C(=O)N[C@@H]4c5ccccc5C[C@H]4O)ccc3OCC2(C)OC)C(=N)N1 ; CC[C@]1(C)CC(=O)N(C2CC(C)Oc3ccc(C(=O)N[C@@H]4c5ccccc5OC[C@@]4(C)O)cc32)C(=N)N1 ; CC[C@]1(C)CC(=O)N(C2CC(C)Oc3ccc(C(=O)N[C@@H]4c5ccccc5OC[C@@]4(C)O)cc32)C(=N)N1 ; CC1(C)CC(=O)N([C@H]2c3cc(C(=O)N[C@@H]4c5ccccc5OC(C)(C)[C@H]4O)ccc3OC2(C)C)C(=N)N1] 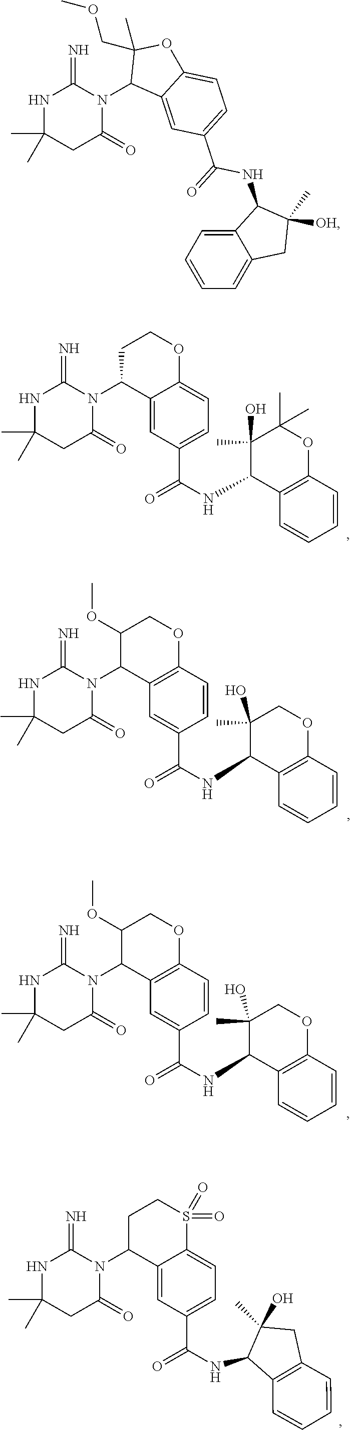 [SMILES: COCC1(C)Oc2ccc(C(=O)N[C@@H]3c4ccccc4C[C@]3(C)O)cc2C1N1C(=N)NC(C)(C)CC1=O ; CC1(C)CC(=O)N([C@@H]2CCOc3ccc(C(=O)N[C@H]4c5ccccc5OC(C)(C)[C@]4(C)O)cc32)C(=N)N1 ; COC1COc2ccc(C(=O)N[C@@H]3c4ccccc4OC[C@]3(C)O)cc2C1N1C(=N)NC(C)(C)CC1=O ; COC1COc2ccc(C(=O)N[C@@H]3c4ccccc4OC[C@@]3(C)O)cc2C1N1C(=N)NC(C)(C)CC1=O ; CC1(C)CC(=O)N(C2CCS(=O)(=O)c3ccc(C(=O)N[C@@H]4c5ccccc5C[C@]4(C)O)cc32)C(=N)N1] 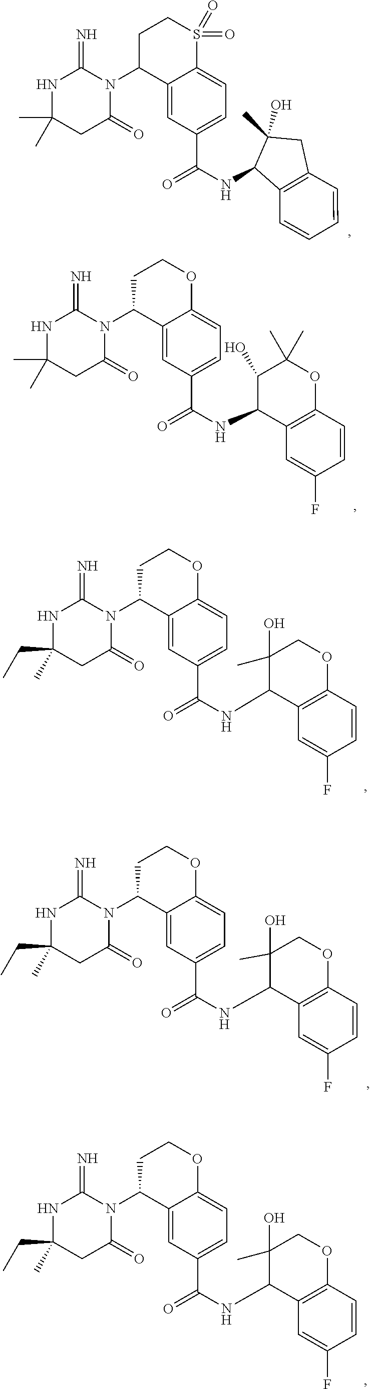 [SMILES: CC1(C)CC(=O)N(C2CCS(=O)(=O)c3ccc(C(=O)N[C@@H]4c5ccccc5C[C@@]4(C)O)cc32)C(=N)N1 ; CC1(C)CC(=O)N([C@@H]2CCOc3ccc(C(=O)N[C@@H]4c5cc(F)ccc5OC(C)(C)[C@H]4O)cc32)C(=N)N1 ; CC[C@]1(C)CC(=O)N([C@@H]2CCOc3ccc(C(=O)NC4c5cc(F)ccc5OCC4(C)O)cc32)C(=N)N1 ; CC[C@]1(C)CC(=O)N([C@@H]2CCOc3ccc(C(=O)NC4c5cc(F)ccc5OCC4(C)O)cc32)C(=N)N1 ; CC[C@]1(C)CC(=O)N([C@@H]2CCOc3ccc(C(=O)NC4c5cc(F)ccc5OCC4(C)O)cc32)C(=N)N1] 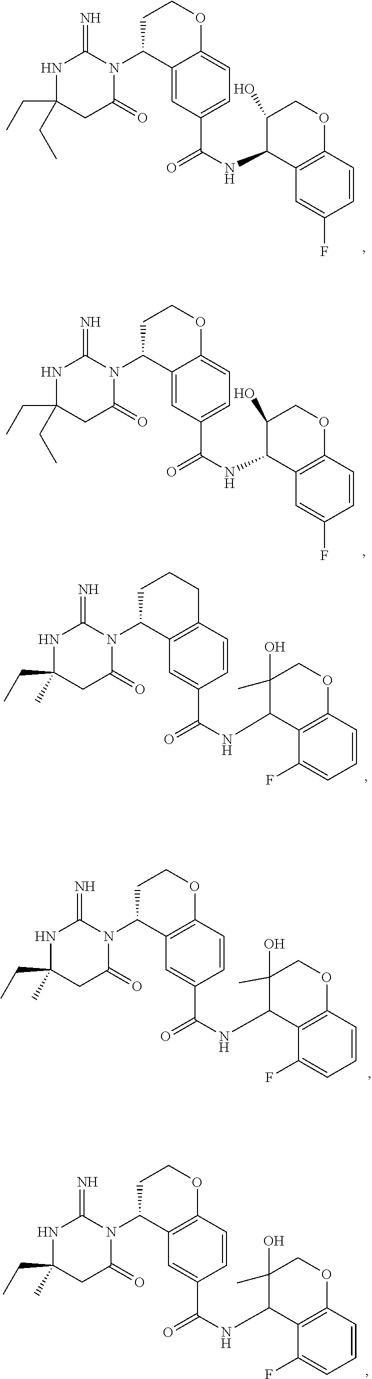 [SMILES: CCC1(CC)CC(=O)N([C@@H]2CCOc3ccc(C(=O)N[C@@H]4c5cc(F)ccc5OC[C@H]4O)cc32)C(=N)N1 ; CCC1(CC)CC(=O)N([C@@H]2CCOc3ccc(C(=O)N[C@H]4c5cc(F)ccc5OC[C@@H]4O)cc32)C(=N)N1 ; CC[C@]1(C)CC(=O)N([C@@H]2CCCc3ccc(C(=O)NC4c5c(F)cccc5OCC4(C)O)cc32)C(=N)N1 ; CC[C@]1(C)CC(=O)N([C@@H]2CCOc3ccc(C(=O)NC4c5c(F)cccc5OCC4(C)O)cc32)C(=N)N1 ; CC[C@]1(C)CC(=O)N([C@@H]2CCOc3ccc(C(=O)NC4c5c(F)cccc5OCC4(C)O)cc32)C(=N)N1] 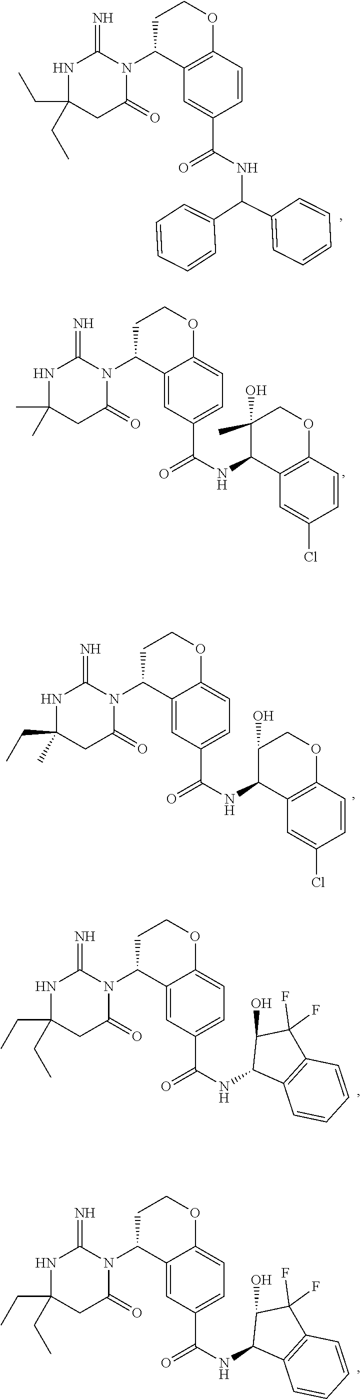 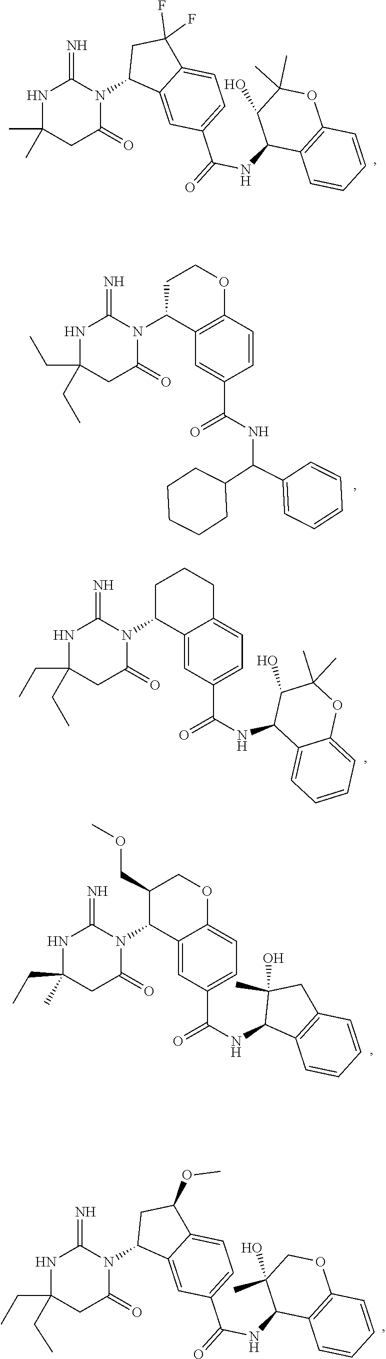 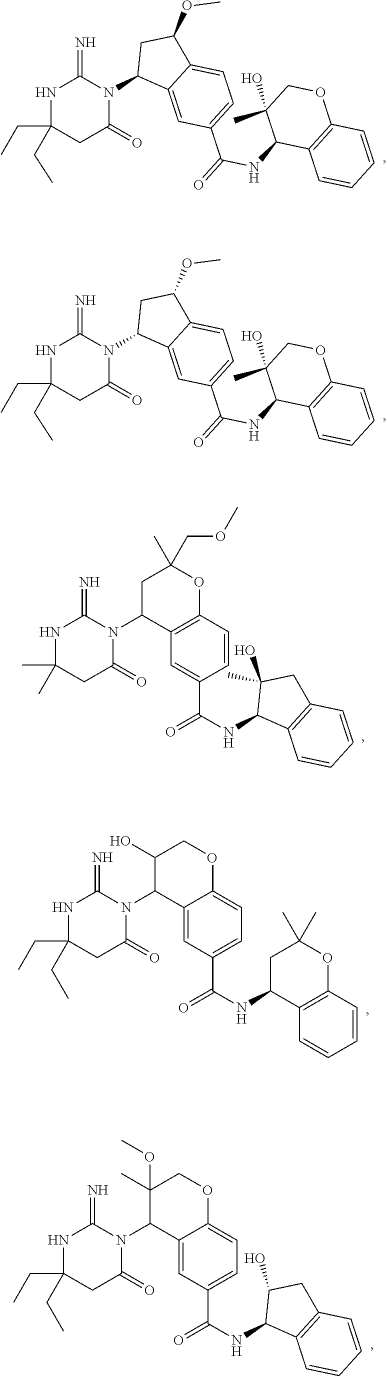 [SMILES: CCC1(CC)CC(=O)N([C@H]2C[C@@H](OC)c3ccc(C(=O)N[C@@H]4c5ccccc5OC[C@@]4(C)O)cc32)C(=N)N1 ; CCC1(CC)CC(=O)N([C@@H]2C[C@H](OC)c3ccc(C(=O)N[C@@H]4c5ccccc5OC[C@@]4(C)O)cc32)C(=N)N1 ; COCC1(C)CC(N2C(=N)NC(C)(C)CC2=O)c2cc(C(=O)N[C@@H]3c4ccccc4C[C@]3(C)O)ccc2O1 ; CCC1(CC)CC(=O)N(C2c3cc(C(=O)N[C@H]4CC(C)(C)Oc5ccccc54)ccc3OCC2O)C(=N)N1 ; CCC1(CC)CC(=O)N(C2c3cc(C(=O)N[C@@H]4c5ccccc5C[C@H]4O)ccc3OCC2(C)OC)C(=N)N1] 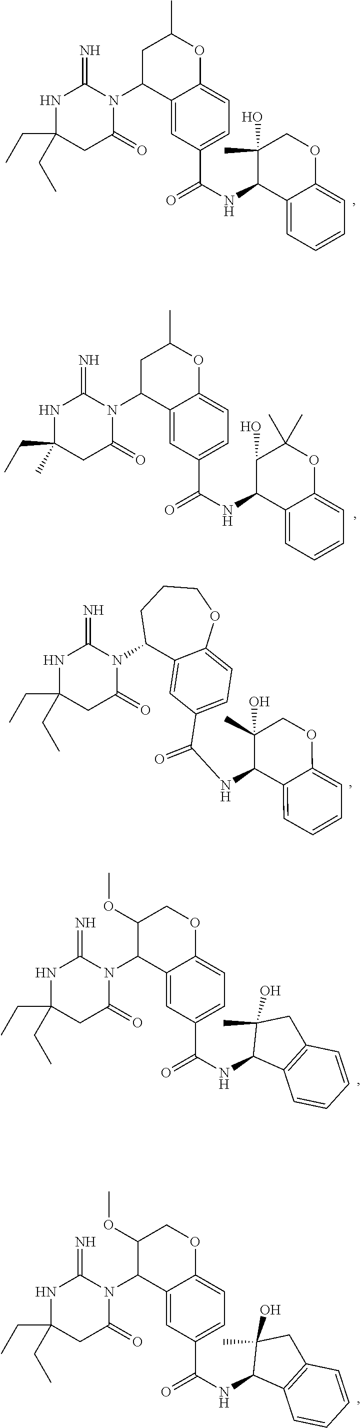 [SMILES: CCC1(CC)CC(=O)N(C2CC(C)Oc3ccc(C(=O)N[C@@H]4c5ccccc5OC[C@@]4(C)O)cc32)C(=N)N1 ; CC[C@]1(C)CC(=O)N(C2CC(C)Oc3ccc(C(=O)N[C@@H]4c5ccccc5OC(C)(C)[C@H]4O)cc32)C(=N)N1 ; CCC1(CC)CC(=O)N([C@@H]2CCCOc3ccc(C(=O)N[C@@H]4c5ccccc5OC[C@@]4(C)O)cc32)C(=N)N1 ; CCC1(CC)CC(=O)N(C2c3cc(C(=O)N[C@@H]4c5ccccc5C[C@@]4(C)O)ccc3OCC2OC)C(=N)N1 ; CCC1(CC)CC(=O)N(C2c3cc(C(=O)N[C@@H]4c5ccccc5C[C@]4(C)O)ccc3OCC2OC)C(=N)N1] 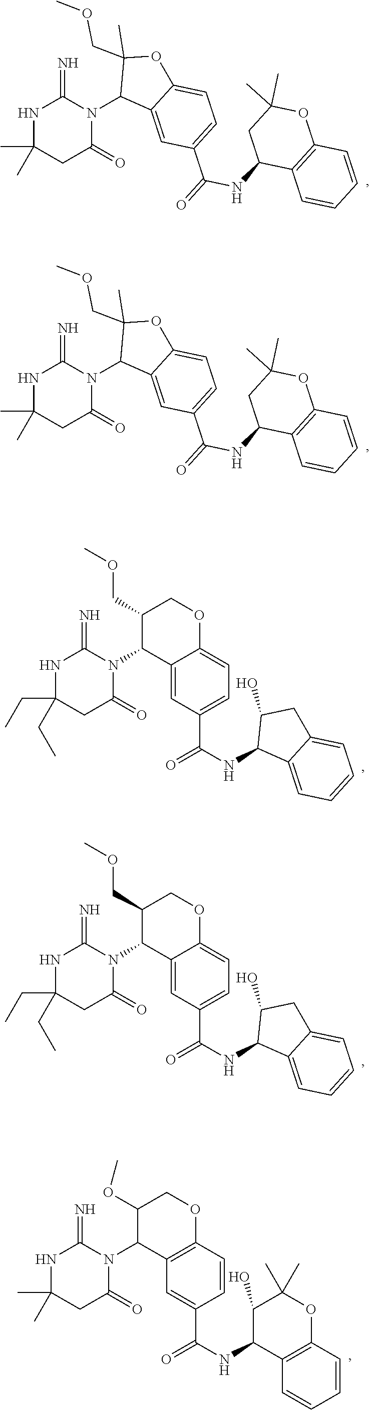 [SMILES: COCC1(C)Oc2ccc(C(=O)N[C@H]3CC(C)(C)Oc4ccccc43)cc2C1N1C(=N)NC(C)(C)CC1=O ; COCC1(C)Oc2ccc(C(=O)N[C@H]3CC(C)(C)Oc4ccccc43)cc2C1N1C(=N)NC(C)(C)CC1=O ; CCC1(CC)CC(=O)N([C@H]2c3cc(C(=O)N[C@@H]4c5ccccc5C[C@H]4O)ccc3OC[C@H]2COC)C(=N)N1 ; CCC1(CC)CC(=O)N([C@H]2c3cc(C(=O)N[C@@H]4c5ccccc5C[C@H]4O)ccc3OC[C@@H]2COC)C(=N)N1 ; COC1COc2ccc(C(=O)N[C@@H]3c4ccccc4OC(C)(C)[C@H]3O)cc2C1N1C(=N)NC(C)(C)CC1=O] 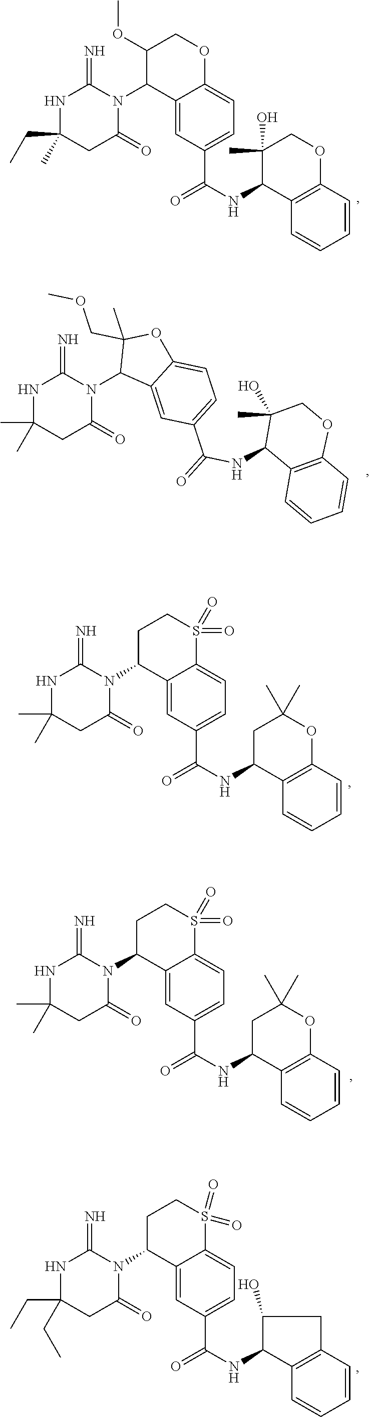 [SMILES: CC[C@]1(C)CC(=O)N(C2c3cc(C(=O)N[C@@H]4c5ccccc5OC[C@@]4(C)O)ccc3OCC2OC)C(=N)N1 ; COCC1(C)Oc2ccc(C(=O)N[C@@H]3c4ccccc4OC[C@@]3(C)O)cc2C1N1C(=N)NC(C)(C)CC1=O ; CC1(C)CC(=O)N([C@@H]2CCS(=O)(=O)c3ccc(C(=O)N[C@H]4CC(C)(C)Oc5ccccc54)cc32)C(=N)N1 ; CC1(C)CC(=O)N([C@H]2CCS(=O)(=O)c3ccc(C(=O)N[C@H]4CC(C)(C)Oc5ccccc54)cc32)C(=N)N1 ; CCC1(CC)CC(=O)N([C@@H]2CCS(=O)(=O)c3ccc(C(=O)N[C@@H]4c5ccccc5C[C@H]4O)cc32)C(=N)N1] 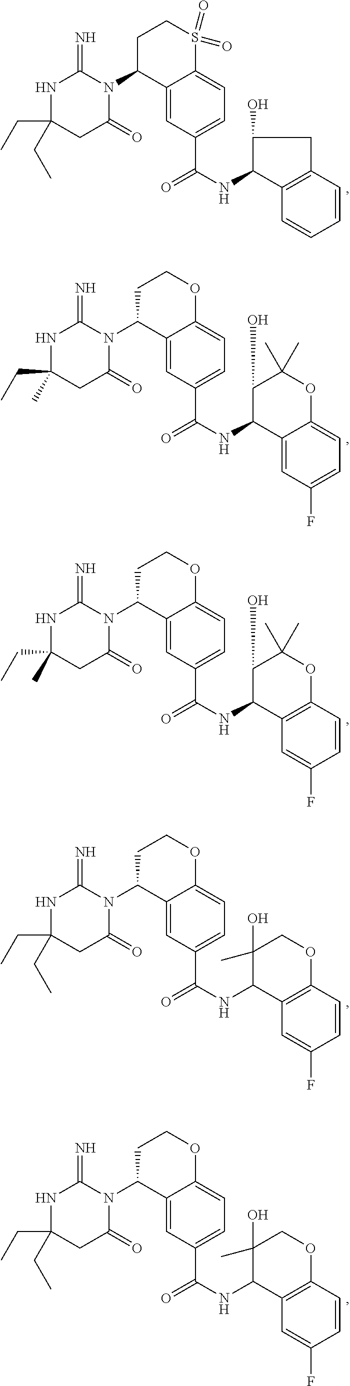 [SMILES: CCC1(CC)CC(=O)N([C@H]2CCS(=O)(=O)c3ccc(C(=O)N[C@@H]4c5ccccc5C[C@H]4O)cc32)C(=N)N1 ; CC[C@]1(C)CC(=O)N([C@@H]2CCOc3ccc(C(=O)N[C@@H]4c5cc(F)ccc5OC(C)(C)[C@H]4O)cc32)C(=N)N1 ; CC[C@@]1(C)CC(=O)N([C@@H]2CCOc3ccc(C(=O)N[C@@H]4c5cc(F)ccc5OC(C)(C)[C@H]4O)cc32)C(=N)N1 ; CCC1(CC)CC(=O)N([C@@H]2CCOc3ccc(C(=O)NC4c5cc(F)ccc5OCC4(C)O)cc32)C(=N)N1 ; CCC1(CC)CC(=O)N([C@@H]2CCOc3ccc(C(=O)NC4c5cc(F)ccc5OCC4(C)O)cc32)C(=N)N1] 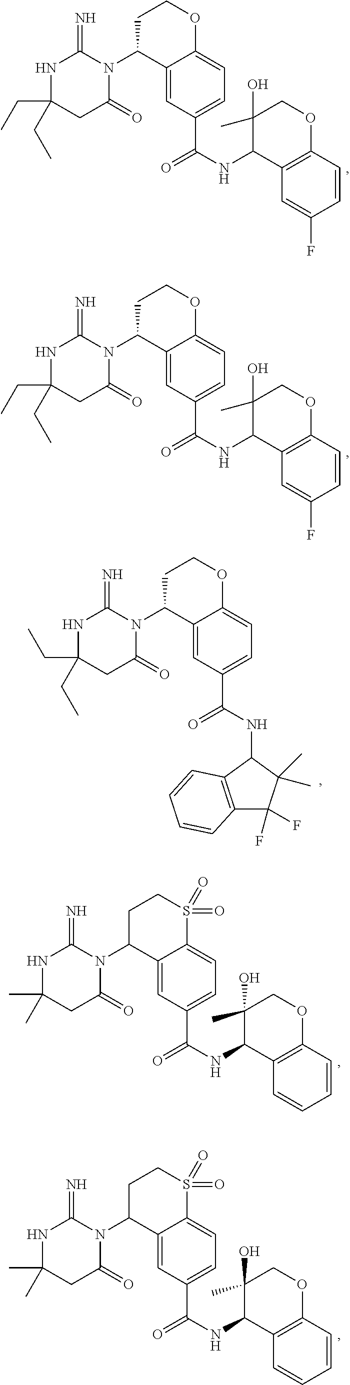 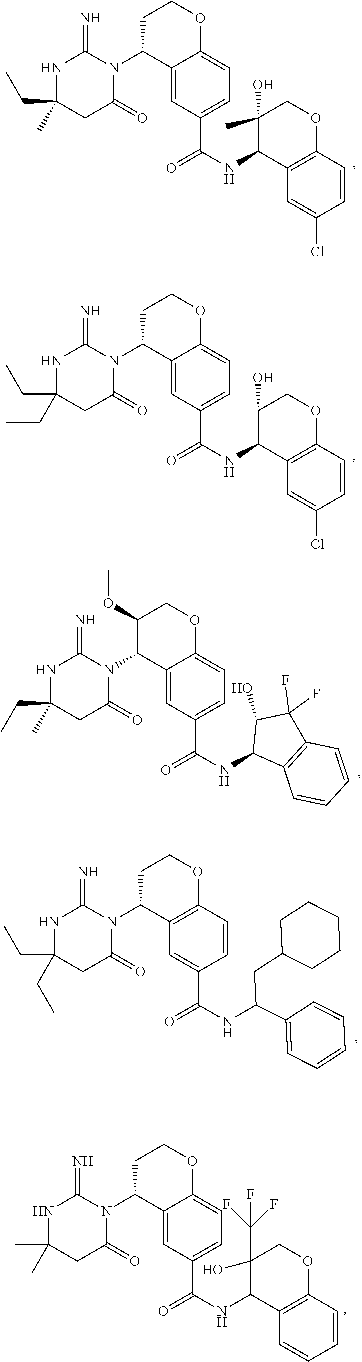 [SMILES: CC[C@]1(C)CC(=O)N([C@@H]2CCOc3ccc(C(=O)N[C@@H]4c5cc(Cl)ccc5OC[C@@]4(C)O)cc32)C(=N)N1 ; CCC1(CC)CC(=O)N([C@@H]2CCOc3ccc(C(=O)N[C@@H]4c5cc(Cl)ccc5OC[C@H]4O)cc32)C(=N)N1 ; CC[C@]1(C)CC(=O)N([C@H]2c3cc(C(=O)N[C@@H]4c5ccccc5C(F)(F)[C@H]4O)ccc3OC[C@@H]2OC)C(=N)N1 ; CCC1(CC)CC(=O)N([C@@H]2CCOc3ccc(C(=O)NC(CC4CCCCC4)c4ccccc4)cc32)C(=N)N1 ; CC1(C)CC(=O)N([C@@H]2CCOc3ccc(C(=O)NC4c5ccccc5OCC4(O)C(F)(F)F)cc32)C(=N)N1] 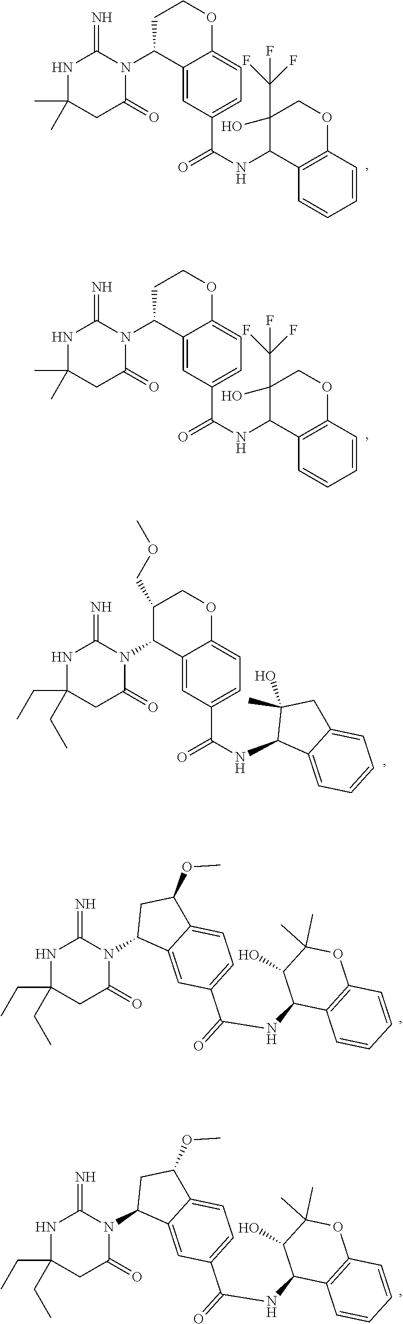 [SMILES: CC1(C)CC(=O)N([C@@H]2CCOc3ccc(C(=O)NC4c5ccccc5OCC4(O)C(F)(F)F)cc32)C(=N)N1 ; CC1(C)CC(=O)N([C@@H]2CCOc3ccc(C(=O)NC4c5ccccc5OCC4(O)C(F)(F)F)cc32)C(=N)N1 ; CCC1(CC)CC(=O)N([C@H]2c3cc(C(=O)N[C@@H]4c5ccccc5C[C@@]4(C)O)ccc3OC[C@H]2COC)C(=N)N1 ; CCC1(CC)CC(=O)N([C@@H]2C[C@@H](OC)c3ccc(C(=O)N[C@@H]4c5ccccc5OC(C)(C)[C@H]4O)cc32)C(=N)N1 ; CCC1(CC)CC(=O)N([C@H]2C[C@H](OC)c3ccc(C(=O)N[C@@H]4c5ccccc5OC(C)(C)[C@H]4O)cc32)C(=N)N1] 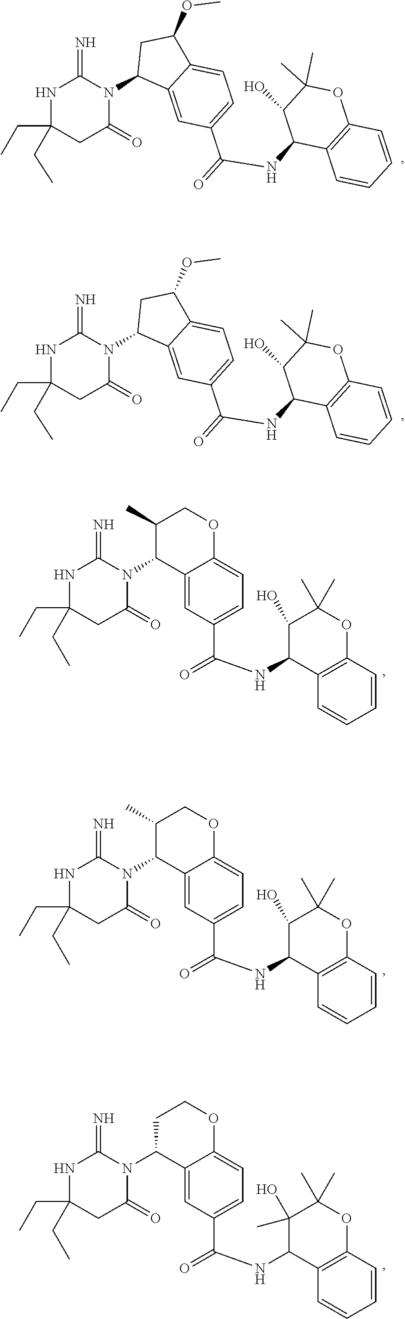 [SMILES: CCC1(CC)CC(=O)N([C@H]2C[C@@H](OC)c3ccc(C(=O)N[C@@H]4c5ccccc5OC(C)(C)[C@H]4O)cc32)C(=N)N1 ; CCC1(CC)CC(=O)N([C@@H]2C[C@H](OC)c3ccc(C(=O)N[C@@H]4c5ccccc5OC(C)(C)[C@H]4O)cc32)C(=N)N1 ; CCC1(CC)CC(=O)N([C@H]2c3cc(C(=O)N[C@@H]4c5ccccc5OC(C)(C)[C@H]4O)ccc3OC[C@@H]2C)C(=N)N1 ; CCC1(CC)CC(=O)N([C@H]2c3cc(C(=O)N[C@@H]4c5ccccc5OC(C)(C)[C@H]4O)ccc3OC[C@H]2C)C(=N)N1 ; CCC1(CC)CC(=O)N([C@@H]2CCOc3ccc(C(=O)NC4c5ccccc5OC(C)(C)C4(C)O)cc32)C(=N)N1] 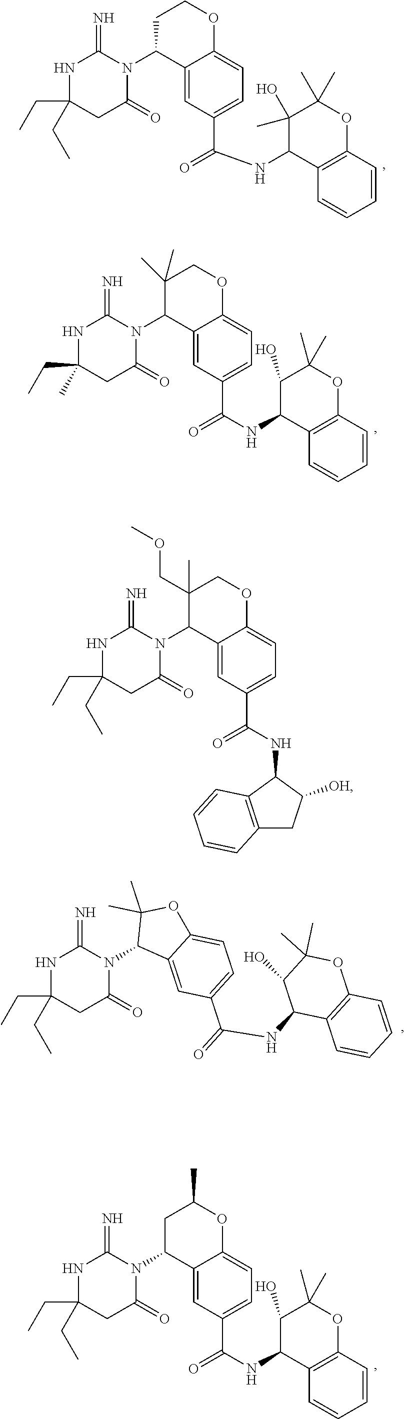 [SMILES: CCC1(CC)CC(=O)N([C@@H]2CCOc3ccc(C(=O)NC4c5ccccc5OC(C)(C)C4(C)O)cc32)C(=N)N1 ; CC[C@]1(C)CC(=O)N(C2c3cc(C(=O)N[C@@H]4c5ccccc5OC(C)(C)[C@H]4O)ccc3OCC2(C)C)C(=N)N1 ; CCC1(CC)CC(=O)N(C2c3cc(C(=O)N[C@@H]4c5ccccc5C[C@H]4O)ccc3OCC2(C)COC)C(=N)N1 ; CCC1(CC)CC(=O)N([C@H]2c3cc(C(=O)N[C@@H]4c5ccccc5OC(C)(C)[C@H]4O)ccc3OC2(C)C)C(=N)N1 ; CCC1(CC)CC(=O)N([C@@H]2C[C@@H](C)Oc3ccc(C(=O)N[C@@H]4c5ccccc5OC(C)(C)[C@H]4O)cc32)C(=N)N1] 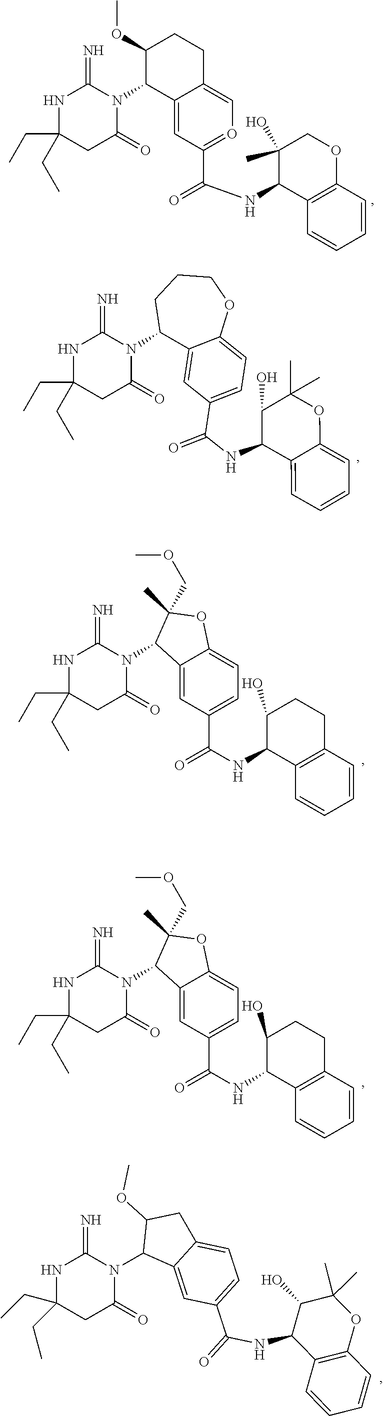 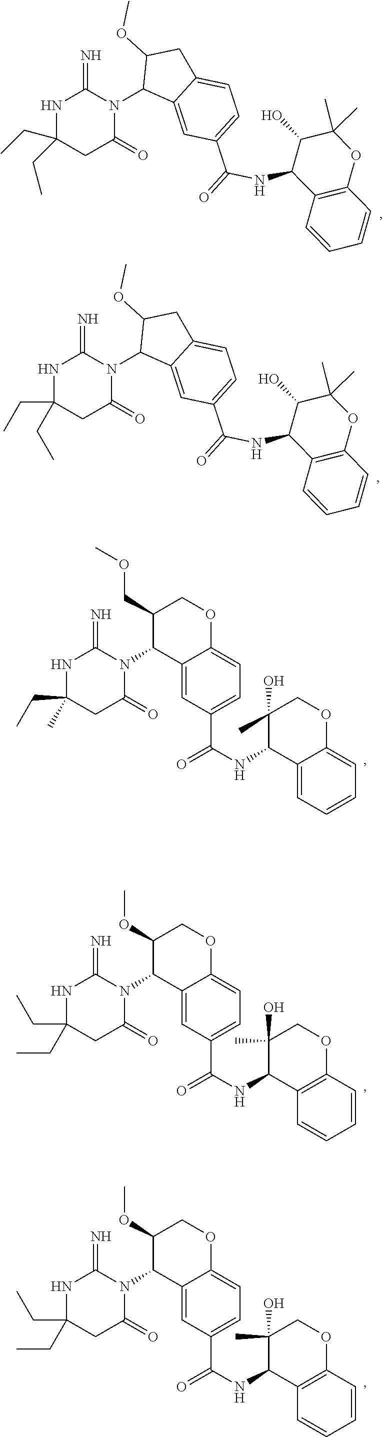 [SMILES: CCC1(CC)CC(=O)N(C2c3cc(C(=O)N[C@@H]4c5ccccc5OC(C)(C)[C@H]4O)ccc3CC2OC)C(=N)N1 ; CCC1(CC)CC(=O)N(C2c3cc(C(=O)N[C@@H]4c5ccccc5OC(C)(C)[C@H]4O)ccc3CC2OC)C(=N)N1 ; CC[C@]1(C)CC(=O)N([C@H]2c3cc(C(=O)N[C@H]4c5ccccc5OC[C@@]4(C)O)ccc3OC[C@@H]2COC)C(=N)N1 ; CCC1(CC)CC(=O)N([C@H]2c3cc(C(=O)N[C@@H]4c5ccccc5OC[C@]4(C)O)ccc3OC[C@@H]2OC)C(=N)N1 ; CCC1(CC)CC(=O)N([C@H]2c3cc(C(=O)N[C@@H]4c5ccccc5OC[C@@]4(C)O)ccc3OC[C@@H]2OC)C(=N)N1] 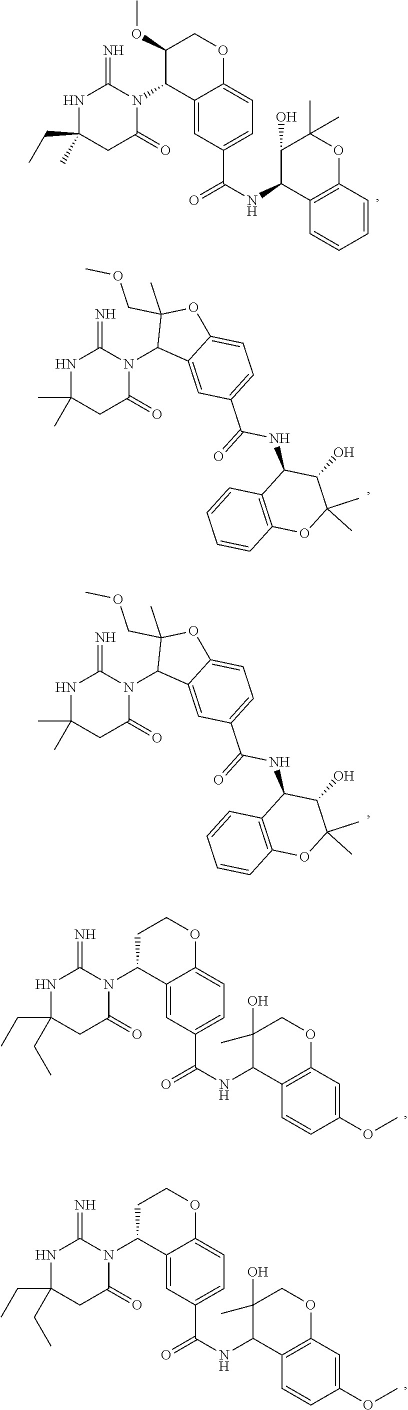 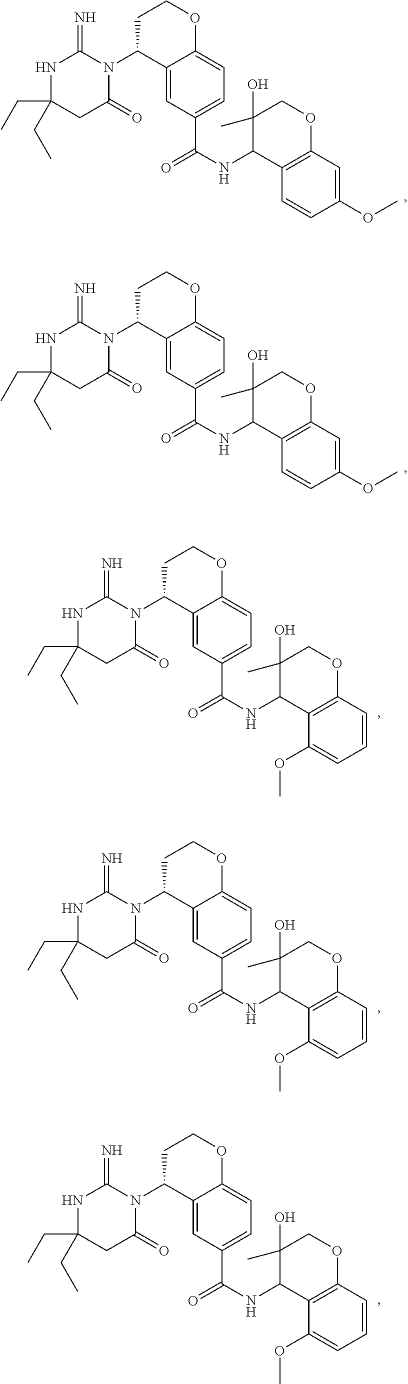 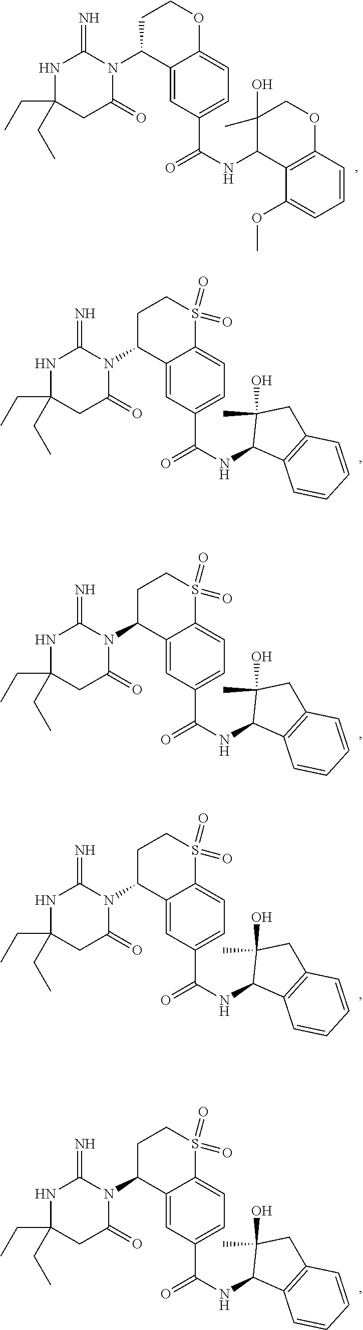 [SMILES: CCC1(CC)CC(=O)N([C@@H]2CCOc3ccc(C(=O)NC4c5c(OC)cccc5OCC4(C)O)cc32)C(=N)N1 ; CCC1(CC)CC(=O)N([C@@H]2CCS(=O)(=O)c3ccc(C(=O)N[C@@H]4c5ccccc5C[C@@]4(C)O)cc32)C(=N)N1 ; CCC1(CC)CC(=O)N([C@H]2CCS(=O)(=O)c3ccc(C(=O)N[C@@H]4c5ccccc5C[C@@]4(C)O)cc32)C(=N)N1 ; CCC1(CC)CC(=O)N([C@@H]2CCS(=O)(=O)c3ccc(C(=O)N[C@@H]4c5ccccc5C[C@]4(C)O)cc32)C(=N)N1 ; CCC1(CC)CC(=O)N([C@H]2CCS(=O)(=O)c3ccc(C(=O)N[C@@H]4c5ccccc5C[C@]4(C)O)cc32)C(=N)N1] 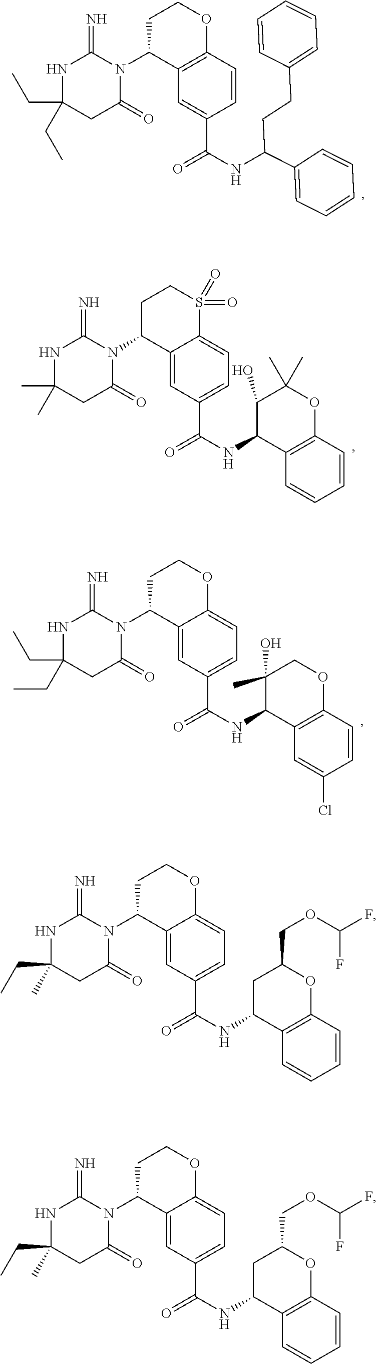 [SMILES: CCC1(CC)CC(=O)N([C@@H]2CCOc3ccc(C(=O)NC(CCc4ccccc4)c4ccccc4)cc32)C(=N)N1 ; CC1(C)CC(=O)N([C@@H]2CCS(=O)(=O)c3ccc(C(=O)N[C@@H]4c5ccccc5OC(C)(C)[C@H]4O)cc32)C(=N)N1 ; CCC1(CC)CC(=O)N([C@@H]2CCOc3ccc(C(=O)N[C@@H]4c5cc(Cl)ccc5OC[C@@]4(C)O)cc32)C(=N)N1 ; CC[C@]1(C)CC(=O)N([C@@H]2CCOc3ccc(C(=O)N[C@@H]4C[C@@H](COC(F)F)Oc5ccccc54)cc32)C(=N)N1 ; CC[C@]1(C)CC(=O)N([C@@H]2CCOc3ccc(C(=O)N[C@@H]4C[C@H](COC(F)F)Oc5ccccc54)cc32)C(=N)N1] 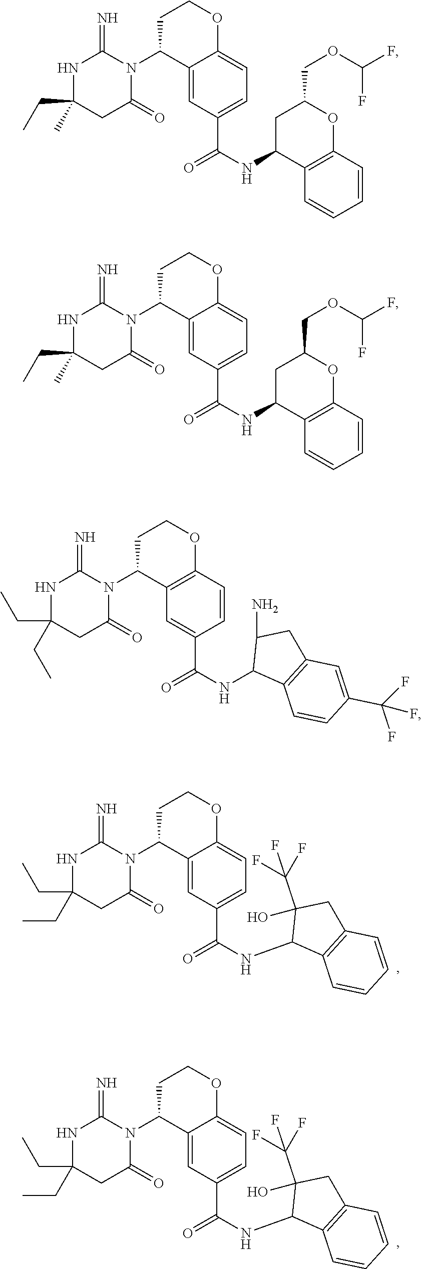 [SMILES: CC[C@]1(C)CC(=O)N([C@@H]2CCOc3ccc(C(=O)N[C@H]4C[C@H](COC(F)F)Oc5ccccc54)cc32)C(=N)N1 ; CC[C@]1(C)CC(=O)N([C@@H]2CCOc3ccc(C(=O)N[C@H]4C[C@@H](COC(F)F)Oc5ccccc54)cc32)C(=N)N1 ; CCC1(CC)CC(=O)N([C@@H]2CCOc3ccc(C(=O)NC4c5ccc(C(F)(F)F)cc5CC4N)cc32)C(=N)N1 ; CCC1(CC)CC(=O)N([C@@H]2CCOc3ccc(C(=O)NC4c5ccccc5CC4(O)C(F)(F)F)cc32)C(=N)N1 ; CCC1(CC)CC(=O)N([C@@H]2CCOc3ccc(C(=O)NC4c5ccccc5CC4(O)C(F)(F)F)cc32)C(=N)N1] 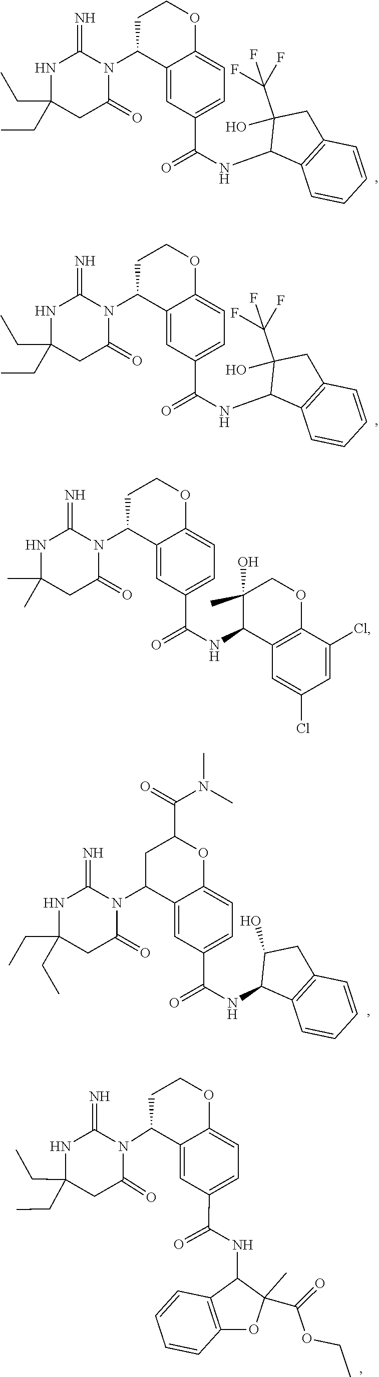 [SMILES: CCC1(CC)CC(=O)N([C@@H]2CCOc3ccc(C(=O)NC4c5ccccc5CC4(O)C(F)(F)F)cc32)C(=N)N1 ; CCC1(CC)CC(=O)N([C@@H]2CCOc3ccc(C(=O)NC4c5ccccc5CC4(O)C(F)(F)F)cc32)C(=N)N1 ; CC1(C)CC(=O)N([C@@H]2CCOc3ccc(C(=O)N[C@@H]4c5cc(Cl)cc(Cl)c5OC[C@@]4(C)O)cc32)C(=N)N1 ; CCC1(CC)CC(=O)N(C2CC(C(=O)N(C)C)Oc3ccc(C(=O)N[C@@H]4c5ccccc5C[C@H]4O)cc32)C(=N)N1 ; CCOC(=O)C1(C)Oc2ccccc2C1NC(=O)c1ccc2c(c1)[C@H](N1C(=N)NC(CC)(CC)CC1=O)CCO2] 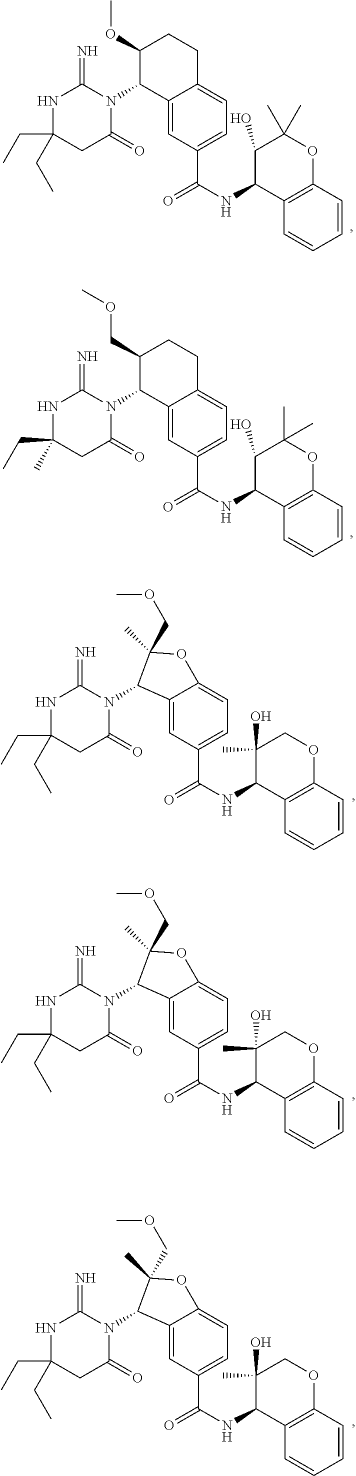 [SMILES: CCC1(CC)CC(=O)N([C@H]2c3cc(C(=O)N[C@@H]4c5ccccc5OC(C)(C)[C@H]4O)ccc3CC[C@@H]2OC)C(=N)N1 ; CC[C@]1(C)CC(=O)N([C@H]2c3cc(C(=O)N[C@@H]4c5ccccc5OC(C)(C)[C@H]4O)ccc3CC[C@@H]2COC)C(=N)N1 ; CCC1(CC)CC(=O)N([C@H]2c3cc(C(=O)N[C@@H]4c5ccccc5OC[C@]4(C)O)ccc3O[C@]2(C)COC)C(=N)N1 ; CCC1(CC)CC(=O)N([C@H]2c3cc(C(=O)N[C@@H]4c5ccccc5OC[C@@]4(C)O)ccc3O[C@]2(C)COC)C(=N)N1 ; CCC1(CC)CC(=O)N([C@H]2c3cc(C(=O)N[C@@H]4c5ccccc5OC[C@]4(C)O)ccc3O[C@@]2(C)COC)C(=N)N1] 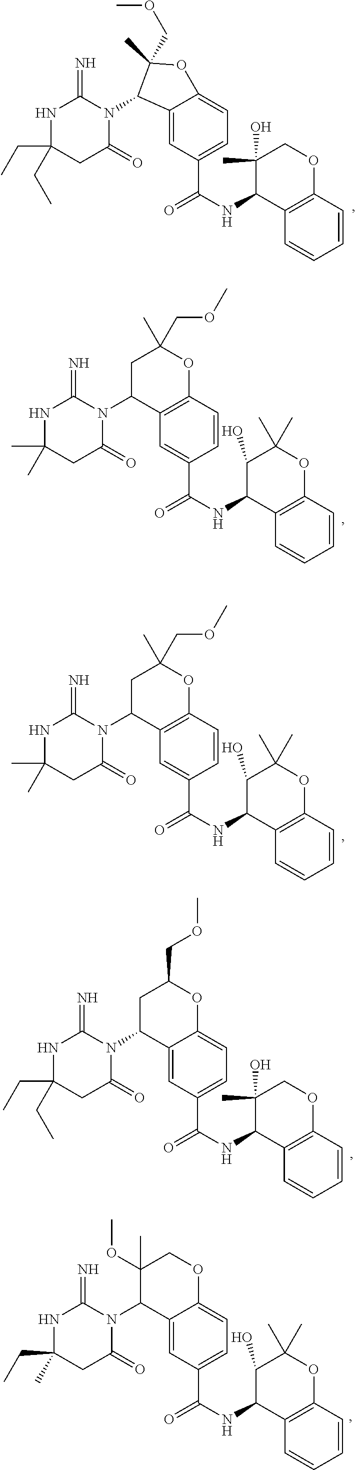 [SMILES: CCC1(CC)CC(=O)N([C@H]2c3cc(C(=O)N[C@@H]4c5ccccc5OC[C@@]4(C)O)ccc3O[C@@]2(C)COC)C(=N)N1 ; COCC1(C)CC(N2C(=N)NC(C)(C)CC2=O)c2cc(C(=O)N[C@@H]3c4ccccc4OC(C)(C)[C@H]3O)ccc2O1 ; COCC1(C)CC(N2C(=N)NC(C)(C)CC2=O)c2cc(C(=O)N[C@@H]3c4ccccc4OC(C)(C)[C@H]3O)ccc2O1 ; CCC1(CC)CC(=O)N([C@@H]2C[C@@H](COC)Oc3ccc(C(=O)N[C@@H]4c5ccccc5OC[C@@]4(C)O)cc32)C(=N)N1 ; CC[C@]1(C)CC(=O)N(C2c3cc(C(=O)N[C@@H]4c5ccccc5OC(C)(C)[C@H]4O)ccc3OCC2(C)OC)C(=N)N1] 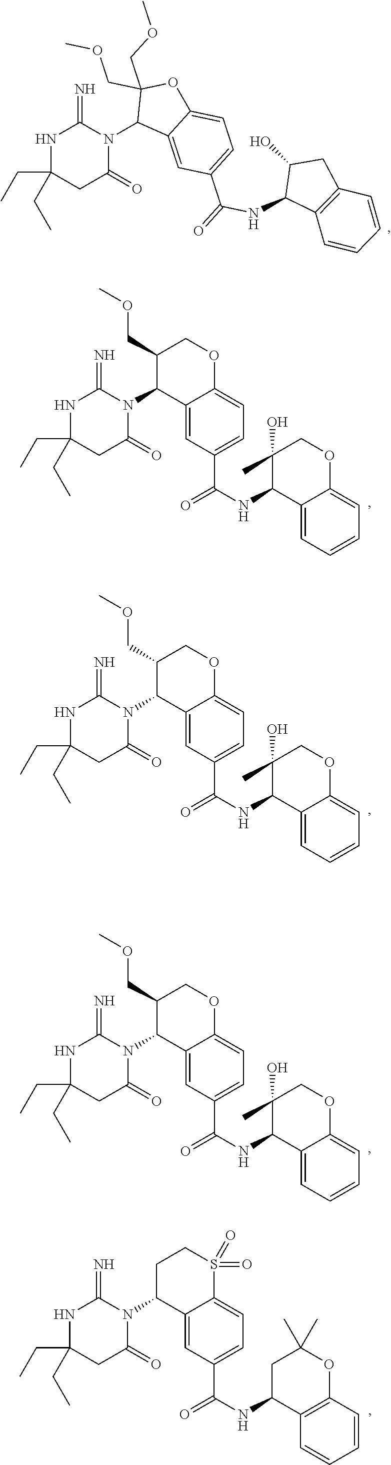 [SMILES: CCC1(CC)CC(=O)N(C2c3cc(C(=O)N[C@@H]4c5ccccc5C[C@H]4O)ccc3OC2(COC)COC)C(=N)N1 ; CCC1(CC)CC(=O)N([C@@H]2c3cc(C(=O)N[C@@H]4c5ccccc5OC[C@@]4(C)O)ccc3OC[C@@H]2COC)C(=N)N1 ; CCC1(CC)CC(=O)N([C@H]2c3cc(C(=O)N[C@@H]4c5ccccc5OC[C@@]4(C)O)ccc3OC[C@H]2COC)C(=N)N1 ; CCC1(CC)CC(=O)N([C@H]2c3cc(C(=O)N[C@@H]4c5ccccc5OC[C@@]4(C)O)ccc3OC[C@@H]2COC)C(=N)N1 ; CCC1(CC)CC(=O)N([C@@H]2CCS(=O)(=O)c3ccc(C(=O)N[C@H]4CC(C)(C)Oc5ccccc54)cc32)C(=N)N1] 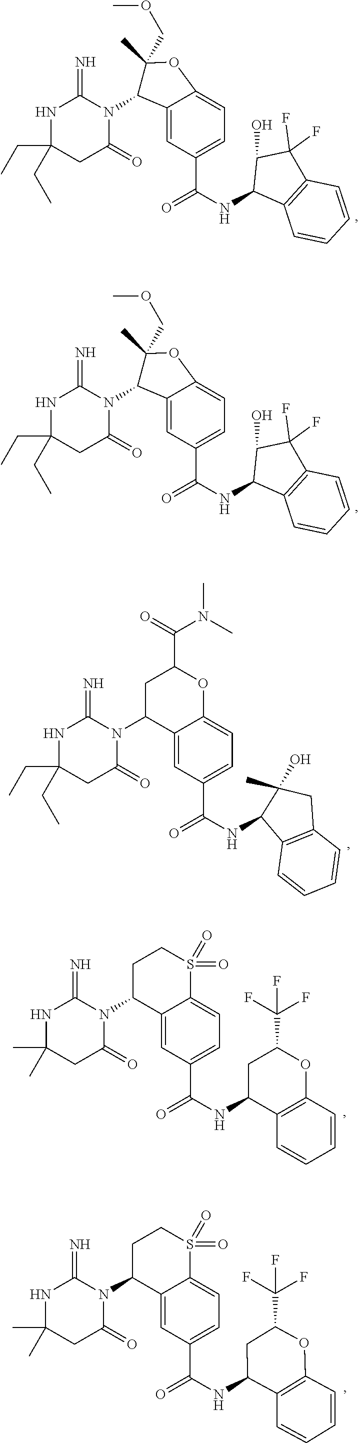 [SMILES: CCC1(CC)CC(=O)N([C@H]2c3cc(C(=O)N[C@@H]4c5ccccc5C(F)(F)[C@H]4O)ccc3O[C@@]2(C)COC)C(=N)N1 ; CCC1(CC)CC(=O)N([C@H]2c3cc(C(=O)N[C@@H]4c5ccccc5C(F)(F)[C@H]4O)ccc3O[C@@]2(C)COC)C(=N)N1 ; CCC1(CC)CC(=O)N(C2CC(C(=O)N(C)C)Oc3ccc(C(=O)N[C@@H]4c5ccccc5C[C@@]4(C)O)cc32)C(=N)N1 ; CC1(C)CC(=O)N([C@@H]2CCS(=O)(=O)c3ccc(C(=O)N[C@H]4C[C@H](C(F)(F)F)Oc5ccccc54)cc32)C(=N)N1 ; CC1(C)CC(=O)N([C@H]2CCS(=O)(=O)c3ccc(C(=O)N[C@H]4C[C@H](C(F)(F)F)Oc5ccccc54)cc32)C(=N)N1] 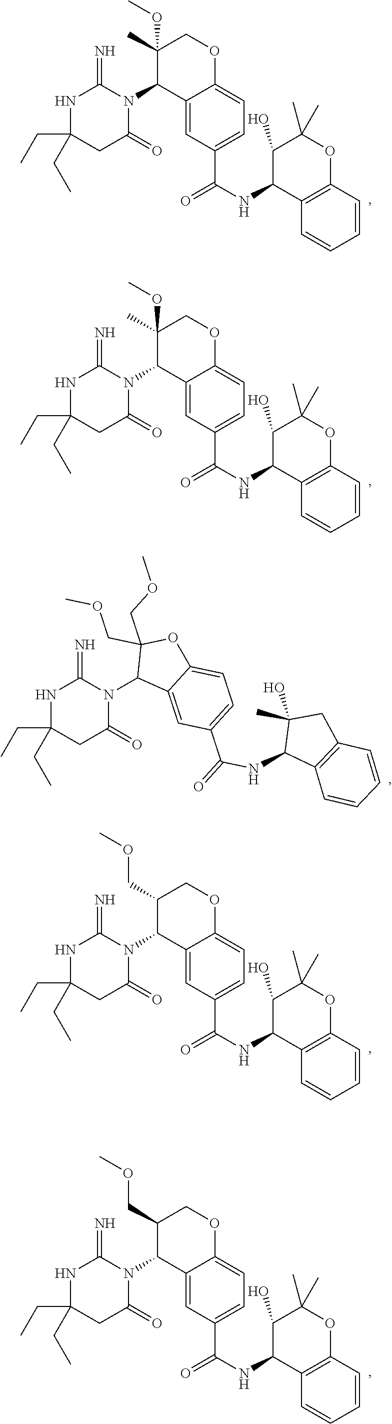 [SMILES: CCC1(CC)CC(=O)N([C@@H]2c3cc(C(=O)N[C@@H]4c5ccccc5OC(C)(C)[C@H]4O)ccc3OC[C@@]2(C)OC)C(=N)N1 ; CCC1(CC)CC(=O)N([C@H]2c3cc(C(=O)N[C@@H]4c5ccccc5OC(C)(C)[C@H]4O)ccc3OC[C@]2(C)OC)C(=N)N1 ; CCC1(CC)CC(=O)N(C2c3cc(C(=O)N[C@@H]4c5ccccc5C[C@@]4(C)O)ccc3OC2(COC)COC)C(=N)N1 ; CCC1(CC)CC(=O)N([C@H]2c3cc(C(=O)N[C@@H]4c5ccccc5OC(C)(C)[C@H]4O)ccc3OC[C@H]2COC)C(=N)N1 ; CCC1(CC)CC(=O)N([C@H]2c3cc(C(=O)N[C@@H]4c5ccccc5OC(C)(C)[C@H]4O)ccc3OC[C@@H]2COC)C(=N)N1] 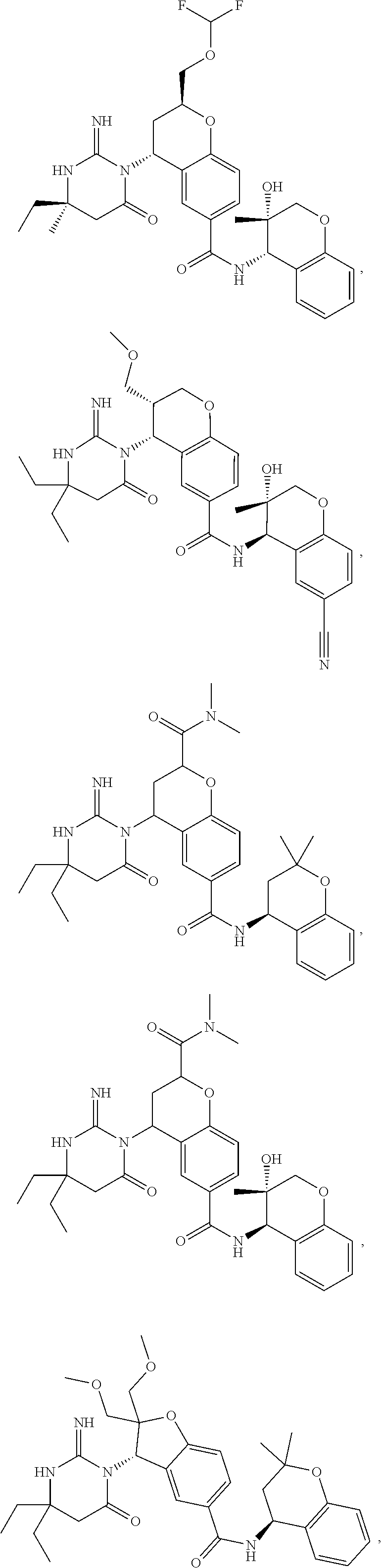 [SMILES: CC[C@]1(C)CC(=O)N([C@@H]2C[C@@H](COC(F)F)Oc3ccc(C(=O)N[C@H]4c5ccccc5OC[C@@]4(C)O)cc32)C(=N)N1 ; CCC1(CC)CC(=O)N([C@H]2c3cc(C(=O)N[C@@H]4c5cc(C#N)ccc5OC[C@@]4(C)O)ccc3OC[C@H]2COC)C(=N)N1 ; CCC1(CC)CC(=O)N(C2CC(C(=O)N(C)C)Oc3ccc(C(=O)N[C@H]4CC(C)(C)Oc5ccccc54)cc32)C(=N)N1 ; CCC1(CC)CC(=O)N(C2CC(C(=O)N(C)C)Oc3ccc(C(=O)N[C@@H]4c5ccccc5OC[C@@]4(C)O)cc32)C(=N)N1 ; CCC1(CC)CC(=O)N([C@H]2c3cc(C(=O)N[C@H]4CC(C)(C)Oc5ccccc54)ccc3OC2(COC)COC)C(=N)N1] 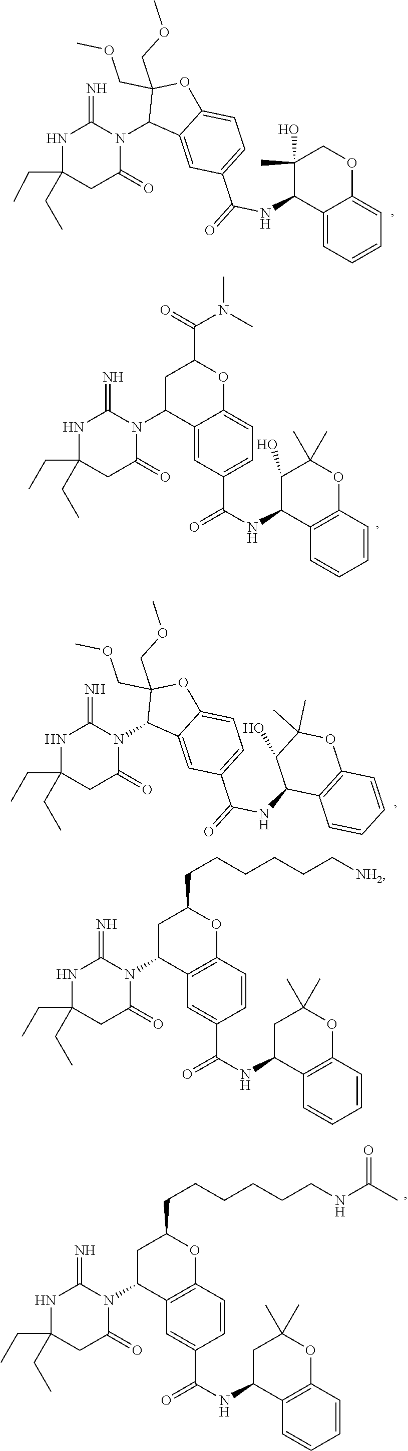 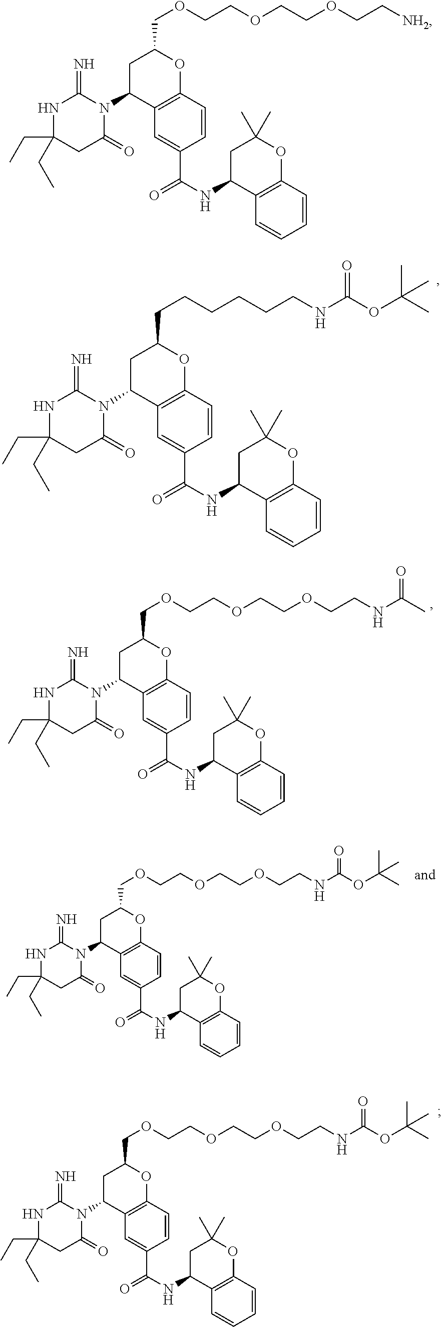
or a pharmaceutically acceptable salt thereof.

29. A method of chemovaccination against Plasmodium infections comprising administering to a patient an effective amount of a compound, or a pharmaceutically acceptable salt thereof, selected from the group consisting of

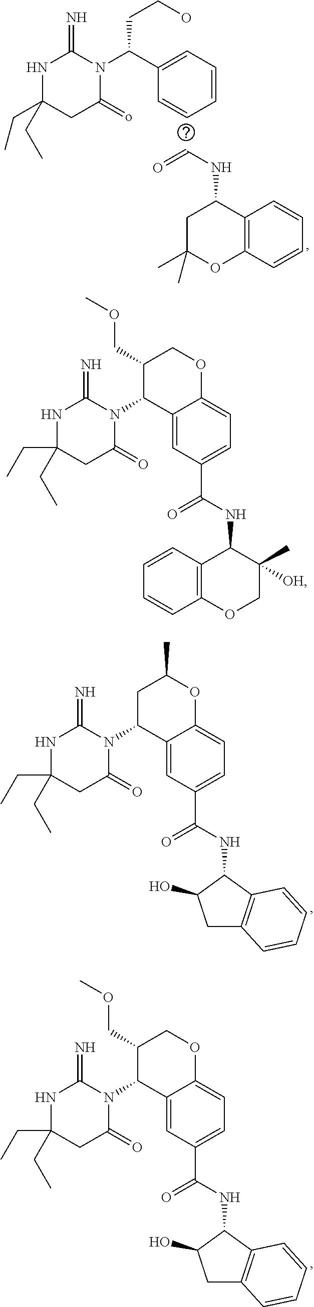

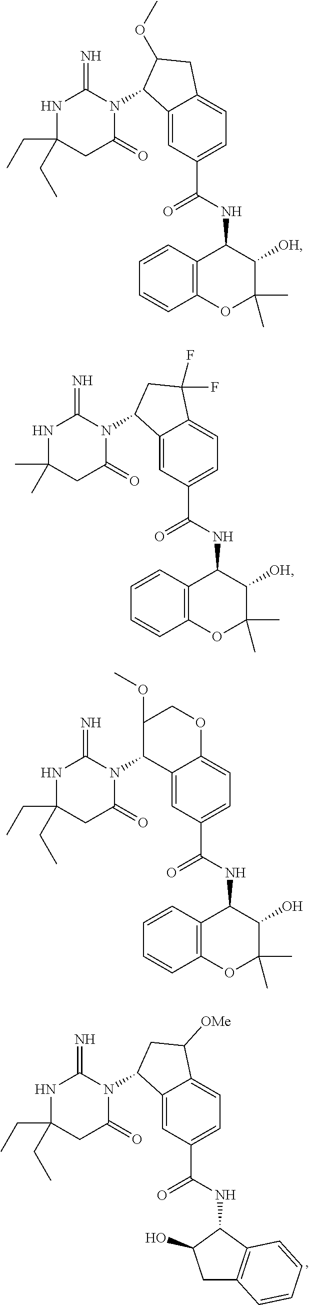

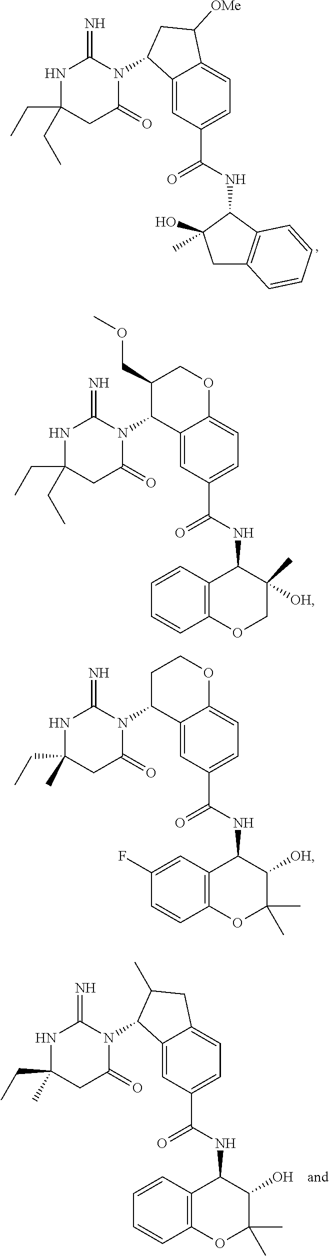

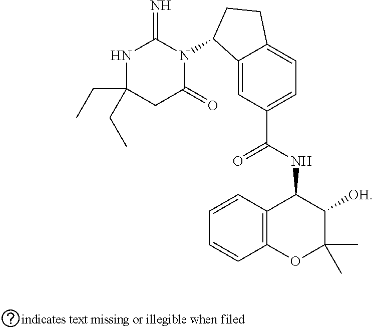 [SMILES: CCC1(CC)CC(=O)N([C@@H]2CCc3ccc(C(=O)N[C@@H]4c5ccccc5OC(C)(C)[C@H]4O)cc32)C(=N)N1]

30. A method of chemovaccination against Plasmodium infections comprising administering to a patient, an effective amount of a compound selected from the group consisting of 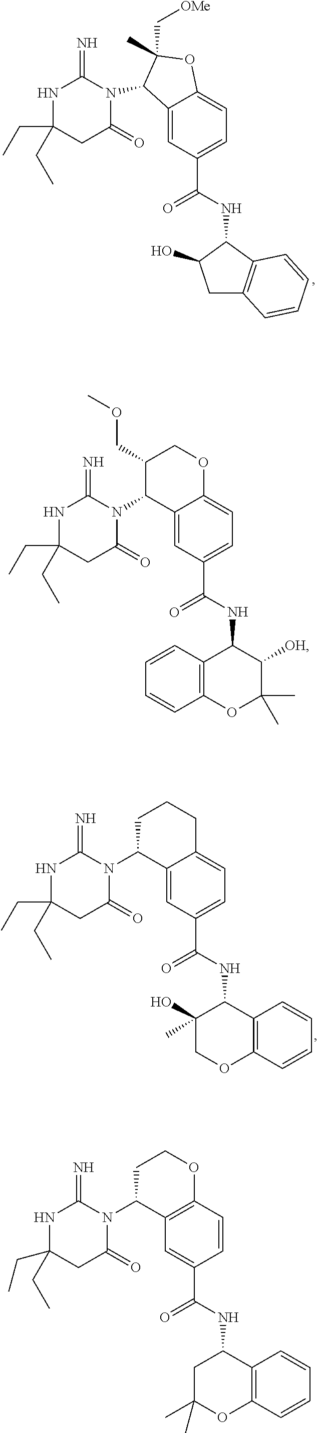
pharmaceutically acceptable salt thereof.

31. The method of chemovaccination against Plasmodium infections according to claim 30 comprising administering to a patient, an effective amount of a compound formula 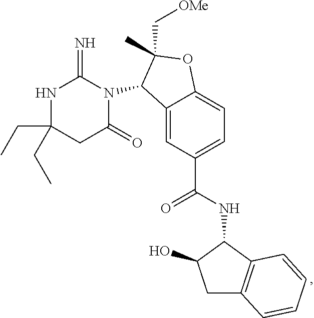 [SMILES: CCC1(CC)CC(=O)N([C@H]2c3cc(C(=O)N[C@@H]4c5ccccc5C[C@H]4O)ccc3O[C@@]2(C)COC)C(=N)N1]
or a pharmaceutically acceptable salt thereof.

32. The method of chemovaccination against Plasmodium infections according to claim 30 comprising administering to a patient, an effective amount of a compound of formula 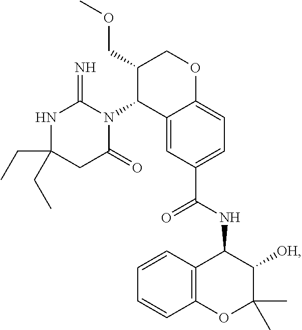 [SMILES: CCC1(CC)CC(=O)N([C@H]2c3cc(C(=O)N[C@@H]4c5ccccc5OC(C)(C)[C@H]4O)ccc3OC[C@H]2COC)C(=N)N1]
or a pharmaceutically acceptable salt thereof.

33. The method of chemovaccination against Plasmodium infections according to claim 30 comprising administering to a patient; an effective amount of a compound of formula 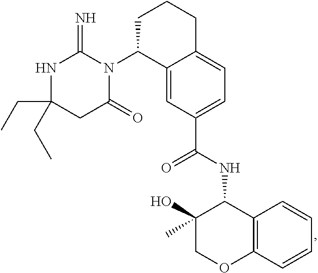 [SMILES: CCC1(CC)CC(=O)N([C@@H]2CCCc3ccc(C(=O)N[C@@H]4c5ccccc5OC[C@@]4(C)O)cc32)C(=N)N1]
or a pharmaceutically acceptable salt thereof.

34. The method of chemovaccination against Plasmodium infections according to claim 1, wherein 0.1-10 mg of the dual inhibitor of plasmepsin IX and X, or a pharmaceutically acceptable salt thereof is administered to the patient.

35. (canceled)

36. (canceled)

37. (canceled)

38. (canceled)

39. The method of chemovaccination of claim 1, wherein the patient does not have a Plasmodium parasite infection, and is simultaneously or sequentially administered a wild-type Plasmodium parasite selected from P. falciparum sporozoites or P. vivax sporozoites, or wherein the patient is later exposed to a wild-type Plasmodium parasite.

40. (canceled)

41. (canceled)

42. (canceled)

43. The method of chemovaccination of claim 1, wherein the patient does not have a Plasmodium parasite infection, and wherein the dual inhibitor of plasmepsin IX and X is administered as a long-acting injectable formulation.

44. The method of chemovaccination of claim 1, wherein the patient does not have a Plasmodium parasite infection, and wherein the patient is simultaneously or sequentially administered a genetically modified Plasmodium parasite.

45. A The method of chemovaccination against Plasmodium infections according to claim 1, additional comprising administering to the patient an effective amount of one or more additional anti-malarial agents.

46. A method of inducing an immune response to a Plasmodium parasite infection, comprising administering to a patient an effective amount of a compound of Formula (I′): 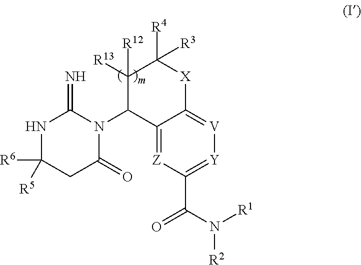
or a pharmaceutically acceptable salt thereof, wherein:
X is a bond, C(R¹⁴)₂, O, S, SO, SO₂ or NH;
Y is CR⁹ or N, wherein when Y is N, Z is CR¹¹ and V is CR¹⁰;
V is CR¹⁰ or N, wherein when V is N, Z is CR¹¹ and Y is CR⁹;
Z is CR¹¹ or N, wherein when Z is N, V is CR¹⁰ and Y is CR⁹;
R¹ is a heterocycloalkyl, C₃-C₁₂cycloalkyl, aryl, C₁-C₆alkylaryl or when taken with R², and the nitrogen which they are bonded, forms nitrogen-containing ring, wherein the heterocycloalkyl, C₃-C₁₂cycloalkyl, aryl, C₁-C₆alkylaryl or nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, aryl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R² is hydrogen, C₁-C₆alkylCOOH, COOH, C₃-C₆cycloalkyl, C₁-C₆alkyl, haloC₁-C₆alkyl or C₁-C₆alkylOH or when taken with R¹, and the nitrogen which they are bonded, forms a nitrogen-containing ring, wherein the nitrogen-containing ring is unsubstituted or substituted with 1 to 5 substituents independently selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl;
R⁴ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl;
R⁵ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁶ forms a C₃-C₆cycloalkyl or C₃-C₆heterocycloalkyl;
R⁶ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R⁵ forms a C₃-C₆cycloalkyl or C₃-C₆heterocycloalkyl;
R⁷ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁸ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹⁰ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹¹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹² is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
each occurrence of R¹⁴ is independently selected from the group consisting of hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
n is 1, 2, 3 or 4; and
m is 0, 1 or 2.

47. The method of chemovaccination against Plasmodium infections according to claim 30 wherein the compound is: 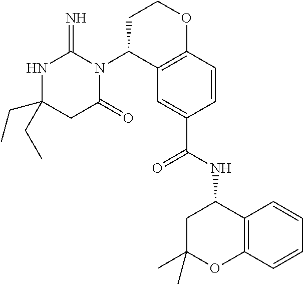 [SMILES: CCC1(CC)CC(=O)N([C@@H]2CCOc3ccc(C(=O)N[C@H]4CC(C)(C)Oc5ccccc54)cc32)C(=N)N1]
or a pharmaceutically acceptable salt thereof.
